(19) United States
(12) Patent Application Publication
(10) Pub. No.: US 2009/0241218 A1
(43) Pub. Date: Sep. 24, 2009

(54) PLANTS HAVING ENHANCED YIELD-RELATED TRAITS AND A METHOD FOR MAKING THE SAME

(75) Inventors: Valerie Frankard, Waterloo (BE); Vladimir Mironov, Gent (BE); Christophe Reuzeau, Tocan Saint Apre (FR)

Correspondence Address: CONNOLLY BOVE LODGE & HUTZ, LLP, P O BOX 2207, WILMINGTON, DE, 19899, US

(73) Assignee: CropDesign N.V., Zwijnaarde (BE)

(21) Appl. No.: 12/302,295

(22) PCT Filed: May 30, 2007
(86) PCT No.: PCT/EP2007/055238

§ 371 Date: Nov. 25, 2008

Related U.S. Application Data
(60) Provisional application No. 60/810,572, filed on Jun. 2, 2006.
(60) Provisional application No. 60/810,749, filed on Jun. 2, 2006.
(60) Provisional application No. 60/810,759, filed on Jun. 2, 2006.
(60) Provisional application No. 60/813,514, filed on Jun. 14, 2006.
(60) Provisional application No. 60/830,551, filed on Jul. 13, 2006.
(60) Provisional application No. 60/831,642, filed on Jul. 18, 2006.
(60) Provisional application No. 60/835,303, filed on Aug. 3, 2006.

(30) Foreign Application Priority Data
May 30, 2006 (EP) 06114726.0
May 30, 2006 (EP) 06114735.1
May 31, 2006 (EP) 06114758.3
Jun. 8, 2006 (EP) 06115142.9
Jun. 30, 2006 (EP) 06116490.1
Jul. 12, 2006 (EP) 06117060.1
Jul. 28, 2006 (EP) 06118060

(51) Int. Cl. C12N 15/82

(52) U.S. Cl. 800/278

(57) ABSTRACT

The present invention relates generally to the field of molecular biology and concerns a method for improving various plant growth characteristics by modulating expression in a plant of a nucleic acid encoding a GRP (Growth-Related Protein). The present invention also concerns plants having modulated expression of a nucleic acid encoding a GRP, which plants have improved growth characteristics relative to corresponding wild type plants or other control plants. The invention also provides constructs useful in the methods of the invention. The GRP may be one of the following: Extensin Receptor-Like Kinase (ERLK), F-Box WD40 (FBXW) polypeptide, RAN-Binding Protein (RANBP), Golden2-like Transcription Factor (GLK), REV delta homeodomain leucine zipper domain polypeptide, CLE protein, and Seed Yield Regulator (SYR) protein.

The present invention relates generally to the field of molecular biology and concerns a method for improving various plant growth characteristics by modulating expression in a plant of a nucleic acid encoding a GRP (Growth Related Protein). The present invention also concerns plants having modulated expression of a nucleic acid encoding a GRP, which plants have improved growth characteristics relative to corresponding wild type plants or other control plants. The invention also provides constructs useful in the methods of the invention.

The ever-increasing world population and the dwindling supply of arable land available for agriculture fuels research towards increasing the efficiency of agriculture. Conventional means for crop and horticultural improvements utilise selective breeding techniques to identify plants having desirable characteristics. However, such selective breeding techniques have several drawbacks, namely that these techniques are typically labour intensive and result in plants that often contain heterogeneous genetic components that may not always result in the desirable trait being passed on from parent plants. Advances in molecular biology have allowed mankind to modify the germplasm of animals and plants. Genetic engineering of plants entails the isolation and manipulation of genetic material (typically in the form of DNA or RNA) and the subsequent introduction of that genetic material into a plant. Such technology has the capacity to deliver crops or plants having various improved economic, agronomic or horticultural traits.

A trait of particular economic interest is increased yield. Yield is normally defined as the measurable produce of economic value from a crop. This may be defined in terms of quantity and/or quality. Yield is directly dependent on several factors, for example, the number and size of the organs, plant architecture (for example, the number of branches), seed production, leaf senescence and more. Root development, nutrient uptake, stress tolerance and early vigour may also be important factors in determining yield. Optimizing the above-mentioned factors may therefore contribute to increasing crop yield.

Seed yield is a particularly important trait, since the seeds of many plants are important for human and animal nutrition. Crops such as corn, rice, wheat, canola and soybean account for over half the total human caloric intake, whether through direct consumption of the seeds themselves or through consumption of meat products raised on processed seeds. They are also a source of sugars, oils and many kinds of metabolites used in industrial processes. Seeds contain an embryo (the source of new shoots and roots) and an endosperm (the source of nutrients for embryo growth during germination and during early growth of seedlings). The development of a seed involves many genes, and requires the transfer of metabolites from the roots, leaves and stems into the growing seed. The endosperm, in particular, assimilates the metabolic precursors of carbohydrates, oils and proteins and synthesizes them into storage macromolecules to fill out the grain.

Another important trait for many crops is early vigour. Improving early vigour is an important objective of modern rice breeding programs in both temperate and tropical rice cultivars. Long roots are important for proper soil anchorage in water-seeded rice. Where rice is sown directly into flooded fields, and where plants must emerge rapidly through water, longer shoots are associated with vigour. Where drill-seeding is practiced, longer mesocotyls and coleoptiles are important for good seedling emergence. The ability to engineer early vigour into plants would be of great importance in agriculture. For example, poor early vigor has been a limitation to the introduction of maize (Zea mays L.) hybrids based on Corn Belt germplasm in the European Atlantic.

A further important trait is that of improved abiotic stress tolerance. Abiotic stress is a primary cause of crop loss worldwide, reducing average yields for most major crop plants by more than 50% (Wang et al., Planta (2003) 218: 1-14). Abiotic stresses may be caused by drought, salinity, extremes of temperature, chemical toxicity and oxidative stress. The ability to improve plant tolerance to abiotic stress would be of great economic advantage to farmers worldwide and would allow for the cultivation of crops during adverse conditions and in territories where cultivation of crops may not otherwise be possible.

Crop yield may therefore be increased by optimising one of the above-mentioned factors.

Depending on the end use, the modification of certain yield traits may be favoured over others. For example for applications such as forage or wood production, or bio-fuel resource, an increase in the vegetative parts of a plant may be desirable, and for applications such as flour, starch or oil production, an increase in seed parameters may be particularly desirable. Even amongst the seed parameters, some may be favoured over others, depending on the application. Various mechanisms may contribute to increasing seed yield, whether that is in the form of increased seed size or increased seed number.

One approach to increasing yield (seed yield and/or biomass) in plants may be through modification of the inherent growth mechanisms of a plant, such as the cell cycle or various signalling pathways involved in plant growth or in defense mechanisms.

It has now been found that various growth characteristics may be improved in plants by modulating expression in a plant of a nucleic acid encoding a GRP (Growth Related Protein Of Interest) in a plant. The GRP may be one of the following: Extensin Receptor-Like Kinase (ERLK), F-Box WD40 (FBXW) polypeptide, RAN-Binding Protein (RANBP), Golden2-like Transcription Factor (GLK), REV delta homeodomain leucine zipper domain polypepetide, CLE protein, and Seed Yield Regulator (SYR) protein.

### BACKGROUND

### Extensin Receptor-Like Kinase

Receptor like kinases (RLKs) are involved in transmission of extracellular signals into the cell. The RLK proteins have a modular structure, starting from the N-terminus with a secretion signal that gets processed, an extracellular domain, a single transmembrane domain and a cytoplasmic kinase domain. Animal receptor-like kinases mostly have tyrosine kinase activity, whereas plant RLKs usually have Ser/Thr kinase specificity, or may sometimes have a dual specificity. In animals, most of the RLKs act as growth factor receptors, whereas plant receptor like kinases may function in various processes, including development, hormone perception or pathogen responses. An overview of developmental functions of plant receptor like kinases such as meristem development, pollen-pistil interactions, hormone signalling, gametophyte development, cell morphogenesis and differentiation, organ shape, organ abscission and somatic embryogenesis is given by Becraft (Annu. Rev. Cell Dev. Biol., 18, 163-192, 2002).

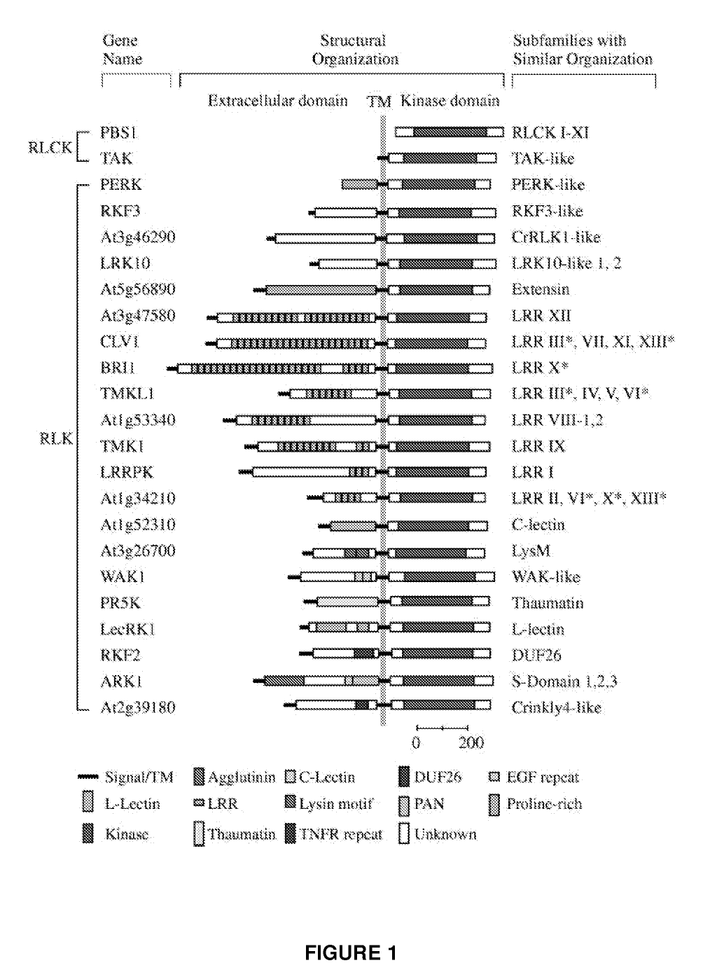

Alternatively, receptor-like kinases may be grouped according to the structure of their extracellular or intracellular domains (Shiu and Bleecker, Proc. Natl. Acad. Sci. USA 98, 10763-10768, 2001). An overview is given in FIG. 1. PERK RLKs and ERLKs have an extracellular domain that is rich in proline and that has motifs typical for extensins and hydroxyproline rich cell wall proteins. Extensins are a group of hydroxyproline rich glycoproteins found in plant cell walls. They are usually rich in hydroxyproline (Hyp), serine and combinations of Val, Tyr, His and Lys. Typical motifs in extensin proteins is the SPx motif, wherein x represents the number of (hydrxy)proline repeats, usually 2, 3, 4 or more. Extensins can account for up to 20% of the dry weight of the cell wall. They are highly glycosylated, possibly reflecting their interactions with cell-wall carbohydrates. Amongst their functions is cell wall strengthening in response to mechanical stress (e.g., during attack by pests, plant-bending in the wind, etc.). Extensin motifs are also found in the small group of extensin receptor like kinases, exemplified by the Arabidopsis At5g56890 gene. Shiu and Bleeker (2001) identified 5 family members in Arabidopsis; various orthologues were found in rice (Shiu et al. Plant Cell 16, 1220-1234, 2004) and in other species. Extensin RLKs have not been characterised yet.

### F-Box WD40 Polypeptide (FBXW)

Plants have to adjust their metabolism to external and internal stimuli to ensure an optimal growth. Levels of regulatory proteins involved in these cellular processes are often controlled by proteolytic mechanisms. Among the most important selective protein degradation pathways in this respect is the ubiquitin-dependent proteolytic pathway. This pathway is conserved among plants, animals and yeast, and it controls the degradation of misfolded polypeptides and of short-lived proteins. Among the latter are important regulatory proteins regulating processes like defence and stress responses, cell cycle progression or signal transduction. Proteins destined to be degraded are covalently labelled with several ubiquitin units. The ubiquitinated protein is subsequently recognised by the 26S proteasome that degrades the target protein but recycles the ubiquitin monomers. The selection and subsequent ubiquitination of a target protein occurs in different steps. Three classes of proteins are involved that have been named E1 to E3, based on their sequential action. The E1 enzyme, also known as the ubiquitin-activating enzyme, “activates” a free ubiquitin molecule at the expense of an ATP and complexes the ubiquitin in a thioester linkage. Next, the activated ubiquitin molecule is transferred from the E1 enzyme to a cysteine of an ubiquitin-conjugating enzyme E2. This E2 enzyme normally is associated with an E3 enzyme called ubiquitin protein ligase. The ubiquitin ligase catalyses the transfer of ubiquitin from the ubiquitin conjugating enzyme to the target protein, which ultimately gets labelled with a poly-ubiquitin chain. The complex of E2 and E3 enzymes determines the specificity for the protein to be ubiquitinated. Whereas in different organisms one or only a few E1 enzymes are present, several E2 species exist that can associate with several E3 enzymes. The ubiquitin protein ligase itself is also a complex of different proteins. To date, five different types of E3 enzymes are known. Among these, the SCF complex plays a prominent role in regulatory processes (Ciechanover (1998) EMBO J. 17: 7151-7160; Hershko and Ciechanover (1998) Annu. Rev. Biochem. 67: 425-479). The complex consists of four subunits: cdc53/cullin, Skp1, Rbxl and an F-box protein. The Skp1 protein, together with cullin and Rbx1 forms the core ligase unit of the SCF complex. Within this complex, Rbxl is responsible for binding the E2 enzyme loaded with an activated ubiquitin. F-box proteins contain N-terminally a degenerate motif of 40 to 50 amino acids, known as the F-box, named after the human cyclin F. This F-box protein is responsible for the association with the Skp1 subunit of the SCF complex. At the C-terminus, a variable protein interaction domain determines the binding of target protein. One such protein interaction domain is a WD40 repeats domain.

In Arabidopsis, F-box proteins represent one of the largest superfamilies found so far in plants, compared to other organisms (Gagne et al. (2002) Proc Natl Acad Science 99: 11519-11524; Kuroda et al. (2002) Plant Cell Physiol 43(10): 1073-85). However, only two F-box proteins contain a WD40 repeats domain, whereas many WD40 repeats domains are found in F-box proteins of other organisms. WD40 repeats (also known as beta-transducin repeats) are short ˜40 amino acid motifs, often terminating in a Trp-Asp (W-D) dipeptide. WD40 repeats containing proteins (or WD40 proteins) have 4 to 16 repeating units (which collectively for the WD40 domain), all of which are thought to form a circularised beta-propeller structure. The underlying common function of all WD40 proteins is coordinating multi-protein complex assemblies, where the repeating units serve as a rigid scaffold for protein interactions. The specificity of the proteins is determined by the sequences outside the repeats themselves.

Published European patent application EP 1033405 provides for a DNA sequence (SEQ ID NO: 39903) from Arabidopsis thaliana encoding a partial FBXW polypeptide (N-terminal amino acid sequence, around 350 nucleotides long).

### RAN-Binding Protein (RANBP)

Ran is a small signalling GTPase (GTP binding protein), which is involved in nucleocytoplasmic transport. Ran binding proteins in Arabidopsis thaliana, At-RanBP1a, At-RanBP1b, AtRanbp1c have been reported to interact with the GTP-bound forms of the Ran1, Ran2 and Ran3 proteins of Arabidopsis thaliana (Haizel T, Merkle T, Pay A, Fejes E, Nagy F. Characterization of proteins that interact with the GTP-bound form of the regulatory GTPase Ran in Arabidopsis. Plant J. 1997 January; 11(1):93-103). All RanBP1 proteins contain an approximately 150 amino acid residue Ran binding domain. Ran BP1 binds directly to RanGTP with high affinity. This domain stabilises the GTP-bound form of Ran (the Ras-like nuclear small GTPase).

International Patent application WO 02/18538 describes transgenic plants having disturbed RAN/Ran-binding protein-mediated cellular processes; this is reported to give plants increased yield and biomass.

### GOLDEN2-Like (GLK) Transcription Factor

In plants, two types of photosynthetic cycles may occur: most common is the Calvin cycle of C3 plants, wherein 3-phosphoglycerate is the first stable product and ribulose bisphosphate is the CO₂ receptor. The second cycle is the Hatch-Smack pathway in C4 plants, in which oxaloacetate is the first stable product and phosphoenolpyruvate is the CO₂ acceptor. In C3 grasses, only the mesophyl cells are photosynthetic, whereas in C4 plants both bundle sheet and mesophyl cells are photosynthetic. C4 plants exhibit compartmentalised photosynthesis with the mesophyl cells performing carbon fixation via phosphoenolpyruvate carboxylase, pyruvate phosphate dikinase and malate dehydrogenase, and shuttling the malate to the bundle sheet cells, in which the malate is decarboxylated to pyruvate, the released carbon is then further processed in the Calvin cycle. As a consequence, three types of chloroplasts are present in C4 plants: typical chloroplasts of the C3 plant exist in certain tissues and at certain developmental stages, while in the bundle sheath and in the mesophyl cells morphologically distinct chloroplasts are present. The genesis of chloroplasts in maize requires the involvement of two transcriptional regulators: Golden2 (G2) and Golden2-like (GLK) (Rossini et al., Plant Cell 13,1231-1244, 2001).

Transcription factors are usually defined as proteins that show sequence-specific DNA binding and that are capable of activating and/or repressing transcription. The Arabidopsis genome codes for at least 1533 transcriptional regulators, which account for ˜5.9% of its estimated total number of genes (Riechmann et al., Science 290, 2105-2109, 2000). The maize GOLDEN2 (G2) gene is a representative of the group of GARP transcription factors defined by, besides GOLDEN2, the Arabidopsis Accepting Response Regulator-B (ARR-B) and Psrl from Chlamydomonas. All GLK proteins classify as members of the GARP family. GLK genes are monophyletic and gene duplications have occurred independently in monocotyledonous and dicotyledonous plants. GLK proteins typically comprise the GARP DNA binding domain and a C-terminal GOLDEN2 box. The Arabidopsis GLK proteins act redundantly in the regulation of chloroplast development (Fitter et al., Plant J. 31, 713-727, 2002). Furthermore, the gene function is conserved between the moss Physcomitrella patens and Arabidopsis thaliana, indicating that GLK-mediated regulation of chloroplast development is an ancient regulatory mechanism among plants (Yasumura et al., Plant Cell 17, 1894-1907, 2005). In the case of G2, three of the four defining features of most transcription factors have been verified experimentally in heterologous systems. G2 is nuclear localized (Hall et al., 1998), is able to transactivate reporter gene expression, and can both homo-dimerize and heterodimerize with ZmGLK1 (Rossini et al., 2001).

### REV Delta (Δ) Homeodomain Leucine Zipper Domain (REV ΔHDZip/START)

The present invention concerns increasing yield in plants using a particular type of transcription factor. Transcription factor polypeptides are usually defined as proteins that show sequence-specific DNA binding affinity and that are capable of activating and/or repressing transcription. Homeodomain leucine zipper (HDZip) polypeptides constitute a family of transcription factors characterized by the presence of a DNA-binding domain (homeodomain, HD) and an adjacent leucine zipper (Zip) motif. The homeodomain usually consists of approximately 60 conserved amino acid residues that form a helix1-loop-helix2-turn-helix3 that binds DNA. This DNA binding site is usually pseudopalindromic. The leucine zipper, adjacent to the C-terminal end of the homeodomain (in some instances, overlapping by a few amino acids), consists of several amino acid heptad repeats (at least four) in which usually a leucine (occasionally a valine or an isoleucine) appears every seventh amino acid. The leucine zipper is important for protein dimerisation. This dimerisation is a prerequisite for DNA binding (Sessa et al. (1993) EMBO J. 12(9): 3507-3517), and may proceed between two identical HDZip polypeptides (homodimer) or between two different HDZip polypeptides (heterodimer).

Homeodomain encoding genes are present in all eucaryotes, and constitute a gene family of at least 89 members in Arabidopsis thaliana. The leucine zipper is also found by itself in polypeptides from eucaryotes other than plants. However, the simultaneous presence of both a homeodomain and a leucine zipper comprised within the same polypeptide is plant-specific (found in at least 47 out of the 89 members in Arabidopsis), and has been encountered in moss in addition to vascular plants (Sakakibara et al. (2001) Mol Biol Evol 18(4): 491-502).

The Arabidopsis HDZip polypeptides have been classified into four different classes, HDZip I to IV, based on sequence similarity criteria (Sessa et al. (1994) In: Puigdomene P, Coruzzi G (ed), Springer, Berlin Heidelberg New York, pp 411-426). In Arabidopsis thaliana, there are at least five class III HDZip polypeptides (REVOLUTA (REV/IFL), PHABULOSA (PHB), PHAVOLUTA (PHV), CORONA (CNA/AtHB15) and AtHB8), all typically quite large (more than 800 amino acids). Similarly, in Oryza sativa, at least 5 class III HDZip polypeptides have been identified.

In addition to the homeodomain and the leucine zipper, class III HDZip polypeptides also comprise C-terminal to the leucine zipper a START (Teroidogenic Acute Regulatory (STAR) related lipid Transfer) domain for lipid/sterol binding and an extensive C-terminal region (CTR, more than half of the polypeptide length) of unknown function (Schrick et al. (2004) Genome Biology 5: R41). Furthermore, a complementary site for microRNA (MIR165/166) is found within the transcript region coding for the START domain of mRNA transcripts coding for class III HDZip polypeptides, for regulation via miRNA-mediated transcript cleavage (Williams et al. (2005) Development 132: 3657-3668).

In Arabidopsis thaliana, REV, PHB and PHV polypeptides have been shown to be involved in formation and function of shoot apical and axillary meristems, patterning of three dimensional structures (such as embryos or leaves), and vascular development (differentiation of lignified conducting and support tissues). In Arabidopsis thaliana, phylogenetic analysis reveals that these three closely related class III HDZip polypeptides form the REV clade, the two remaining class III HDZip polypeptides (CNA and AtHB8) belonging to the CNA clade (Floyd et al. (2006) Genetics 173: 373-388). When combining rice and Arabidopsis genes in a phylogenetic analysis, two rice class III HDZip polypeptides cluster with REV polypeptide, the OsHoxl0 and OsREV polypeptides.

Loss-of-function phb, phv, cna and athb8 Arabidopsis thaliana mutants are aphenotypic (Baima et al. (2001) Plant Physiol 126: 643-655; Prigge et al. (2005) Plant Cell 17: 61-76), but rev mutants form defective lateral and floral meristems and develop aberrant stem vasculature as well as curly (revolute) leaves (Otsuga et al. (2001) Plant J. 25, 223-236; Talbert et al. (1995) Development 121: 2723-2735).

The dominant alleles rolled leaf1 (rld1) in corn (Juarez et al. (2004) Nature 428: 84-88), phb-d and phv-d in Arabidopsis (McConnell et al. (2001) Nature 411: 709-713), and rev-d in Arabidopsis (Emery et al. (2003) Curr Biol 13: 1768-1774) present similar mutant phenotypes (rolled leaves), due to a nucleotide substitution in the sequence spanning the MIR165/166 binding site (in the START domain).

In granted U.S. Pat. No. 7,056,739 (and corresponding international patent application WO01/33944), are described plants and plant cells transformed with a transgene comprising an Arabidopsis thaliana REV nucleic acid sequence encoding a REV polypeptide represented by SEQ ID NO: 2 (of the granted patent). The transgene is reported to further comprise a heterologous promoter operably linked to a nucleic acid sequence encoding the polypeptide of SEQ ID NO: 2. Transgenic Arabidopsis plants overexpressing the REV nucleic acid sequence by using the CaMV promoter, presented increased leaf, stem and seed size. Partial class III HDZip genomic and cDNA 5′ terminal sequences from tomato, rice, maize and barley are provided. Examples of vectors designed to reduce the expression of endogenous Arabidopsis, rice and corn class III HDZip polypeptides in respectively rice and corn are described, to reproduce the rev loss-of-function phenotype.

In international patent application WO2004/063379, are provided nucleic acid sequences of two corn class III HDZip polypeptides orthologous to the Arabidopsis REV polypeptide. A method to modulate the level of class III HDZip polypeptides by inhibiting expression of the mRNA transcripts in the plant cell is described.

In a number of international and US patent applications deposited by Mendel Biotechnology, Inc., the Arabidopsis REV polypeptide has as internal reference G438. Reduced REVOLUTA activity after T-DNA insertion into the REV locus resulted in transgenic Arabidopsis plants with reduced branching and reduced lignin content, whereas increased REVOLUTA activity (using the viral CaMV promoter) resulted in transgenic Arabidopsis plants of which around half developed slightly larger flatter leaves than wild type plants at late stages.

### CLE-Like Polypeptide

Although cell to cell communication in plants occurs mostly through the phytohormones, such as auxin, cytokinin, abscisic acid, brassinostroids, giberellic acid, ethylene, peptide hormones are now recognised as important mediators of signalling events. The group of peptide hormones includes for example systemin, phytosulfokines, ENOD40, RALF, CLAVATA3, SCR peptides and POLARIS.

CLE-like polypeptides form a family of polypeptides that encompass and share homology with the Arabidopsis CLAVATA3 and maize ESR polypeptides. These polypeptides are postulated to be involved as ligands in signalling events.

The root and aerial parts of a plant are derived from the activity of respectively the root apical meristem (RAM) and the shoot apical meristem (SAM). These structures contain pluripotent cells that allow the production of all plant cell types and organs. Within the SAM, a balance exists between the division of the stem cells in the central zone and the differentiating cells in the peripheral zone, though the cell in the central zone divide slower than in the peripheral zone. CLAVATA3 (CLV3) is a member of the CLV3/ESR (CLE) ligand gene family and is secreted by the stem cells of the SAM thereby activating the CLAVATA1-CLAVATA2 receptor dimer and leading to restricted expression of the WUSCHEL (WUS) gene. WUS is a homeodomain transcription factor and promotes stem cell formation; it is required to maintain the stem cell population. CLV3 on the other hand is required to prevent uncontrolled proliferation of stem cells. CLV3 and WUS thus form a feedback loop that controls the number of stem cells and the organisation of the SAM. wus mutants fail to develop a shoot apical meristem, whereas c/v3 mutants develop a greatly enlarged shoot apical meristem.

Another member of the CLE ligand gene family is CLE2, which may function like CLV3 as secreted signalling molecule acting in diverse pathways during growth and development. CLE2 and other CLE family members were first characterised by Cock and McCormick (Plant Physiol. 126, 939-942, 2001). All CLE family members are short polypeptides (around 7 to 9 kDa) with hydrophobic N-terminal sequence (postulated signal peptides or signal anchors). The majority of the predicted mature polypeptides are highly basic (average pl 9.49±1.57) and hydrophilic throughout their length with a conserved region at or near the C-terminal end. This conserved region may be involved in protein-protein interactions. It is postulated that members of the CLE family, such as CLV3 and CLE2, are processed by a protease into a short peptide that is secreted. Although the CLE proteins share an overall resemblance in length, charge, and hydrophilicity, at the amino acid sequence level they are highly divergent. CLE2 was reported to be induced by NO₃⁻ addition (Scheible et al., Plant Physiol. 136, 2483-2499, 2004). Further functional characterisation (Strabala et al., Plant Physiol. 140, 1331-1344, 2006) revealed that CLE2 overexpression in Arabidopsis resulted in dwarfed plants with a strong delay in flowering time (approximately 40 days vs 20 days for control plants), but with longer roots compared to the controls. Overexpression of CLE2 also mimicked a wus phenotype. A similar phenotype as for CLE2 was observed for CLE3, CLE5, CLE6 and CLE7. These proteins are also structurally related. However, no phenotypic information is available to date on cle2 mutants or on downregulation of CLE2.

WO 01/96582 discloses the use of ligand-like proteins (LLPs) or functional fragments thereof for modulating plant phenotype or architecture. Preferred LLPs or fragments comprise the amino acid motif XRXXXXGXXXXHX (wherein X may be any amino acid), more preferred LLPs of fragments comprise the amino acid motif KRXXXXGXXPXHX. The document also describes that ectopic expression of various LLPs results in sterile transgenic plants, or at best in plants with reduced fertility. Also antisense expression of an LLP resulted in a transgenic plant with reduced fertility (greatly reduced number of seed per silique).

WO 03/093450 discloses the use of CLAVATA3-like peptides for modulating cell division, differentiation and development in plants, in particular for modulating meristem development. It was postulated that decreased activity of the CLV3-like peptide might result in generation of additional leaves before flowering begins, thereby providing plants having greater energy production and thus increasing yield, or in an increased number of seed-bearing carpels, or in the generation of a thicker stem, or in an alteration of the fruit of the plants. However, only hypothetical examples were provided with respect to downregulation of CLV3 expression, no real experimental data were given. Furthermore, the disclosed CLV3-like peptides fall within the class of LLPs described in WO 01/96582, since they comprise the motif XRXXXXGXXXXHX, for which it was shown that downregulated expression resulted in plants with reduced fertility.

Similarly, when a CLE-like protein from the plant parasitic nematode Heterodera glycines was overexpressed in Arabidopsis, the transgenic plants produced flowers that did not open or lacked the central gynoecium, and the root system was stunted.

The problem thus remains how CLE-like polypeptides, such as LLPs or CLV3-like, can be used for increasing yield related traits.

### Seed Yield Regulator

SYR is a new protein that hitherto has not been characterised. SYR shows some homology (around 48% sequence identity on DNA level, around 45% at protein level) to an Arabidopsis protein named ARGOS (Hu et al., Plant Cell 15, 1951-1961, 2003; US 2005/0108793). Hu et al. postulated that ARGOS is a protein of unique function and is encoded by a single gene. The major phenotypes of ARGOS overexpression in Arabidopsis are increased leafy biomass and delayed flowering. In contrast, overexpression of SYR in rice primarily increases seed yield, whereas the leafy biomass and flowering time are not obviously affected.

### SUMMARY OF THE INVENTION

Surprisingly, it has now been found that modulating expression in a plant of a nucleic acid encoding an extensin receptor-like kinase (ERLK) or a part thereof comprising at least the kinase domain and the transmembrane domain, gives plants having enhanced yield-related traits relative to control plants.

Therefore, the invention provides a method for enhancing yield-related traits in plants relative to control plants, comprising modulating expression in a plant of a nucleic acid encoding an ERLK protein, or a part thereof.

Surprisingly, it has now also been found that increasing expression in a plant of a nucleic acid encoding an FBXW polypeptide gives plants having increased yield relative to suitable control plants. Therefore, according to another embodiment of the invention there is provided a method for increasing yield in plants relative to suitable control plants, comprising increasing expression in a plant of a nucleic acid encoding an FBXW polypeptide.

Upon investigating the use of RAN-binding proteins to enhance yield-related traits, the inventors found the choice of promoter to be an important consideration. They found that expressing RAN-binding proteins in a (rice) plant under the control of a constitute promoter did not have any effect on yield-related phenotypes. They surprisingly found that plant yield could successfully be increased by expressing RAN-binding proteins in a plant under the control of a seed-specific promoter, particularly an endosperm-specific promoter.

The present invention therefore also provides a method for enhancing yield-related traits in plants relative to control plants, comprising preferentially modulating expression in plant seed or seed parts of a nucleic acid encoding a RANBP.

It has furthermore been found that modulating expression in a plant of a nucleic acid encoding a Golden2-like (GLK) protein gives plants having enhanced yield-related traits relative to control plants.

Therefore, in yet another embodiment of the invention there is provided a method for enhancing yield-related traits in plants relative to control plants, comprising modulating expression in a plant of a nucleic acid encoding a GLK protein, or a part thereof.

It has also surprisingly been found that reducing the expression in a plant of an endogenous REV gene using a REV delta homeodomain leucine zipper domain (HDZip) /STeroidogenic Acute Regulatory (STAR) related lipid Transfer domain (START) nucleic acid sequence gives plants having increased yield relative to control plants. The present invention therefore provides in another embodiment methods for increasing yield in plants relative to control plants, by reducing the expression in a plant of an endogenous REV gene using a REV ΔHDZip/START nucleic acid sequence.

It has also been found that modulating expression in a plant of a nucleic acid encoding a CLE-like polypeptide gives plants having enhanced yield-related traits relative to control plants.

Therefore, the invention provides in a further embodiment a method for enhancing yield-related traits in plants relative to control plants, comprising modulating expression in a plant of a nucleic acid encoding a CLE-like polypeptide, or a part thereof.

Surprisingly, it has now been found that modulating expression in a plant of a nucleic acid encoding a Seed Yield Regulator protein (hereafter named SYR) gives plants, when grown under abiotic stress conditions, having enhanced abiotic stress tolerance relative to control plants.

Therefore, the present invention provides a method for enhancing yield-related traits in plants grown under abiotic stress conditions, relative to control plants, comprising modulating expression in a plant of a nucleic acid encoding a SYR polypeptide.

### DEFINITIONS

### Polypeptide(s)/Protein(s)

The terms “polypeptide” and “protein” are used interchangeably herein and refer to amino acids in a polymeric form of any length, linked together by peptide bonds.

### Polynucleotide(s)/Nucleic Acid(s)/Nucleic Acid Sequence(s)/Nucleotide Sequence(s)

The terms “polynucleotide(s)”, “nucleic acid sequence(s)”, “nucleotide sequence(s)”, “nucleic acid(s)” “nucleic acid molecule” are used interchangeably herein and refer to nucleotides, either ribonucleotides or deoxyribonucleotides or a combination of both, in a polymeric unbranched form of any length.

### Control Plant(s)

The choice of suitable control plants is a routine part of an experimental setup and may include corresponding wild type plants or corresponding plants without the gene of interest. The control plant is typically of the same plant species or even of the same variety as the plant to be assessed. The control plant may also be a nullizygote of the plant to be assessed. Nullizygotes are individuals missing the transgene by segregation. A “control plant” as used herein refers not only to whole plants, but also to plant parts, including seeds and seed parts.

### Homoloque(s)

“Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived.

A deletion refers to removal of one or more amino acids from a protein.

An insertion refers to one or more amino acid residues being introduced into a predetermined site in a protein. Insertions may comprise N-terminal and/or C-terminal fusions as well as intra-sequence insertions of single or multiple amino acids. Generally, insertions within the amino acid sequence will be smaller than N- or C-terminal fusions, of the order of about 1 to 10 residues. Examples of N- or C-terminal fusion proteins or peptides include the binding domain or activation domain of a transcriptional activator as used in the yeast two-hybrid system, phage coat proteins, (histidine)-6-tag, glutathione S-transferase-tag, protein A, maltose-binding protein, dihydrofolate reductase, Tag-100 epitope, c-myc epitope, FLAG®-epitope, lacZ, CMP (calmodulin-binding peptide), HA epitope, protein C epitope and VSV epitope.

A substitution refers to replacement of amino acids of the protein with other amino acids having similar properties (such as similar hydrophobicity, hydrophilicity, antigenicity, propensity to form or break α-helical structures or β-sheet structures). Amino acid substitutions are typically of single residues, but may be clustered depending upon functional constraints placed upon the polypeptide; insertions will usually be of the order of about 1 to 10 amino acid residues. The amino acid substitutions are preferably conservative amino acid substitutions. Conservative substitution tables are well known in the art (see for example Creighton (1984) Proteins. W.H. Freeman and Company (Eds) and Table 1 below).

TABLE 1
Examples of conserved amino acid substitutions

| | Conservative |
|---|---|
| Residue | Substitutions |
| Ala | Ser |
| Arg | Lys |
| Asn | Gln; His |
| Asp | Glu |
| Gln | Asn |
| Cys | Ser |
| Glu | Asp |
| Gly | Pro |
| His | Asn; Gln |
| Ile | Leu, Val |
| Leu | Ile; Val |
| Lys | Arg; Gln |
| Met | Leu; Ile |
| Phe | Met; Leu; Tyr |
| Ser | Thr; Gly |
| Thr | Ser; Val |
| Trp | Tyr |
| Tyr | Trp; Phe |
| Val | Ile; Leu |

Amino acid substitutions, deletions and/or insertions may readily be made using peptide synthetic techniques well known in the art, such as solid phase peptide synthesis and the like, or by recombinant DNA manipulation. Methods for the manipulation of DNA sequences to produce substitution, insertion or deletion variants of a protein are well known in the art. For example, techniques for making substitution mutations at predetermined sites in DNA are well known to those skilled in the art and include M13 mutagenesis, T7-Gen in vitro mutagenesis (USB, Cleveland, Ohio), QuickChange Site Directed mutagenesis (Stratagene, San Diego, Calif.), PCR-mediated site-directed mutagenesis or other site-directed mutagenesis protocols.

### Derivatives

“Derivatives” include peptides, oligopeptides, polypeptides which may, compared to the amino acid sequence of the naturally-occurring form of the protein, such as the protein of interest, comprise substitutions of amino acids with non-naturally occurring amino acid residues, or additions of non-naturally occurring amino acid residues. “Derivatives” of a protein also encompass peptides, oligopeptides, polypeptides which comprise naturally occurring altered (glycosylated, acylated, prenylated, phosphorylated, myristoylated, sulphated etc.) or non-naturally altered amino acid residues compared to the amino acid sequence of a naturally-occurring form of the polypeptide. A derivative may also comprise one or more non-amino acid substituents or additions compared to the amino acid sequence from which it is derived, for example a reporter molecule or other ligand, covalently or non-covalently bound to the amino acid sequence, such as a reporter molecule which is bound to facilitate its detection, and non-naturally occurring amino acid residues relative to the amino acid sequence of a naturally-occurring protein. Furthermore, “derivatives” also include fusions of the naturally-occurring form of the protein with tagging peptides such as FLAG, HIS6 or thioredoxin (for a review of tagging peptides, see Terpe, Appl. Microbiol. Biotechnol. 60, 523-533, 2003).

### Ortholoque(s)/Paraloque(s)

Orthologues and paralogues encompass evolutionary concepts used to describe the ancestral relationships of genes. Paralogues are genes within the same species that have originated through duplication of an ancestral gene; orthologues are genes from different organisms that have originated through speciation, and are also derived from a common ancestral gene.

### Domain

The term “domain” refers to a set of amino acids conserved at specific positions along an alignment of sequences of evolutionarily related proteins. While amino acids at other positions can vary between homologues, amino acids that are highly conserved at specific positions indicate amino acids that are likely essential in the structure, stability or function of a protein. Identified by their high degree of conservation in aligned sequences of a family of protein homologues, they can be used as identifiers to determine if any polypeptide in question belongs to a previously identified polypeptide family.

### Motif/Consensus Sequence/Signature

The term “motif” or “consensus sequence” or “signature” refers to a short conserved region in the sequence of evolutionarily related proteins. Motifs are frequently highly conserved parts of domains, but may also include only part of the domain, or be located outside of conserved domain (if all of the amino acids of the motif fall outside of a defined domain).

### Hybridisation

The term “hybridisation” as defined herein is a process wherein substantially homologous complementary nucleotide sequences anneal to each other. The hybridisation process can occur entirely in solution, i.e. both complementary nucleic acids are in solution. The hybridisation process can also occur with one of the complementary nucleic acids immobilised to a matrix such as magnetic beads, Sepharose beads or any other resin. The hybridisation process can furthermore occur with one of the complementary nucleic acids immobilised to a solid support such as a nitro-cellulose or nylon membrane or immobilised by e.g. photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids.

The term “stringency” refers to the conditions under which a hybridisation takes place. The stringency of hybridisation is influenced by conditions such as temperature, salt concentration, ionic strength and hybridisation buffer composition. Generally, low stringency conditions are selected to be about 30° C. lower than the thermal melting point (Tₘ) for the specific sequence at a defined ionic strength and pH. Medium stringency conditions are when the temperature is 20° C. below Tₘ, and high stringency conditions are when the temperature is 10° C. below Tₘ. High stringency hybridisation conditions are typically used for isolating hybridising sequences that have high sequence similarity to the target nucleic acid sequence. However, nucleic acids may deviate in sequence and still encode a substantially identical polypeptide, due to the degeneracy of the genetic code. Therefore medium stringency hybridisation conditions may sometimes be needed to identify such nucleic acid molecules.

The Tm is the temperature under defined ionic strength and pH, at which 50% of the target sequence hybridises to a perfectly matched probe. The Tₘ is dependent upon the solution conditions and the base composition and length of the probe. For example, longer sequences hybridise specifically at higher temperatures. The maximum rate of hybridisation is obtained from about 16° C. up to 32° C. below Tₘ. The presence of monovalent cations in the hybridisation solution reduce the electrostatic repulsion between the two nucleic acid strands thereby promoting hybrid formation; this effect is visible for sodium concentrations of up to 0.4M (for higher concentrations, this effect may be ignored). Formamide reduces the melting temperature of DNA-DNA and DNA-RNA duplexes with 0.6 to 0.7° C. for each percent formamide, and addition of 50% formamide allows hybridisation to be performed at 30 to 45° C., though the rate of hybridisation will be lowered. Base pair mismatches reduce the hybridisation rate and the thermal stability of the duplexes. On average and for large probes, the Tm decreases about 1° C. per % base mismatch. The Tm may be calculated using the following equations, depending on the types of hybrids:

1) DNA-DNA hybrids (Meinkoth and Wahl, Anal. Biochem., 138: 267-284, 1984):

Tₘ=81.5° C.+16.6×log₁₀[Na⁺]^{a}+0.41×%[G/C^{b}]−500×[L^{c}]⁻¹−0.61×% formamide

2) DNA-RNA or RNA-RNA hybrids:

Tₘ=79.8+18.5 (log₁₀[Na⁺]^{a}+0.58 (% G/C^{b})+11.8 (% G/C^{b})²−820/L^{c}

3) oligo-DNA or oligo-RNA^{d} hybrids:
For <20 nucleotides: Tₘ=2 (lₙ)
For 20-35 nucleotides: Tₘ=22+1.46(lₙ)
^{a} or for other monovalent cation, but only accurate in the 0.01-0.4 M range.
^{b} only accurate for % GC in the 30% to 75% range.
^{c}L=length of duplex in base pairs.
^{d}oligo, oligonucleotide; lₙ, =effective length of primer=2×(no. of G/C)+(no. of A/T).

Non-specific binding may be controlled using any one of a number of known techniques such as, for example, blocking the membrane with protein containing solutions, additions of heterologous RNA, DNA, and SDS to the hybridisation buffer, and treatment with Rnase. For non-homologous probes, a series of hybridizations may be performed by varying one of (i) progressively lowering the annealing temperature (for example from 68° C. to 42° C.) or (ii) progressively lowering the formamide concentration (for example from 50% to 0%). The skilled artisan is aware of various parameters which may be altered during hybridisation and which will either maintain or change the stringency conditions.

Besides the hybridisation conditions, specificity of hybridisation typically also depends on the function of post-hybridisation washes. To remove background resulting from non-specific hybridisation, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridisation stringency. A positive hybridisation gives a signal that is at least twice of that of the background. Generally, suitable stringent conditions for nucleic acid hybridisation assays or gene amplification detection procedures are as set forth above. More or less stringent conditions may also be selected. The skilled artisan is aware of various parameters which may be altered during washing and which will either maintain or change the stringency conditions.

For example, typical high stringency hybridisation conditions for DNA hybrids longer than 50 nucleotides encompass hybridisation at 65° C. in 1×SSC or at 42° C. in 1×SSC and 50% formamide, followed by washing at 65° C. in 0.3×SSC. Examples of medium stringency hybridisation conditions for DNA hybrids longer than 50 nucleotides encompass hybridisation at 50° C. in 4×SSC or at 40° C. in 6×SSC and 50% formamide, followed by washing at 50° C. in 2×SSC. The length of the hybrid is the anticipated length for the hybridising nucleic acid. When nucleic acids of known sequence are hybridised, the hybrid length may be determined by aligning the sequences and identifying the conserved regions described herein. 1×SSC is 0.15M NaCl and 15 mM sodium citrate; the hybridisation solution and wash solutions may additionally include 5×Denhardt's reagent, 0.5-1.0% SDS, 100 μg/ml denatured, fragmented salmon sperm DNA, 0.5% sodium pyrophosphate.

For the purposes of defining the level of stringency, reference can be made to Sambrook et al. (2001) Molecular Cloning: a laboratory manual, 3^{rd} Edition, Cold Spring Harbor Laboratory Press, CSH, New York or to Current Protocols in Molecular Biology, John Wiley & Sons, N.Y. (1989 and yearly updates).

### Splice Variant

The term “splice variant” as used herein encompasses variants of a nucleic acid sequence in which selected introns and/or exons have been excised, replaced, displaced or added, or in which introns have been shortened or lengthened. Such variants will be ones in which the biological activity of the protein is substantially retained; this may be achieved by selectively retaining functional segments of the protein. Such splice variants may be found in nature or may be manmade. Methods for predicting and isolating such splice variants are well known in the art (see for example Foissac and Schiex (2005) BMC Bioinformatics 6: 25).

### Allelic Variant

Alleles or allelic variants are alternative forms of a given gene, located at the same chromosomal position. Allelic variants encompass Single Nucleotide Polymorphisms (SNPs), as well as Small Insertion/Deletion Polymorphisms (INDELs). The size of INDELs is usually less than 100 bp. SNPs and INDELs form the largest set of sequence variants in naturally occurring polymorphic strains of most organisms.

### Gene Shuffling/Directed Evolution

Gene shuffling or directed evolution consists of iterations of DNA shuffling followed by appropriate screening and/or selection to generate variants of nucleic acids or portions thereof encoding proteins having a modified biological activity (Castle et al., (2004) Science 304(5674): 1151-4; U.S. Pat. Nos. 5,811,238 and 6,395,547).

### Regulatory Element/Control Sequence/Promoter

The terms “regulatory element”, “control sequence” and “promoter” are all used interchangeably herein and are to be taken in a broad context to refer to regulatory nucleic acid sequences capable of effecting expression of the sequences to which they are ligated. The term “promoter” typically refers to a nucleic acid control sequence located upstream from the transcriptional start of a gene and which is involved in recognising and binding of RNA polymerase and other proteins, thereby directing transcription of an operably linked nucleic acid. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences. The term “regulatory element” also encompasses a synthetic fusion molecule or derivative that confers, activates or enhances expression of a nucleic acid molecule in a cell, tissue or organ.

A “plant promoter” comprises regulatory elements, which mediate the expression of a coding sequence segment in plant cells. Accordingly, a plant promoter need not be of plant origin, but may originate from viruses or micro-organisms, for example from viruses which attack plant cells. The “plant promoter” can also originate from a plant cell, e.g. from the plant which is transformed with the nucleic acid sequence to be expressed in the inventive process and described herein. This also applies to other “plant” regulatory signals, such as “plant” terminators. The promoters upstream of the nucleotide sequences useful in the methods of the present invention can be modified by one or more nucleotide substitution(s), insertion(s) and/or deletion(s) without interfering with the functionality or activity of either the promoters, the open reading frame (ORF) or the 3′-regulatory region such as terminators or other 3′ regulatory regions which are located away from the ORF. It is furthermore possible that the activity of the promoters is increased by modification of their sequence, or that they are replaced completely by more active promoters, even promoters from heterologous organisms. For expression in plants, the nucleic acid molecule must, as described above, be linked operably to or comprise a suitable promoter which expresses the gene at the right point in time and with the required spatial expression pattern.

For the identification of functionally equivalent promoters, the promoter strength and/or expression pattern of a candidate promoter may be analysed for example by operably linking the promoter to a reporter gene and assaying the expression level and pattern of the reporter gene in various tissues of the plant. Suitable well-known reporter genes include for example beta-glucuronidase or beta-galactosidase. The promoter activity is assayed by measuring the enzymatic activity of the beta-glucuronidase or beta-galactosidase. The promoter strength and/or expression pattern may then be compared to that of a reference promoter (such as the one used in the methods of the present invention). Alternatively, promoter strength may be assayed by quantifying mRNA levels or by comparing mRNA levels of the nucleic acid used in the methods of the present invention, with mRNA levels of housekeeping genes such as 18S rRNA, using methods known in the art, such as Northern blotting with densitometric analysis of autoradiograms, quantitative real-time PCR or RT-PCR (Heid et al., 1996 Genome Methods 6: 986-994). Generally by “weak promoter” is intended a promoter that drives expression of a coding sequence at a low level. By “low level” is intended at levels of about 1/10,000 transcripts to about 1/100,000 transcripts, to about 1/500,0000 transcripts per cell. Conversely, a “strong promoter” drives expression of a coding sequence at high level, or at about 1/10 transcripts to about 1/100 transcripts to about 1/1000 transcripts per cell.

### Operably Linked

The term “operably linked” as used herein refers to a functional linkage between the promoter sequence and the gene of interest, such that the promoter sequence is able to initiate transcription of the gene of interest.

### Constitutive Promoter

A “constitutive promoter” refers to a promoter that is transcriptionally active during most, but not necessarily all, phases of growth and development and under most environmental conditions, in at least one cell, tissue or organ. Table 2a below gives examples of constitutive promoters.

TABLE 2a
Examples of constitutive promoters

| Gene Source | Reference |
|---|---|
| Actin | McElroy et al, Plant Cell, 2: 163-171, 1990 |
| HMGB | WO 2004/070039 |
| CAMV 35S | Odell et al, Nature, 313: 810-812, 1985 |
| CaMV 19S | Nilsson et al., Physiol. Plant. 100: 456-462, 1997 |
| GOS2 | de Pater et al, Plant J Nov; 2(6): 837-44, 1992, |
| | WO 2004/065596 |
| Ubiquitin | Christensen et al, Plant Mol. Biol. 18: 675-689, |
| | 1992 |
| Rice cyclophilin | Buchholz et al, Plant Mol Biol. 25(5): 837-43, 1994 |
| Maize H3 histone | Lepetit et al, Mol. Gen. Genet. 231: 276-285, 1992 |
| Alfalfa H3 histone | Wu et al. Plant Mol. Biol. 11: 641-649, 1988 |
| Actin 2 | An et al, Plant J. 10(1); 107-121, 1996 |
| 34S FMV | Sanger et al., Plant. Mol. Biol., 14, 1990: 433-443 |
| Rubisco small | U.S. Pat. No. 4,962,028 |
| subunit | |
| OCS | Leisner (1988) Proc Natl Acad Sci USA 85(5): 2553 |
| SAD1 | Jain et al., Crop Science, 39 (6), 1999: 1696 |
| SAD2 | Jain et al., Crop Science, 39 (6), 1999: 1696 |
| nos | Shaw et al. (1984) Nucleic Acids Res. |
| | 12(20): 7831-7846 |
| V-ATPase | WO 01/14572 |
| Super promoter | WO 95/14098 |
| G-box proteins | WO 94/12015 |

### Ubiquitous Promoter

A ubiquitous promoter is active in substantially all tissues or cells of an organism.

### Developmentally-Regulated Promoter

A developmentally-regulated promoter is active during certain developmental stages or in parts of the plant that undergo developmental changes.

### Inducible Promoter

An inducible promoter has induced or increased transcription initiation in response to a chemical (for a review see Gatz 1997, Annu. Rev. Plant Physiol. Plant Mol. Biol., 48:89-108), environmental or physical stimulus, or may be “stress-inducible”, i.e. activated when a plant is exposed to various stress conditions, or a “pathogen-inducible” i.e. activated when a plant is exposed to exposure to various pathogens.

### Organ-Specific/Tissue-Specific Promoter

An organ-specific or tissue-specific promoter is one that is capable of preferentially initiating transcription in certain organs or tissues, such as the leaves, roots, seed tissue etc. For example, a “root-specific promoter” is a promoter that is transcriptionally active predominantly in plant roots, substantially to the exclusion of any other parts of a plant, whilst still allowing for any leaky expression in these other plant parts. Promoters able to initiate transcription in certain cells only are referred to herein as “cell-specific”.

A seed-specific promoter is transcriptionally active predominantly in seed tissue, but not necessarily exclusively in seed tissue (in cases of leaky expression). The seed-specific promoter may be active during seed development and/or during germination. Some seed specific promoters may be specific for the endosperm, aleurone and/or embryo. Examples of seed-specific promoters are shown in Table 2b below and of endosperm-specific promoters in Table 2c. Further examples of seed-specific promoters are given in Qing Qu and Takaiwa (Plant Biotechnol. J. 2, 113-125, 2004), which disclosure is incorporated by reference herein as if fully set forth.

TABLE 2b
Examples of seed-specific promoters

| Gene source | Reference |
|---|---|
| seed-specific genes | Simon et al., Plant Mol. Biol. 5: 191, 1985; |
| | Scofield et al., J. Biol. Chem. 262: 12202, 1987.; |
| | Baszczynski et al., Plant Mol. Biol. 14: 633, 1990. |
| Brazil Nut albumin | Pearson et al., Plant Mol. Biol. 18: 235-245, 1992. |
| legumin | Ellis et al., Plant Mol. Biol. 10: 203-214, 1988. |
| glutelin (rice) | Takaiwa et al., Mol. Gen. Genet. 208: 15-22, 1986; |
| | Takaiwa et al., FEBS Letts. 221: 43-47, 1987. |
| zein | Matzke et al Plant Mol Biol, 14(3): 323-32 1990 |
| napA | Stalberg et al, Planta 199: 515-519, 1996. |
| wheat LMW and HMW glutenin-1 | Mol Gen Genet 216: 81-90, 1989; NAR 17: 461-2, 1989 |
| wheat SPA | Albani et al, Plant Cell, 9: 171-184, 1997 |
| wheat α, β, γ-gliadins | EMBO J. 3: 1409-15, 1984 |
| barley Itr1 promoter | Diaz et al. (1995) Mol Gen Genet 248(5): 592-8 |
| barley B1, C, D, hordein | Theor Appl Gen 98: 1253-62, 1999; Plant J 4: 343-55, 1993; |
| | Mol Gen Genet 250: 750-60, 1996 |
| barley DOF | Mena et al, The Plant Journal, 116(1): 53-62, 1998 |
| blz2 | EP99106056.7 |
| synthetic promoter | Vicente-Carbajosa et al., Plant J. 13: 629-640, 1998. |
| rice prolamin NRP33 | Wu et al, Plant Cell Physiology 39(8) 885-889, 1998 |
| rice α-globulin Glb-1 | Wu et al, Plant Cell Physiology 39(8) 885-889, 1998 |
| rice OSH1 | Sato et al, Proc. Natl. Acad. Sci. USA, 93: 8117-8122, 1996 |
| rice α-globulin REB/OHP-1 | Nakase et al. Plant Mol. Biol. 33: 513-522, 1997 |
| rice ADP-glucose pyrophosphorylase | Trans Res 6: 157-68, 1997 |
| maize ESR gene family | Plant J 12: 235-46, 1997 |
| sorghum α-kafirin | DeRose et al., Plant Mol. Biol 32: 1029-35, 1996 |
| KNOX | Postma-Haarsma et al, Plant Mol. Biol. 39: 257-71, 1999 |
| rice oleosin | Wu et al, J. Biochem. 123: 386, 1998 |
| sunflower oleosin | Cummins et al., Plant Mol. Biol. 19: 873-876, 1992 |
| PRO0117, putative rice 40S | WO 2004/070039 |
| ribosomal protein | |
| PRO0136, rice alanine | unpublished |
| aminotransferase | |
| PRO0147, trypsin inhibitor ITR1 | unpublished |
| (barley) | |
| PRO0151, rice WSI18 | WO 2004/070039 |
| PRO0175, rice RAB21 | WO 2004/070039 |
| PRO005 | WO 2004/070039 |
| PRO0095 | WO 2004/070039 |
| α-amylase (Amy32b) | Lanahan et al, Plant Cell 4: 203-211, 1992; Skriver et al, |
| | Proc Natl Acad Sci USA 88: 7266-7270, 1991 |
| cathepsin β-like gene | Cejudo et al, Plant Mol Biol 20: 849-856, 1992 |
| Barley Ltp2 | Kalla et al., Plant J. 6: 849-60, 1994 |
| Chi26 | Leah et al., Plant J. 4: 579-89, 1994 |
| Maize B-Peru | Selinger et al., Genetics 149; 1125-38, 1998 |

TABLE 2c
Examples of endosperm-specific promoters

| Gene source | Reference |
|---|---|
| glutelin (rice) | Takaiwa et al. (1986) Mol Gen Genet 208: 15-22; Takaiwa et al. |
| | (1987) FEBS Letts. 221: 43-47 |
| zein | Matzke et al., (1990) Plant Mol Biol 14(3): 323-32 |
| wheat LMW and HMW | Colot et al. (1989) Mol Gen Genet 216: 81-90, Anderson et al. |
| glutenin-1 | (1989) NAR 17: 461-2 |
| wheat SPA | Albani et al. (1997) Plant Cell 9: 171-184 |
| wheat gliadins | Rafalski et al. (1984) EMBO 3: 1409-15 |
| barley Itr1 promoter | Diaz et al. (1995) Mol Gen Genet 248(5): 592-8 |
| barley B1, C, D, hordein | Cho et al. (1999) Theor Appl Genet 98: 1253-62; Muller et al. |
| | (1993) Plant J 4: 343-55; Sorenson et al. (1996) Mol Gen Genet |
| | 250: 750-60 |
| barley DOF | Mena et al, (1998) Plant J 116(1): 53-62 |
| blz2 | Onate et al. (1999) J Biol Chem 274(14): 9175-82 |
| synthetic promoter | Vicente-Carbajosa et al. (1998) Plant J 13: 629-640 |
| rice prolamin NRP33 | Wu et al, (1998) Plant Cell Physiol 39(8) 885-889 |
| rice globulin Glb-1 | Wu et al. (1998) Plant Cell Physiol 39(8) 885-889 |
| rice globulin REB/OHP-1 | Nakase et al. (1997) Plant Molec Biol 33: 513-522 |
| rice ADP-glucose | Russell et al. (1997) Trans Res 6: 157-68 |
| pyrophosphorylase | |
| maize ESR gene family | Opsahl-Ferstad et al. (1997) Plant J 12: 235-46 |
| sorghum kafirin | DeRose et al. (1996) Plant Mol Biol 32: 1029-35 |

A green tissue-specific promoter as defined herein is a promoter that is transcriptionally active predominantly in green tissue, substantially to the exclusion of any other parts of a plant, whilst still allowing for any leaky expression in these other plant parts.

Another example of a tissue-specific promoter is a meristem-specific promoter, which is transcriptionally active predominantly in meristematic tissue, substantially to the exclusion of any other parts of a plant, whilst still allowing for any leaky expression in these other plant parts.

### Terminator

The term “terminator” encompasses a control sequence which is a DNA sequence at the end of a transcriptional unit which signals 3′ processing and polyadenylation of a primary transcript and termination of transcription. The terminator can be derived from the natural gene, from a variety of other plant genes, or from T-DNA. The terminator to be added may be derived from, for example, the nopaline synthase or octopine synthase genes, or alternatively from another plant gene, or less preferably from any other eukaryotic gene.

### Modulation

The term “modulation” means in relation to expression or gene expression, a process in which the expression level is changed by said gene expression in comparison to the control plant, preferably the expression level is increased. The original, unmodulated expression may be of any kind of expression of a structural RNA (rRNA, tRNA) or mRNA with subsequent translation. The term “modulating the activity” shall mean any change of the expression of the inventive nucleic acid sequences or encoded proteins, which leads to increased yield and/or increased growth of the plants.

### Expression

The term “expression” or “gene expression” means the transcription of a specific gene or specific genes or specific genetic construct. The term “expression” or “gene expression” in particular means the transcription of a gene or genes or genetic construct into structural RNA (rRNA, tRNA) or mRNA with or without subsequent translation of the latter into a protein. The process includes transcription of DNA and processing of the resulting mRNA product.

### Increased Expression/Overexpression

The term “increased expression” or “overexpression” as used herein means any form of expression that is additional to the original wild-type expression level.

Methods for increasing expression of genes or gene products are well documented in the art and include, for example, overexpression driven by appropriate promoters, the use of transcription enhancers or translation enhancers. Isolated nucleic acids which serve as promoter or enhancer elements may be introduced in an appropriate position (typically upstream) of a non-heterologous form of a polynucleotide so as to upregulate expression of a nucleic acid encoding the polypeptide of interest. For example, endogenous promoters may be altered in vivo by mutation, deletion, and/or substitution (see, Kmiec, U.S. Pat. No. 5,565,350; Zarling et al., WO9322443), or isolated promoters may be introduced into a plant cell in the proper orientation and distance from a gene of the present invention so as to control the expression of the gene.

If polypeptide expression is desired, it is generally desirable to include a polyadenylation region at the 3′-end of a polynucleotide coding region. The polyadenylation region can be derived from the natural gene, from a variety of other plant genes, or from T-DNA. The 3′ end sequence to be added may be derived from, for example, the nopaline synthase or octopine synthase genes, or alternatively from another plant gene, or less preferably from any other eukaryotic gene.

An intron sequence may also be added to the 5′ untranslated region (UTR) or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg (1988) Mol. Cell biol. 8: 4395-4405; Callis et al. (1987) Genes Dev 1:1183-1200). Such intron enhancement of gene expression is typically greatest when placed near the 5′ end of the transcription unit. Use of the maize introns Adhl-S intron 1, 2, and 6, the Bronze-1 intron are known in the art. For general information see: The Maize Handbook, Chapter 116, Freeling and Walbot, Eds., Springer, N.Y. (1994).

### Endogenous Gene

Reference herein to an “endogenous” gene not only refers to the gene in question as found in a plant in its natural form (i.e., without there being any human intervention), but also refers to that same gene (or a substantially homologous nucleic acid/gene) in an isolated form subsequently (re)introduced into a plant (a transgene). For example, a transgenic plant containing such a transgene may encounter a substantial reduction of the transgene expression and/or substantial reduction of expression of the endogenous gene.

### Isolated Gene

The isolated gene may be isolated from an organism or may be manmade, for example by chemical synthesis.

### Decreased Expression

Reference herein to “decreased epression” or “reduction or substantial elimination” of expression is taken to mean a decrease in endogenous gene expression and/or polypeptide levels and/or polypeptide activity relative to control plants. The reduction or substantial elimination is in increasing order of preference at least 10%, 20%, 30%, 40% or 50%, 60%, 70%, 80%, 85%, 90%, or 95%, 96%, 97%, 98%, 99% or more reduced compared to that of control plants.

For the reduction or substantial elimination of expression an endogenous gene in a plant, a sufficient length of substantially contiguous nucleotides of a nucleic acid sequence is required. In order to perform gene silencing, this may be as little as 20, 19, 18, 17, 16, 15, 14, 13, 12, 11, 10 or fewer nucleotides, alternatively this may be as much as the entire gene (including the 5′ and/or 3′ UTR, either in part or in whole). The stretch of substantially contiguous nucleotides may be derived from the nucleic acid encoding the protein of interest (target gene), or from any nucleic acid capable of encoding an orthologue, paralogue or homologue of the protein of interest. Preferably, the stretch of substantially contiguous nucleotides is capable of forming hydrogen bonds with the target gene (either sense or antisense strand), more preferably, the stretch of substantially contiguous nucleotides has, in increasing order of preference, 50%, 60%, 70%, 80%, 85%, 90%, 95%, 96%, 97%, 98%, 99%, 100% sequence identity to the target gene (either sense or antisense strand). A nucleic acid sequence encoding a (functional) polypeptide is not a requirement for the various methods discussed herein for the reduction or substantial elimination of expression of an endogenous gene.

This reduction or substantial elimination of expression may be achieved using routine tools and techniques. A preferred method for the reduction or substantial elimination of endogenous gene expression is by introducing and expressing in a plant a genetic construct into which the nucleic acid (in this case a stretch of substantially contiguous nucleotides derived from the gene of interest, or from any nucleic acid capable of encoding an orthologue, paralogue or homologue of any one of the protein of interest) is cloned as an inverted repeat (in part or completely), separated by a spacer (non-coding DNA).

In such a preferred method, expression of the endogenous gene is reduced or substantially eliminated through RNA-mediated silencing using an inverted repeat of a nucleic acid or a part thereof (in this case a stretch of substantially contiguous nucleotides derived from the gene of interest, or from any nucleic acid capable of encoding an orthologue, paralogue or homologue of the protein of interest), preferably capable of forming a hairpin structure. The inverted repeat is cloned in an expression vector comprising control sequences. A non-coding DNA nucleic acid sequence (a spacer, for example a matrix attachment region fragment (MAR), an intron, a polylinker, etc.) is located between the two inverted nucleic acids forming the inverted repeat. After transcription of the inverted repeat, a chimeric RNA with a self-complementary structure is formed (partial or complete). This double-stranded RNA structure is referred to as the hairpin RNA (hpRNA). The hpRNA is processed by the plant into siRNAs that are incorporated into an RNA-induced silencing complex (RISC). The RISC further cleaves the mRNA transcripts, thereby substantially reducing the number of mRNA transcripts to be translated into polypeptides. For further general details see for example, Grierson et al. (1998) WO 98/53083; Waterhouse et al. (1999) WO 99/53050).

Performance of the methods of the invention does not rely on introducing and expressing in a plant a genetic construct into which the nucleic acid is cloned as an inverted repeat, but any one or more of several well-known “gene silencing” methods may be used to achieve the same effects.

One such method for the reduction of endogenous gene expression is RNA-mediated silencing of gene expression (downregulation). Silencing in this case is triggered in a plant by a double stranded RNA sequence (dsRNA) that is substantially similar to the target endogenous gene. This dsRNA is further processed by the plant into about 20 to about 26 nucleotides called short interfering RNAs (siRNAs). The siRNAs are incorporated into an RNA-induced silencing complex (RISC) that cleaves the mRNA transcript of the endogenous target gene, thereby substantially reducing the number of mRNA transcripts to be translated into a polypeptide. Preferably, the double stranded RNA sequence corresponds to a target gene.

Another example of an RNA silencing method involves the introduction of nucleic acid sequences or parts thereof (in this case a stretch of substantially contiguous nucleotides derived from the gene of interest, or from any nucleic acid capable of encoding an orthologue, paralogue or homologue of the protein of interest) in a sense orientation into a plant. “Sense orientation” refers to a DNA sequence that is homologous to an mRNA transcript thereof. Introduced into a plant would therefore be at least one copy of the nucleic acid sequence. The additional nucleic acid sequence will reduce expression of the endogenous gene, giving rise to a phenomenon known as co-suppression. The reduction of gene expression will be more pronounced if several additional copies of a nucleic acid sequence are introduced into the plant, as there is a positive correlation between high transcript levels and the triggering of co-suppression.

Another example of an RNA silencing method involves the use of antisense nucleic acid sequences. An “antisense” nucleic acid sequence comprises a nucleotide sequence that is complementary to a “sense” nucleic acid sequence encoding a protein, i.e. complementary to the coding strand of a double-stranded cDNA molecule or complementary to an mRNA transcript sequence. The antisense nucleic acid sequence is preferably complementary to the endogenous gene to be silenced. The complementarity may be located in the “coding region” and/or in the “non-coding region” of a gene. The term “coding region” refers to a region of the nucleotide sequence comprising codons that are translated into amino acid residues. The term “non-coding region” refers to 5′ and 3′ sequences that flank the coding region that are transcribed but not translated into amino acids (also referred to as 5′ and 3′ untranslated regions).

Antisense nucleic acid sequences can be designed according to the rules of Watson and Crick base pairing. The antisense nucleic acid sequence may be complementary to the entire nucleic acid sequence (in this case a stretch of substantially contiguous nucleotides derived from the gene of interest, or from any nucleic acid capable of encoding an orthologue, paralogue or homologue of the protein of interest), but may also be an oligonucleotide that is antisense to only a part of the nucleic acid sequence (including the mRNA 5′ and 3′ UTR). For example, the antisense oligonucleotide sequence may be complementary to the region surrounding the translation start site of an mRNA transcript encoding a polypeptide. The length of a suitable antisense oligonucleotide sequence is known in the art and may start from about 50, 45, 40, 35, 30, 25, 20, 15 or 10 nucleotides in length or less. An antisense nucleic acid sequence according to the invention may be constructed using chemical synthesis and enzymatic ligation reactions using methods known in the art. For example, an antisense nucleic acid sequence (e.g., an antisense oligonucleotide sequence) may be chemically synthesized using naturally occurring nucleotides or variously modified nucleotides designed to increase the biological stability of the molecules or to increase the physical stability of the duplex formed between the antisense and sense nucleic acid sequences, e.g., phosphorothioate derivatives and acridine substituted nucleotides may be used. Examples of modified nucleotides that may be used to generate the antisense nucleic acid sequences are well known in the art. Known nucleotide modifications include methylation, cyclization and ‘caps’ and substitution of one or more of the naturally occurring nucleotides with an analogue such as inosine. Other modifications of nucleotides are well known in the art.

The antisense nucleic acid sequence can be produced biologically using an expression vector into which a nucleic acid sequence has been subcloned in an antisense orientation (i.e., RNA transcribed from the inserted nucleic acid will be of an antisense orientation to a target nucleic acid of interest). Preferably, production of antisense nucleic acid sequences in plants occurs by means of a stably integrated nucleic acid construct comprising a promoter, an operably linked antisense oligonucleotide, and a terminator.

The nucleic acid molecules used for silencing in the methods of the invention (whether introduced into a plant or generated in situ) hybridize with or bind to mRNA transcripts and/or genomic DNA encoding a polypeptide to thereby inhibit expression of the protein, e.g., by inhibiting transcription and/or translation. The hybridization can be by conventional nucleotide complementarity to form a stable duplex, or, for example, in the case of an antisense nucleic acid sequence which binds to DNA duplexes, through specific interactions in the major groove of the double helix. Antisense nucleic acid sequences may be introduced into a plant by transformation or direct injection at a specific tissue site. Alternatively, antisense nucleic acid sequences can be modified to target selected cells and then administered systemically. For example, for systemic administration, antisense nucleic acid sequences can be modified such that they specifically bind to receptors or antigens expressed on a selected cell surface, e.g., by linking the antisense nucleic acid sequence to peptides or antibodies which bind to cell surface receptors or antigens. The antisense nucleic acid sequences can also be delivered to cells using the vectors described herein.

According to a further aspect, the antisense nucleic acid sequence is an a-anomeric nucleic acid sequence. An a-anomeric nucleic acid sequence forms specific double-stranded hybrids with complementary RNA in which, contrary to the usual b-units, the strands run parallel to each other (Gaultier et al. (1987) Nucl Ac Res 15: 6625-6641). The antisense nucleic acid sequence may also comprise a 2′-o-methylribonucleotide (Inoue et al. (1987) Nucl Ac Res 15, 6131-6148) or a chimeric RNA-DNA analogue (Inoue et al. (1987) FEBS Lett. 215, 327-330).

The reduction or substantial elimination of endogenous gene expression may also be performed using ribozymes. Ribozymes are catalytic RNA molecules with ribonuclease activity that are capable of cleaving a single-stranded nucleic acid sequence, such as an mRNA, to which they have a complementary region. Thus, ribozymes (e.g., hammerhead ribozymes (described in Haselhoff and Gerlach (1988) Nature 334, 585-591) can be used to catalytically cleave mRNA transcripts encoding a polypeptide, thereby substantially reducing the number of mRNA transcripts to be translated into a polypeptide. A ribozyme having specificity for a nucleic acid sequence can be designed (see for example: Cech et al. U.S. Pat. No. 4,987,071; and Cech et al. U.S. Pat. No. 5,116,742). Alternatively, mRNA transcripts corresponding to a nucleic acid sequence can be used to select a catalytic RNA having a specific ribonuclease activity from a pool of RNA molecules (Bartel and Szostak (1993) Science 261, 1411-1418). The use of ribozymes for gene silencing in plants is known in the art (e.g., Atkins et al. (1994) WO 94/00012; Lenne et al. (1995) WO 95/03404; Lutziger et al. (2000) WO 00/00619; Prinsen et al. (1997) WO 97/13865 and Scott et al. (1997) WO 97/38116).

Gene silencing may also be achieved by insertion mutagenesis (for example, T-DNA insertion or transposon insertion) or by strategies as described by, among others, Angell and Baulcombe ((1999) Plant J 20(3): 357-62), (Amplicon VIGS WO 98/36083), or Baulcombe (WO 99/15682).

Gene silencing may also occur if there is a mutation on an endogenous gene and/or a mutation on an isolated gene/nucleic acid subsequently introduced into a plant. The reduction or substantial elimination may be caused by a non-functional polypeptide. For example, a polypeptide may bind to various interacting proteins; one or more mutation(s) and/or truncation(s) may therefore provide for a polypeptide that is still able to bind interacting proteins (such as receptor proteins) but that cannot exhibit its normal function (such as signalling ligand).

A further approach to gene silencing is by targeting nucleic acid sequences complementary to the regulatory region of the gene (e.g., the promoter and/or enhancers) to form triple helical structures that prevent transcription of the gene in target cells. See Helene, C., Anticancer Drug Res. 6, 569-84, 1991; Helene et al., Ann. N.Y. Acad. Sci. 660, 27-36 1992; and Maher, L. J. Bioassays 14, 807-15,1992.

Other methods, such as the use of antibodies directed to an endogenous polypeptide for inhibiting its function in planta, or interference in the signalling pathway in which a polypeptide is involved, will be well known to the skilled man. In particular, it can be envisaged that manmade molecules may be useful for inhibiting the biological function of a target polypeptide, or for interfering with the signalling pathway in which the target polypeptide is involved.

Alternatively, a screening program may be set up to identify in a plant population natural variants of a gene, which variants encode polypeptides with reduced activity. Such natural variants may also be used for example, to perform homologous recombination.

Artificial and/or natural microRNAs (miRNAs) may be used to knock out gene expression and/or mRNA translation. Endogenous miRNAs are single stranded small RNAs of typically 19-24 nucleotides long. They function primarily to regulate gene expression and/or mRNA translation. Most plant microRNAs (miRNAs) have perfect or near-perfect complementarity with their target sequences. However, there are natural targets with up to five mismatches. They are processed from longer non-coding RNAs with characteristic fold-back structures by double-strand specific RNases of the Dicer family. Upon processing, they are incorporated in the RNA-induced silencing complex (RISC) by binding to its main component, an Argonaute protein. mRNAs serve as the specificity components of RISC, since they base-pair to target nucleic acids, mostly mRNAs, in the cytoplasm. Subsequent regulatory events include target mRNA cleavage and destruction and/or translational inhibition. Effects of miRNA overexpression are thus often reflected in decreased mRNA levels of target genes.

Artificial microRNAs (amiRNAs), which are typically 21 nucleotides in length, can be genetically engineered specifically to negatively regulate gene expression of single or multiple genes of interest. Determinants of plant microRNA target selection are well known in the art. Empirical parameters for target recognition have been defined and can be used to aid in the design of specific amiRNAs, (Schwab et al., Dev. Cell 8, 517-527, 2005). Convenient tools for design and generation of amiRNAs and their precursors are also available to the public (Schwab et al., Plant Cell 18, 1121-1133, 2006).

For optimal performance, the gene silencing techniques used for reducing expression in a plant of an endogenous gene requires the use of nucleic acid sequences from monocotyledonous plants for transformation of monocotyledonous plants, and from dicotyledonous plants for transformation of dicotyledonous plants. Preferably, a nucleic acid sequence from any given plant species is introduced into that same species. For example, a nucleic acid sequence from rice is transformed into a rice plant. However, it is not an absolute requirement that the nucleic acid sequence to be introduced originates from the same plant species as the plant in which it will be introduced. It is sufficient that there is substantial homology between the endogenous target gene and the nucleic acid to be introduced.

Described above are examples of various methods for the reduction or substantial elimination of expression in a plant of an endogenous gene. A person skilled in the art would readily be able to adapt the aforementioned methods for silencing so as to achieve reduction of expression of an endogenous gene in a whole plant or in parts thereof through the use of an appropriate promoter, for example.

### Selectable Marker (Gene)/Reporter Gene

“Selectable marker”, “selectable marker gene” or “reporter gene” includes any gene that confers a phenotype on a cell in which it is expressed to facilitate the identification and/or selection of cells that are transfected or transformed with a nucleic acid construct of the invention. These marker genes enable the identification of a successful transfer of the nucleic acid molecules via a series of different principles. Suitable markers may be selected from markers that confer antibiotic or herbicide resistance, that introduce a new metabolic trait or that allow visual selection. Examples of selectable marker genes include genes conferring resistance to antibiotics (such as nptII that phosphorylates neomycin and kanamycin, or hpt, phosphorylating hygromycin, or genes conferring resistance to, for example, bleomycin, streptomycin, tetracyclin, chloramphenicol, ampicillin, gentamycin, geneticin (G418), spectinomycin or blasticidin), to herbicides (for example bar which provides resistance to Basta®; aroA or gox providing resistance against glyphosate, or the genes conferring resistance to, for example, imidazolinone, phosphinothricin or sulfonylurea), or genes that provide a metabolic trait (such as manA that allows plants to use mannose as sole carbon source or xylose isomerase for the utilisation of xylose, or antinutritive markers such as the resistance to 2-deoxyglucose). Expression of visual marker genes results in the formation of colour (for example β-glucuronidase, GUS or β-galactosidase with its coloured substrates, for example X-Gal), luminescence (such as the luciferin/luceferase system) or fluorescence (Green Fluorescent Protein, GFP, and derivatives thereof). This list represents only a small number of possible markers. The skilled worker is familiar with such markers. Different markers are preferred, depending on the organism and the selection method.

It is known that upon stable or transient integration of nucleic acids into plant cells, only a minority of the cells takes up the foreign DNA and, if desired, integrates it into its genome, depending on the expression vector used and the transfection technique used. To identify and select these integrants, a gene coding for a selectable marker (such as the ones described above) is usually introduced into the host cells together with the gene of interest. These markers can for example be used in mutants in which these genes are not functional by, for example, deletion by conventional methods. Furthermore, nucleic acid molecules encoding a selectable marker can be introduced into a host cell on the same vector that comprises the sequence encoding the polypeptides of the invention or used in the methods of the invention, or else in a separate vector. Cells which have been stably transfected with the introduced nucleic acid can be identified for example by selection (for example, cells which have integrated the selectable marker survive whereas the other cells die).

Since the marker genes, particularly genes for resistance to antibiotics and herbicides, are no longer required or are undesired in the transgenic host cell once the nucleic acids have been introduced successfully, the process according to the invention for introducing the nucleic acids advantageously employs techniques which enable the removal or excision of these marker genes. One such a method is what is known as co-transformation. The co-transformation method employs two vectors simultaneously for the transformation, one vector bearing the nucleic acid according to the invention and a second bearing the marker gene(s). A large proportion of transformants receives or, in the case of plants, comprises (up to 40% or more of the transformants), both vectors. In case of transformation with Agrobacteria, the transformants usually receive only a part of the vector, i.e. the sequence flanked by the T-DNA, which usually represents the expression cassette. The marker genes can subsequently be removed from the transformed plant by performing crosses. In another method, marker genes integrated into a transposon are used for the transformation together with desired nucleic acid (known as the Ac/Ds technology). The transformants can be crossed with a transposase source or the transformants are transformed with a nucleic acid construct conferring expression of a transposase, transiently or stable. In some cases (approx. 10%), the transposon jumps out of the genome of the host cell once transformation has taken place successfully and is lost. In a further number of cases, the transposon jumps to a different location. In these cases the marker gene must be eliminated by performing crosses. In microbiology, techniques were developed which make possible, or facilitate, the detection of such events. A further advantageous method relies on what is known as recombination systems; whose advantage is that elimination by crossing can be dispensed with. The best-known system of this type is what is known as the Cre/lox system. Cre1 is a recombinase that removes the sequences located between the loxP sequences. If the marker gene is integrated between the loxP sequences, it is removed once transformation has taken place successfully, by expression of the recombinase. Further recombination systems are the HIN/HIX, FLP/FRT and REP/STB system (Tribble et al., J. Biol. Chem., 275, 2000: 22255-22267; Velmurugan et al., J. Cell Biol., 149, 2000: 553-566). A site-specific integration into the plant genome of the nucleic acid sequences according to the invention is possible. Naturally, these methods can also be applied to microorganisms such as yeast, fungi or bacteria.

### Transgenic/Transgene/Recombinant

For the purposes of the invention, “transgenic”, “transgene” or “recombinant” means with regard to, for example, a nucleic acid sequence, an expression cassette, gene construct or a vector comprising the nucleic acid sequence or an organism transformed with the nucleic acid sequences, expression cassettes or vectors according to the invention, all those constructions brought about by recombinant methods in which either
(a) the nucleic acid sequences encoding proteins useful in the methods of the invention, or
(b) genetic control sequence(s) which is operably linked with the nucleic acid sequence according to the invention, for example a promoter, or
(c) a) and b)
   are not located in their natural genetic environment or have been modified by recombinant methods, it being possible for the modification to take the form of, for example, a substitution, addition, deletion, inversion or insertion of one or more nucleotide residues. The natural genetic environment is understood as meaning the natural genomic or chromosomal locus in the original plant or the presence in a genomic library. In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp. A naturally occurring expression cassette—for example the naturally occurring combination of the natural promoter of the nucleic acid sequences with the corresponding nucleic acid sequence encoding a polypeptide useful in the methods of the present invention, as defined above—becomes a transgenic expression cassette when this expression cassette is modified by non-natural, synthetic (“artificial”) methods such as, for example, mutagenic treatment. Suitable methods are described, for example, in U.S. Pat. No. 5,565,350 or WO 00/15815.

A transgenic plant for the purposes of the invention is thus understood as meaning, as above, that the nucleic acids used in the method of the invention are not at their natural locus in the genome of said plant, it being possible for the nucleic acids to be expressed homologously or heterologously. However, as mentioned, transgenic also means that, while the nucleic acids according to the invention or used in the inventive method are at their natural position in the genome of a plant, the sequence has been modified with regard to the natural sequence, and/or that the regulatory sequences of the natural sequences have been modified. Transgenic is preferably understood as meaning the expression of the nucleic acids according to the invention at an unnatural locus in the genome, i.e. homologous or, preferably, heterologous expression of the nucleic acids takes place. Preferred transgenic plants are mentioned herein.

### Transformation

The term “introduction” or “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

The transfer of foreign genes into the genome of a plant is called transformation. Transformation of plant species is now a fairly routine technique. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, F. A. et al., (1982) Nature 296, 72-74; Negrutiu I et al. (1987) Plant Mol Biol 8: 363-373); electroporation of protoplasts (Shillito R. D. et al. (1985) Bio/Technol 3, 1099-1102); microinjection into plant material (Crossway A et al., (1986) Mol. Gen. Genet 202: 179-185); DNA or RNA-coated particle bombardment (Klein T M et al., (1987) Nature 327: 70) infection with (non-integrative) viruses and the like. Transgenic plants, including transgenic crop plants, are preferably produced via Agrobacterium-mediated transformation. An advantageous transformation method is the transformation in planta. To this end, it is possible, for example, to allow the agrobacteria to act on plant seeds or to inoculate the plant meristem with agrobacteria. It has proved particularly expedient in accordance with the invention to allow a suspension of transformed agrobacteria to act on the intact plant or at least on the flower primordia. The plant is subsequently grown on until the seeds of the treated plant are obtained (Clough and Bent, Plant J. (1998) 16, 735-743). Methods for Agrobacterium-mediated transformation of rice include well known methods for rice transformation, such as those described in any of the following: European patent application EP 1198985 A1, Aldemita and Hodges (Planta 199: 612-617, 1996); Chan et al. (Plant Mol Biol 22 (3): 491-506, 1993), Hiei et al. (Plant J 6 (2): 271-282, 1994), which disclosures are incorporated by reference herein as if fully set forth. In the case of corn transformation, the preferred method is as described in either Ishida et al. (Nat. Biotechnol 14(6): 745-50, 1996) or Frame et al. (Plant Physiol 129(1): 13-22, 2002), which disclosures are incorporated by reference herein as if fully set forth. Said methods are further described by way of example in B. Jenes et al., Techniques for Gene Transfer, in: Transgenic Plants, Vol. 1, Engineering and Utilization, eds. S. D. Kung and R. Wu, Academic Press (1993) 128-143 and in Potrykus Annu. Rev. Plant Physiol. Plant Molec. Biol. 42 (1991) 205-225). The nucleic acids or the construct to be expressed is preferably cloned into a vector, which is suitable for transforming Agrobacterium tumefaciens, for example pBinl9 (Bevan et al., Nucl. Acids Res. 12 (1984) 8711). Agrobacteria transformed by such a vector can then be used in known manner for the transformation of plants, such as plants used as a model, like Arabidopsis (Arabidopsis thaliana is within the scope of the present invention not considered as a crop plant), or crop plants such as, by way of example, tobacco plants, for example by immersing bruised leaves or chopped leaves in an agrobacterial solution and then culturing them in suitable media. The transformation of plants by means of Agrobacterium tumefaciens is described, for example, by Höfgen and Willmitzer in Nucl. Acid Res. (1988) 16, 9877 or is known inter alia from F. F. White, Vectors for Gene Transfer in Higher Plants; in Transgenic Plants, Vol. 1, Engineering and Utilization, eds. S. D. Kung and R. Wu, Academic Press, 1993, pp. 15-38.

In addition to the transformation of somatic cells, which then have to be regenerated into intact plants, it is also possible to transform the cells of plant meristems and in particular those cells which develop into gametes. In this case, the transformed gametes follow the natural plant development, giving rise to transgenic plants. Thus, for example, seeds of Arabidopsis are treated with agrobacteria and seeds are obtained from the developing plants of which a certain proportion is transformed and thus transgenic [Feldman, K A and Marks M D (1987). Mol Gen Genet. 208:274-289; Feldmann K (1992). In: C Koncz, N-H Chua and J Shell, eds, Methods in Arabidopsis Research. Word Scientific, Singapore, pp. 274-289]. Alternative methods are based on the repeated removal of the inflorescences and incubation of the excision site in the center of the rosette with transformed agrobacteria, whereby transformed seeds can likewise be obtained at a later point in time (Chang (1994). Plant J. 5: 551-558; Katavic (1994). Mol Gen Genet, 245: 363-370). However, an especially effective method is the vacuum infiltration method with its modifications such as the “floral dip” method. In the case of vacuum infiltration of Arabidopsis, intact plants under reduced pressure are treated with an agrobacterial suspension [Bechthold, N (1993). C R Acad Sci Paris Life Sci, 316: 1194-1199], while in the case of the “floral dip” method the developing floral tissue is incubated briefly with a surfactant-treated agrobacterial suspension [Clough, S J and Bent A F (1998) The Plant J. 16, 735-743]. A certain proportion of transgenic seeds are harvested in both cases, and these seeds can be distinguished from non-transgenic seeds by growing under the above-described selective conditions. In addition the stable transformation of plastids is of advantages because plastids are inherited maternally is most crops reducing or eliminating the risk of transgene flow through pollen. The transformation of the chloroplast genome is generally achieved by a process which has been schematically displayed in Klaus et al., 2004 [Nature Biotechnology 22 (2), 225-229]. Briefly the sequences to be transformed are cloned together with a selectable marker gene between flanking sequences homologous to the chloroplast genome. These homologous flanking sequences direct site specific integration into the plastome. Plastidal transformation has been described for many different plant species and an overview is given in Bock (2001) Transgenic plastids in basic research and plant biotechnology. J Mol. Biol. 2001 Sep. 21; 312 (3):425-38 or Maliga, P (2003) Progress towards commercialization of plastid transformation technology. Trends Biotechnol. 21, 20-28. Further biotechnological progress has recently been reported in form of marker free plastid transformants, which can be produced by a transient co-integrated maker gene (Klaus et al., 2004, Nature Biotechnology 22(2), 225-229).

### T-DNA Activation Tagging

T-DNA activation tagging (Hayashi et al. Science (1992) 1350-1353), involves insertion of T-DNA, usually containing a promoter (may also be a translation enhancer or an intron), in the genomic region of the gene of interest or 10 kb up- or downstream of the coding region of a gene in a configuration such that the promoter directs expression of the targeted gene. Typically, regulation of expression of the targeted gene by its natural promoter is disrupted and the gene falls under the control of the newly introduced promoter. The promoter is typically embedded in a T-DNA. This T-DNA is randomly inserted into the plant genome, for example, through Agrobacterium infection and leads to modified expression of genes near the inserted T-DNA. The resulting transgenic plants show dominant phenotypes due to modified expression of genes close to the introduced promoter.

### Tilling

The term “TILLING” is an abbreviation of “Targeted Induced Local Lesions In Genomes” and refers to a mutagenesis technology useful to generate and/or identify nucleic acids encoding proteins with modified expression and/or activity. TILLING also allows selection of plants carrying such mutant variants. These mutant variants may exhibit modified expression, either in strength or in location or in timing (if the mutations affect the promoter for example). These mutant variants may exhibit higher activity than that exhibited by the gene in its natural form. TILLING combines high-density mutagenesis with high-throughput screening methods. The steps typically followed in TILLING are: (a) EMS mutagenesis (Redei GP and Koncz C (1992) In Methods in Arabidopsis Research, Koncz C, Chua N H, Schell J, eds. Singapore, World Scientific Publishing Co, pp. 16-82; Feldmann et al., (1994) In Meyerowitz E M, Somerville CR, eds, Arabidopsis. Cold Spring Harbor Laboratory Press, Cold Spring Harbor, N.Y., pp 137-172; Lightner J and Caspar T (1998) In J Martinez-Zapater, J Salinas, eds, Methods on Molecular Biology, Vol. 82. Humana Press, Totowa, N.J., pp 91-104); (b) DNA preparation and pooling of individuals; (c) PCR amplification of a region of interest; (d) denaturation and annealing to allow formation of heteroduplexes; (e) DHPLC, where the presence of a heteroduplex in a pool is detected as an extra peak in the chromatogram; (f) identification of the mutant individual; and (g) sequencing of the mutant PCR product. Methods for TILLING are well known in the art (McCallum et al., (2000) Nat Biotechnol 18: 455-457; reviewed by Stemple (2004) Nat Rev Genet. 5(2): 145-50).

### Homologous Recombination

Homologous recombination allows introduction in a genome of a selected nucleic acid at a defined selected position. Homologous recombination is a standard technology used routinely in biological sciences for lower organisms such as yeast or the moss Physcomitrella. Methods for performing homologous recombination in plants have been described not only for model plants (Offring a et al. (1990) EMBO J. 9(10): 3077-84) but also for crop plants, for example rice (Terada et al. (2002) Nat Biotech 20(10): 1030-4; lida and Terada (2004) Curr Opin Biotech 15(2): 132-8).

### Yield

The term “yield” in general means a measurable produce of economic value, typically related to a specified crop, to an area, and to a period of time. Individual plant parts directly contribute to yield based on their number, size and/or weight, or the actual yield is the yield per acre for a crop and year, which is determined by dividing total production (includes both harvested and appraised production) by planted acres. The term “yield” of a plant may relate to vegetative biomass (root and/or shoot biomass), to reproductive organs, and/or to propagules (such as seeds) of that plant.

### Early Vigour

“Early vigour” refers to active healthy well-balanced growth especially during early stages of plant growth, and may result from increased plant fitness due to, for example, the plants being better adapted to their environment (i.e. optimizing the use of energy resources and partitioning between shoot and root). Plants having early vigour also show increased seedling survival and a better establishment of the crop, which often results in highly uniform fields (with the crop growing in uniform manner, i.e. with the majority of plants reaching the various stages of development at substantially the same time), and often better and higher yield. Therefore, early vigour may be determined by measuring various factors, such as thousand kernel weight, percentage germination, percentage emergence, seedling growth, seedling height, root length, root and shoot biomass and many more.

### Increase/Improve/Enhance

The terms “increase”, “improve” or “enhance” are interchangeable and shall mean in the sense of the application at least a 5%, 6%, 7%, 8%, 9% or 10%, preferably at least 15% or 20%, more preferably 25%, 30%, 35% or 40% more yield and/or growth in comparison to control plants as defined herein.

### Seed Yield

Increased seed yield may manifest itself as one or more of the following: a) an increase in seed biomass (total seed weight) which may be on an individual seed basis and/or per plant and/or per hectare or acre; b) increased number of flowers per plant; c) increased number of (filled) seeds; d) increased seed filling rate (which is expressed as the ratio between the number of filled seeds divided by the total number of seeds); e) increased harvest index, which is expressed as a ratio of the yield of harvestable parts, such as seeds, divided by the total biomass; and f) increased thousand kernel weight (TKW), which is extrapolated from the number of filled seeds counted and their total weight. An increased TKW may result from an increased seed size and/or seed weight, and may also result from an increase in embryo and/or endosperm size.

An increase in seed yield may also be manifested as an increase in seed size and/or seed volume. Furthermore, an increase in seed yield may also manifest itself as an increase in seed area and/or seed length and/or seed width and/or seed perimeter. Increased yield may also result in modified architecture, or may occur because of modified architecture.

### Greenness Index

The “greenness index” as used herein is calculated from digital images of plants. For each pixel belonging to the plant object on the image, the ratio of the green value versus the red value (in the RGB model for encoding color) is calculated. The greenness index is expressed as the percentage of pixels for which the green-to-red ratio exceeds a given threshold. Under normal growth conditions, under salt stress growth conditions, and under reduced nutrient availability growth conditions, the greenness index of plants is measured in the last imaging before flowering. In contrast, under drought stress growth conditions, the greenness index of plants is measured in the first imaging after drought.

### Plant

The term “plant” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprises the gene/nucleic acid of interest.

Plants that are particularly useful in the methods of the invention include all plants which belong to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Triticosecale rimpaui, Triticum spp. (e.g. Triticum aestivum, Triticum durum, Triticum turgidum, Triticum hybernum, Triticum macha, Triticum sativum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amongst others.

### DETAILED DESCRIPTION OF THE INVENTION

### ERLK

It has now been found that modulating expression in a plant of a nucleic acid encoding an extensin receptor-like kinase (ERLK) or a part thereof comprising at least the kinase domain and the transmembrane domain, gives plants having enhanced yield-related traits relative to control plants.

Therefore, according to a first embodiment, the invention provides a method for enhancing yield-related traits in plants relative to control plants, comprising modulating expression in a plant of a nucleic acid encoding an ERLK protein, or a part thereof.

A preferred method for modulating (preferably, increasing) expression of a nucleic acid encoding an extensin receptor-like kinase (ERLK) or a part thereof comprising at least the kinase domain and the transmembrane domain, is by introducing and expressing in a plant a nucleic acid encoding such an ERLK protein.

Any reference hereinafter to a “protein useful in the methods of the invention” is taken to mean an ERLK polypeptide as defined herein. Any reference hereinafter to a “nucleic acid useful in the methods of the invention” is taken to mean a nucleic acid capable of encoding such an ERLK polypeptide. The nucleic acid to be introduced into a plant (and therefore useful in performing the methods of the invention) is any nucleic acid encoding the type of protein which will now be described, hereafter also named “ERLK nucleic acid” or “ERLK gene”.

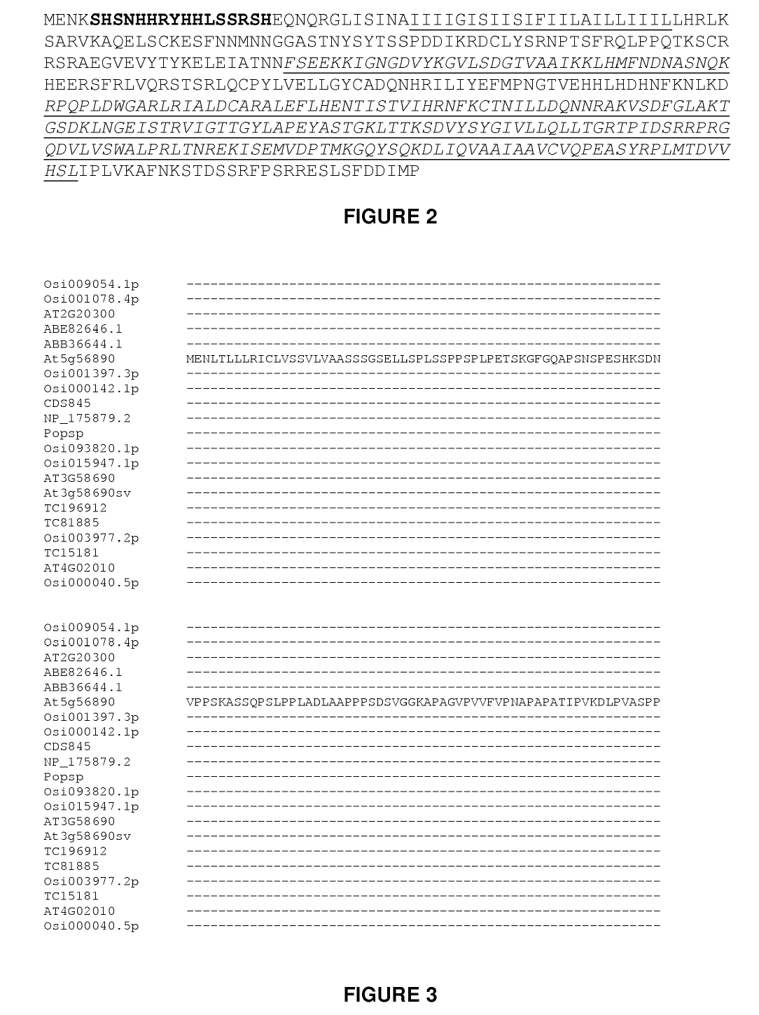

The ERLK protein useful in the methods of the present invention is an ERLK protein as defined by Shiu and Bleeker (2001). The term “ERLK protein” or “extensin receptor-like kinase” refers to a protein comprising a kinase domain and N-terminally thereof a transmembrane domain (see FIG. 1 and FIG. 2 for a schematic overview). ERLK proteins preferably also comprise an N-terminal secretion signal sequence and optionally an extracellular domain. Preferably the kinase domain (and/or other domains) of the ERLK protein useful in the present invention classifies as an extensin receptor-like kinase as defined by Shiu and Bleeker (2001).

The term “domain” and “motif” is defined in the “definitions” section herein. Specialist databases exist for the identification of domains, for example, SMART (Schultz et al. (1998) Proc. Natl. Acad. Sci. USA 95, 5857-5864; Letunic et al. (2002) Nucleic Acids Res 30, 242-244, InterPro (Mulder et al., (2003) Nucl. Acids. Res. 31, 315-318, Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAI Press, Menlo Park; Hulo et al., Nucl. Acids. Res. 32:D134-D137, (2004), or Pfam (Bateman et al., Nucleic Acids Research 30(1): 276-280 (2002). A set of tools for in silico analysis of protein sequences is available on the ExPASy proteomics server (Swiss Institute of Bioinformatics (Gasteiger et al., ExPASy: the proteomics server for in-depth protein knowledge and analysis, Nucleic Acids Res. 31:3784-3788 (2003)). Domains may also be identified using routine techniques, such as by sequence alignment.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e. spanning the complete sequences) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences.). Minor manual editing may be performed to optimise alignment between conserved motifs, as would be apparent to a person skilled in the art. Furthermore, instead of using full-length sequences for the identification of homologues, specific domains (such as the kinase domain) may also be used. The sequence identity values may be determined over the entire nucleic acid or amino acid sequence or over selected domains or conserved motif(s), using the programs mentioned above using the default parameters.

The kinase domain in ERLK proteins useful in the methods of the present invention is a protein Tyr kinase type domain (Pfam entry PF07714, InterPro entry IPR001245). The active site corresponds to the PROSITE signature PS00109, with the following consensus pattern: [LIVMFYC]-{A}-[HY]-x-D -[LIVMFY]-[RSTAC]-{D}-{PF}-N -[LIVMFYC](3), wherein D is part of the active site. The syntax of this pattern is according to the conventions used in the Prosite database and is explained in the PROSITE manual. Preferably, the kinase domain is furthermore characterised by the presence of sequence motif 1 (SEQ ID NO: 6):

(M/L) L (S/G/R) R (L/M) (H/R/Q) (H/S/C) (R/P)
(N/Y) L (L/V) XL (I/L/V) G

wherein X may be any amino acid, preferably one of K, N, A, S, G, E. Preferably, motif 1 has the sequence LLSR(L/M) (H/R/Q) (C/S)PYL(L/V) (E/G/A)L(L/I); most preferably motif 1 has the sequence LLSRLQCPYLVELLG.

Preferably, the kinase domain also comprises one or more of sequence motif 2 (SEQ ID NO: 7):

L (Y/D/N) (W/F) X (A/V/T) R (L/M) (L/R/G) IA (L/V)

wherein X may be any amino acid, preferably one of D, N, E, K, P, Q, or G, sequence motif 3 (SEQ ID NO: 8):

A (R/K) (A/G) L (A/E) (Y/F) LHE,

sequence motif 4 (SEQ ID NO: 9):

(V/I) IHR (D/N) (F/L) K (S/A/G/C) (S/T) N (I/V) LL
(E/D)

wherein the amino acid on position 6 is preferably not I, V or M, sequence motif 5 (SEQ ID NO: 10):

(K/R) V (S/A/T) DFG (L/M/S)

Preferably, sequence motif 2 has the sequence LDW(G/Q/P/E) (A/T)R(L/M) (R/G) IA(L/V), more preferably, sequence motif 2 has the sequence LDW (G/Q) (T/A) RL (R/G) IAL, most preferably, sequence motif 2 has the sequence LDWGARLRIAL. Sequence motif 3 preferably has the sequence ARALEFLHE. Sequence motif 4 preferably has the sequence VIHR (D/N) (F/L) K (S/C) (S/T) NILLD, most preferably, the sequence is VIHRNFKCTNILLD. Sequence motif 5 preferably has the sequence (K/R) VSDFG (L/M), most preferably the sequence is KVSDFGL.

Preferably, the kinase domain of ERLK proteins useful in the methods of the present invention have, in increasing order of preference, at least 39%, 40%, 45%, 50%, 55%, 60%, 65%, 70%, 75%, 80%, 85%, 90%, 95%, 96%, 97%, 98% or 99% sequence identity to the kinase domain of SEQ ID NO: 2 (as given in SEQ ID NO: 57). A kinase domain may be identified using the databases and tools for protein identification listed above, and/or methods for the alignment of sequences for comparison. In some instances, default parameters may be adjusted to modify the stringency of the search. For example using BLAST, the statistical significance threshold (called “expect” value) for reporting matches against database sequences may be increased to show less stringent matches. In this way, short nearly exact matches may be identified.

Transmembrane domains are about 15 to 30 amino acids long and are usually composed of hydrophobic residues that form an alpha helix. They are usually predicted on the basis of hydrophobicity (for example Klein et al., Biochim. Biophys. Acta 815, 468, 1985; or Sonnhammer et al., In J. Glasgow, T. Littlejohn, F. Major, R. Lathrop, D. Sankoff, and C. Sensen, editors, Proceedings of the Sixth International Conference on Intelligent Systems for Molecular Biology, pages 175-182, Menlo Park, Calif., 1998, AAAI Press).

The extracellular domain of an ERLK protein, if present, may (but not necessarily need to) have one or more SPx motifs. The structure of secretion signal sequences and the prediction of its cleavage sites are well known in the art.

Furthermore, ERLK proteins useful in the methods of the present invention (at least in their native form) typically, but not necessarily, have kinase activity. Therefore, ERLK proteins with reduced kinase activity or without kinase activity may equally be useful in the methods of the present invention. A person skilled in the art may easily determine the presence of kinase activity using routine tools and techniques. To determine the kinase activity of receptor like kinases, several assays are available (for example Current Protocols in Molecular Biology, Volumes 1 and 2, Ausubel et al. (1994), Current Protocols). In brief, a kinase assay generally involves (1) bringing the kinase protein into contact with a substrate polypeptide containing the target site to be phosphorylated; (2) allowing phosphorylation of the target site in an appropriate kinase buffer under appropriate conditions; (3) separating phosphorylated products from non-phosphorylated substrate after a suitable reaction period. The presence or absence of kinase activity is determined by the presence or absence of a phosphorylated target. In addition, quantitative measurements can be performed. Purified receptor like kinase, or cell extracts containing or enriched in the receptor like kinase could be used as source for the kinase protein. Alternatively, the approach of Zhao et al. (Plant Mol. Biol. 26, 791-803, 1994) could be used, where the cytoplasmic domain of a rice receptor like kinase was expressed in Escherichia coli and assayed for kinase activity. As a substrate, small peptides are particularly well suited. The peptide must comprise one or more serine, threonine or tyrosine residues in a phosphorylation site motif. A compilation of phosphorylation sites can be found in Biochimica et Biophysica Acta 1314, 191-225, (1996). In addition, the peptide substrates may advantageously have a net positive charge to facilitate binding to phosphocellulose filters, (allowing to separate the phosphorylated from non-phosphorylated peptides and to detect the phosphorylated peptides). If a phosphorylation site motif is not known, a general tyrosine kinase substrate can be used. For example, “Src-related peptide” (RRLIEDAEYAARG) is a substrate for many receptor and non-receptor tyrosine kinases). To determine the kinetic parameters for phosphorylation of the synthetic peptide, a range of peptide concentrations is required. For initial reactions, a peptide concentration of 0.7-1.5 mM could be used. For each kinase enzyme, it is important to determine the optimal buffer, ionic strength, and pH for activity. A standard 5×Kinase Buffer generally contains 5 mg/ml BSA (Bovine Serum Albumin preventing kinase adsorption to the assay tube), 150 mM Tris-Cl (pH 7.5), 100 mM MgCl₂. Divalent cations are required for most tyrosine kinases, although some tyrosine kinases (for example, insulin-, IGF-1-, and PDGF receptor kinases) require MnCl₂ instead of MgCl₂ (or in addition to MgCl₂). The optimal concentrations of divalent cations must be determined empirically for each protein kinase. A commonly used donor for the phosphoryl group is radio-labelled [gamma-³²P]ATP (normally at 0.2 mM final concentration). The amount of ³²P incorporated in the peptides may be determined by measuring activity on the nitrocellulose dry pads in a scintillation counter.

The present invention is illustrated by transforming plants with the nucleic acid sequence represented by SEQ ID NO: 1, encoding the polypeptide sequence of SEQ ID NO: 2. However, performance of the invention is not restricted to these sequences; the methods of the invention may advantageously be performed using any ERLK-encoding nucleic acid or ERLK polypeptide as defined herein.

Examples of nucleic acids encoding ERLK polypeptides are given in Table A of Example 1 herein. Such nucleic acids are useful in performing the methods of the invention. The amino acid sequences given in Table A of Example 1 are example sequences of orthologues and paralogues of the ERLK polypeptide represented by SEQ ID NO: 2, the terms “orthologues” and “paralogues” being as defined herein. Further orthologues and paralogues may readily be identified by performing a so-called reciprocal blast search. Typically, this involves a first BLAST involving BLASTing a query sequence (for example using any of the sequences listed in Table A of Example 1) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 1, SEQ ID NO: 2, SEQ ID NO: 11, SEQ ID NO: 12, SEQ ID NO: 13 or SEQ ID NO: 14, the second BLAST would therefore be against Arabidopsis sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

High-ranking hits are those having a low E-value. The lower the E-value, the more significant the score (or in other words the lower the chance that the hit was found by chance). Computation of the E-value is well known in the art. In addition to E-values, comparisons are also scored by percentage identity. Percentage identity refers to the number of identical nucleotides (or amino acids) between the two compared nucleic acid (or polypeptide) sequences over a particular length. In the case of large families, ClustalW may be used, followed by a neighbour joining tree, to help visualize clustering of related genes and to identify orthologues and paralogues.

Nucleic acid variants may also be useful in practising the methods of the invention. Examples of such variants include nucleic acids encoding homologues and derivatives of any one of the amino acid sequences given in Table A of Example 1, the terms “homologue” and “derivative” being as defined herein. Also useful in the methods of the invention are nucleic acids encoding homologues and derivatives of orthologues or paralogues of any one of the amino acid sequences given in Table A of Example 1. Homologues and derivatives useful in the methods of the present invention have substantially the same biological and functional activity as the unmodified protein from which they are derived.

Further nucleic acid variants useful in practising the methods of the invention include portions of nucleic acids encoding ERLK polypeptides, nucleic acids hybridising to nucleic acids encoding ERLK polypeptides, splice variants of nucleic acids encoding ERLK polypeptides, allelic variants of nucleic acids encoding ERLK polypeptides and variants of nucleic acids encoding ERLK polypeptides obtained by gene shuffling. The terms hybridising sequence, splice variant, allelic variant and gene shuffling are as described herein.

Nucleic acids encoding ERLK polypeptides need not be full-length nucleic acids, since performance of the methods of the invention does not rely on the use of full-length nucleic acid sequences. According to the present invention, there is provided a method for enhancing yield-related traits in plants, comprising introducing and expressing in a plant a portion of any one of the nucleic acid sequences given in Table A of Example 1, or a portion of a nucleic acid encoding an orthologue, paralogue or homologue of any of the amino acid sequences given in Table A of Example 1. Preferably, the portion encodes a polypeptide comprising at least, from N-terminus to C-terminus, (i) a transmembrane domain and (ii) an extensin receptor-like kinase-type (ERLK-type) kinase domain.

A portion of a nucleic acid may be prepared, for example, by making one or more deletions to the nucleic acid. The portions may be used in isolated form or they may be fused to other coding (or non-coding) sequences in order to, for example, produce a protein that combines several activities. When fused to other coding sequences, the resultant polypeptide produced upon translation may be bigger than that predicted for the protein portion.

Portions useful in the methods of the invention, encode a ERLK polypeptide as defined herein, having a kinase domain (as described above) and having substantially the same biological activity as the ERLK protein represented by any of the amino acid sequences given in Table A of Example 1. Preferably, the portion is a portion of any one of the nucleic acids given in Table

A of Example 1, or is a portion of a nucleic acid encoding an orthologue or paralogue of any one of the amino acid sequences given in Table A of Example 1. Preferably the portion is at least 800, 850, 900, 950, 1000, 1050, 1100, 1150, 1200, 1250, 1300 consecutive nucleotides in length, the consecutive nucleotides being of any one of the nucleic acid sequences given in Table A of Example 1, or of a nucleic acid encoding an orthologue or paralogue of any one of the amino acid sequences given in Table A of Example 1. Most preferably the portion is a portion of the nucleic acid of SEQ ID NO: 1.

Another nucleic acid variant useful in the methods of the invention is a nucleic acid capable of hybridising, under reduced stringency conditions, preferably under stringent conditions, with a nucleic acid encoding an ERLK polypeptide as defined herein, or with a portion as defined herein.

According to the present invention, there is provided a method for enhancing yield-related traits in plants, comprising introducing and expressing in a plant a nucleic acid capable of hybridizing to any one of the nucleic acids given in Table A of Example 1, or comprising introducing and expressing in a plant a nucleic acid capable of hybridising to a nucleic acid encoding an orthologue, paralogue or homologue of any of the nucleic acid sequences given in Table A of Example 1.

Hybridising sequences useful in the methods of the invention encode an ERLK polypeptide as defined herein, having an ERLK-type kinase domain and a transmembrane domain (as described above), and having substantially the same biological activity as the amino acid sequences given in Table A of Example 1. The hybridising sequence is typically at least 800 nucleotides in length, preferably at least 1000 nucleotides in length, more preferably at least 1200 nucleotides in length and most preferably at least 1300 nucleotides in length. Preferably, the hybridising sequence is capable of hybridising to any one of the nucleic acids given in Table A of Example 1, or to a portion of any of these sequences, a portion being as defined above, or the hybridising sequence is capable of hybridising to a nucleic acid encoding an orthologue or paralogue of any one of the amino acid sequences given in Table A of Example 1, or to probes, or to probes derived therefrom. Most preferably, the hybridising sequence is capable of hybridising to a nucleic acid as represented by SEQ ID NO: 1 or to a portion or probe thereof.

Methods for designing probes are well known in the art. Probes are generally less than 1000 bp in length, preferably less than 500 bp in length. Commonly, probe lengths for DNA-DNA hybridisations such as Southern blotting, vary between 100 and 500 bp, whereas the hybridising region in probes for DNA-DNA hybridisations such as in PCR amplification generally are shorter than 50 but longer than 10 nucleotides.

Another nucleic acid variant useful in the methods of the invention is a splice variant encoding an ERLK polypeptide as defined hereinabove, a splice variant being as defined herein.

According to the present invention, there is provided a method for enhancing yield-related traits in plants, comprising introducing and expressing in a plant a splice variant of any one of the nucleic acid sequences given in Table A of Example 1, or a splice variant of a nucleic acid encoding an orthologue, paralogue or homologue of any of the amino acid sequences given in Table A of Example 1.

Preferred splice variants are splice variants of a nucleic acid represented by SEQ ID NO: 1, or a splice variant of a nucleic acid encoding an orthologue or paralogue of SEQ ID NO: 2.

Another nucleic acid variant useful in performing the methods of the invention is an allelic variant of a nucleic acid encoding an ERLK polypeptide as defined hereinabove, an allelic variant being as defined herein.

According to the present invention, there is provided a method for enhancing yield-related traits in plants, comprising introducing and expressing in a plant an allelic variant of any one of the nucleic acids given in Table A of Example 1, or comprising introducing and expressing in a plant an allelic variant of a nucleic acid encoding an orthologue, paralogue or homologue of any of the amino acid sequences given in Table A of Example 1.

The allelic variants useful in the methods of the present invention have substantially the same biological activity as the ERLK polypeptide of SEQ ID NO: 2 and any of the amino acids depicted in Table A of Example 1. Allelic variants exist in nature, and encompassed within the methods of the present invention is the use of these natural alleles. Preferably, the allelic variant is an allelic variant of SEQ ID NO: 1 or an allelic variant of a nucleic acid encoding an orthologue or paralogue of SEQ ID NO: 2.

A further nucleic acid variant useful in the methods of the invention is a nucleic acid variant obtained by gene shuffling. Gene shuffling or directed evolution may also be used to generate variants of nucleic acids encoding ERLK polypeptides as defined above; the term “gene shuffling” being as defined herein.

According to the present invention, there is provided a method for enhancing yield-related traits in plants, comprising introducing and expressing in a plant a variant of any one of the nucleic acid sequences given in Table A of Example 1, or comprising introducing and expressing in a plant a variant of a nucleic acid encoding an orthologue, paralogue or homologue of any of the amino acid sequences given in Table A of Example 1, which variant nucleic acid is obtained by gene shuffling.

Furthermore, nucleic acid variants may also be obtained by site-directed mutagenesis. Several methods are available to achieve site-directed mutagenesis, the most common being PCR based methods (Current Protocols in Molecular Biology. Wiley Eds.).

Nucleic acids encoding ERLK polypeptides may be derived from any natural or artificial source. The nucleic acid may be modified from its native form in composition and/or genomic environment through deliberate human manipulation. Preferably the ERLK polypeptide-encoding nucleic acid is from a plant, further preferably from a dicotyledonous plant, more preferably from the family Brassicaceae, most preferably the nucleic acid is from Arabidopsis thaliana.

Performance of the methods of the invention gives plants having enhanced yield-related traits. In particular performance of the methods of the invention gives plants having increased yield, especially increased seed yield relative to control plants. The terms “yield” and “seed yield” are described in more detail in the “definitions” section herein.

Reference herein to enhanced yield-related traits is taken to mean an increase in biomass (weight) of one or more parts of a plant, which may include aboveground (harvestable) parts and/or (harvestable) parts below ground. In particular, such harvestable parts are seeds and leafy biomass, and performance of the methods of the invention results in plants having increased leafy biomass and increased seed yield, relative to control plants.

Taking corn as an example, a yield increase may be manifested as one or more of the following: increase in the number of plants established per hectare or acre, an increase in the number of ears per plant, an increase in the number of rows, number of kernels per row, kernel weight, thousand kernel weight, ear length/diameter, increase in the seed filling rate (which is the number of filled seeds divided by the total number of seeds and multiplied by 100), among others. Taking rice as an example, a yield increase may manifest itself as an increase in one or more of the following: number of plants per hectare or acre, number of panicles per plant, number of spikelets per panicle, number of flowers (florets) per panicle (which is expressed as a ratio of the number of filled seeds over the number of primary panicles), increase in the seed filling rate (which is the number of filled seeds divided by the total number of seeds and multiplied by 100), increase in thousand kernel weight, among others.

The present invention provides a method for increasing yield, especially increased leafy biomass and increased seed yield of plants, relative to control plants, which method comprises modulating expression, preferably increasing expression, in a plant of a nucleic acid encoding a ERLK polypeptide as defined herein.

Since the transgenic plants according to the present invention have increased yield, it is likely that these plants exhibit an increased growth rate (during at least part of their life cycle), relative to the growth rate of control plants at a corresponding stage in their life cycle.

The increased growth rate may be specific to one or more parts of a plant (including seeds), or may be throughout substantially the whole plant. Plants having an increased growth rate may have a shorter life cycle. The life cycle of a plant may be taken to mean the time needed to grow from a dry mature seed up to the stage where the plant has produced dry mature seeds, similar to the starting material. This life cycle may be influenced by factors such as early vigour, growth rate, greenness index, flowering time and speed of seed maturation. The increase in growth rate may take place at one or more stages in the life cycle of a plant or during substantially the whole plant life cycle. Increased growth rate during the early stages in the life cycle of a plant may reflect enhanced vigour. Increased growth rate during the early stages in the life cycle of a plant may reflect enhanced vigour. The increase in growth rate may alter the harvest cycle of a plant allowing plants to be sown later and/or harvested sooner than would otherwise be possible (a similar effect may be obtained with earlier flowering time). If the growth rate is sufficiently increased, it may allow for the further sowing of seeds of the same plant species (for example sowing and harvesting of rice plants followed by sowing and harvesting of further rice plants all within one conventional growing period). Similarly, if the growth rate is sufficiently increased, it may allow for the further sowing of seeds of different plants species (for example the sowing and harvesting of corn plants followed by, for example, the sowing and optional harvesting of soybean, potato or any other suitable plant). Harvesting additional times from the same rootstock in the case of some crop plants may also be possible. Altering the harvest cycle of a plant may lead to an increase in annual biomass production per acre (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and T-90 (time taken for plants to reach 90% of their maximal size), amongst others.

According to a preferred feature of the present invention, performance of the methods of the invention gives plants having an increased growth rate relative to control plants. Therefore, according to the present invention, there is provided a method for increasing the growth rate of plants, which method comprises modulating expression, preferably increasing expression, in a plant of a nucleic acid encoding an ERLK polypeptide as defined herein.

An increase in yield and/or growth rate occurs whether the plant is under non-stress conditions or whether the plant is exposed to various stresses compared to control plants. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35% or 30%, preferably less than 25%, 20% or 15%, more preferably less than 14%, 13%, 12%, 11% or 10% or less in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the everyday biotic and/or abiotic (environmental) stresses to which a plant is exposed. Abiotic stresses may be due to drought or excess water, anaerobic stress, salt stress, chemical toxicity, oxidative stress and hot, cold or freezing temperatures. The abiotic stress may be an osmotic stress caused by a water stress (particularly due to drought), salt stress, oxidative stress or an ionic stress. Biotic stresses are typically those stresses caused by pathogens, such as bacteria, viruses, fungi and insects.

In particular, the methods of the present invention may be performed under non-stress conditions or under conditions of mild drought to give plants having increased yield relative to control plants. As reported in Wang et al. (Planta (2003) 218: 1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani et al. (Plant Physiol (2003) 133: 1755-1767) describes a particularly high degree of “cross talk” between drought stress and high-salinity stress. For example, drought and/or salinisation are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signalling pathways and cellular responses, such as the production of stress proteins, up-regulation of anti-oxidants, accumulation of compatible solutes and growth arrest. The term “non-stress” conditions as used herein are those environmental conditions that allow optimal growth of plants. Persons skilled in the art are aware of normal soil conditions and climatic conditions for a given location.

Performance of the methods of the invention gives plants grown under non-stress conditions or under mild drought conditions increased yield relative to control plants grown under comparable conditions. Therefore, according to the present invention, there is provided a method for increasing yield in plants grown under non-stress conditions or under mild drought conditions, which method comprises increasing expression in a plant of a nucleic acid encoding an ERLK polypeptide.

Performance of the methods of the invention gives plants grown under conditions of nutrient deficiency, particularly under conditions of nitrogen deficiency, increased yield relative to control plants grown under comparable conditions. Therefore, according to the present invention, there is provided a method for increasing yield in plants grown under conditions of nutrient deficiency, which method comprises increasing expression in a plant of a nucleic acid encoding an ERLK polypeptide. Nutrient deficiency may result from a lack or excess of nutrients such as nitrogen, phosphates and other phosphorous-containing compounds, potassium, calcium, cadmium, magnesium, manganese, iron and boron, amongst others.

The present invention encompasses plants or parts thereof (including seeds) obtainable by the methods according to the present invention. The plants or parts thereof comprise a nucleic acid transgene encoding an ERLK polypeptide as defined above.

The invention also provides genetic constructs and vectors to facilitate introduction and/or expression in plants of nucleic acids encoding ERLK polypeptides. The gene constructs may be inserted into vectors, which may be commercially available, suitable for transforming into plants and suitable for expression of the gene of interest in the transformed cells. The invention also provides use of a gene construct as defined herein in the methods of the invention.

More specifically, the present invention provides a construct comprising:
(a) a nucleic acid encoding an ERLK polypeptide as defined above;
(b) one or more control sequences capable of driving expression of the nucleic acid sequence of (a); and optionally
(c) a transcription termination sequence.

Preferably, the nucleic acid encoding an ERLK polypeptide is as defined above. The term “control sequence” and “termination sequence” are as defined herein.

Plants are transformed with a vector comprising any of the nucleic acids described above. The skilled artisan is well aware of the genetic elements that must be present on the vector in order to successfully transform, select and propagate host cells containing the sequence of interest. The sequence of interest is operably linked to one or more control sequences (at least to a promoter).

Advantageously, any type of promoter, whether natural or synthetic, may be used to drive expression of the nucleic acid sequence. A constitutive promoter is particularly useful in the methods. See the “Definitions” section herein for definitions of the various promoter types. A preferred constitutive promoter is a constitutive promoter that is also substantially ubiquitously expressed. Further preferably the promoter is derived from a plant, more preferably a monocotyledonous plant. Most preferred is use of a GOS2 promoter, substantially similar or identical to the GOS2 promoter from rice (SEQ ID NO: 5 or SEQ ID NO: 58).

It should be clear that the applicability of the present invention is not restricted to the ERLK polypeptide-encoding nucleic acid represented by SEQ ID NO: 1, nor is the applicability of the invention restricted to expression of an ERLK polypeptide-encoding nucleic acid when driven by a constitutive promoter. See Table 2a in the “Definitions” section herein for further examples of constitutive promoters.

Optionally, one or more terminator sequences may be used in the construct introduced into a plant. Additional regulatory elements may include transcriptional as well as translational enhancers. Those skilled in the art will be aware of terminator and enhancer sequences that may be suitable for use in performing the invention. An intron sequence may also be added to the 5′ untranslated region (UTR) or in the coding sequence to increase the amount of the mature message that accumulates in the cytosol, as described in the definitions section. Other control sequences (besides promoter, enhancer, silencer, intron sequences, 3′UTRand/or 5′UTR regions) may be protein and/or RNA stabilizing elements. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

For the detection of the successful transfer of the nucleic acid sequences as used in the methods of the invention and/or selection of transgenic plants comprising these nucleic acids, it is advantageous to use marker genes (or reporter genes). Therefore, the genetic construct may optionally comprise a selectable marker gene. Selectable markers are described in more detail in the “definitions” section herein. The marker genes may be removed or excised from the transgenic cell once they are no longer needed. Techniques for marker gene removal are known in the art, useful techniques are described above in the definitions section.

The invention also provides a method for the production of transgenic plants having enhanced yield-related traits relative to control plants, comprising introduction and expression in a plant of any nucleic acid encoding an ERLK polypeptide as defined hereinabove.

More specifically, the present invention provides a method for the production of transgenic plants having increased enhanced yield-related traits, particularly increased leafy biomass and seed yield, which method comprises:
(i) introducing and expressing in a plant or plant cell an ERLK polypeptide-encoding nucleic acid; and
(ii) cultivating the plant cell under conditions promoting plant growth and development.

The nucleic acid of (i) may be any of the nucleic acids capable of encoding an ERLK polypeptide as defined herein.

The nucleic acid may be introduced directly into a plant cell or into the plant itself (including introduction into a tissue, organ or any other part of a plant). According to a preferred feature of the present invention, the nucleic acid is preferably introduced into a plant by transformation. The term “transformation” is described in more detail in the “definitions” section herein.

The genetically modified plant cells can be regenerated via all methods with which the skilled worker is familiar. Suitable methods can be found in the abovementioned publications by S. D. Kung and R. Wu, Potrykus or Höfgen and Willmitzer.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

The invention also includes host cells containing an isolated nucleic acid encoding an ERLK polypeptide as defined hereinabove. Preferred host cells according to the invention are plant cells. Host plants for the nucleic acids or the vector used in the method according to the invention, the expression cassette or construct or vector are, in principle, advantageously all plants, which are capable of synthesizing the polypeptides used in the inventive method.

The methods of the invention are advantageously applicable to any plant. The present invention also encompasses plants obtainable by the methods according to the present invention. The present invention therefore provides plants, plant parts or plant cells thereof obtainable by the method according to the present invention, which plants or parts or cells thereof comprise a nucleic acid transgene encoding an ERLK protein as defined above. Plants that are particularly useful in the methods of the invention include all plants which belong to the superfamily Viridiplantae, in particular monocotyledonous and dicotyledonous plants including fodder or forage legumes, ornamental plants, food crops, trees or shrubs. According to a preferred embodiment of the present invention, the plant is a crop plant. Examples of crop plants include soybean, sunflower, canola, alfalfa, rapeseed, cotton, tomato, potato and tobacco. Further preferably, the plant is a monocotyledonous plant. Examples of monocotyledonous plants include sugarcane. More preferably the plant is a cereal. Examples of cereals include rice, maize, wheat, barley, millet, rye, triticale, sorghum and oats.

The invention also extends to harvestable parts of a plant such as, but not limited to seeds, leaves, fruits, flowers, stems, roots, rhizomes, tubers and bulbs. The invention furthermore relates to products derived, preferably directly derived, from a harvestable part of such a plant, such as dry pellets or powders, oil, fat and fatty acids, starch or proteins.

According to a preferred feature of the invention, the modulated expression is increased expression. Methods for increasing expression of nucleic acids or genes, or gene products, are well documented in the art and examples are provided in the definitions section.

As mentioned above, a preferred method for modulating (preferably, increasing) expression of a nucleic acid encoding an ERLK polypeptide is by introducing and expressing in a plant a nucleic acid encoding an ERLK polypeptide; however the effects of performing the method, i.e. enhancing yield-related traits may also be achieved using other well known techniques, including but not limited to T-DNA activation tagging, TILLING, homologous recombination. A description of these techniques is provided in the definitions section.

The present invention also encompasses use of nucleic acids encoding ERLK polypeptides as described herein and use of these ERLK polypeptides in enhancing any of the aforementioned yield-related traits in plants.

Nucleic acids encoding ERLK polypeptide described herein, or the ERLK polypeptides themselves, may find use in breeding programmes in which a DNA marker is identified which may be genetically linked to an ERLK polypeptide-encoding gene. The nucleic acids/genes, or the ERLK polypeptides themselves may be used to define a molecular marker. This DNA or protein marker may then be used in breeding programmes to select plants having enhanced yield-related traits as defined hereinabove in the methods of the invention.

Allelic variants of an ERLK polypeptide-encoding nucleic acid/gene may also find use in marker-assisted breeding programmes. Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give increased yield. Selection is typically carried out by monitoring growth performance of plants containing different allelic variants of the sequence in question. Growth performance may be monitored in a greenhouse or in the field. Further optional steps include crossing plants in which the superior allelic variant was identified with another plant. This could be used, for example, to make a combination of interesting phenotypic features.

Nucleic acids encoding ERLK polypeptides may also be used as probes for genetically and physically mapping the genes that they are a part of, and as markers for traits linked to those genes. Such information may be useful in plant breeding in order to develop lines with desired phenotypes. Such use of ERLK polypeptide-encoding nucleic acids requires only a nucleic acid sequence of at least 15 nucleotides in length. The ERLK polypeptide-encoding nucleic acids may be used as restriction fragment length polymorphism (RFLP) markers. Southern blots (Sambrook J, Fritsch E F and Maniatis T (1989) Molecular Cloning, A Laboratory Manual) of restriction-digested plant genomic DNA may be probed with the ERLK-encoding nucleic acids. The resulting banding patterns may then be subjected to genetic analyses using computer programs such as MapMaker (Lander et al. (1987) Genomics 1: 174-181) in order to construct a genetic map. In addition, the nucleic acids may be used to probe Southern blots containing restriction endonuclease-treated genomic DNAs of a set of individuals representing parent and progeny of a defined genetic cross. Segregation of the DNA polymorphisms is noted and used to calculate the position of the ERLK polypeptide-encoding nucleic acid in the genetic map previously obtained using this population (Botstein et al. (1980) Am. J. Hum. Genet. 32:314-331).

The production and use of plant gene-derived probes for use in genetic mapping is described in Bernatzky and Tanksley (1986) Plant Mol. Biol. Reporter 4: 37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid probes may be used in direct fluorescence in situ hybridisation (FISH) mapping (Trask (1991) Trends Genet. 7:149-154). Although current methods of FISH mapping favour use of large clones (several kb to several hundred kb; see Laan et al. (1995) Genome Res. 5:13-20), improvements in sensitivity may allow performance of FISH mapping using shorter probes.

A variety of nucleic acid amplification-based methods for genetic and physical mapping may be carried out using the nucleic acids. Examples include allele-specific amplification (Kazazian (1989) J. Lab. Clin. Med. 11:95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic Acid Res. 18:3671), Radiation Hybrid Mapping (Walter et al. (1997) Nat. Genet. 7:22-28) and Happy Mapping (Dear and Cook (1989) Nucleic Acid Res. 17:6795-6807). For these methods, the sequence of a nucleic acid is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

The methods according to the present invention result in plants having enhanced yield-related traits, as described hereinbefore. These traits may also be combined with other economically advantageous traits, such as further yield-enhancing traits, tolerance to other abiotic and biotic stresses, traits modifying various architectural features and/or biochemical and/or physiological features.

### FBW40

Surprisingly, it has now been found that increasing expression in a plant of a nucleic acid encoding an FBXW polypeptide gives plants having enhanced yield-related traits relative to control plants. Therefore, the invention provides a method for enhancing yield-related in plants relative to control plants, comprising increasing expression in a plant of a nucleic acid encoding an FBXW polypeptide.

A preferred method for increasing expression of a nucleic acid encoding a FBXW polypeptide is by introducing and expressing in a plant a nucleic acid encoding a FBXW polypeptide.

Any reference hereinafter to a “protein useful in the methods of the invention” is taken to mean a FBXW polypeptide as defined herein. Any reference hereinafter to a “nucleic acid useful in the methods of the invention” is taken to mean a nucleic acid capable of encoding such a FBXW polypeptide. The nucleic acid to be introduced into a plant (and therefore useful in performing the methods of the invention) is any nucleic acid encoding the type of protein which will now be described, hereafter also named “FBXWnucleic acid” or “FBXWgene”.

The term “FBXW polypeptide” as defined herein refers to a polypeptide comprising: (i) an F-box; (ii) a WD40 domain comprising at least one WD40 repeat; (iii) Motif 1 as represented by SEQ ID NO: 97; and (iv) Motif 2 as represented by SEQ ID NO: 98. Preferably, the sequence of Motif 1 is: WK (E/K) (F/V/L) Y (C/R/G) ERWGXP, X representing any amino acid.

The most conserved amino acids within Motif 1 are XLXFGXXXYFXWKXXYXERWGXP, and within Motif 2 SLXFEXPWLVSXSXDG (where X is a specified subset of amino acids differing for each position, as presented in SEQ ID NO: 97 and SEQ ID NO: 98). Within Motif 1 and Motif 2, are allowed one or more conservative change at any position, and/or one, two or three non-conservative change(s) at any position.

Optionally, the FBXW polypeptide may comprise any one or more of the following: (a) Motif 3 as represented by SEQ ID NO: 99; (b) Motif 4 as represented by SEQ ID NO: 100; and (c) Motif 5 as represented by SEQ ID NO: 101. Within Motifs 3 to 5, are allowed one or more conservative change at any position, and/or one or two non-conservative change(s) at any position.

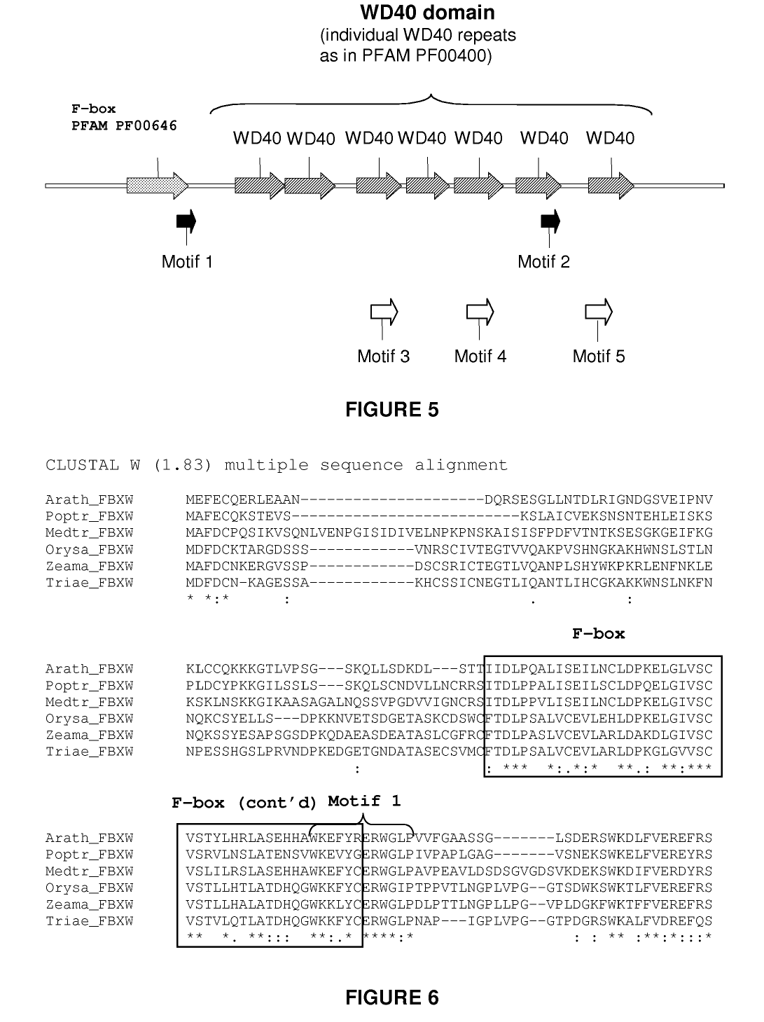

An example of an FBXW polypeptide as defined hereinabove comprising (i) an F-box; (ii) a WD40 domain comprising at least one WD40 repeat; (iii) Motif 1 as represented by SEQ ID NO: 97; and (iv) Motif 2 as represented by SEQ ID NO: 98; and optionally comprising any one or more of the following: (a) Motif 3 as represented by SEQ ID NO: 99; (b) Motif 4 as represented by SEQ ID NO: 100; and (c) Motif 5 as represented by SEQ ID NO: 101, is represented as in SEQ ID NO: 60 (FIG. 5 is a cartoon representing the different domains and their relative position in SEQ ID NO: 60). Further such examples are represented by any one of SEQ ID NO: 62, SEQ ID NO: 64, SEQ ID NO: 66, SEQ ID NO: 68 or SEQ ID NO: 70, or orthologues or paralogues of any of the aforementioned SEQ ID NOs. The invention is illustrated by transforming plants with the Arabidopsis thaliana sequence represented by SEQ ID NO: 59, encoding the polypeptide of SEQ ID NO: 60. SEQ ID NO: 62 (encoded by SEQ ID NO: 61, from Oryza sativa), SEQ ID NO: 64 (encoded by SEQ ID NO: 63, from Medicago trunculata), SEQ ID NO: 66 (encoded by SEQ ID NO: 65, from Triticum aestivum), SEQ ID NO: 68 (encoded by SEQ ID NO: 67, from Populus tremuloides) and SEQ ID NO: 70 (encoded by SEQ ID NO: 69, from Zea mays) are orthologues of the polypeptide of SEQ ID NO: 60.

Orthologues and paralogues (the terms being as defined above) may easily be found by performing a so-called reciprocal blast search. This may be done by a first BLAST involving BLASTing a query sequence (for example, SEQ ID NO: 59 or SEQ ID NO: 60) against any sequence database, such as the publicly available NCBI database. BLASTN or TBLASTX (using standard default values) may be used when starting from a nucleotide sequence and BLASTP or TBLASTN (using standard default values) may be used when starting from a polypeptide sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 59 or SEQ ID NO: 60, the second BLAST would therefore be against Arabidopsis sequences). The results of the first and second BLASTs are then compared. A paralogue is identified if a high-ranking hit from the first BLAST is from the same species as from which the query sequence is derived, a BLAST back then ideally results in the query sequence as highest hit (besides itself); an orthologue is identified if a high-ranking hit in the first BLAST is not from the same species as from which the query sequence is derived and preferably results upon BLAST back in the query sequence amongst the highest hits. High-ranking hits are those having a low E-value. The lower the E-value, the more significant the score (or in other words the lower the chance that the hit was found by chance). Computation of the E-value is well known in the art. In addition to E-values, comparisons are also scored by percentage identity. Percentage identity refers to the number of identical nucleotides (or amino acids) between the two compared nucleic acid (or polypeptide) sequences over a particular length. An example detailing the identification of orthologues and paralogues is given in Example 8. In the case of large families, ClustalW may be used, followed by a neighbour joining tree, to help visualize clustering of related genes and to identify orthologues and paralogues. Preferably, FBXW polypeptides useful in the methods of the invention comprise, in increasing order of preference, at least 45%, 50%, 55%, 65%, 70%, 75%, 80%, 85%, 90%, 95% or 98% sequence identity to SEQ ID NO: 60 (calculations shown in Example 9). FBXW polypeptides present relatively low amino acid sequence identity conservation between them, although their polypeptide structure (including the F-box and the WD40 domain) is well conserved. Sequence conservation between two more conserved regions of FBXW polypeptides as represented by SEQ ID NO: 102 and SEQ ID NO: 103 (both comprised within SEQ ID NO: 60) is in increasing order of preference, of at least 60%, 65%, 70%, 75%, 80%, 85%, 90%, 95% or 98% identity (calculations shwon in Example 9).

The polypeptides represented by any one of SEQ ID NO: 60, SEQ ID NO: 62, SEQ ID NO: 64,

SEQ ID NO: 66, SEQ ID NO: 68, SEQ ID NO: 70 or orthologues or paralogues of any of the aforementioned SEQ ID NOs, all comprise (i) an F-box; (ii) a WD40 domain comprising at least one WD40 repeat; (iii) Motif 1 as represented by SEQ ID NO: 97; and (iv) Motif 2 as represented by SEQ ID NO: 98.

The terms “domain” and “motif” are defined above. Special databases exisit for the identification of domains. The F-box and the WD40 repeats in a FBXW polypeptide may be identified using, for example, SMART (Schultz et al. (1998) Proc. Natl. Acad. Sci. USA 95, 5857-5864; Letunic et al. (2002) Nucleic Acids Res 30, 242-244; hosted by the EMBL at Heidelberg, Germany), InterPro (Mulder et al., (2003) Nucl. Acids. Res. 31, 315-318; hosted by the European Bioinformatics Institute (EBI) in the United Kingdom), Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAlPress, Menlo Park; Hulo et al., Nucl. Acids. Res. 32: D134-D137, (2004), The ExPASy proteomics server is provided as a service to the scientific community (hosted by the Swiss Institute of Bioinformatics (SIB) in Switzerland) or Pfam (Bateman et al., Nucleic Acids Research 30(1): 276-280 (2002), hosted by the Sanger Institute in the United Kingdom). The F-box comprises 40 to 50 residues, in which there are very few invariant positions. This lack of strict consensus makes identification using search algorithms essential. In the InterPro database, the F-box is designated by IPR001810, PF00646 in the Pfam database and PS50181 in the PROSITE database. The WD40 repeats comprised within the WD40 domain are typically of around 40 amino acids. Just as for the F-box, there are few invariant positions except that the repeat often (but not necessarily) ends with the Trp-Asp (W-D) dipeptide. Identification using search algorithms is equally essentially. In the InterPro database, the WD40 repeat is designated by IPR001680, PF00400 in the Pfam database and PS50082 in the PROSITE database. The WD40 domain typically comprise 4 to 16 repeats, preferably 5 to 10, more preferably 6 to 8, most preferably 7 repeats according to the PFAM algorithm (PF00400 repeats). The WD40 domain is designated by IPR0011046 in the InterPro database.

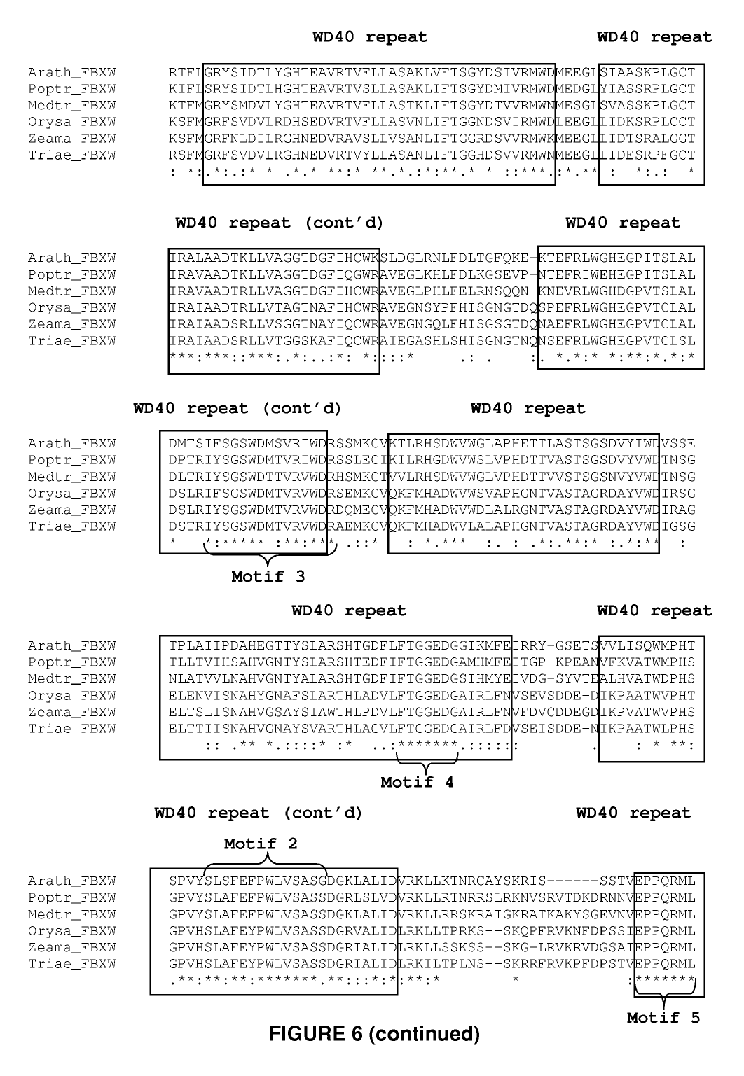

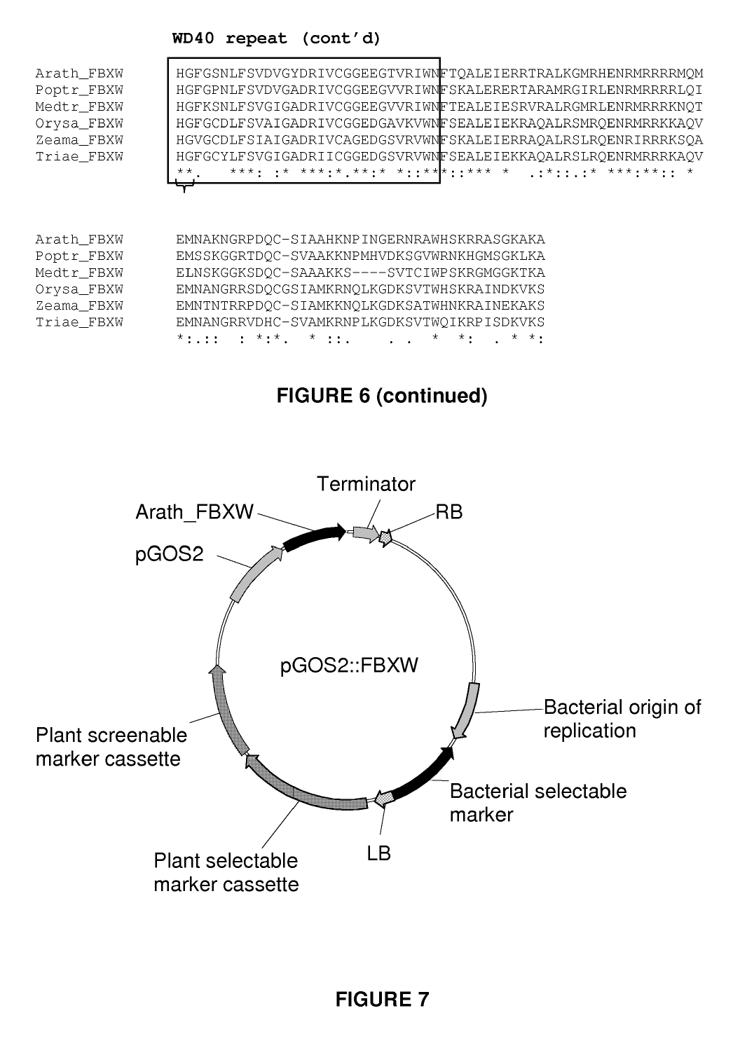

Methods for the alignment of sequences for comparison include GAP, BESTFIT, BLAST, FASTA and TFASTA. GAP uses the algorithm of Needleman and Wunsch ((1970) J Mol Biol 48: 443-453) to find the alignment of two complete sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information. Homologues may readily be identified using, for example, the ClustalW multiple sequence alignment algorithm (version 1.83) available at GenomeNet service at the Kyoto University Bioinformatics Center, with the default pairwise alignment parameters, and a scoring method in percentage. Minor manual editing may be performed to optimise alignment between conserved motifs, as would be apparent to a person skilled in the art. In some instances, default parameters may be adjusted to modify the stringency of the search. For example using BLAST, the statistical significance threshold (called “expect” value) for reporting matches against database sequences may be increased to show less stringent matches. In this way, short nearly exact matches may be identified. Motif 1 as represented by SEQ ID NO: 97 and Motif 2 as represented by SEQ ID NO: 98 both comprised in the FBXW polypeptides useful in the methods of the invention may be identified this way (FIG. 6). Within Motif 1 and Motif 2, are allowed one or more conservative change at any position, and/or one, two or three non-conservative change(s) at any position. The Motifs 3 to 5 (represented respectively by SEQ ID NO: 99, SEQ ID NO: 100 and SEQ ID NO: 101) may likewise be identified (FIG. 6). Within Motifs 3 to 5, are allowed one or more conservative change at any position, and/or one or two non-conservative change(s) at any position.

The nucleic acid encoding the polypeptides represented by any one of SEQ ID NO: 60, SEQ ID NO: 62, SEQ ID NO: 64, SEQ ID NO: 66, SEQ ID NO: 68, SEQ ID NO: 70, or orthologues or paralogues of any of the aforementioned SEQ ID NOs, need not be full-length nucleic acids, since performance of the methods of the invention does not rely on the use of full length nucleic acid sequences. Furthermore, examples of nucleic acids suitable for use in performing the methods of the invention include but are not limited to those represented by any one of: SEQ ID NO: 59, SEQ ID NO: 61, SEQ ID NO: 63, SEQ ID NO: 65, SEQ ID NO: 67 or SEQ ID NO: 69. Nucleic acid variants may also be useful in practising the methods of the invention. Examples of such variants include portions of nucleic acids, hybridising sequences, splice variants, allelic variants either naturally occurring or obtained by DNA manipulation.

A portion may be prepared, for example, by making one or more deletions to a nucleic acid encoding a FBXW polypeptide as defined hereinabove. The portions may be used in isolated form or they may be fused to other coding (or non coding) sequences in order to, for example, produce a protein that combines several activities. When fused to other coding sequences, the resultant polypeptide produced upon translation may be bigger than that predicted for the FBXW portion. Portions useful in the methods of the invention, encode an FBXW polypeptide (as described above) and having substantially the same biological activity as the FBXW polypeptide represented by any of SEQ ID NO: 60, SEQ ID NO: 62, SEQ ID NO: 64, SEQ ID NO: 66, SEQ ID NO: 68, SEQ ID NO: 70 or orthologues or paralogues of any of the aforementioned SEQ ID NOs. Examples of portions may include the nucleotides encoding a polypeptide comprising: (i) an F-box; (ii) a WD40 domain comprising at least one WD40 repeat; (iii) Motif 1 as represented by SEQ ID NO: 97; and (iv) Motif 2 as represented by SEQ ID NO: 98. Portions may optionally comprise any one or more of the following: (a) Motif 3 as represented by SEQ ID NO: 99; (b) Motif 4 as represented by SEQ ID NO: 100; and (c) Motif 5 as represented by SEQ ID NO: 101. The portion is typically at least 500 nucleotides in length, preferably at least 750 nucleotides in length, more preferably at least 1000 nucleotides in length and most preferably at least 1500 nucleotides in length. Preferably, the portion is a portion of a nucleic acid as represented by any one of SEQ ID NO: 59, SEQ ID NO: 61, SEQ ID NO: 63, SEQ ID NO: 65, SEQ ID NO: 67 or SEQ ID NO: 69. Most preferably the portion is a portion of a nucleic acid as represented by SEQ ID NO: 59.

Another nucleic acid variant useful in the methods of the invention, is a nucleic acid capable of hybridising under reduced stringency conditions, preferably under stringent conditions, with a nucleic acid encoding a FBXW polypeptide as defined hereinabove, or a with a portion as defined hereinabove.

The term “hybridisation” is defined in the Definitions section above. Hybridising sequences useful in the methods of the invention, encode a polypeptide comprising: (i) an F-box; (ii) a WD40 domain comprising at least one WD40 repeat; (iii) Motif 1 as represented by SEQ ID NO: 97; and (iv) Motif 2 as represented by SEQ ID NO: 98, and having substantially the same biological activity as the FBXW polypeptides represented by SEQ ID NO: 60, SEQ ID NO: 62, SEQ ID NO: 64, SEQ ID NO: 66, SEQ ID NO: 68, SEQ ID NO: 70, or orthologues or paralogues of any of the aforementioned SEQ ID NOs. The hybridising sequence is typically at least 250 nucleotides in length, preferably at least 500 nucleotides in length, more preferably at least 750 nucleotides in length, further preferably at least 1000 nucleotides in length, most preferably the hybridizing sequence is 1500 nucleotides in length. Preferably, the hybridising sequence is one that is capable of hybridising to any of the nucleic acids represented by SEQ ID NO: 59, SEQ ID NO: 61, SEQ ID NO: 63, SEQ ID NO: 65, SEQ ID NO: 67 or SEQ ID NO: 69 or to a portion of any of the aforementioned sequences, a portion being as defined above. Most preferably the hybridising sequence is capable of hybridising to SEQ ID NO: 59, or to portions thereof.

Portions encoding a FBXW polypeptide lacking one or more or part of: (i) an F-box; (ii) a WD40 domain comprising at least one WD40 repeat; (iii) Motif 1 as represented by SEQ ID NO: 97; and (iv) Motif 2 as represented by SEQ ID NO: 98, may be used for example, as a probe in the hybridisation process as described below, to obtain portions useful in performing the methods of the invention, comprising all of: (i) an F-box; (ii) a WD40 domain comprising at least one WD40 repeat; (iii) Motif 1 as represented by SEQ ID NO: 97; and (iv) Motif 2 as represented by SEQ ID NO: 98. Examples useful for the hybridisation process are represented by SEQ ID NO: 71 from Vitis vinifera (contig of NCBI ESTs CF210354, CF413646 and CF213082), SEQ ID NO: 73 from Senecio cambrensis (NCBI EST DY662683.1), SEQ ID NO: 75 from Helianthus annuus (NCBI EST DY916708), SEQ ID NO: 77 from Euphorbia esula (NCBI EST DV129599), SEQ ID NO: 79 from Lycopersicon esculentum (NCBI EST B1931509), SEQ ID NO: 81 from Aquilegia formosa×Aquilegia pubescens (NCBI EST DT753991.1), SEQ ID NO: 83 from Gossypium hirsutum (NCBI EST DT466-472), SEQ ID NO: 85 from Sorghum bicolor (NCBI EST CF770159), SEQ ID NO: 87 from Ipomea nil (NCBI EST BJ574759.1), SEQ ID NO: 89 from Solanum tuberosum (NCBI EST CX161187), SEQ ID NO: 91 from Zamia flscheri (NCBI EST DY032229), SEQ ID NO: 93 from Persea americana (NCBI EST CK756534) and SEQ ID NO: 95 from Glycine max (NCBI EST CD418593.1).

Another nucleic acid variant useful in the methods of the invention is a splice variant encoding a FBXW polypeptide as defined hereinabove. Preferred splice variants are splice variants of a nucleic acid encoding FBXW polypeptide represented by any of SEQ ID NO: 60, SEQ ID NO: 62, SEQ ID NO: 64, SEQ ID NO: 66, SEQ ID NO: 68, SEQ ID NO: 70, or splice variants encoding orthologues or paralogues of any of the aforementioned SEQ ID NOs. Further preferred are splice variants of nucleic acids represented by any one of SEQ ID NO: 59, SEQ ID NO: 61, SEQ ID NO: 63, SEQ ID NO: 65, SEQ ID NO: 67 or SEQ ID NO: 69. Most preferred is a splice variant of a nucleic acid as represented by SEQ ID NO: 59.

Another nucleic acid variant useful in performing the methods of the invention is an allelic variant of a nucleic acid encoding a FBXW polypeptide as defined hereinabove. Allelic variants exist in nature, and encompassed within the methods of the present invention is the use of these natural alleles. The allelic variants useful in the methods of the present invention have substantially the same biological activity as the FBXW polypeptide of SEQ ID NO: 60 and any of the amino acids depicted in Table G of Example 8. The allelic variant may be an allelic variant of a nucleic acid encoding a FBXW polypeptide represented by any of SEQ ID NO: 60, SEQ ID NO: 62, SEQ ID NO: 64, SEQ ID NO: 66, SEQ ID NO: 68, SEQ ID NO: 70, or an allelic variant of a nucleic acid encoding orthologues or paralogues of any of the aforementioned SEQ ID NOs. Further preferred are allelic variants of nucleic acids represented by any one of SEQ ID NO: 59, SEQ ID NO: 61, SEQ ID NO: 63, SEQ ID NO: 65, SEQ ID NO: 67 or SEQ ID NO: 69. Most preferred is an allelic variant of a nucleic acid as represented by SEQ ID NO: 59.

A further nucleic acid variant useful in the methods of the invention is a nucleic acid variant obtained by gene shuffling. Gene shuffling or directed evolution may also be used to generate variants of nucleic acids encoding FBXW polypeptides as defined above.

Furthermore, nucleic acid variants may also be obtained for example by site-directed mutagenesis. Several methods are available to achieve site-directed mutagenesis, the most common being PCR based methods (Current Protocols in Molecular Biology. Wiley (Eds)).

Also useful in the methods of the invention are nucleic acids encoding homologues of any one of the amino acids represented by SEQ ID NO: 60, SEQ ID NO: 62, SEQ ID NO: 64, SEQ ID NO: 66, SEQ ID NO: 68, SEQ ID NO: 70, or orthologues or paralogues of any of the aforementioned SEQ ID NOs. The terms “homologues”, “orthologues” and “paralogues” are as defined above.

Also useful in the methods of the invention are nucleic acids encoding derivatives of any one of the amino acids represented by SEQ ID NO: 60, SEQ ID NO: 62, SEQ ID NO: 64, SEQ ID NO: 66, SEQ ID NO: 68, SEQ ID NO: 70, or orthologues or paralogues of any of the aforementioned SEQ ID NOs.

Nucleic acids encoding FBXW polypeptides may be derived from any natural or artificial source. The nucleic acid may be modified from its native form in composition and/or genomic environment through deliberate human manipulation. Preferably the FBXW polypeptide-encoding nucleic acid is from a plant, further preferably from a dicotyledonous plant, more preferably from the Brassicaceae family, most preferably the nucleic acid is from Arabidopsis thaliana.

The invention also provides genetic constructs and vectors to facilitate introduction and/or expression of the nucleic acid sequences useful in the methods according to the invention, in a plant.

Therefore, there is provided a gene construct comprising:
(i) A nucleic acid encoding a FBXW polypeptide as defined hereinabove;
(ii) One or more control sequences operably liked to the nucleic acid of (i).

Constructs useful in the methods according to the present invention may be constructed using recombinant DNA technology well known to persons skilled in the art. The gene constructs may be inserted into vectors, which may be commercially available, suitable for transforming into plants and suitable for expression of the gene of interest in the transformed cells. The invention therefore provides use of a gene construct as defined hereinabove in the methods of the invention.

Plants are transformed with a vector comprising the sequence of interest (i.e., a nucleic acid encoding a FBXW polypeptide). The skilled artisan is well aware of the genetic elements that must be present on the vector in order to successfully transform, select and propagate host cells containing the sequence of interest. The sequence of interest is operably linked to one or more control sequences (at least to a promoter).

Advantageously, any type of promoter, whether natural or synthetic, may be used to drive expression of the nucleic acid sequence.

According to a preferred aspect of the invention, the nucleic acid encoding a FBXW polypeptide is operably linked to a constitutive promoter (a control sequence). The constitutive promoter is preferably a GOS2 (also named SUl1 or elF1 (eukaryotic initiation factor 1) promoter, more preferably the constitutive promoter is a rice GOS2 promoter, further preferably the constitutive promoter is represented by a nucleic acid sequence substantially similar to SEQ ID NO: 104 or SEQ ID NO: 58, most preferably the constitutive promoter is as represented by SEQ ID NO: 104 or SEQ ID NO: 58.

It should be clear that the applicability of the present invention is not restricted to the nucleic acid encoding an FBXW polypeptide as represented by SEQ ID NO: 59, nor is the applicability of the invention restricted to expression of a such nucleic acid encoding an FBXW polypeptide when driven by a GOS2 promoter.

Additional regulatory elements for increasing expression of nucleic acids or genes, or gene products, may include transcriptional as well as translational enhancers. Those skilled in the art will be aware of terminator and enhancer sequences that may be suitable for use in performing the invention. An example of such regulatory element is an intron introduced in the 5′ untranslated region. Optionally, one or more terminator sequences (also a control sequence) may be used in the construct introduced into a plant.

Other control sequences (besides promoter, enhancer, silencer, intron sequences, 3′UTR and/or 5′UTR regions) may be protein and/or RNA stabilizing elements. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

For the detection of the successful transfer of the nucleic acid sequences as used in the methods of the invention and/or selection of transgenic plants comprising these nucleic acids, it is advantageous to use marker genes (or reporter genes). Therefore genetic construct may optionally comprise a selectable marker gene. The marker genes may be removed or excised from the transgenic cell once they are no longer needed. Techniques for marker removal are known in the art, useful techniques are described above in the definitions section.

The invention also provides a method for the production of transgenic plants having increased yield relative to suitable control plants, comprising introduction and expression in a plant of a nucleic acid encoding a FBXW polypeptide as defined hereinabove.

More specifically, the present invention provides a method for the production of transgenic plants having increased yield relative to suitable control plants, which method comprises:
(i) introducing and expressing a nucleic acid encoding a FBXW polypeptide in a plant cell; and
(ii) cultivating the plant cell under conditions promoting plant growth and development.

The nucleic acid may be introduced directly into a plant cell or into the plant itself (including introduction into a tissue, organ or any other part of a plant). According to a preferred feature of the present invention, the nucleic acid is preferably introduced into a plant by transformation. The term “transformation” as referred to herein is defined above.

Generally after transformation, plant cells or cell groupings are selected for the presence of one or more markers which are encoded by plant-expressible genes co-transferred with the gene of interest, following which the transformed material is regenerated into a whole plant. The genetically modified plant cells can be regenerated via all methods with which the skilled worker is familiar. Suitable methods can be found in the abovementioned publications by S. D. Kung and R. Wu, Potrykus or Höfgen and Willmitzer. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may be evaluated, for instance using Southern analysis, for the presence of the gene of interest, copy number and/or genomic organisation. Alternatively or additionally, expression levels of the newly introduced DNA may be monitored using Northern and/or Western analysis, or quantitative PCR, all techniques being well known to persons having ordinary skill in the art.

The generated transformed plants may be propagated by a variety of means, such as by clonal propagation or classical breeding techniques. For example, a first generation (or T1) transformed plant may be self-pollinated to give homozygous second generation (or T2) transformants, and the T2 plants further propagated through classical breeding techniques.

The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

The invention also includes host cells containing an isolated nucleic acid encoding a FBXW polypeptide as defined hereinabove. Preferred host cells according to the invention are plant cells. Host plants for the nucleic acids or the vector used in the method according to the invention, the expression cassette or construct or vector are, in principle, advantageously all plants, which are capable of synthesizing the polypeptides used in the inventive method.

The invention also extends to harvestable parts of a plant such as, but not limited to seeds, leaves, fruits, flowers, stems, roots, rhizomes, tubers and bulbs. The invention furthermore relates to products derived, preferably directly derived, from a harvestable part of such a plant, such as dry pellets or powders, oil, fat and fatty acids, starch or proteins.

According to a preferred feature of the invention, the modulated expression is increased expression. Methods for increasing expression of nucleic acids or genes, or gene products, are well documented in the art and examples are provided in the definitions section.

As mentioned above, a preferred method for modulating (preferably, increasing) expression of a nucleic acid encoding a FBXW polypeptide is by introducing and expressing in a plant a nucleic acid encoding a FBXW polypeptide; however the effects of performing the method, i.e. enhancing yield-related traits may also be achieved using other well known techniques, including but not limited to T-DNA activation tagging, TILLING, homologous recombination. A description of these techniques is provided in the definitions section.

Performance of the methods of the invention gives plants having enhanced yield-related traits. In particular performance of the methods of the invention gives plants having increased yield, especially increased seed yield relative to control plants. The terms “yield” and “seed yield” are described in more detail in the “definitions” section herein.

Reference herein to enhanced yield-related traits is taken to mean an increase in biomass (weight) of one or more parts of a plant, which may include aboveground (harvestable) parts and/or (harvestable) parts below ground. In particular, such harvestable parts are seeds, and performance of the methods of the invention results in plants having increased seed yield relative to the seed yield of control plants.

Taking corn as an example, a yield increase may be manifested as one or more of the following: increase in the number of plants per hectare or acre, an increase in the number of ears per plant, an increase in the number of rows, number of kernels per row, kernel weight, thousand kernel weight, ear length/diameter, increase in the seed filling rate (which is the number of filled seeds divided by the total number of seeds and multiplied by 100), among others. Taking rice as an example, a yield increase may manifest itself as an increase in one or more of the following: number of plants per hectare or acre, number of panicles per plant, number of spikelets per panicle, number of flowers (florets) per panicle (which is expressed as a ratio of the number of filled seeds over the number of primary panicles), increase in the seed filling rate (which is the number of filled seeds divided by the total number of seeds and multiplied by 100), increase in thousand kernel weight, among others.

Since the transgenic plants according to the present invention have increased yield, it is likely that these plants exhibit an increased growth rate (during at least part of their life cycle), relative to the growth rate of control plants at a corresponding stage in their life cycle.

The increased growth rate may be specific to one or more parts of a plant (including seeds), or may be throughout substantially the whole plant. Plants having an increased growth rate may have a shorter life cycle. The life cycle of a plant may be taken to mean the time needed to grow from a dry mature seed up to the stage where the plant has produced dry mature seeds, similar to the starting material. This life cycle may be influenced by factors such as early vigour, growth rate, greenness index, flowering time and speed of seed maturation. The increase in growth rate may take place at one or more stages in the life cycle of a plant or during substantially the whole plant life cycle. Increased growth rate during the early stages in the life cycle of a plant may reflect enhanced vigour. The increase in growth rate may alter the harvest cycle of a plant allowing plants to be sown later and/or harvested sooner than would otherwise be possible (a similar effect may be obtained with earlier flowering time). If the growth rate is sufficiently increased, it may allow for the further sowing of seeds of the same plant species (for example sowing and harvesting of rice plants followed by sowing and harvesting of further rice plants all within one conventional growing period). Similarly, if the growth rate is sufficiently increased, it may allow for the further sowing of seeds of different plants species (for example the sowing and harvesting of corn plants followed by, for example, the sowing and optional harvesting of soybean, potato or any other suitable plant). Harvesting additional times from the same rootstock in the case of some crop plants may also be possible. Altering the harvest cycle of a plant may lead to an increase in annual biomass production per acre (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and T-90 (time taken for plants to reach 90% of their maximal size), amongst others.

According to a preferred feature of the present invention, performance of the methods of the invention gives plants having an increased growth rate relative to control plants. Therefore, according to the present invention, there is provided a method for increasing the growth rate of plants, which method comprises modulating expression, preferably increasing expression, in a plant of a nucleic acid encoding a FBXW polypeptide as defined herein.

An increase in yield and/or growth rate occurs whether the plant is under non-stress conditions or whether the plant is exposed to various stresses compared to control plants. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35% or 30%, preferably less than 25%, 20% or 15%, more preferably less than 14%, 13%, 12%, 11% or 10% or less in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the everyday biotic and/or abiotic (environmental) stresses to which a plant is exposed. Abiotic stresses may be due to drought or excess water, anaerobic stress, salt stress, chemical toxicity, oxidative stress and hot, cold or freezing temperatures. The abiotic stress may be an osmotic stress caused by a water stress (particularly due to drought), salt stress, oxidative stress or an ionic stress. Biotic stresses are typically those stresses caused by pathogens, such as bacteria, viruses, fungi and insects.

In particular, the methods of the present invention may be performed under non-stress conditions or under conditions of mild drought to give plants having increased yield relative to control plants. As reported in Wang et al. (Planta (2003) 218: 1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani et al. (Plant Physiol (2003) 133: 1755-1767) describes a particularly high degree of “cross talk” between drought stress and high-salinity stress. For example, drought and/or salinisation are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signalling pathways and cellular responses, such as the production of stress proteins, up-regulation of anti-oxidants, accumulation of compatible solutes and growth arrest. The term “non-stress” conditions as used herein are those environmental conditions that allow optimal growth of plants. Persons skilled in the art are aware of normal soil conditions and climatic conditions for a given location.

Performance of the methods of the invention gives plants grown under non-stress conditions or under mild drought conditions increased yield relative to control plants grown under comparable conditions. Therefore, according to the present invention, there is provided a method for increasing yield in plants grown under non-stress conditions or under mild drought conditions, which method comprises increasing expression in a plant of a nucleic acid encoding a FBXW polypeptide.

Performance of the methods of the invention gives plants grown under conditions of nutrient deficiency, particularly under conditions of nitrogen deficiency, increased yield relative to control plants grown under comparable conditions. Therefore, according to the present invention, there is provided a method for increasing yield in plants grown under conditions of nutrient deficiency, which method comprises increasing expression in a plant of a nucleic acid encoding a FBXW polypeptide. Nutrient deficiency may result from a lack of nutrients such as nitrogen, phosphates and other phosphorous-containing compounds, potassium, calcium, cadmium, magnesium, manganese, iron and boron, amongst others.

The methods of the invention are advantageously applicable to any plant. Plants that are particularly useful in the methods of the invention include all plants which belong to the superfamily Viridiplantae, in particular monocotyledonous and dicotyledonous plants including fodder or forage legumes, ornamental plants, food crops, trees or shrubs. According to a preferred embodiment of the present invention, the plant is a crop plant. Examples of crop plants include soybean, sunflower, canola, alfalfa, rapeseed, cotton, tomato, potato and tobacco. Further preferably, the plant is a monocotyledonous plant. Examples of monocotyledonous plants include sugarcane. More preferably the plant is a cereal. Examples of cereals include rice, maize, wheat, barley, millet, rye, triticale, sorghum and oats.

The present invention also encompasses plants obtainable by the methods according to the present invention. The present invention therefore provides plants, parts and cells from such plants obtainable by the methods according to the present invention, which plants or parts or cells comprise a nucleic acid transgene encoding a FBXW polypeptide as defined above.

The present invention also encompasses use of nucleic acids encoding FBXW polypeptides in increasing yield in a plant compared to yield in a suitable control plant.

One such use relates to increasing yield of plants, yield being defined as defined herein above. Yield may in particular include one or more of the following: increased seed yield, increased number of (filled) seeds, increased thousand kernel weight (TKW), increased harvest index and increased seed fill rate.

Nucleic acids encoding FBXW polypeptides may find use in breeding programmes in which a DNA marker is identified which may be genetically linked to a gene encoding FBXW polypeptide. Nucleic acids encoding FBXW polypeptides may be used to define a molecular marker. This marker may then be used in breeding programmes to select plants having increased seed yield. The nucleic acids encoding FBXW polypeptides may be, for example, a nucleic acid as represented by any one of SEQ ID NO: 59, SEQ ID NO: 61, SEQ ID NO: 63, SEQ ID NO: 65, SEQ ID NO: 67 or SEQ ID NO: 69.

Allelic variants of a nucleic acid encoding an FBXW polypeptide may also find use in marker-assisted breeding programmes. Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give increased seed yield. Selection is typically carried out by monitoring growth performance of plants containing different allelic variants of the sequence in question, for example, different allelic variants of any one of SEQ ID NO: 59, SEQ ID NO: 61, SEQ ID NO: 63, SEQ ID NO: 65, SEQ ID NO: 67 or SEQ ID NO: 69. Growth performance may be monitored in a greenhouse or in the field. Further optional steps include crossing plants, in which the superior allelic variant was identified, with another plant. This could be used, for example, to make a combination of interesting phenotypic features.

Nucleic acids encoding FBXW polypeptides may also be used as probes for genetically and physically mapping the genes that they are a part of, and as markers for traits linked to those genes. Such information may be useful in plant breeding in order to develop lines with desired phenotypes. Such use of nucleic acids encoding FBXW polypeptides requires only a nucleic acid sequence of at least 15 nucleotides in length. The nucleic acids encoding FBXW polypeptides may be used as restriction fragment length polymorphism (RFLP) markers. Southern blots (Sambrook J, Fritsch E F and Maniatis T (1989) Molecular Cloning, A Laboratory Manual) of restriction-digested plant genomic DNA may be probed with a nucleic acid encoding FBXW polypeptide. The resulting banding patterns may then be subjected to genetic analyses using computer programs such as MapMaker (Lander et al. (1987) Genomics 1: 174-181) in order to construct a genetic map. In addition, the nucleic acid may be used to probe Southern blots containing restriction endonuclease-treated genomic DNAs of a set of individuals representing parent and progeny of a defined genetic cross. Segregation of the DNA polymorphisms is noted and used to calculate the position of the nucleic acid encoding FBXW polypeptide in the genetic map previously obtained using this population (Botstein et al. (1980) Am. J. Hum. Genet. 32: 314-331).

The production and use of plant gene-derived probes for use in genetic mapping is described in Bernatzky and Tanksley (GENETICS112 (4): 887-898, 1986). Numerous publications describe genetic mapping of specific cDNA clones using the methodology outlined above or variations thereof. For example, F2 intercross populations, backcross populations, randomly mated populations, near isogenic lines (NIL), and other sets of individuals may be used for mapping. Such methodologies are well known to those skilled in the art.

The nucleic acid probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid probes may be used in direct fluorescence in situ hybridization (FISH) mapping (Trask (1991) Trends Genet. 7:149-154). Although current methods of FISH mapping favour use of large clones (several kb to several hundred kb; see Laan et al. (1995) Genome Res. 5:13-20), improvements in sensitivity may allow performance of FISH mapping using shorter probes.

A variety of nucleic acid amplification-based methods for genetic and physical mapping may be carried out using the nucleic acids. Examples include allele-specific amplification (Kazazian (1989) J. Lab. Clin. Med. 11:95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic Acid Res. 18:3671), Radiation Hybrid Mapping (Walter et al. (1997) Nat. Genet. 7:22-28) and Happy Mapping (Dear and Cook (1989) Nucleic Acid Res. 17:6795-6807). For these methods, the sequence of a nucleic acid is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

The methods according to the present invention result in plants having increased yield, as described hereinbefore. These traits may also be combined with other economically advantageous traits, such as further yield-increasing traits, tolerance to other abiotic and biotic stresses, traits modifying various architectural features and/or biochemical and/or physiological features.

### RANBP

Upon investigating the use of RAN-binding proteins to enhance yield-related traits, the inventors named in this application found the choice of promoter to be an important consideration. They found that expressing RAN-binding proteins in a (rice) plant under the control of a constitute promoter did not have any effect on yield-related phenotypes. They surprisingly found that plant yield could successfully be increased by expressing RAN-binding proteins in a plant under the control of a seed-specific promoter, particularly an endosperm-specific promoter.

The present invention therefore provides a method for enhancing yield-related traits in plants relative to control plants, comprising preferentially modulating expression in plant seed or seed parts of a nucleic acid encoding a RANBP.

A preferred method for modulating (preferably, increasing) expression in plant seed or seed parts of a nucleic acid encoding a RANBP is by introducing and expressing in a plant a nucleic acid encoding a RANBP under the control of a seed-specific promoter.

Any reference hereinafter to a “protein useful in the methods of the invention” is taken to mean a RANBP polypeptide as defined herein. Any reference hereinafter to a “nucleic acid useful in the methods of the invention” is taken to mean a nucleic acid capable of encoding such a RANBP polypeptide. The nucleic acid to be introduced into a plant (and therefore useful in performing the methods of the invention) is any nucleic acid encoding the type of protein which will now be described, hereafter also named “RANBP nucleic acid” or “RANBP gene”.

Nucleic acids suitable for introducing into a plant (and therefore useful in performing the methods of the invention) include any nucleic acid encoding a RANBP having motif I: KSC V/L WHAXDF A/S DGELK D/E EXF, where ‘X’ is any amino acid, allowing zero or one conservative change at any position and/or zero one, two or three non-conservative change(s) at any position.

In the case of RANBPs from monocotyledonous plants, the C-terminus of Motif I often ends in ‘AIRFG’, and in the case of RANBPs from dicotyledonous plants, the C-terminus of Motif I often ends in ‘CIRFA’.

RANBP-encoding nucleic acids useful in the methods of the invention may also comprise (in addition to Motif I) any one or more of the following motifs.
1. Motif II as represented by SEQ ID NO: 139 or 145 or a motif having in increasing order of preference at least 60%, 70%, 80%, 90% or more percentage sequence identity to
Motif II represented by SEQ ID NO: 139 or 145; 2. Motif III as represented by represented by SEQ ID NO: 140 or 146 or a motif having in increasing order of preference at least 70%, 80%, 90% or more percentage sequence identity to Motif III as represented by SEQ ID NO: 140 or 146;
3. Motif IV as represented by SEQ ID NO: 141 or 147 allowing for zero or one conservative change at any position and/or zero or one non-conservative change at any position;
4. Motif V as represented by SEQ ID NO: 142 or 148 or a motif having in increasing order of preference at least 70%, 80%, 90% or more percentage sequence identity to Motif V as represented by SEQ ID NO: 142 or 148;
5. Motif VI as represented by SEQ ID NO: 143 or 149 allowing for zero or one conservative change at any position and/or zero or one non-conservative change at any position;
6. Motif VII as represented by SEQ ID NO: 144 or 150 or a motif having in increasing order of preference at least 60%, 70%, 80%, 90% or more percentage sequence identity to Motif VII represented by SEQ ID NO: 144 or 150.

The aforementioned motifs represent amino acids conserved at specific positions along an alignment of sequences of evolutionarily related proteins. Whilst amino acids at other positions may vary between homologues, amino acids that are highly conserved at specific positions indicate amino acids that are likely essential to the structure, stability or activity of the protein. Identified by their high degree of conservation in aligned sequences of a family of protein homologues, they can be used as identifiers to determine if any polypeptide in question belongs to a previously identified polypeptide family (in this case, the family of RNABPs).

The various motifs mentioned above may readily be identified using methods for the alignment of sequences for comparison. In some instances, default parameters may be adjusted to modify the stringency of the search. For example using BLAST, the statistical significance threshold (called E-value) for reporting matches against database sequences may be increased to show less stringent matches. In this way, short nearly exact matches may be identified.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (over the whole the sequence) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences.). Minor manual editing may be performed to optimise alignment between conserved motifs, as would be apparent to a person skilled in the art. Furthermore, instead of using full-length sequences for the identification of homologues, specific domains may also be used. The sequence identity values may be determined over the entire nucleic acid or amino acid sequence or over selected domains or conserved motif(s), using the programs mentioned above using the default parameters.

All RanBP1 proteins contain an approximately 150 amino acid residue Ran binding domain. Specialist databases exist for the identification of domains. Domains in RANBPs may be identified using, for example, SMART (Schultz et al. (1998) Proc. Natl. Acad. Sci. USA 95, 5857-5864; Letunic et al. (2002) Nucleic Acids Res 30, 242-244), InterPro (Mulder et al., (2003) Nucl. Acids. Res. 31, 315-318), Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAIPress, Menlo Park; Hulo et al., Nucl. Acids. Res. 32:D134-D137, (2004),) or Pfam (Bateman et al., Nucleic Acids Research 30(1): 276-280 (2002). A set of tools for in silico analysis of protein sequences is available on the ExPASy proteomics server (Swiss Institute of Bioinformatics (Gasteiger et al., ExPASy: the proteomics server for in-depth protein knowledge and analysis, Nucleic Acids Res. 31:3784-3788 (2003)). Domains or motifs may also be identified using routine techniques, such as by sequence alignment.

The invention is illustrated (see the Examples section) by transforming plants with a RANBP from Zea mays as represented by SEQ ID NO: 113, encoding the polypeptide of SEQ ID NO: 114 or SEQ ID NO: 115. The invention is also illustrated by transforming plants with a RANBP from Arabidopsis thaliana as represented by SEQ ID NO: 116, encoding the polypeptide of SEQ ID NO: 117 or SEQ ID NO: 118.

Of course performance of the methods of the invention is not restricted to the use of the aforementioned sequences, but may be performed using any nucleic acid encoding a RANBP comprising Motif I, as defined hereinabove. Examples of such nucleic acids encoding RANBPs comprising Motif I include nucleic acids encoding homologues, orthologues and paralogues of SEQ ID NO: 114, SEQ ID NO: 115, SEQ ID NO: 117 and SEQ ID NO: 118; the terms homologues, orthologues and paralogues being as defined above. Examples of such homologues, orthologues and paralogues include the sequences listed in Table P of Example 14.

Orthologues and paralogues may easily be found by performing a so-called reciprocal blast search. Typically this involves a first BLAST involving BLASTing a query sequence (for example, SEQ ID NO: 113, SEQ ID NO: 114, SEQ ID NO: 115, SEQ ID NO: 116, SEQ ID NO: 117 or SEQ ID NO: 118) against any sequence database, such as the publicly available NCBI database. BLASTN or TBLASTX (using standard default values) is generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 113, SEQ ID NO: 114 or SEQ ID NO: 115, the second BLAST would be against Zea mays sequences; where the query sequence is SEQ ID NO: 116, SEQ ID NO: 117 or SEQ ID NO: 118, the second BLAST would be against Arabidopsis thaliana sequences). The results of the first and second BLASTs are then compared. A paralogue is identified if a high-ranking hit from the first blast is from the same species as from which the query sequence is derived, a BLAST back then ideally results in the query sequence as highest hit; an orthologue is identified if a high-ranking hit in the first BLAST is not from the same species as from which the query sequence is derived, and preferably results upon BLAST back in the query sequence being among the highest hits.

High-ranking hits are those having a low E-value. The lower the E-value, the more significant the score (or in other words the lower the chance that the hit was found by chance). Computation of the E-value is well known in the art. In addition to E-values, comparisons are also scored by percentage identity. Percentage identity refers to the number of identical nucleotides (or amino acids) between the two compared nucleic acid (or polypeptide) sequences over a particular length. In the case of large families, ClustalW may be used, followed by a neighbour joining tree, to help visualize clustering of related genes and to identify orthologues and paralogues.

Nucleic acid variants may also be useful in practising the methods of the invention. Examples of such variants include nucleic acids encoding homologues and derivatives of any one of the amino acid sequences given in Table P of Example 14, the terms “homologue” and “derivative” being as defined herein. Also useful in the methods of the invention are nucleic acids encoding homologues and derivatives of orthologues or paralogues of any one of the amino acid sequences given in Table P of Example 14. Homologues and derivatives useful in the methods of the present invention have substantially the same biological and functional activity as the unmodified protein from which they are derived.

Typically, nucleic acids encoding RANBPs comprising at least Motif I have, in increasing order of preference, at least 50%, 55%, 60%, 65%, 70%, 75%, 80%, 85%, 90%, 95% or more sequence identity to the nucleic acid sequence represented by SEQ ID NO: 113 or SEQ ID NO: 116.

Also useful in the methods of the invention are nucleic acids encoding derivatives of the amino acids represented by SEQ ID NO 114, SEQ ID NO: 115, SEQ ID NO: 117 or SEQ ID NO 118 or nucleic acids encoding derivatives of the orthologues or paralogues of SEQ ID NO 114, SEQ ID NO: 115, SEQ ID NO: 117 or SEQ ID NO 118.

Nucleic acids encoding the polypeptides represented by the sequences in Table P, or nucleic acids encoding orthologues or paralogues of any of these SEQ ID NOs, need not be full-length nucleic acids, since performance of the methods of the invention does not rely on the use of full length nucleic acid sequences. Examples of nucleic acids suitable for use in performing the methods of the invention include, but are not limited to those represented in Table P of Example 14. Nucleic acid variants may also be useful in practising the methods of the invention. Examples of such nucleic acid variants include portions of nucleic acids encoding a RANBP, splice variants of nucleic acids encoding a RANBP, sequences hybridising to nucleic acids encoding a RANBP, allelic variants of nucleic acids encoding a RANBP and variants of nucleic acids encoding a RANBP designed by gene shuffling. The terms splice variant and allelic variant are described above.

A portion of a nucleic acid encoding a RANBP may be prepared, for example, by making one or more deletions to the nucleic acid. The portions may be used in isolated form or they may be fused to other coding (or non-coding) sequences in order to, for example, produce a protein that combines several activities. When fused to other coding sequences, the resultant polypeptide produced upon translation may be bigger than that predicted for the RANBP portion.

Portions useful in the methods of the invention, encode a polypeptide comprising Motif I as described above and having substantially the same biological activity as the RANBP represented by the sequences listed in Table P, or orthologues or paralogues of any of the aforementioned SEQ ID NOs. The portion is typically at least 200 consecutive nucleotides in length, preferably at least 300 consecutive nucleotides in length, more preferably at least 400 consecutive nucleotides in length. Preferably, the portion is a portion of a nucleic acid as represented by any one of the sequences listed in Table P. Most preferably the portion is a portion of a nucleic acid as represented by SEQ ID NO: 113 or SEQ ID NO: 116.

According to the present invention, there is provided a method for enhancing yield-related traits in plants, comprising introducing and preferentially expressing in plant seed or seed parts a portion of a nucleic acid represented by any of the sequences listed in Table P.

Another nucleic acid variant useful in the methods of the invention, is a nucleic acid capable of hybridising under reduced stringency conditions, preferably under stringent conditions, with a nucleic acid encoding a RANBP as defined herein, or a with a portion as defined herein.

Hybridising sequences useful in the methods of the invention, encode a polypeptide comprising Motif I and having substantially the same biological activity as the RANBP represented by any of the sequences listed in Table P, or having substantially the same biological activity as orthologues or paralogues of any of the aforementioned SEQ ID NOs. The hybridising sequence is typically at least 200 consecutive nucleotides in length, preferably at least 300 consecutive nucleotides in length, more preferably at least 400 consecutive nucleotides in length. Preferably, the hybridising sequence is one that is capable of hybridising to any of the nucleic acids represented by the sequences listed in Table P, or to a portion of any of the aforementioned sequences, a portion being as defined above. Most preferably the hybridising sequence is capable of hybridising to a nucleic acid as represented by SEQ ID NO: 113 or 116, or to portions thereof.

According to the present invention, there is provided a method for enhancing yield-related traits in plants, comprising introducing and preferentially expressing in plant seed or seed parts a nucleic acid capable of hybridizing to a nucleic acid encoding a RANBP represented by any of the sequences listed in Table P, or comprising introducing and preferentially expressing in plant seed or seed parts a nucleic acid capable of hybridising to a nucleic acid encoding an orthologue, paralogue or homologue of any of the aforementioned SEQ ID NOs.

Another nucleic acid variant useful in the methods of the invention is a splice variant encoding a RANBP as defined hereinabove. According to the present invention, there is provided a method for enhancing yield-related traits in plants, comprising introducing and preferentially expressing in plant seed or seed parts a splice variant of a nucleic acid encoding a RANBP represented by any of the sequences listed in Table P, or a splice variant of a nucleic acid encoding an orthologue, paralogue or homologue of any of the aforementioned SEQ ID NOs.

Preferred splice variants are splice variants of a nucleic acid encoding a RANBP represented by any of SEQ ID NO: 114, SEQ ID NO 115, SEQ ID NO: 117 or SEQ ID NO: 118. Further preferred are splice variants of nucleic acids represented by any one of the sequences listed in Table P. Most preferred is a splice variant of a nucleic acid as represented by SEQ ID NO: 113 or SEQ ID NO: 116.

Another nucleic acid variant useful in performing the methods of the invention is an allelic variant of a nucleic acid encoding a RANBP as defined hereinabove. Allelic variants exist in nature, and encompassed within the methods of the present invention is the use of these natural alleles.

According to the present invention, there is provided a method for enhancing yield-related traits in plants, comprising introducing and preferentially expressing in plant seed or seed parts an allelic variant of a nucleic acid encoding a RANBP represented by any of the sequences listed in Table P, or comprising introducing and expressing in a plant an allelic variant of a nucleic acid encoding an orthologue, paralogue or homologue of any of the aforementioned SEQ ID NOs.

The allelic variant may be an allelic variant of a nucleic acid encoding a RANBP represented by any of SEQ ID NO: 114, SEQ ID NO 115, SEQ ID NO: 117 or SEQ ID NO: 118, or an allelic variant of a nucleic acid encoding orthologues or paralogues of any of the aforementioned SEQ ID NOs. Further preferred are allelic variants of nucleic acids represented by any one of the sequences listed in Table P. Most preferred is an allelic variant of a nucleic acid as represented by SEQ ID NO: 113 or 116.

A further nucleic acid variant useful in the methods of the invention is a nucleic acid variant designed and/or obtained by gene shuffling. Gene shuffling or directed evolution may be used to generate variants of nucleic acids encoding RANBPs as defined above.

According to the present invention, there is provided a method for enhancing yield-related traits in plants, comprising introducing and preferentially expressing in plant seed or seed parts a variant of a nucleic acid represented by any of the sequences listed in Table P, which variant nucleic acid is designed and/or obtained by gene shuffling.

Furthermore, nucleic acid variants may also be obtained by site-directed mutagenesis. Several methods are available to achieve site-directed mutagenesis, the most common being PCR based methods (current protocols in molecular biology. Wiley Eds.).

Nucleic acids encoding RANBPs may be derived from any natural or artificial source. The nucleic acid may be modified from its native form in composition and/or genomic environment through deliberate human manipulation. According to one preferred embodiment the RANBP-encoding nucleic acid is from a plant, further preferably from a monocot, more preferably from the family Poaceae, more preferably from the genus Zea, most preferably from Zea mays.

According to a further preferred embodiment, the RANBP-encoding nucleic acid is from a plant, further preferably from a dicotyledonous plant, further preferably from the family Brassicaceae, more preferably the nucleic acid is from Arabidopsis thaliana.

The present invention also encompasses plants or parts thereof obtainable by the methods according to the present invention. The plants or parts thereof comprise a nucleic acid transgene encoding a RANBP operably linked to a seed-specific promoter.

The invention also provides genetic constructs and vectors to facilitate introduction and/or expression of the nucleic acid sequences useful in the methods according to the invention, in a plant.

Therefore, there is provided a gene construct comprising:
(i) A nucleic acid encoding a RANBP comprising Motif I as defined hereinabove;
(ii) A seed-specific promoter operably liked to the nucleic acid of (i).

Constructs useful in the methods according to the present invention may be constructed using recombinant DNA technology well known to persons skilled in the art. The gene constructs may be inserted into vectors, which may be commercially available, suitable for transforming into plants and suitable for expression of the gene of interest in the transformed cells. The invention therefore provides use of a gene construct as defined hereinabove in the methods of the invention.

Plants are transformed with a vector comprising the sequence of interest (i.e., a nucleic acid encoding a RANBP). The skilled artisan is well aware of the genetic elements that must be present on the vector in order to successfully transform, select and propagate host cells containing the sequence of interest. The sequence of interest is operably linked to one or more control sequences (at least to a promoter). The terms “regulatory element”, “control sequence” and “promoter” are all used interchangeably herein and are defined above.

The nucleic acid encoding a RANBP is operably linked to a seed-specific promoter, i.e. a promoter that is expressed predominantly in seed tissue, but which may have residual expression elsewhere in the plant due to leaky promoter expression. Further preferably, the seed-specific promoter is isolated from a gene encoding a seed-storage protein, especially an endosperm-specific promoter. An endosperm-specific promoter refers to any promoter able to preferentially drive expression of the gene of interest in endosperm tissue. Reference herein to “preferentially” driving expression in endosperm tissue is taken to mean driving expression of any sequence operably linked thereto in endosperm tissue substantially to the exclusion of driving expression elsewhere in the plant, apart from any residual expression due to leaky promoter expression. For example, the prolamin promoter shows strong expression in the endosperm, with leakiness in meristem, more specifically the shoot meristem and/or discrimination centre in the meristem. Most preferably the endosperm-specific promoter is isolated from a prolamin gene, such as a rice prolamin RP6 (Wen et al., (1993) Plant Physiol 101(3): 1115-6) promoter as represented by SEQ ID NO: 155, or a promoter of similar strength and/or a promoter with a similar expression pattern as the rice prolamin promoter. Examples of other endosperm-specific promoters which may also be used perform the methods of the invention are shown in Table 2c above.

It should be clear that the applicability of the present invention is not restricted to the RANBP-encoding nucleic acid represented by SEQ ID NO: 113 or SEQ ID NO: 116, nor is the applicability of the invention restricted to expression of a such a RANBP-encoding nucleic acid when driven by a prolamin promoter. Examples of other seed-specific promoters which may also be used perform the methods of the invention are shown in Table 2b above.

Optionally, one or more terminator sequences (also a control sequence) may be used in the construct introduced into a plant. Additional regulatory elements may include transcriptional as well as translational enhancers. Those skilled in the art will be aware of terminator and enhancer sequences that may be suitable for use in performing the invention. Other control sequences, besides promoter, enhancer, silencer, intron sequences, 3′UTR and/or 5′UTR regions, may be protein and/or RNA stabilizing elements. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

For the detection of the successful transfer of the nucleic acid sequences as used in the methods of the invention and/or selection of transgenic plants comprising these nucleic acids, it is advantageous to use marker genes (or reporter genes). Therefore, the genetic construct may optionally comprise a selectable marker gene. The marker genes may be removed or excised from the transgenic cell once they are no longer needed. Techniques for marker removal are known in the art, useful techniques are described above in the definitions section.

The invention also provides a method for the production of transgenic plants having enhanced yield-related traits relative to control plants, comprising introduction and preferential expression in plant seed or seed parts of a nucleic acid encoding a RANBP comprising Motif I as defined hereinabove.

More specifically, the present invention provides a method for the production of transgenic plants having enhanced yield-related traits, which method comprises:
(i) introducing and expressing in a plant cell a nucleic acid encoding a RANBP comprising Motif I (as defined herein) operably linked to seed-specific promoter; and
(ii) cultivating the plant cell under conditions promoting plant growth and development.

The nucleic acid may be introduced directly into a plant cell or into the plant itself (including introduction into a tissue, organ or any other part of a plant). According to a preferred feature of the present invention, the nucleic acid is preferably introduced into a plant by transformation.

The term “transformation” as referred to herein is described above.

The genetically modified plant cells can be regenerated via all methods with which the skilled worker is familiar. Suitable methods can be found in the abovementioned publications by S. D. Kung and R. Wu, Potrykus or Höfgen and Willmitzer.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may be evaluated, for instance using Southern analysis, for the presence of the gene of interest, copy number and/or genomic organisation. Alternatively or additionally, expression levels of the newly introduced DNA may be monitored using Northern and/or Western analysis, or quantitative PCR, all techniques being well known to persons having ordinary skill in the art.

The generated transformed plants may be propagated by a variety of means, such as by clonal propagation or classical breeding techniques. For example, a first generation (or T1) transformed plant may be selfed to give homozygous second generation (or T2) transformants, and the T2 plants further propagated through classical breeding techniques.

The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

The invention also includes host cells containing an isolated nucleic acid encoding a RANBP comprising Motif I as defined hereinabove. Preferred host cells according to the invention are plant cells. Host plants for the nucleic acids or the vector used in the method according to the invention, the expression cassette or construct or vector are, in principle, advantageously all plants, which are capable of synthesizing the polypeptides used in the inventive method.

The invention also extends to harvestable parts of a plant such as, but not limited to seeds, leaves, fruits, flowers, stems, roots, rhizomes, tubers and bulbs. The invention furthermore relates to products derived, preferably directly derived, from a harvestable part of such a plant, such as dry pellets or powders, oil, fat and fatty acids, starch or proteins.

According to a preferred feature of the invention, the modulated expression is increased expression. Methods for increasing expression of nucleic acids or genes, or gene products, are well documented in the art and examples are provided in the definitions section.

As mentioned above, a preferred method for preferentially modulating (preferably, increasing) expression in plant seed or seed parts of a nucleic acid encoding a RANBP is by introducing and expressing in a plant a nucleic acid encoding a RANBP comprising Motif I; however the effects of performing the method, i.e. enhancing yield-related traits may also be achieved using other well known techniques, including but not limited to T-DNA activation tagging, TILLING, homologous recombination. A description of some of these techniques is provided in the definitions section.

The effects of the invention may also be reproduced using homologous recombination. The nucleic acid to be targeted is preferably the region controlling the natural expression of a nucleic acid encoding a RANBP in a plant. A seed-specific promoter is introduced into this region, replacing substantially part or all of it.

Performance of the methods of the invention gives plants having enhanced yield-related traits. In particular performance of the methods of the invention gives plants having increased yield, especially increased biomass and seed yield relative to control plants. The terms “yield” and “seed yield” are described in more detail in the “definitions” section herein.

Reference herein to enhanced yield-related traits is taken to mean an increase in biomass (weight) of one or more parts of a plant, which may include aboveground (harvestable) parts and/or (harvestable) parts below ground. In particular, such harvestable parts are above ground biomass and seeds, and performance of the methods of the invention results in plants having increased biomass and increased seed yield relative to the seed yield of control plants.

Taking corn as an example, a yield increase may be manifested as one or more of the following: increase in the number of plants established per hectare or acre, an increase in the number of ears per plant, an increase in the number of rows, number of kernels per row, kernel weight, thousand kernel weight, ear length/diameter, increase in the seed filling rate (which is the number of filled seeds divided by the total number of seeds and multiplied by 100), among others. Taking rice as an example, a yield increase may manifest itself as an increase in one or more of the following: number of plants per hectare or acre, number of panicles per plant, number of spikelets per panicle, number of flowers (florets) per panicle (which is expressed as a ratio of the number of filled seeds over the number of primary panicles), increase in the seed filling rate (which is the number of filled seeds divided by the total number of seeds and multiplied by 100), increase in thousand kernel weight, among others.

The present invention provides a method for increasing yield, especially seed yield of plants, relative to control plants, which method comprises modulating expression, preferably increasing expression, in a plant of a nucleic acid encoding a RANBP polypeptide as defined herein.

Since the transgenic plants according to the present invention have increased yield, it is likely that these plants exhibit an increased growth rate (during at least part of their life cycle), relative to the growth rate of control plants at a corresponding stage in their life cycle.

The increased growth rate may be specific to one or more parts of a plant (including seeds), or may be throughout substantially the whole plant. Plants having an increased growth rate may have a shorter life cycle. The life cycle of a plant may be taken to mean the time needed to grow from a dry mature seed up to the stage where the plant has produced dry mature seeds, similar to the starting material. This life cycle may be influenced by factors such as early vigour, growth rate, greenness index, flowering time and speed of seed maturation. The increase in growth rate may take place at one or more stages in the life cycle of a plant or during substantially the whole plant life cycle. Increased growth rate during the early stages in the life cycle of a plant may reflect enhanced vigour. The increase in growth rate may alter the harvest cycle of a plant allowing plants to be sown later and/or harvested sooner than would otherwise be possible (a similar effect may be obtained with earlier flowering time). If the growth rate is sufficiently increased, it may allow for the further sowing of seeds of the same plant species (for example sowing and harvesting of rice plants followed by sowing and harvesting of further rice plants all within one conventional growing period). Similarly, if the growth rate is sufficiently increased, it may allow for the further sowing of seeds of different plants species (for example the sowing and harvesting of corn plants followed by, for example, the sowing and optional harvesting of soybean, potato or any other suitable plant). Harvesting additional times from the same rootstock in the case of some crop plants may also be possible. Altering the harvest cycle of a plant may lead to an increase in annual biomass production per acre (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and T-90 (time taken for plants to reach 90% of their maximal size), amongst others.

According to a preferred feature of the present invention, performance of the methods of the invention gives plants having an increased growth rate relative to control plants. Therefore, according to the present invention, there is provided a method for increasing the growth rate of plants, which method comprises modulating expression, preferably increasing expression, in a plant of a nucleic acid encoding a RANBP polypeptide as defined herein.

An increase in yield and/or growth rate occurs whether the plant is under non-stress conditions or whether the plant is exposed to various stresses compared to control plants. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35% or 30%, preferably less than 25%, 20% or 15%, more preferably less than 14%, 13%, 12%, 11% or 10% or less in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the everyday biotic and/or abiotic (environmental) stresses to which a plant is exposed. Abiotic stresses may be due to drought or excess water, anaerobic stress, salt stress, chemical toxicity, oxidative stress and hot, cold or freezing temperatures. The abiotic stress may be an osmotic stress caused by a water stress (particularly due to drought), salt stress, oxidative stress or an ionic stress. Biotic stresses are typically those stresses caused by pathogens, such as bacteria, viruses, fungi and insects.

In particular, the methods of the present invention may be performed under non-stress conditions or under conditions of mild drought to give plants having increased yield relative to control plants. As reported in Wang et al. (Planta (2003) 218: 1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani et al. (Plant Physiol (2003) 133: 1755-1767) describes a particularly high degree of “cross talk” between drought stress and high-salinity stress. For example, drought and/or salinisation are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signalling pathways and cellular responses, such as the production of stress proteins, up-regulation of anti-oxidants, accumulation of compatible solutes and growth arrest. The term “non-stress” conditions as used herein are those environmental conditions that allow optimal growth of plants. Persons skilled in the art are aware of normal soil conditions and climatic conditions for a given location.

Performance of the methods of the invention gives plants grown under non-stress conditions or under mild drought conditions increased yield relative to control plants grown under comparable conditions. Therefore, according to the present invention, there is provided a method for increasing yield in plants grown under non-stress conditions or under mild drought conditions, which method comprises increasing expression in a plant of a nucleic acid encoding a RANBP polypeptide.

Performance of the methods of the invention gives plants grown under conditions of nutrient deficiency, particularly under conditions of nitrogen deficiency, increased yield relative to control plants grown under comparable conditions. Therefore, according to the present invention, there is provided a method for increasing yield in plants grown under conditions of nutrient deficiency, which method comprises increasing expression in a plant of a nucleic acid encoding a RANBP polypeptide. Nutrient deficiency may result from a lack of nutrients such as nitrogen, phosphates and other phosphorous-containing compounds, potassium, calcium, cadmium, magnesium, manganese, iron and boron, amongst others.

The methods of the invention are advantageously applicable to any plant. Plants that are particularly useful in the methods of the invention include all plants which belong to the superfamily Viridiplantae, in particular monocotyledonous and dicotyledonous plants including fodder or forage legumes, ornamental plants, food crops, trees or shrubs. According to a preferred embodiment of the present invention, the plant is a crop plant. Examples of crop plants include soybean, sunflower, canola, alfalfa, rapeseed, cotton, tomato, potato and tobacco. Further preferably, the plant is a monocotyledonous plant. Examples of monocotyledonous plants include sugarcane. More preferably the plant is a cereal. Examples of cereals include rice, maize, wheat, barley, millet, rye, triticale, sorghum and oats.

The present invention also encompasses use of nucleic acids encoding RANBPs and use of RANBPs themselves in enhancing yield-related traits in plants.

Nucleic acids encoding RANBPs, or RANBPs themselves, may find use in breeding programmes in which a DNA marker is identified which may be genetically linked to a RANBP-encoding gene. The nucleic acids/genes, or the RANBPs themselves may be used to define a molecular marker. This DNA or protein marker may then be used in breeding programmes to select plants having increased yield as defined hereinabove in the methods of the invention.

Allelic variants of a RANBP-encoding acid/gene may also find use in marker-assisted breeding programmes. Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give increased yield. Selection is typically carried out by monitoring growth performance of plants containing different allelic variants of the sequence in question. Growth performance may be monitored in a greenhouse or in the field. Further optional steps include crossing plants in which the superior allelic variant was identified with another plant. This could be used, for example, to make a combination of interesting phenotypic features.

A nucleic acid encoding a RANBP may also be used as probes for genetically and physically mapping the genes that they are a part of, and as markers for traits linked to those genes. Such information may be useful in plant breeding in order to develop lines with desired phenotypes. Such use of RANBP-encoding nucleic acids requires only a nucleic acid sequence of at least 15 nucleotides in length. The RANBP-encoding nucleic acids may be used as restriction fragment length polymorphism (RFLP) markers. Southern blots (Sambrook J, Fritsch EF and Maniatis T (1989) Molecular Cloning, A Laboratory Manual) of restriction-digested plant genomic DNA may be probed with the RANBP-encoding nucleic acids. The resulting banding patterns may then be subjected to genetic analyses using computer programs such as MapMaker (Lander et al. (1987) Genomics 1: 174-181) in order to construct a genetic map. In addition, the nucleic acids may be used to probe Southern blots containing restriction endonuclease-treated genomic DNAs of a set of individuals representing parent and progeny of a defined genetic cross. Segregation of the DNA polymorphisms is noted and used to calculate the position of the RANBP-encoding nucleic acid in the genetic map previously obtained using this population (Botstein et al. (1980) Am. J. Hum. Genet. 32:314-331).

The production and use of plant gene-derived probes for use in genetic mapping is described in Bematzky and Tanksley (1986) Plant Mol. Biol. Reporter 4: 37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid probes may be used in direct fluorescence in situ hybridisation (FISH) mapping (Trask (1991) Trends Genet. 7:149-154). Although current methods of FISH mapping favor use of large clones (several kb to several hundred kb; see Laan et al. (1995) Genome Res. 5:13-20), improvements in sensitivity may allow performance of FISH mapping using shorter probes.

A variety of nucleic acid amplification-based methods for genetic and physical mapping may be carried out using the nucleic acids. Examples include allele-specific amplification (Kazazian (1989) J. Lab. Clin. Med 11:95-96), polymorphism of PCR-amplified fragments (CAPS; Sheffield et al. (1993) Genomics 16:325-332), allele-specific ligation Landegren et al. (1988) Science 241:1077-1080), nucleotide extension reactions (Sokolov (1990) Nucleic Acid Res. 18:3671), Radiation Hybrid Mapping (Walter et al. (1997) Nat. Genet. 7:22-28) and Happy Mapping (Dear and Cook (1989) Nucleic Acid Res. 17:6795-6807). For these methods, the sequence of a nucleic acid is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

The methods according to the present invention result in plants having enhnaced yield-related traits, as described hereinbefore. These traits may also be combined with other economically advantageous traits, such as further yield-enhancing traits, tolerance to other abiotic and biotic stresses, traits modifying various architectural features and/or biochemical and/or physiological features.

### GLK

It has now been found that modulating expression in a plant of a nucleic acid encoding a Golden2-like (GLK) protein gives plants having enhanced yield-related traits relative to control plants.

Therefore, the invention provides a method for enhancing yield-related traits in plants relative to control plants, comprising modulating expression in a plant of a nucleic acid encoding a GLK protein, or a part thereof.

A preferred method for modulating (preferably, increasing) expression of a nucleic acid encoding an Golden2-like protein (GLK) is by introducing and expressing in a plant a nucleic acid encoding such a GLK protein.

Any reference hereinafter to a “protein useful in the methods of the invention” is taken to mean a GLK polypeptide as defined herein. Any reference hereinafter to a “nucleic acid useful in the methods of the invention” is taken to mean a nucleic acid capable of encoding such a GLK polypeptide. The nucleic acid to be introduced into a plant (and therefore useful in performing the methods of the invention) is any nucleic acid encoding the type of protein which will now be described, hereafter also named “GLK nucleic acid” or “GLK gene”.

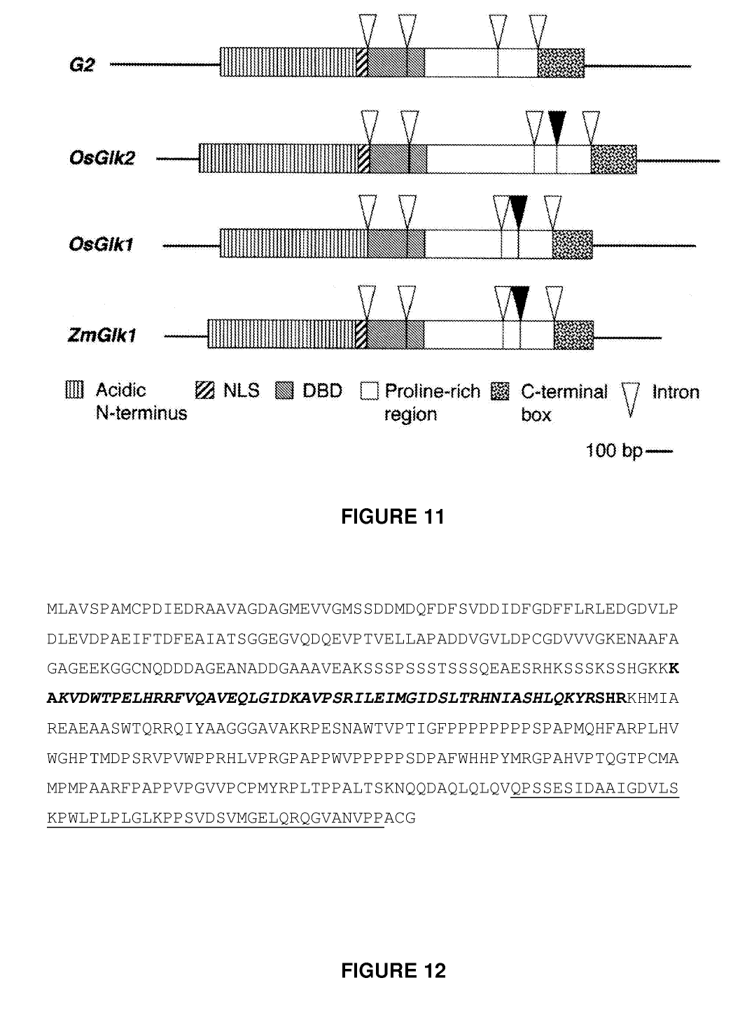

The nucleic acid to be introduced into a plant (and therefore useful in performing the methods of the invention) is any nucleic acid encoding a GLK protein (FIG. 11). The term “GLK protein” or “Golden2-like protein” refers to transcriptional regulator proteins comprising a GARP DNA-binding domain (Tamai et al., Plant Cell Physiol. 43, 99-107, 2002). It is postulated that the GARP domain is a multifunctional domain responsible for both nuclear localization and DNA binding (Hosoda et al., Plant Cell 14, 2015-2021, 2002). GLK proteins preferably also comprise an N-terminal region that is rich in acidic amino acids, a central part of about 100 amino acids enriched in basic amino acids and a C-terminal domain enriched in Pro residues. The C-terminal region preferably also comprises a GARP C-Terminal (GCT) domain (Rossini et al. 2001).

The terms “domain” and “motif” are defined in the definitions section herein. Specialist databases exist for the identification of domains. The GARP domain in a Golden2-like transcriptional regulator may be identified using, for example, SMART (Schultz et al. (1998) Proc. Natl. Acad. Sci. USA 95, 5857-5864; Letunic et al. (2002) Nucl. Acids Res 30, 242-244), InterPro (Mulder et al., (2003) Nucl. Acids. Res. 31, 315-318), Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp. 53-61, AAAI Press, Menlo Park; Hulo et al., Nucl. Acids. Res. 32:D134-D137, (2004)) or Pfam (Bateman et al., Nucleic Acids Research 30(1): 276-280 (2002)). A set of tools for in silico analysis of protein sequences is available on the ExPASY proteomics server (hosted by the Swiss Institute of Bioinformatics (Gasteiger et al., ExPASy: the proteomics server for in-depth protein knowledge and analysis, Nucleic Acids Res. 31:3784-3788 (2003)). Domains or motifs may also be identified using routine techniques, such as by sequence alignment.

The GARP DNA binding domain (Tamai et al. 2002) preferably has three or more of the following consensus sequences:

GARP consensus sequence 1 (SEQ ID NO: 161):
(K/R) (P/M/V/A) (R/K/M) (V/L) (V/D) W (S/T/I/N)
(V/AP/S/T/C/H/Q/D) (E/Q/T/D/S) L (H/D)
(R/K/Q/A/H/D/E/L/I) (K/R/Q/S/C/V/A/H) F (V/L/I)
(A/K/Q/E/H/D/N/R/S) (A/V/C) (V/G/L/I)
(N/E/A/Q/D/G/T/I/K/H) (Q/E/H/L/I/M/K/R/S)L
GARP consensus sequence 2 (SEQ ID NO: 162):
G (I/V/L/P/S/H/Q/A/G) (D/E/K/H/Q/N/A)
GARP consensus sequence 3 (SEQ ID NO: 163):
(A/T) (v/I/Y/F/T) P (K/S) (K/R/T/Q/L/S/G/A)
(I/V/L) (L/M/R/K) (D/E/Q/K/R/S)
(L/I/F/H/V/M/T/R/A) (M/I/L) (N/K/G/S/D/Q/E)
GARP consensus sequence 4 (SEQ ID NO: 164):
(V/I/M/T/E/L/S/N) (E/D/G/N/Y/K/H/Q/P)
(N/G/K/T/S/C/R/D) (I/L) (T/D/A/S) (R/N/I/L/V)
(E/H/D/S/A/Y/F) (N/E/H)
GARP consensus sequence 5 (SEQ ID NO: 165):
(V/I/L) (A/K) SHLQ (K/M/I) (Y/F) (R/V)

More preferably, the GARP consensus sequences have respectively the following sequences:

1: (K/R) (P/M/V/A) (R/K/M) (V/L) (V/D) W (S/T/I)
(V/A/P) (E/Q) LH (R/K/Q) (K/R/Q) FV (A/K/Q/E/H/D)
A (V/G) (N/E/A) (Q/E/H) L
2: G (I/V/L) (D/E/K)
3: A (V/I/Y/F) P (K/S) (K/R/T) I (L/M) (D/E/Q)
(L/I) M (N/K/G/S)
4: (V/I/M/T/E) (E/D/G/N/Y/K/H/Q/P) (N/G/K/T/S/C/R)
(I/L) (T/D) R (E/H) N
5: (V/I) ASHLQK (Y/F) R

Furthermore preferably, the GARP consensus sequences have respectively the following sequences:

1: K (P/V/A) KVDWTPELHR (K/R) FV (Q/E/H) A (V/G) E
(Q/E) L
2: G (I/V/L) (D/E)
3: A (V/Y/F) PSRILE (L/I) M (N/G)
4: (V/I/M/T/E) (E/D/N/Y/K/H/Q) (S/C/R) LTRHN
5: (V/I) ASHLQKYR

Even furthermore preferably, the GARP consensus sequences have respectively the following sequences:

1: K (V/A) KVDWTPELHRRFVQA (V/G) E (Q/E) L
2: G (I/V/L) D
3: AVPSRILE (L/I) MG
4: (I/M/T/E) (E/D/N/Y) (S/C/R) LTRHN
5: IASHLQKYR

Most preferably, the GARP consensus sequences have respectively the following sequences:

1: KAKVDWTPELHRRFVQAVEQL
2: GID
3: AVPSRILEIMG
4: IDSLTRHN
5: IASHLQKYR

Optionally, the GARP consensus sequence 5 is followed by another conserved motif (consensus sequence 6, SEQ ID NO: 166):

SHR (K/R) H (L/M) (L/A/M/I) ARE (A/G/V) EA (A/G)
(S/N/T) W

Preferably this consensus sequence 6 has the sequence:

SHRKH (L/M) (L/M/I) ARE (A/G/V) EA (A/G) (S/N) W

More preferably consensus sequence 6 has the sequence:

SHRKHMIAREAEAASW

A MYB domain motif may, but does not need to, be present in the GARP domain (FIG. 12). This MYB domain may correspond to the Pfam entry PF00249 and InterPro entry IPROO1005, and may comprise the Prosite pattern PS00037 (W-[ST]-{W}-{PTLN}-E-[DE]-{GIYS}-{GYPH}-[LIV].) or Prosite pattern PS00334 (W-x(2)-[LI]-[SAG]-x(4,5)-R-{RE}-x(3)-{AG}-x(3)-[YW]-x(3)-[LIVM].) or Prosite pattern PS50090.

GLK proteins useful in the present invention preferably (but not necessarily) also comprise a GCT domain (Rossini et al., 2001). A consensus sequences for this GCT domain is given in SEQ ID NO: 167:

(H/Q) (P/L) S (N/K/S) E (S/V) (I/V/L) DAAIG (D/E)
(V/A) (I/L) (S/T/A/V) (N/K/R) PW (L/T) P (L/P) PLG
L (K/N) PP (S/A) (V/M/L) (D/E/G) (G/S) V (M/I) (T/
A/S/G) EL (Q/H/E) (R/K) (Q/H) G (V/I) (S/N/P/A)
(N/E/T/K) (V/I) P (P/Q)

Preferably, this GCT consensus domain has the sequence:

(H/Q) PS (N/K/S) ESIDAAIGD (V/A) L (S/T/V) KPW
(L/T) PLPLGLKPPS (V/L) (D/G)SV (M/I) (S/G) EL
(Q/H/E) RQG (V/I) (P/A) (N/K) (V/I) P (P/Q)

More preferably, this GCT consensus domain has the sequence:

QPSSESIDAAIGDVLSKPWLPLPLGLKPPSVDSVMGELQRQGVANVPP

GLK proteins are known to have a higher than average content of acidic amino acids (D and E) in the N-terminal region (from N-terminus to the start of the GARP domain, FIG. 11, FIG. 12), preferably the content is in increasing order of preference, higher than 12%, 15%, 20%, but lower than 30%. Typically the content of D and E in the N-terminal region is around 23%, whereas the average content of D and E in proteins is around 11.9% (Table 3). Similarly, the C-terminal region starting at the end of the GARP domain and including the GCT domain is enriched in Pro residues. Whereas an average protein has a Pro content of 4.8%, the Pro content in this C-terminal region is 25.4% for SEQ ID NO: 157. The P content may vary in this region between 10 and 30%. (range: PpGLK1:11.23, PpGLK2: 11.17, ZmG2: 20.73, ZmGLK1: 23.30, AtGLK2, 17.6%, AtGLK1: 20.13

TABLE 3
Mean amino acid composition of proteins in SWISS PROT
(July 2004):

| Residue | Mole % |
|---|---|
| A = Ala | 7.80 |
| C = Cys | 1.57 |
| D = Asp | 5.30 |
| E = Glu | 6.59 |
| F = Phe | 4.02 |
| G = Gly | 6.93 |
| H = His | 2.27 |
| I = Ile | 5.91 |
| K = Lys | 5.93 |
| L = Leu | 9.62 |
| M = Met | 2.37 |
| N = Asn | 4.22 |
| P = Pro | 4.85 |
| Q = Gln | 3.93 |
| R = Arg | 5.29 |
| S = Ser | 6.89 |
| T = Thr | 5.46 |
| V = Val | 6.69 |
| W = Trp | 1.16 |
| Y = Tyr | 3.09 |

Examples of GLK proteins as defined herein include the protein represented by SEQ ID NO 157, but the term “GLK proteins” also encompasses orthologues or paralogues of the aforementioned SEQ ID NO: 157. The invention is illustrated by transforming plants with the Oryza sativa sequence represented by SEQ ID NO: 156, encoding the polypeptide of SEQ ID NO: 157. SEQ ID NO: 169 (from Oryza sativa, encoded by SEQ ID NO: 168) is a paralogue of the polypeptide of SEQ ID NO: 157 whereas SEQ ID NO: 171 and 173 from Arabidopsis thaliana (encoded by SEQ ID NO: 170 and 172), SEQ ID NO: 175 and 177 from Physcomitrella patens (encoded by SEQ ID NO: 174 and 176), SEQ ID NO: 179 and 181 from Zea mays (encoded by SEQ ID NO: 178 and 180), SEQ ID NO: 183, a partial sequence from Triticum aestivum, and SEQ ID NO: 189, a partial sequence from Sorghum bicolor, are examples of orthologues of the protein of SEQ ID NO: 157. SEQ ID NO: 193 represents a variant of the protein of SEQ ID NO: 157.

Orthologues and paralogues may easily be found by performing a so-called reciprocal blast search. This may be done by a first BLAST involving BLASTing a query sequence (for example, SEQ ID NO: 156 or SEQ ID NO: 157) against any sequence database, such as the publicly available NCBI database. BLASTN or TBLASTX (using standard default values) may be used when starting from a nucleotide sequence and BLASTP or TBLASTN (using standard default values) may be used when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 156 or SEQ ID NO: 157, the second BLAST would therefore be against rice sequences). The results of the first and second BLASTs are then compared. A paralogue is identified if a high-ranking hit from the second BLAST is from the same species as from which the query sequence is derived; an orthologue is identified if a high-ranking hit is not from the same species as from which the query sequence is derived. Preferred orthologues are orthologues of SEQ ID NO: 156 or SEQ ID NO: 157. High-ranking hits are those having a low E-value. The lower the E-value, the more significant the score (or in other words the lower the chance that the hit was found by chance). Computation of the E-value is well known in the art. In addition to E-values, comparisons are also scored by percentage identity. Percentage identity refers to the number of identical nucleotides (or amino acids) between the two compared nucleic acid (or polypeptide) sequences over a particular length. Preferably the score is greater than 50, more preferably greater than 100; and preferably the E-value is less than e-5, more preferably less than e-6. In the case of large families, ClustalW may be used, followed by the generation of a neighbour joining tree, to help visualize clustering of related genes and to identify orthologues and paralogues.

Homologues (or homologous proteins, encompassing orthologues and paralogues) may readily be identified using routine techniques well known in the art, such as by sequence alignment. Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the alignment of two complete sequences that maximizes the number of matches and minimizes the number of gaps. The BLAST algorithm (Altschul et al. (1990) J Mol Biol 215: 403-410) calculates percent sequence identity and performs a statistical analysis of the similarity between the two sequences. The software for performing BLAST analysis is publicly available through the National Centre for Biotechnology Information. Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 4, 29, 2003). Minor manual editing may be performed to optimise alignment between conserved motifs, as would be apparent to a person skilled in the art. Furthermore, instead of using full-length sequences for the identification of homologues, specific domains (such as the GARP domain or the GCT domain) may be used as well.

Preferably, the GLK proteins useful in the methods of the present invention have, in increasing order of preference, at least 30%, 35%, 40%, 45%, 50%, 55%, 60%, 65%, 70%, 75%, 80%, 85%, 90%, 95%, 96%, 97%, 98% or 99% sequence identity to the protein of SEQ ID NO: 157. Alternatively, the sequence identity among homologues may be determined using a specific domain (such as the GARP domain or the GCT domain). A GARP or GCT domain may be identified and delineated using the databases and tools for protein identification listed above, and/or methods for the alignment of sequences for comparison. In some instances, default parameters may be adjusted to modify the stringency of the search. For example using BLAST, the statistical significance threshold (called “expect” value) for reporting matches against database sequences may be increased to show less stringent matches. In this way, short nearly exact matches may be identified.

An example detailing the identification of homologues is given in Example 21. The matrices shown in Example 22 shows similarities and identities (in bold) over the GARP or GCT domain, where of course the values are higher than when considering the full-length protein.

The nucleic acid encoding the polypeptide represented by any one of SEQ ID NO 157, SEQ ID NO: 193, or orthologues or paralogues of any of the aforementioned SEQ ID NOs, need not be full-length nucleic acids, since performance of the methods of the invention does not rely on the use of full length nucleic acid sequences. Furthermore, examples of nucleic acids suitable for use in performing the methods of the invention include but are not limited to those listed in Table Q of Example 21. Nucleic acid variants may also be useful in practising the methods of the invention. Examples of such variants include portions of nucleic acids, hybridising sequences, splice variants, allelic variants either naturally occurring or by DNA manipulation.

The term “portion” as used herein refers to a piece of DNA encoding a polypeptide comprising at least a GARP domain as described above, and preferably also, from N-terminus to C-terminus, (i) a region enriched in acidic nucleic acids (D or E), preceding the GARP domain and (ii) a region C-terminal of the GARP domain which is enriched in Pro residues and preferably comprises a GCT domain.

A portion may be prepared, for example, by making one or more deletions to a nucleic acid encoding a GLK protein as defined hereinabove. The portions may be used in isolated form or they may be fused to other coding (or non coding) sequences in order to, for example, produce a protein that combines several activities. When fused to other coding sequences, the resultant polypeptide produced upon translation may be bigger than that predicted for the GLK portion. Portions useful in the methods of the invention, encode a polypeptide having a GARP domain (as described above) and having substantially the same biological activity as the GLK protein represented by any of SEQ ID NO: 157, SEQ ID NO: 193 or orthologues or paralogues of any of the aforementioned SEQ ID NOs. The portion is typically at least 800 nucleotides in length, preferably at least 900 nucleotides in length, more preferably at least 1000 nucleotides in length and most preferably at least 1100 nucleotides in length. Preferably, the portion is a portion of a nucleic acid as represented by any one of the sequences listed in Table Q of Example 21. Most preferably the portion is a portion of a nucleic acid as represented by SEQ ID NO: 156.

According to the present invention, there is provided a method for enhancing yield-related traits in plants, comprising introducing and expressing in a plant a portion of any one of the nucleic acid sequences given in Table Q of Example 21, or a portion of a nucleic acid encoding an orthologue, paralogue or homologue of any of the amino acid sequences given in Table Q of Example 21.

Another nucleic acid variant useful in the methods of the invention is a nucleic acid capable of hybridising under reduced stringency conditions, preferably under stringent conditions, with a nucleic acid encoding a GLK protein as defined hereinabove, or a with a portion as defined hereinabove.

Hybridising sequences useful in the methods of the invention, encode a polypeptide having a N-terminal region enriched in acidic nucleic acids (D or E), a GARP domain and a region C-terminal of the GARP domain which is enriched in Pro residues and which preferably comprises a GCT domain (as described above) and having substantially the same biological activity as the GLK protein represented by any of SEQ ID NO: 157, SEQ ID NO 193 or orthologues or paralogues of any of the aforementioned SEQ ID NOs. The hybridising sequence is typically at least 800 nucleotides in length, preferably at least 900 nucleotides in length, more preferably at least 1000 nucleotides in length and most preferably at least 1100 nucleotides in length. Preferably, the hybridising sequence is one that is capable of hybridising to any of the nucleic acids represented by (or to probes derived from) the sequences listed in Table Q of Example 21, or to a portion of any of the aforementioned sequences, a portion being as defined above. Most preferably the hybridising sequence is capable of hybridising to SEQ ID NO: 156, or to portions (or probes) thereof. Methods for designing probes are well known in the art. Probes are generally less than 1000 bp in length, preferably less than 500 bp in length. Commonly, probe lengths for DNA-DNA hybridisations such as Southern blotting, vary between 100 and 500 bp, whereas the hybridising region in probes for DNA-DNA hybridisations such as in PCR amplification generally are shorter than 50 but longer than 10 nucleotides. According to the present invention, there is provided a method for enhancing yield-related traits in plants, comprising introducing and expressing in a plant a nucleic acid capable of hybridizing to any one of the nucleic acids given in Table Q of Example 21, or comprising introducing and expressing in a plant a nucleic acid capable of hybridising to a nucleic acid encoding an orthologue, paralogue or homologue of any of the nucleic acid sequences given in Table Q of Example 21.

Another nucleic acid variant useful in the methods of the invention is a splice variant encoding a GLK protein as defined hereinabove.

Preferred splice variants are splice variants of a nucleic acid encoding GLK proteins represented by any of SEQ ID NO: 157, SEQ ID NO 193, or splice variants encoding orthologues or paralogues of any of the aforementioned SEQ ID NOs. Further preferred are splice variants of nucleic acids represented by any one of the sequences listed in Table Q of Example 21. Most preferred is a splice variant of a nucleic acid as represented by SEQ ID NO: 156.

According to the present invention, there is provided a method for enhancing yield-related traits in plants, comprising introducing and expressing in a plant a splice variant of any one of the nucleic acid sequences given in Table Q of Example 21, or a splice variant of a nucleic acid encoding an orthologue, paralogue or homologue of any of the amino acid sequences given in Table Q of Example 21.

Another nucleic acid variant useful in performing the methods of the invention is an allelic variant of a nucleic acid encoding a GLK protein as defined hereinabove. Allelic variants exist in nature, and encompassed within the methods of the present invention is the use of these natural alleles. The allelic variant may be an allelic variant of a nucleic acid encoding a GLK protein represented by any of SEQ ID NO: 157, SEQ ID NO 193, or an allelic variant of a nucleic acid encoding orthologues or paralogues of any of the aforementioned SEQ ID NOs.

Further preferred are allelic variants of nucleic acids represented by any one of the sequences listed in Table Q of Example 21. Most preferred is an allelic variant of a nucleic acid as represented by SEQ ID NO: 156, such as SEQ ID NO: 192.

According to the present invention, there is provided a method for enhancing yield-related traits in plants, comprising introducing and expressing in a plant an allelic variant of any one of the nucleic acids given in Table Q of Example 21, or comprising introducing and expressing in a plant an allelic variant of a nucleic acid encoding an orthologue, paralogue or homologue of any of the amino acid sequences given in Table Q of Example 21.

A further nucleic acid variant useful in the methods of the invention is a nucleic acid variant obtained by gene shuffling. Gene shuffling or directed evolution may also be used to generate variants of nucleic acids encoding GLK proteins as defined above. According to the present invention, there is provided a method for enhancing yield-related traits in plants, comprising introducing and expressing in a plant a variant of any one of the nucleic acid sequences given in Table Q of Example 21, or comprising introducing and expressing in a plant a variant of a nucleic acid encoding an orthologue, paralogue or homologue of any of the amino acid sequences given in Table Q of Example 21, which variant nucleic acid is obtained by gene shuffling.

Furthermore, nucleic acid variants may also be obtained by site-directed mutagenesis. Several methods are available to achieve site-directed mutagenesis, the most common being PCR based methods (Current Protocols in Molecular Biology. Wiley Eds.). Preferred mutants are those that result in a tissue identity switch from C3 tissue structure to the Kranz anatomy of C4 plants.

Also useful in the methods of the invention are nucleic acids encoding homologues of any one of the amino acids represented by SEQ ID NO 157, SEQ ID NO 193, or orthologues or paralogues of any of the aforementioned SEQ ID NOs.

Also useful in the methods of the invention are nucleic acids encoding derivatives of any one of the amino acid sequences represented by SEQ ID NO 157, SEQ ID NO 193 or of orthologues or paralogues of any of the aforementioned SEQ ID NOs.

Furthermore, GLK proteins useful in the methods of the present invention (at least in their native form) typically, but not necessarily, have transcriptional regulatory activity. Therefore, GLK proteins with reduced transcriptional regulatory activity or without transcriptional regulatory activity may equally be useful in the methods of the present invention. A person skilled in the art may easily determine the presence of DNA binding activity or transcriptional activation using routine tools and techniques. To determine the DNA binding activity of GLK proteins, several assays are available (for example Current Protocols in Molecular Biology, Volumes 1 and 2, Ausubel et al. (1994), Current Protocols). In particular, a DNA binding assay for transcription factors comprising a GARP domain is described by Hosoda et al. (2002), including a PCR-assisted DNA binding site selection and a DNA binding gel-shift assay. Alternatively, the approach of Tamai et al. (2002) could be used, where the Arabidopsis GPRI1 was used in an assay for driving transcription of a lacZ reporter gene; Rossini et al 2001 furthermore describe a yeast GAL4 transactivation assay.

Nucleic acids encoding GLK proteins may be derived from any natural or artificial source. The nucleic acid may be modified from its native form in composition and/or genomic environment through deliberate human manipulation. Preferably the GLK protein-encoding nucleic acid is from a plant, further preferably from a monocotyledonous plant, more preferably from the family of Poaceae, most preferably the nucleic acid is from Oryza sativa.

The invention also provides genetic constructs and vectors to facilitate introduction and/or expression of the nucleic acid sequences useful in the methods according to the invention, in a plant.

Therefore, there is provided a gene construct comprising:
(i) a nucleic acid encoding a GLK protein as defined hereinabove;
(ii) one or more control sequences operably linked to the nucleic acid of (i).

Constructs useful in the methods according to the present invention may be constructed using recombinant DNA technology well known to persons skilled in the art. The gene constructs may be inserted into vectors, which may be commercially available, suitable for transforming into plants and suitable for expression of the gene of interest in the transformed cells. The invention therefore provides use of a gene construct as defined hereinabove in the methods of the invention. Preferably, the gene construct is for driving GLK expression in plants.

Plants are transformed with a vector comprising the sequence of interest (i.e., a nucleic acid encoding a GLK protein). The skilled artisan is well aware of the genetic elements that must be present on the vector in order to successfully transform, select and propagate host cells containing the sequence of interest. The sequence of interest is operably linked to one or more control sequences (at least to a promoter).

Advantageously, any type of promoter may be used to drive expression of the nucleic acid sequence. Preferably, the GLK encoding nucleic acid or variant thereof is operably linked to a constitutive promoter. A constitutive promoter is transcriptionally active during most, but not necessarily all, phases of its growth and development and under most environmental conditions in at least one cell, tissue or organ. A preferred constitutive promoter is a constitutive promoter that is also substantially ubiquitously expressed. Further preferably the promoter is derived from a plant, more preferably a monocotyledonous plant. Most preferred is use of a GOS2 promoter (from rice) (SEQ ID NO: 160 or SEQ ID NO: 58). It should be clear that the applicability of the present invention is not restricted to the GLK encoding nucleic acid represented by SEQ ID NO: 156 or SEQ ID NO: 192, nor is the applicability of the invention restricted to expression of a nucleic acid encoding a GLK protein when driven by a GOS2 promoter. Examples of other constitutive promoters which may also be used to drive expression of a nucleic acid encoding a GLK protein are shown in the Definitions section above.

Optionally, one or more terminator sequences may be used in the construct introduced into a plant. Additional regulatory elements may include transcriptional as well as translational enhancers. Those skilled in the art will be aware of terminator and enhancer sequences that may be suitable for use in performing the invention. An intron sequence may also be added to the 5′ untranslated region (UTR) or in the coding sequence to increase the amount of the mature message that accumulates in the cytosol, as described in the definitions section. Other control sequences (besides promoter, enhancer, silencer, intron sequences, 3′UTRand/or 5′UTR regions) may be protein and/or RNA stabilizing elements. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

For the detection of the successful transfer of the nucleic acid sequences as used in the methods of the invention and/or selection of transgenic plants comprising these nucleic acids, it is advantageous to use marker genes (or reporter genes). Therefore, the genetic construct may optionally comprise a selectable marker gene. Selectable markers are described in more detail in the “definitions” section herein. The marker genes may be removed or excised from the transgenic cell once they are no longer needed. Techniques for marker removal are known in the art, useful techniques are described above in the definitions section.

The invention also provides a method for the production of transgenic plants having altered yield-related trait relative to control plants, comprising introduction and expression in a plant of a nucleic acid encoding a GLK polypeptide as defined hereinabove.

More specifically, the present invention provides a method for the production of transgenic plants having altered yield-related traits, which method comprises:
(i) introducing and expressing a nucleic acid encoding a GLK protein in a plant cell; and
(ii) cultivating the plant cell under conditions promoting plant growth and development.

The nucleic acid may be introduced directly into a plant cell or into the plant itself (including introduction into a tissue, organ or any other part of a plant). According to a preferred feature of the present invention, the nucleic acid is preferably introduced into a plant by transformation. The term “transformation” is described in more detail in the definitions section herein.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may be evaluated, for instance using Southern analysis, for the presence of the gene of interest, copy number and/or genomic organisation. Alternatively or additionally, expression levels of the newly introduced DNA may be monitored using Northern and/or Western analysis, or quantitative PCR, all techniques being well known to persons having ordinary skill in the art.

The generated transformed plants may be propagated by a variety of means, such as by clonal propagation or classical breeding techniques. For example, a first generation (or T1) transformed plant may be selfed to give homozygous second generation (or T2) transformants, and the T2 plants further propagated through classical breeding techniques.

The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

The invention also includes host cells containing an isolated nucleic acid encoding a GLK protein as defined hereinabove. Preferred host cells according to the invention are plant cells. Host plants for the nucleic acids or the vector used in the method according to the invention, the expression cassette or construct or vector are, in principle, advantageously all plants, which are capable of synthesizing the polypeptides used in the inventive method.

The invention also extends to harvestable parts of a plant such as, but not limited to seeds, leaves, fruits, flowers, stems, roots, rhizomes, roots, tubers and bulbs. The invention furthermore relates to products derived, preferably directly derived, from a harvestable part of such a plant, such as dry pellets or powders, oil, fat and fatty acids, starch or proteins.

According to a preferred feature of the invention, the modulated expression is increased expression. Methods for increasing expression of nucleic acids or genes, or gene products, are well documented in the art and examples are provided in the definitions section.

As mentioned above, a preferred method for modulating (preferably, increasing) expression of a nucleic acid encoding a GLK protein is by introducing and expressing in a plant a nucleic acid encoding a GLK protein; however the effects of performing the method, i.e. altering yield-related traits may also be achieved using other well known techniques, including but not limited to T-DNA activation tagging, TILLING, homologous recombination. A description of some of these techniques is provided in the definitions section.

Performance of the methods of the invention gives plants having enhanced yield-related traits. In particular performance of the methods of the invention gives plants having increased yield, especially increased seed yield relative to control plants. The terms “yield” and “seed yield” are described in more detail in the “definitions” section herein.

Reference herein to enhanced yield-related traits is taken to mean an increase in biomass (weight) of one or more parts of a plant, which may include aboveground (harvestable) parts and/or (harvestable) parts below ground. In particular, such harvestable parts are above ground biomass and/or seeds, and performance of the methods of the invention results in plants having increased seed yield and/or increased above ground biomass, relative to the seed yield and/or biomass of control plants.

Taking corn as an example, a yield increase may be manifested as one or more of the following: increase in the number of plants established per hectare or acre, an increase in the number of ears per plant, an increase in the number of rows, number of kernels per row, kernel weight, thousand kernel weight, ear length/diameter, increase in the seed filling rate (which is the number of filled seeds divided by the total number of seeds and multiplied by 100), among others. Taking rice as an example, a yield increase may manifest itself as an increase in one or more of the following: number of plants per hectare or acre, number of panicles per plant, number of spikelets per panicle, number of flowers (florets) per panicle (which is expressed as a ratio of the number of filled seeds over the number of primary panicles), increase in the seed filling rate (which is the number of filled seeds divided by the total number of seeds and multiplied by 100), increase in thousand kernel weight, among others.

The present invention provides a method for increasing yield, especially above ground biomass and/or seed yield of plants, relative to control plants, which method comprises modulating expression, preferably increasing expression, in a plant of a nucleic acid encoding a GLK polypeptide as defined herein.

Since the transgenic plants according to the present invention have increased yield, it is likely that these plants exhibit an increased growth rate (during at least part of their life cycle), relative to the growth rate of control plants at a corresponding stage in their life cycle.

The increased growth rate may be specific to one or more parts of a plant (including seeds), or may be throughout substantially the whole plant. Plants having an increased growth rate may have a shorter life cycle. The life cycle of a plant may be taken to mean the time needed to grow from a dry mature seed up to the stage where the plant has produced dry mature seeds, similar to the starting material. This life cycle may be influenced by factors such as early vigour, growth rate, greenness index, flowering time and speed of seed maturation. The increase in growth rate may take place at one or more stages in the life cycle of a plant or during substantially the whole plant life cycle. Increased growth rate during the early stages in the life cycle of a plant may reflect enhanced vigour. The increase in growth rate may alter the harvest cycle of a plant allowing plants to be sown later and/or harvested sooner than would otherwise be possible (a similar effect may be obtained with earlier flowering time). If the growth rate is sufficiently increased, it may allow for the further sowing of seeds of the same plant species (for example sowing and harvesting of rice plants followed by sowing and harvesting of further rice plants all within one conventional growing period). Similarly, if the growth rate is sufficiently increased, it may allow for the further sowing of seeds of different plants species (for example the sowing and harvesting of corn plants followed by, for example, the sowing and optional harvesting of soybean, potato or any other suitable plant). Harvesting additional times from the same rootstock in the case of some crop plants may also be possible. Altering the harvest cycle of a plant may lead to an increase in annual biomass production per acre (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and T-90 (time taken for plants to reach 90% of their maximal size), amongst others.

According to a preferred feature of the present invention, performance of the methods of the invention gives plants having an increased growth rate relative to control plants. Therefore, according to the present invention, there is provided a method for increasing the growth rate of plants, which method comprises modulating expression, preferably increasing expression, in a plant of a nucleic acid encoding a GLK polypeptide as defined herein.

An increase in yield and/or growth rate occurs whether the plant is under non-stress conditions or whether the plant is exposed to various stresses compared to control plants. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35% or 30%, preferably less than 25%, 20% or 15%, more preferably less than 14%, 13%, 12%, 11% or 10% or less in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the everyday biotic and/or abiotic (environmental) stresses to which a plant is exposed. Abiotic stresses may be due to drought or excess water, anaerobic stress, salt stress, chemical toxicity, oxidative stress and hot, cold or freezing temperatures. The abiotic stress may be an osmotic stress caused by a water stress (particularly due to drought), salt stress, oxidative stress or an ionic stress. Biotic stresses are typically those stresses caused by pathogens, such as bacteria, viruses, fungi and insects.

In particular, the methods of the present invention may be performed under non-stress conditions or under conditions of mild drought to give plants having increased yield relative to control plants. As reported in Wang et al. (Planta (2003) 218: 1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani et al. (Plant Physiol (2003) 133: 1755-1767) describes a particularly high degree of “cross talk” between drought stress and high-salinity stress. For example, drought and/or salinisation are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signalling pathways and cellular responses, such as the production of stress proteins, up-regulation of anti-oxidants, accumulation of compatible solutes and growth arrest. The term “non-stress” conditions as used herein are those environmental conditions that allow optimal growth of plants. Persons skilled in the art are aware of normal soil conditions and climatic conditions for a given location.

Performance of the methods of the invention gives plants grown under non-stress conditions or under mild drought conditions increased yield relative to control plants grown under comparable conditions. Therefore, according to the present invention, there is provided a method for increasing yield in plants grown under non-stress conditions or under mild drought conditions, which method comprises increasing expression in a plant of a nucleic acid encoding a GLK polypeptide.

Performance of the methods of the invention gives plants grown under conditions of nutrient deficiency, particularly under conditions of nitrogen deficiency, increased yield relative to control plants grown under comparable conditions. Therefore, according to the present invention, there is provided a method for increasing yield in plants grown under conditions of nutrient deficiency, which method comprises increasing expression in a plant of a nucleic acid encoding a GLK polypeptide. Nutrient deficiency may result from a lack of nutrients such as nitrogen, phosphates and other phosphorous-containing compounds, potassium, calcium, cadmium, magnesium, manganese, iron and boron, amongst others.

The methods of the invention are advantageously applicable to any plant. Plants that are particularly useful in the methods of the invention include all plants which belong to the superfamily Viridiplantae, in particular monocotyledonous and dicotyledonous plants including fodder or forage legumes, ornamental plants, food crops, trees or shrubs. According to a preferred embodiment of the present invention, the plant is a crop plant. Examples of crop plants include soybean, sunflower, canola, alfalfa, rapeseed, cotton, tomato, potato and tobacco. Further preferably, the plant is a monocotyledonous plant. Examples of monocotyledonous plants include sugarcane. More preferably the plant is a cereal. Examples of cereals include rice, maize, wheat, barley, millet, rye, triticale, sorghum and oats.

The present invention also encompasses plants obtainable by the methods according to the present invention. The present invention therefore provides plants, plant parts or plant cells thereof obtainable by the method according to the present invention, which plants or parts or cells thereof comprise a nucleic acid transgene encoding a GLK protein as defined above.

The present invention also encompasses use of nucleic acids encoding GLK proteins and use of GLK polypeptides in altering yield-related traits.

Nucleic acids encoding GLK polypeptides, or GLK proteins themselves, may find use in breeding programmes in which a DNA marker is identified which may be genetically linked to a GLK protein-encoding gene. The nucleic acids/genes, or the GLK proteins themselves may be used to define a molecular marker. This DNA or protein marker may then be used in breeding programmes to select plants having increased yield as defined hereinabove in the methods of the invention.

Allelic variants of a GLK protein-encoding acid/gene may also find use in marker-assisted breeding programmes. Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give increased yield. Selection is typically carried out by monitoring growth performance of plants containing different allelic variants of the sequence in question. Growth performance may be monitored in a greenhouse or in the field. Further optional steps include crossing plants in which the superior allelic variant was identified with another plant. This could be used, for example, to make a combination of interesting phenotypic features.

A nucleic acid encoding a GLK protein may also be used as probes for genetically and physically mapping the genes that they are a part of, and as markers for traits linked to those genes. Such information may be useful in plant breeding in order to develop lines with desired phenotypes. Such use of GLK encoding nucleic acids requires only a nucleic acid sequence of at least 15 nucleotides in length. The GLK encoding nucleic acids may be used as restriction fragment length polymorphism (RFLP) markers. Southern blots (Sambrook J, Fritsch EF and Maniatis T (1989) Molecular Cloning, A Laboratory Manual) of restriction-digested plant genomic DNA may be probed with the GLK encoding nucleic acids. The resulting banding patterns may then be subjected to genetic analyses using computer programs such as MapMaker (Lander et al. (1987) Genomics 1: 174-181) in order to construct a genetic map. In addition, the nucleic acids may be used to probe Southern blots containing restriction endonuclease-treated genomic DNAs of a set of individuals representing parent and progeny of a defined genetic cross. Segregation of the DNA polymorphisms is noted and used to calculate the position of the GLK encoding nucleic acid in the genetic map previously obtained using this population (Botstein et al. (1980) Am. J. Hum. Genet. 32:314-331).

The production and use of plant gene-derived probes for use in genetic mapping is described in Bernatzky and Tanksley (Plant Mol. Biol. Reporter 4: 37-41, 1986). Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid probes may be used in direct fluorescence in situ hybridisation (FISH) mapping (Trask (1991) Trends Genet. 7:149-154). Although current methods of FISH mapping favour use of large clones (several kb to several hundred kb; see Laan et al. (1995) Genome Res. 5:13-20), improvements in sensitivity may allow performance of FISH mapping using shorter probes.

A variety of nucleic acid amplification-based methods for genetic and physical mapping may be carried out using the nucleic acids. Examples include allele-specific amplification (Kazazian (1989) J. Lab. Clin. Med. 11:95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic Acid Res. 18:3671), Radiation Hybrid Mapping (Walter et al. (1997) Nat. Genet. 7:22-28) and Happy Mapping (Dear and Cook (1989) Nucleic Acid Res. 17:6795-6807). For these methods, the sequence of a nucleic acid is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

The methods according to the present invention result in plants having altered yield-related traits, as described hereinbefore. These traits may also be combined with other economically advantageous traits, such as further yield-enhancing traits, tolerance to other abiotic and biotic stresses, traits modifying various architectural features and/or biochemical and/or physiological features.

### REV DHDZip/START

It has now surprisingly been found that reducing the expression in a plant of an endogenous REV gene using a REV delta homeodomain leucine zipper domain (HDZip)/STeroidogenic Acute Regulatory (STAR) related lipid Transfer domain (START) nucleic acid sequence gives plants having increased yield relative to control plants. The present invention therefore provides methods for increasing yield in plants relative to control plants, by reducing the expression in a plant of an endogenous REV gene using a REV ΔHDZip/START nucleic acid sequence.

Advantageously, performance of the methods according to the present invention results in plants having enhanced yield related traits, particularly increased yield, more particularly increased seed yield and/or increased biomass, relative to control plants. The terms “yield” and “seed yield” are described in more detail in the “definitions” section herein. Increased biomass may manifest itself as increased root biomass. Increased root biomass may be due to increased number of roots, increased root thickness and/or increased root length.

The term “increased yield” also refers to an increase in biomass (weight) of one or more parts of a plant, which may include aboveground (harvestable) parts and/or (harvestable) parts below ground. Such harvestable parts include vegetative biomass and/or seeds, and performance of the methods of the invention results in plants having increased yield (in vegetative biomass and/or seed) relative to the yield of control plants.

Therefore, according to the present invention, there is provided a method for increasing seed yield and/or plant biomass, which method comprises reducing the expression in a plant of an endogenous REV gene using a REV ΔHDZip/START nucleic acid sequence.

In particular, the increased seed yield is selected from one or more of the following: (i) increased seed weight; (ii) increased number of filled seeds; (iii) increased seed fill rate; (iv) increased harvest index; and (v) increased individual seed length.

Taking corn as an example, a yield increase may be manifested as one or more of the following: increase in the number of plants established per hectare or acre, an increase in the number of ears per plant, an increase in the number of rows, number of kernels per row, kernel weight, thousand kernel weight, ear length/diameter, increase in the seed filling rate (which is the number of filled seeds divided by the total number of seeds and multiplied by 100), among others. Taking rice as an example, a yield increase may manifest itself as an increase in one or more of the following: number of plants per hectare or acre, number of panicles per plant, number of spikelets per panicle, number of flowers (florets) per panicle (which is expressed as a ratio of the number of filled seeds over the number of primary panicles), increase in the seed filling rate (which is the number of filled seeds divided by the total number of seeds and multiplied by 100), increase in thousand kernel weight, among others.

The present invention provides a method for increasing yield, especially seed yield of plants, relative to control plants, which method comprises modulating expression, preferably increasing expression, in a plant of a nucleic acid encoding a REV ΔHDZip/START polypeptide as defined herein.

Since the transgenic plants according to the present invention have increased yield, it is likely that these plants exhibit an increased growth rate (during at least part of their life cycle), relative to the growth rate of control plants at a corresponding stage in their life cycle.

The increased growth rate may be specific to one or more parts of a plant (including seeds), or may be throughout substantially the whole plant. Plants having an increased growth rate may have a shorter life cycle. The life cycle of a plant may be taken to mean the time needed to grow from a dry mature seed up to the stage where the plant has produced dry mature seeds, similar to the starting material. This life cycle may be influenced by factors such as early vigour, growth rate, greenness index, flowering time and speed of seed maturation. The increase in growth rate may take place at one or more stages in the life cycle of a plant or during substantially the whole plant life cycle. Increased growth rate during the early stages in the life cycle of a plant may reflect enhanced vigour. The increase in growth rate may alter the harvest cycle of a plant allowing plants to be sown later and/or harvested sooner than would otherwise be possible (a similar effect may be obtained with earlier flowering time). If the growth rate is sufficiently increased, it may allow for the further sowing of seeds of the same plant species (for example sowing and harvesting of rice plants followed by sowing and harvesting of further rice plants all within one conventional growing period). Similarly, if the growth rate is sufficiently increased, it may allow for the further sowing of seeds of different plants species (for example the sowing and harvesting of corn plants followed by, for example, the sowing and optional harvesting of soybean, potato or any other suitable plant). Harvesting additional times from the same rootstock in the case of some crop plants may also be possible. Altering the harvest cycle of a plant may lead to an increase in annual biomass production per acre (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and T-90 (time taken for plants to reach 90% of their maximal size), amongst others.

According to a preferred feature of the present invention, performance of the methods of the invention gives plants having an increased growth rate relative to control plants. Therefore, according to the present invention, there is provided a method for increasing the growth rate of plants, which method comprises modulating expression, preferably increasing expression, in a plant of a nucleic acid encoding a REV ΔHDZip/START polypeptide as defined herein.

An increase in yield and/or growth rate occurs whether the plant is under non-stress conditions or whether the plant is exposed to various stresses compared to control plants. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35% or 30%, preferably less than 25%, 20% or 15%, more preferably less than 14%, 13%, 12%, 11% or 10% or less in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the everyday biotic and/or abiotic (environmental) stresses to which a plant is exposed. Abiotic stresses may be due to drought or excess water, anaerobic stress, salt stress, chemical toxicity, oxidative stress and hot, cold or freezing temperatures. The abiotic stress may be an osmotic stress caused by a water stress (particularly due to drought), salt stress, oxidative stress or an ionic stress. Biotic stresses are typically those stresses caused by pathogens, such as bacteria, viruses, fungi and insects.

In particular, the methods of the present invention may be performed under non-stress conditions or under conditions of mild drought to give plants having increased yield relative to control plants. As reported in Wang et al. (Planta (2003) 218: 1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani et al. (Plant Physiol (2003) 133: 1755-1767) describes a particularly high degree of “cross talk” between drought stress and high-salinity stress. For example, drought and/or salinisation are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signalling pathways and cellular responses, such as the production of stress proteins, up-regulation of anti-oxidants, accumulation of compatible solutes and growth arrest. The term “non-stress” conditions as used herein are those environmental conditions that allow optimal growth of plants. Persons skilled in the art are aware of normal soil conditions and climatic conditions for a given location.

Performance of the methods of the invention gives plants grown under non-stress conditions or under mild drought conditions increased yield relative to control plants grown under comparable conditions. Therefore, according to the present invention, there is provided a method for increasing yield in plants grown under non-stress conditions or under mild drought conditions, which method comprises increasing expression in a plant of a nucleic acid encoding a REV ΔHDZip/START polypeptide.

Performance of the methods of the invention gives plants grown under conditions of nutrient deficiency, particularly under conditions of nitrogen deficiency, increased yield relative to control plants grown under comparable conditions. Therefore, according to the present invention, there is provided a method for increasing yield in plants grown under conditions of nutrient deficiency, which method comprises increasing expression in a plant of a nucleic acid encoding a REV ΔHDZip/START polypeptide. Nutrient deficiency may result from a lack of nutrients such as nitrogen, phosphates and other phosphorous-containing compounds, potassium, calcium, cadmium, magnesium, manganese, iron and boron, amongst others.

Reference herein to an “endogenous” REV gene not only refers to a REV gene as found in a plant in its natural form (i.e., without there being any human intervention), but also refers to isolated REV nucleic acid sequences subsequently introduced into a plant. For example, a transgenic plant containing a REV transgene may encounter a substantial reduction of the transgene expression and/or substantial reduction of expression of an endogenous REV gene, according to the methods of the invention.

The term “expression” or “gene expression” means the transcription of a specific gene or specific genes or specific genetic construct. The term “expression” or “gene expression” in particular means the transcription of a gene or genes or genetic construct into structural RNA (rRNA, tRNA) or mRNA with or without subsequent translation of the latter into a protein. The process includes transcription of DNA and processing of the resulting mRNA product.

“Reduction” or “decrease” of expression are used interchangeably herein, and refer, for the methods of the present invention, to a diminution, but not the elimination, of endogenous REV gene expression and/or REV polypeptide levels and/or REV polypeptide activity, using a REV ΔHDZip/START nucleic acid sequence, relative respectively to REV gene expression and/or REV polypeptide level and/or REV polypeptide activity found in control plants. The reduction of REV gene expression and/or REV polypeptide level and/or REV polypeptide activity is taken to mean in the sense of the application at least 10%, 20%, 30%, 40% or 50%, preferably at least 60%, 70 or 80%, more preferably 85%, 90%, or 95% less REV gene expression and/or REV polypeptide level and/or REV polypeptide activity in comparison to a control plant as defined herein. Preferably, reducing the expression of the endogenous REV gene using a REV ΔHDZip/START nucleic acid sequence leads to the appearance of one or more phenotypic traits.

This reduction of endogenous REV gene expression may be achieved by using any one or more of several well-known “gene silencing” methods (see definitions section for more details). The term “silencing” of a gene as used herein refers to the reduction, but not the elimination, of endogenous REV gene expression.

A preferred method for reducing expression in a plant of an endogenous REV gene via RNA-mediated silencing is by using an inverted repeat of a REV ΔHDZip/START nucleic acid sequence, preferably capable of forming a hairpin structure. The inverted repeat is cloned into an expression vector comprising control sequences. A non-coding DNA nucleic acid sequence (a spacer, for example a matrix attachment region fragment (MAR), an intron, a polylinker, etc.) is located between the two inverted REV ΔHDZip/START nucleic acid sequences forming the inverted repeat. After transcription of the inverted repeat, a chimeric RNA with a self-complementary structure is formed (partial or complete). This double-stranded RNA structure is referred to as the hairpin RNA (hpRNA). The hpRNA is processed by the plant into siRNAs that are incorporated into a RISC. The RISC further cleaves the mRNA transcripts encoding a REV polypeptide, thereby reducing the number of mRNA transcripts to be translated into a REV polypeptide. See for example, Grierson et al. (1998) WO 98/53083; Waterhouse et al. (1999) WO 99/53050).

The expression of an endogenous REV gene may also be reduced by introducing a genetic modification, within the locus of a REV gene or elsewhere in the genome. The locus of a gene as defined herein is taken to mean a genomic region, which includes the gene of interest and 10 kb up- or down stream of the coding region.

The genetic modification may be introduced, for example, by any one (or more) of the following methods: T-DNA tagging, TILLING, site-directed mutagenesis, directed evolution, homologous recombination. Following introduction of the genetic modification, there follows a step of selecting for reduced expression of an endogenous REV gene, which reduction in expression gives plants having increased yield compared to control plants.

T-DNA tagging involves insertion of a T-DNA, in the genomic region of the gene of interest or 10 kb up- or downstream of the coding region of a gene in a configuration such that the T-DNA reduces (but does not eliminate) the expression of the targeted gene.

Homologous recombination allows introduction in a genome of a selected nucleic acid at a defined selected position. Alternatively, a screening program may be set up to identify in a plant population natural variants of a REV gene which variants encode REV polypeptides with reduced activity. Such natural variants may also be used for example, to perform homologous recombination.

T-DNA tagging, TILLING, site-directed mutagenesis and directed evolution are examples of technologies that enable the generation of novel alleles and variants of REV nucleic acid sequences which variants encode REV polypeptides with reduced activity.

Other methods, such as the use of antibodies directed to an endogenous REV polypeptide for inhibiting its function in planta, or interference in the signalling pathway in which a REV polypeptide is involved, will be well known to the skilled man.

For optimal performance, the RNA-mediated silencing techniques used for reducing expression in a plant of an endogenous REV gene using a REV ΔHDZip/START nucleic acid sequence, requires the use of nucleic acid sequences from monocotyledonous plants for transformation of monocotyledonous plants, and from dicotyledonous plants for transformation of dicotyledonous plants. Preferably, a REV ΔHDZip/START nucleic acid sequence from any given plant species is introduced into that same species. For example, a REV ΔHDZip/START nucleic acid sequence from rice is transformed into a rice plant. The REV ΔHDZip/START nucleic acid sequence need not be introduced into the same plant variety.

Reference herein to a “nucleic acid sequence” is taken to mean a polymeric form of a deoxyribonucleotide or a ribonucleotide polymer of any length, either double- or single-stranded, or analogues thereof, that has the essential characteristic of a natural ribonucleotide in that it can hybridise to nucleic acid sequences in a manner similar to naturally occurring polynucleotides.

Reference herein to a “REV ΔHDZip/START” nucleic acid sequence is taken to mean a sufficient length of substantially contiguous nucleotides from a REV nucleic acid sequence substantially excluding the part encoding the HDZip and START domains. In order to perform gene silencing, this may be as little as 20 or fewer nucleotides, alternatively this may be as much as the REV ΔHDZip/START nucleic acid sequence (including the 5′ and/or 3′ UTR, either in part or in whole. A person skilled in the art would be aware that a sufficient length of substantially contiguous nucleotides from the REV nucleic acid sequence encoding the HDZip and START domains is to be excluded in performing the methods of the invention. This may be as little as 10, 11, 12, 13, 14, 15, 16, 17, 18, 19, 20 or more nucleotides. This may be as much as the complete nucleic acid sequence encoding the HDZip and START domains. By nucleic acid sequence “encoding the HDZip and START domains” is meant herein the region of the nucleic acid sequence comprising codons that are translated into amino acid residues of the HDZip and START domains (which domains do not need to be complete and/or functional). A person skilled in the art would be aware that substantially contiguous nucleotides from a REV nucleic acid sequence may overlap HDZip and START domain boundaries by a few nucleotides, typically by not more than 20 nucleotides. Also excluded in performing the methods of the invention are nucleic acid sequences that would simultaneously reduce expression of at least one other endogenous gene, regardless whether the other endogenous gene encodes for a polypeptide comprising an HDZip and START domain or not. A nucleic acid sequence encoding a (functional) polypeptide is not a requirement for the various methods discussed above for the substantial reduction of expression of an endogenous REV gene.

REV genes are well known in the art (described recently in Floyd et al. ((2006) Genetics 173: 373-388) and useful in the methods of the invention are REV ΔHDZip/START nucleic acid sequences.

Other REV ΔHDZip/START nucleic acid sequences may also be used in the methods of the invention, and may readily be identified by a person skilled in the art. REV polypeptides may be identified by the presence of one or more of several well-known features (see below). Upon identification of a REV polypeptide, a person skilled in the art could easily derive, using routine techniques, the corresponding encoding REV ΔHDZip/START nucleic acid sequence, and use a sufficient length of contiguous nucleotides of the same to perform any one or more of the gene silencing methods described above.

The term “REV polypeptide” as defined herein refers to a polypeptide that falls into the class III of the HDZip polypeptides as delineated by Sessa et al. ((1994) In: Puigdomene P, Coruzzi G (ed), Springer, Berlin Heidelberg New York, pp 411-426). REV polypeptides comprise from N-terminus to C-terminus: (i) a homeodomain (HD) domain, for DNA binding; (ii) a leucine zipper, for protein-protein interaction; (iii) a START domain for lipid/sterol binding, and (iv) a C-terminal region (CTR), of undefined function.

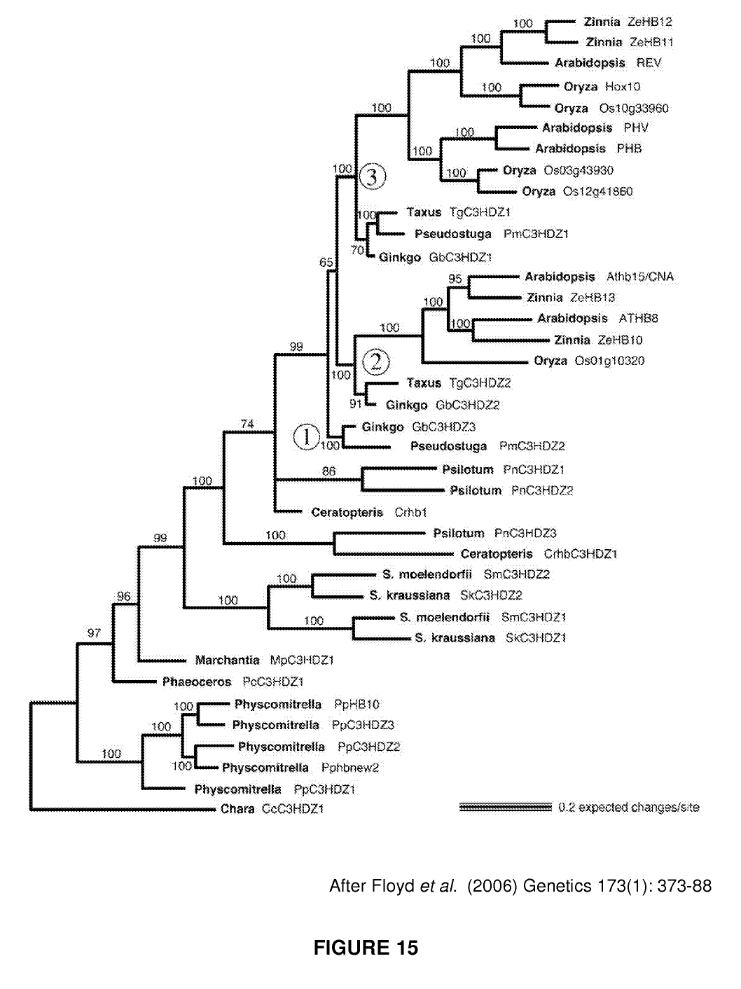

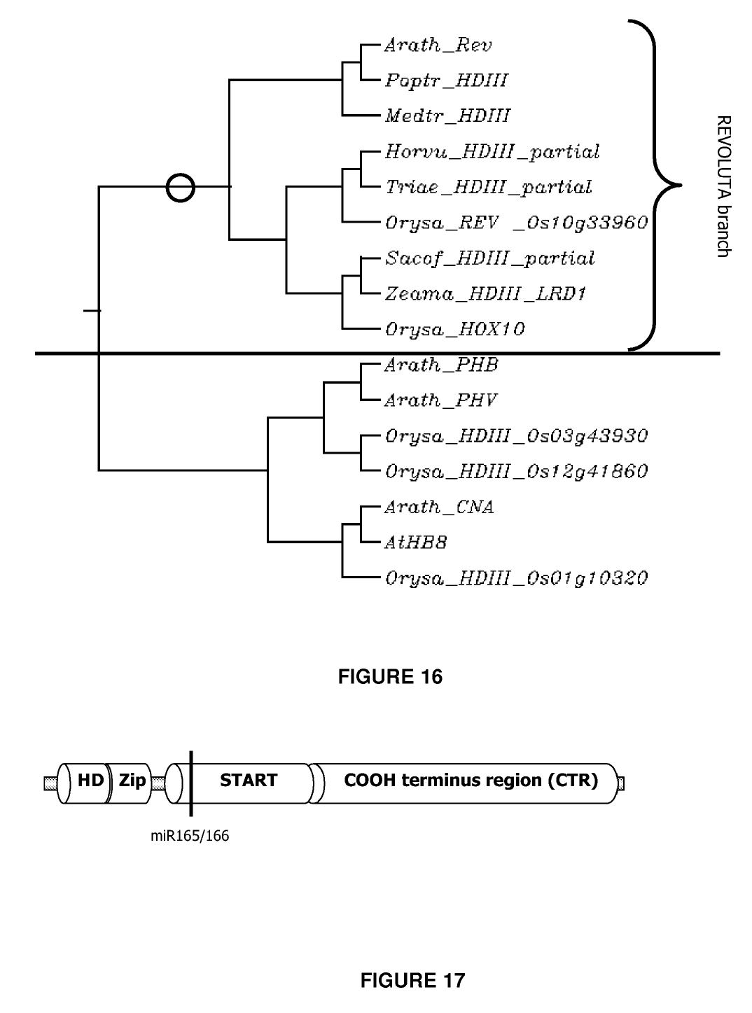

REV polypeptides may readily be identified using routine techniques well known in the art, such as by sequence alignment. Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the alignment of two complete sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information. REV polypeptides comprising a homeodomain, a leucine zipper, a START domain and a CTR may readily be identified using, for example, the ClustalW multiple sequence alignment algorithm (version 1.83), with the default pairwise alignment parameters, and a scoring method in percentage. Minor manual editing may be performed to optimise the alignment, as would be apparent to a person skilled in the art. A phylogenetic tree, which is an estimate of phylogeny (or common ancestry), may be constructed using the Neighbour-Joining tree building algorithm (at EBI), to help visualize clustering of related genes and to identify orthologues and paralogues. REV polypeptides as defined herein refers to any polypeptide which, when used in the construction of a class III HDZip polypeptide phylogenetic tree, such as the one depicted in FIGS. 15 and 16, falls into the REV clade (comprising REV, PHB and PHV) and not the CORONA clade (comprising ATHb8 and CNA), and more specifically, which falls into the REV branch (and not the PHB/PHV branch). Upon identification of a REV polypeptide (falling into the REV branch), a person skilled in the art could easily derive, using routine techniques, the corresponding encoding REV ΔHDZip/START nucleic acid sequence and use a sufficient length of contiguous nucleotides of the same to perform any one or more of the gene silencing methods described above.

Orthologues and paralogues may easily be found by performing a so-called reciprocal blast search. This may be done by a first BLAST involving BLASTing a query sequence (for example, SEQ ID NO: 198 or SEQ ID NO: 199) against any sequence database, such as the publicly available NCBI database. BLASTN or TBLASTX (using standard default values) may be used when starting from a nucleotide sequence and BLASTP or TBLASTN (using standard default values) may be used when starting from a polypeptide sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 198 or SEQ ID NO: 199, the second BLAST would therefore be against rice sequences). High-ranking hits are those having a low E-value. In addition to E-values, comparisons are also scored by percentage identity. Percentage identity refers to the number of identical nucleotides (or amino acids) between the two compared nucleic acid (or polypeptide) sequences over a particular length. An example detailing the identification of orthologues and paralogues is given in Example 27. All REV polypeptides comprise a CTR having, in increasing order of preference, at least 50%, 55%, 60%, 65%, 70%, 75%, 80%, 85%, 90%, 95% or 98% sequence identity to the CTR of a REV polypeptide as represented by SEQ ID NO: 197. Preferably, the CTR of a REV polypeptide is as represented by SEQ ID NO: 197. More preferably, SEQ ID NO: 194 encoding a part of the CTR of a REV polypeptide is used in performing the methods according the invention. In FIG. 16, the REV polypeptide paralogues and orthologues cluster together.

The terms “domain” and “motif” are defined above. The term “region” as defined herein refers to the amino acid sequence starting at the end of the START domain and ending at the stop codon of the REV polypeptide.

Special databases exist for the identification of domains. The HD and the START domains in a REV polypeptide may be identified using, for example, SMART, InterPro, Prosite, or Pfam. The HD comprises about 60 residues, the START domain about 210 residues. In the InterPro database, the HD is designated by IPR001356, PF00046 in the Pfam database and PS50071 in the PROSITE database. In the InterPro database, the START domain is designated by IPR002913, PF01852 in the Pfam database and PS50848 in the PROSITE database. For example in SEQ ID NO: 199, the HD Pfam entry spans from amino acids 27 to 87, and the START domain Pfam entry from 163 to 376. The CTR therefore begins at amino acid 377 and ends at the stop codon (at 840). Leucine zipper prediction and heptad identification may be done using specialised software such as 2ZIP, which combines a standard coiled coil prediction algorithm with an approximate search for the characteristic leucine repeat (Bornberg-Bauer et al. (1998) Computational Approaches to Identify Leucine Zippers, Nucleic Acids Res., 26(11): 2740-2746), hosted on a server at Max Planck Institute for Molecular Genetics in Berlin. For example, the leucine zipper of SEQ ID NO: 199 comprises five leucine repeats (heptads), and spans amino acids 91 to 127.

Furthermore, a REV polypeptide may also be identifiable by its ability to bind DNA and to interact with other proteins. DNA-binding activity and protein-protein interactions may readily be determined in vitro or in vivo using techniques well known in the art. Examples of in vitro assays for DNA binding activity include: gel retardation analysis using the HD DNA binding domain (West et al. (1998) Nucl Acid Res 26(23): 5277-87), or yeast one-hybrid assays. An example of an in vivo assay for protein-protein interactions is the yeast two-hybrid analysis (Fields and Song (1989) Nature 340:245-6).

Therefore upon identification of a REV polypeptide using one or several of the features described above, a person skilled in the art may easily derive the corresponding REV ΔHDZip/START nucleic acid sequence and use a sufficient length of substantially contiguous nucleotides of the same to perform any one or more of the gene silencing methods described above (for the substantial reduction of an endogenous REV gene expression).

Preferred for use in the methods of the invention is a nucleic acid sequence as represented by SEQ ID: 194, encoding part of the CTR of an Oryza sativa REV polypeptide, or SEQ ID NO: 196, encoding the CTR of the same Oryza sativa REV polypeptide. REV ΔHDZip/START nucleic acid sequences are comprised in the nucleic acid sequences encoding REV polypeptide orthologues or paralogues. An example of a REV polypeptide paralogue to SEQ ID NO: 199 is represented by SEQ ID NO: 201, Oryza sativa Orysa_HOX10 (encoded by SEQ ID NO: 200, NCBI accession number AY425991.1). Examples of REV polypeptide orthologues are represented by SEQ ID NO: 203 Arabidopsis thaliana Arath_REV (encoded by SEQ ID NO: 202, NCBI accession number AF188994), SEQ ID NO: 205 Zea mays Zeama_HDIII RLD1 (rolled leaf1; encoded by SEQ ID NO: 204, NCBI accession number AY501430.1), SEQ ID NO: 207 Populus trichocarpa Poptr_HDIII (encoded by SEQ ID NO: 206, NCBI accession number AY919617), SEQ ID NO: 209 Medicago trunculata Medtr_HDIII (encoded by SEQ ID NO: 208, NCBI accession number AC138171.17), SEQ ID NO: 211 Saccharum officinarum Sacof_HDIlipartial (encoded by SEQ ID NO: 210, contig of NCBI accession numbers CA125167.1 CA217027.1 CA241276.1 CA124509.1), SEQ ID NO: 213 Triticum aestivum Triae_HDIII (partial; encoded by SEQ ID NO: 212, contig of NCBI accession numbers CD905903 BM135681.1 BQ578798.1 CJ565259.1), SEQ ID NO: 215 Hordeum vulgare Horvu_HDIII (partial, encoded by SEQ ID NO: 214, contig of NCBI accession numbers BU996988.1 BJ452342.1 BJ459891.1), and SEQ ID NO: 217 Phyllostachys praecox PhyprHDIII (partial; encoded by SEQ ID NO: 216, NCBI accession number DQ013803). In example 27 (and table Z) of the present application is described a method to identify nucleic acid sequences useful in performing the methods of the invention.

The source of the REV ΔHDZip/START nucleic acid sequence useful in performing the methods of the invention may be any plant source or artificial source. For optimal performance, the gene silencing techniques used for the reduction of an endogenous REV gene expression requires the use of REV ΔHDZip/START nucleic acid sequences from monocotyledonous plants for transformation of monocotyledonous plants, and use of REV ΔHDZip/START nucleic acid sequences from dicotyledonous plants for transformation of dicotyledonous plants. Preferably, REV ΔHDZip/START nucleic acid sequences from plants of the family Poaceae are transformed into plants of the family Poaceae. Further preferably, a REV ΔHDZip/START nucleic acid sequence from rice is transformed into a rice plant. The REV ΔHDZip/START nucleic acid sequence need not be introduced into the same plant variety. Most preferably, the REV ΔHDZip/START from rice is a sufficient length of substantially contiguous nucleotides of SEQ ID NO: 194 or SEQ ID NO: 196, or a sufficient length of substantially contiguous nucleotides of a REV ΔHDZip/START nucleic acid sequence from nucleic acid sequences encoding REV polypeptide orthologues or paralogues. As mentioned above, a person skilled in the art would be well aware of what would constitute a sufficient length of substantially contiguous nucleotides to perform any of the gene silencing methods defined hereinabove, this may be as little as 20 or fewer substantially contiguous nucleotides in some cases.

The invention also provides genetic constructs and vectors to facilitate introduction and/or expression of the nucleotide sequences useful in the methods according to the invention.

Therefore, there is provided a genetic construct for reduced expression in a plant of an endogenous REV gene comprising one or more control sequences, a REV ΔHDZip/START nucleic acid sequence, and optionally a transcription termination sequence. Preferably, the control sequence is a constitutive promoter.

A preferred construct for reducing expression in a plant of an endogenous REV gene is one comprising an inverted repeat of a REV ΔHDZip/START nucleic acid sequence, preferably capable of forming a hairpin structure, which inverted repeat is under the control of a constitutive promoter.

Constructs useful in the methods according to the present invention may be created using recombinant DNA technology well known to persons skilled in the art. The genetic constructs may be inserted into vectors, which may be commercially available, suitable for transforming into plants and suitable for expression of the gene of interest in the transformed cells. The invention therefore provides use of a genetic construct as defined hereinabove in the methods of the invention.

The sequence of interest is operably linked to one or more control sequences (at least to a promoter) capable of increasing expression in a plant. Advantageously, any type of promoter may be used to drive expression of the nucleic acid sequence.

In one embodiment, the REV ΔHDZip/START nucleic acid sequence is operably linked to a constitutive promoter. Preferably the promoter is a ubiquitous promoter and is expressed predominantly throughout the plant. Preferably, the constitutive promoter is substantially as represented by SEQ ID NO: 218 or SEQ ID NO: 58, further preferably the promoter capable of preferentially expressing the nucleic acid sequence throughout the plant is a GOS2 promoter, most preferably the GOS2 promoter is from rice (SEQ ID NO: 218 or SEQ ID NO: 58). It should be clear that the applicability of the present invention is not restricted to the REV ΔHDZip/START nucleic acid as represented by SEQ ID NO: 194, nor is the applicability of the invention restricted to expression of a REV ΔHDZip/START nucleic acid sequence when driven by a GOS2 promoter. An alternative constitutive promoter that is useful in the methods of the present invention is the high mobility group protein promoter (SEQ ID NO: 293, PRO0170). Examples of other constitutive promoters that may also be used to drive expression of a REV ΔHDZip/START nucleic acid sequence are shown in the definitions section.

Optionally, one or more terminator sequences may also be used in the construct introduced into a plant.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

For the detection of the successful transfer of the nucleic acid sequences as used in the methods of the invention and/or selection of transgenic plants comprising these nucleic acids, it is advantageous to use marker genes (or reporter genes). Therefore, the genetic construct may optionally comprise a selectable marker gene. Selectable markers are described in more detail in the “definitions” section herein. The marker genes may be removed or excised from the transgenic cell once they are no longer needed. Techniques for marker removal are known in the art, useful techniques are described above in the definitions section.

The present invention also encompasses plants including plant parts and plant cells obtainable by the methods according to the present invention having reduced expression of an endogenous REV gene using a REV ΔHDZip/START nucleic acid sequence and which have increased yield relative to control plants.

The invention also provides a method for the production of transgenic plants having increased yield relative to control plants, which transgenic plants have reduced expression of an endogenous REV gene using a REV ΔHDZip/START nucleic acid sequence.

More specifically, the present invention provides a method for the production of transgenic plants having increased yield relative to control plants, which method comprises:
(i) introducing and expressing in a plant, plant part or plant cell a genetic construct comprising one or more control sequences for reducing expression in a plant of an endogenous REV gene using a REV ΔHDZip/START nucleic acid sequence; and
(ii) cultivating the plant, plant part or plant cell under conditions promoting plant growth and development.

Preferably, the construct introduced into a plant is one comprising an inverted repeat (in part or complete) of a REV ΔHDZip/START nucleic acid sequence, preferably capable of forming a hairpin structure, which inverted repeat is under the control of a constitutive promoter.

The nucleic acid may be introduced directly into a plant cell or into the plant itself (including introduction into a tissue, organ or any other part of a plant). According to a preferred feature of the present invention, the construct is introduced into a plant by transformation.

The genetically modified plant cells can be regenerated via all methods with which the skilled worker is familiar. Suitable methods can be found in the abovementioned publications by S. D. Kung and R. Wu, Potrykus or Höfgen and Willmitzer.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may be evaluated, for instance using Southern analysis, for the presence of the gene of interest, copy number and/or genomic organisation. Alternatively or additionally, expression levels of the newly introduced DNA may be monitored using Northern and/or Western analysis, or quantitative PCR, all techniques being well known to persons having ordinary skill in the art.

The generated transformed plants may be propagated by a variety of means, such as by clonal propagation or classical breeding techniques. For example, a first generation (or T1) transformed plant may be selfed to give homozygous second generation (or T2) transformants, and the T2 plants further propagated through classical breeding techniques.

The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The abovementioned growth characteristics may advantageously be modified in any plant. The methods of the invention are advantageously applicable to any plant. Plants that are particularly useful in the methods of the invention include all plants which belong to the superfamily Viridiplantae, in particular monocotyledonous and dicotyledonous plants including fodder or forage legumes, ornamental plants, food crops, trees or shrubs. According to a preferred embodiment of the present invention, the plant is a crop plant. Examples of crop plants include soybean, sunflower, canola, alfalfa, rapeseed, cotton, tomato, potato and tobacco. Further preferably, the plant is a monocotyledonous plant. Examples of monocotyledonous plants include sugarcane. More preferably the plant is a cereal. Examples of cereals include rice, maize, wheat, barley, millet, rye, triticale, sorghum and oats.

Other advantageous plants are selected from the group consisting of Asteraceae such as the genera Helianthus, Tagetes e.g. the species Helianthus annus [sunflower], Tagetes lucida, Tagetes erecta or Tagetes tenuifolia [Marigold], Brassicaceae such as the genera Brassica, Arabadopsis e.g. the species Brassica napus, Brassica rapa ssp. [canola, oilseed rape, turnip rape] or Arabidopsis thaliana; Fabaceae such as the genera Glycine e.g. the species Glycine max, Soja hispida or Soja max [soybean]; Linaceae such as the genera Linum e.g. the species Linum usitatissimum, [flax, linseed]; Poaceae such as the genera Hordeum, Secale, Avena, Sorghum, Oryza, Zea, Triticum e.g. the species Hordeum vulgare [barley]; Secale cereale [rye], Avena sativa, Avena fatua, Avena byzantina, Avena fatua var. sativa, Avena hybrida [oat], Sorghum bicolor [Sorghum, millet], Oryza sativa, Oryza latifolia [rice], Zea mays [corn, maize] Triticum aestivum, Triticum durum, Triticum turgidum, Triticum hybernum, Triticum macha, Triticum sativum or Triticum vulgare [wheat, bread wheat, common wheat]; Solanaceae such as the genera Solanum, Lycopersicon e.g. the species Solanum tuberosum [potato], Lycopersicon esculentum, Lycopersicon lycopersicum, Lycopersicon pyriforme, Solanum integrifolium or Solanum lycopersicum [tomato].

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

The invention also extends to harvestable parts of a plant such as, but not limited to seeds, leaves, fruits, flowers, stems, roots, rhizomes, tubers and bulbs. The invention furthermore relates to products derived, preferably directly derived, from a harvestable part of such a plant, such as dry pellets or powders, oil, fat and fatty acids, starch or proteins.

The present invention also encompasses use of a REV ΔHDZip/START nucleic acid sequence, for reduction of endogenous REV gene expression in a plant, plant part, or plant cell for increasing plant yield as defined hereinabove.

### CLE

It has now been found that modulating expression in a plant of a nucleic acid encoding a CLE-like polypeptide gives plants having enhanced yield-related traits relative to control plants.

Therefore, the invention provides a method for enhancing yield-related traits in plants relative to control plants, comprising modulating expression in a plant of a nucleic acid encoding a CLE-like polypeptide, or a part thereof.

A “reference”, “reference plant”, “control”, “control plant”, “wild type” or “wild type plant” is in particular a cell, a tissue, an organ, a plant, or a part thereof, which was not produced according to the method of the invention. Accordingly, the terms “wild type”, “control” or “reference” are exchangeable and can be a cell or a part of the plant such as an organelle or tissue, or a plant, which was not modified or treated according to the herein described method according to the invention. Accordingly, the cell or a part of the plant such as an organelle or a plant used as wild type, control or reference corresponds to the cell, plant or part thereof as much as possible and is in any other property but in the result of the process of the invention as identical to the subject matter of the invention as possible. Thus, the wild type, control or reference is treated identically or as identical as possible, saying that only conditions or properties might be different which do not influence the quality of the tested property. That means in other words that the wild type denotes (1) a plant, which carries the unaltered or not modulated form of a gene or allele or (2) the starting material/plant from which the plants produced by the process or method of the invention are derived.

Preferably, any comparison between the wild type plants and the plants produced by the method of the invention is carried out under analogous conditions. The term “analogous conditions” means that all conditions such as, for example, culture or growing conditions, assay conditions (such as buffer composition, temperature, substrates, pathogen strain, concentrations and the like) are kept identical between the experiments to be compared.

The “reference”, “control”, or “wild type” is preferably a subject, e.g. an organelle, a cell, a tissue, a plant, which was not modulated, modified or treated according to the herein described process of the invention and is in any other property as similar to the subject matter of the invention as possible. The reference, control or wild type is in its genome, transcriptome, proteome or metabolome as similar as possible to the subject of the present invention. Preferably, the term “reference-” “control-” or “wild type-”-organelle, -cell, -tissue or plant, relates to an organelle, cell, tissue or plant, which is nearly genetically identical to the organelle, cell, tissue or plant, of the present invention or a part thereof preferably 95%, more preferred are 98%, even more preferred are 99.00%, in particular 99.10%, 99.30%, 99.50%, 99.70%, 99.90%, 99.99%, 99.999% or more. Most preferable the “reference”, “control”, or “wild type” is preferably a subject, e.g. an organelle, a cell, a tissue, a plant, which is genetically identical to the plant, cell organelle used according to the method of the invention except that nucleic acid molecules or the gene product encoded by them are changed, modulated or modified according to the inventive method.

The term “modulation” means in relation to expression or gene expression, a process in which the expression level is changed by said gene expression in comparison to the control plant, preferably the expression level is decreased. The original, unmodulated expression may be of any kind of expression of a structural RNA (rRNA, tRNA) or mRNA with subsequent translation. The term “modulating the activity” shall mean any change of the expression of the inventive nucleic acid sequences or encoded proteins, which leads to increased yield and/or increased growth of the plants.

A preferred method for modulating (preferably, decreasing) expression of a nucleic acid encoding a CLE-like polypeptide is by introducing and expressing in a plant a genetic construct into which the nucleic acid encoding such a CLE-like polypeptide is cloned as an inverted repeat (in part or completely), separated by a spacer (non-coding DNA).

Any reference hereinafter to a “protein useful in the methods of the invention” is taken to mean a POI polypeptide as defined herein. Any reference hereinafter to a “nucleic acid useful in the methods of the invention” is taken to mean a nucleic acid capable of encoding such a POI polypeptide. The nucleic acid to be introduced into a plant (and therefore useful in performing the methods of the invention) is any nucleic acid encoding the type of protein which will now be described, hereafter also named “POI nucleic acid” or “POI gene”.

CLE-like polypeptide encoding genes are known in the art (see for example Cock and McCormick (Plant Physiol. 126, 939-942, 2001) and useful in the methods of the invention are nucleic acids encoding a CLE-like polypeptide, or a part thereof.

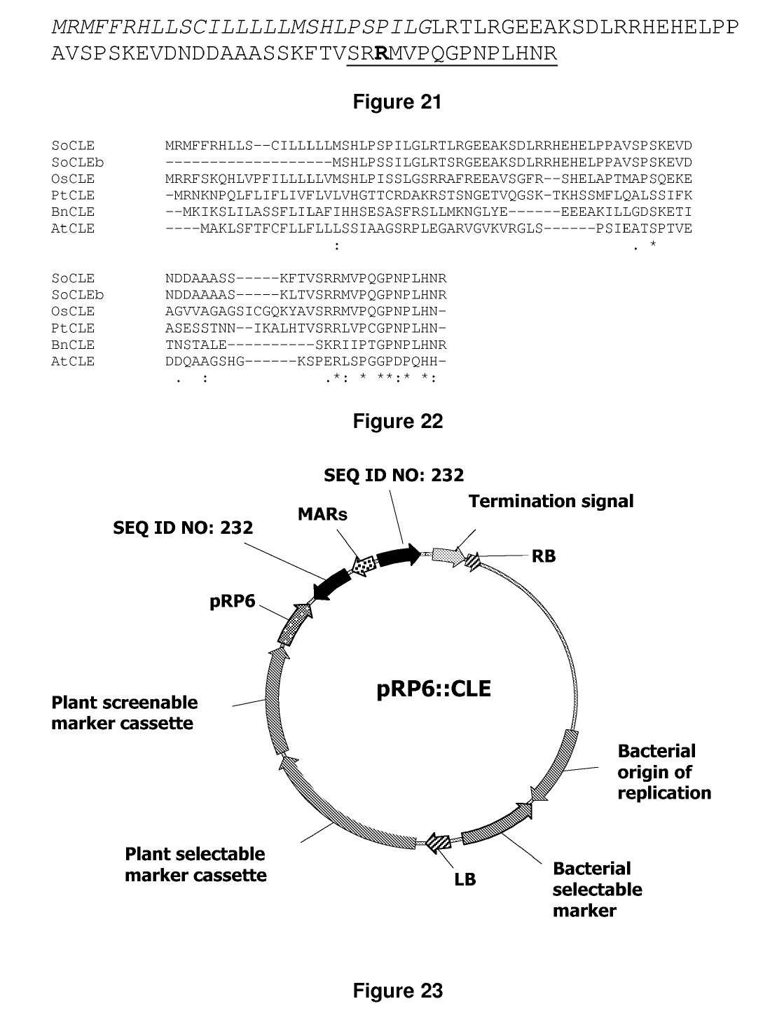

The term “CLE-like polypeptide” as defined herein refers to a polypeptide homologous to SEQ ID NO: 233. CLE-like polypeptides comprise an N-terminal signal sequence and a conserved motif (Cock and McCormick, 2001: FIG. 21), also known as CLE domain (Strabala et al., 2006), which is located at or near the C-terminus of the polypeptide. The unprocessed polypeptides are generally between 60 to 140 amino acids long and have a high isoelectric point, preferably above pl 7.0, more preferably above pl 8.0, most preferably above pl 9.0 (for example, the protein represented by SEQ ID NO: 233 has a pl of 10.46). After cleavage of signal sequence, the CLE-like polypeptide may be further processed by proteolytic cleavage in the C-terminal part, preferably at a conserved Arg residue in the N-terminal part of the CLE domain, thereby generating a biologically active short peptide encompassing most of the CLE domain (Ni and Clark, Plant Physiol. 140, 726-733, 2006).

The terms “domain” and “motif” are defined in the definitions section herein. Specialist databases exist for the identification of domains. The CLE domain in a CLE-like polypeptide may be identified using, for example, SMART (Schultz et al. (1998) Proc. Natl. Acad. Sci. USA 95, 5857-5864; Letunic et al. (2002) Nucleic Acids Res 30, 242-244), InterPro (Mulder et al., (2003) Nucl. Acids. Res. 31, 315-318), Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp. 53-61, AAAI Press, Menlo Park; Hulo et al., Nucl. Acids. Res. 32:D134-D137, (2004)) or Pfam (Bateman et al., Nucleic Acids Research 30(1): 276-280 (2002)). A set of tools for in silico analysis of protein sequences is available on the ExPASY proteomics server (hosted by the Swiss Institute of Bioinformatics (Gasteiger et al., ExPASy: the proteomics server for in-depth protein knowledge and analysis, Nucleic Acids Res. 31:3784-3788 (2003)). However, the CLE domain may also easily be identified upon sequence alignment of a putative CLE-like polypeptide with CLE-like polypeptides known in the art (such as those disclosed in Cock and McCormick (2001) or Strabala et al (2006).

The CLE domain preferably has the following consensus sequence (SEQ ID NO: 237):

(S/E/R/M/P/L) (K/E/D/R/S) R (K/I/L/R/F/Q/V/M)
(V/I/S/L) (P/L/R) (R/T/Q/C/N/G/K/S) (N/G) (S/P)
(D/N/Y) P (I/L/Q/R/Y/H) (H/L/I) (H/N).

More preferably, the CLE domain has the following sequence (SEQ ID NO: 238):

(S/P) (R/E/K) R (M/L/I) (V/S/I) P (Q/G/C/T/S) GP
(N/D) P (L/Q/H) H (H/N).

Most preferably, the CLE domain has the following sequence:

SRRMVPQGPNPLHN.

The CLE domain comprises a number of highly conserved amino acids, including the Arg residue necessary for proteolytic processing (Arg73 in SEQ ID NO: 233, see FIGS. 21 and 22), and two or three Pro residues.

Preferred for use in the methods of the invention is a nucleic acid encoding at least part of the CLE-like polypeptide, as represented by SEQ ID: 233, or a nucleic acid encoding at least part of a homologue of SEQ ID NO: 233. Examples of CLE-like polypeptides include SEQ ID NO: 233 and also encompasses homologues (including orthologues and paralogues) of SEQ ID NO: 233. The invention is illustrated by transforming rice plants with the Saccharum officinarum sequence represented by SEQ ID NO: 232, encoding the polypeptide of SEQ ID NO: 233. SEQ ID NO: 240 from Populus, SEQ ID NO: 242 from rice, SEQ ID NO: 246 from Arabidopsis and SEQ ID NO: 248 from Brassica napus represent orthologues of SEQ ID NO: 233. SEQ ID NO: 246 and SEQ ID NO: 250 are paralogues of each other.

Orthologues and paralogues may easily be found by performing a so-called reciprocal blast search. This may be done by a first BLAST involving BLASTing a query sequence (for example, SEQ ID NO: 241 or SEQ ID NO: 242) against any sequence database, such as the publicly available NCBI database. BLASTN or TBLASTX (using standard default values) may be used when starting from a nucleotide sequence and BLASTP or TBLASTN (using standard default values) may be used when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 241 or SEQ ID NO: 242, the second BLAST would therefore be against rice sequences). The results of the first and second BLASTs are then compared. A paralogue is identified if a high-ranking hit from the second BLAST is from the same species as from which the query sequence is derived; an orthologue is identified if a high-ranking hit is not from the same species as from which the query sequence is derived. Preferred orthologues are orthologues of SEQ ID NO: 232 or SEQ ID NO: 233. High-ranking hits are those having a low E-value. The lower the E-value, the more significant the score (or in other words the lower the chance that the hit was found by chance). Computation of the E-value is well known in the art. In addition to E-values, comparisons are also scored by percentage identity. Percentage identity refers to the number of identical nucleotides (or amino acids) between the two compared nucleic acid (or polypeptide) sequences over a particular length. Preferably the score is greater than 50, more preferably greater than 100; and preferably the E-value is less than e-5, more preferably less than e-6. In the case of large families, ClustalW may be used, followed by the generation of a neighbour joining tree, to help visualize clustering of related genes and to identify orthologues and paralogues.

Homologues (or homologous proteins, encompassing orthologues and paralogues) may readily be identified using routine techniques well known in the art, such as by sequence alignment. Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the alignment of two complete sequences that maximizes the number of matches and minimizes the number of gaps. The BLAST algorithm (Altschul et al. (1990) J Mol Biol 215: 403-410) calculates percent sequence identity and performs a statistical analysis of the similarity between the two sequences. The software for performing BLAST analysis is publicly available through the National Centre for Biotechnology Information. Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 4, 29, 2003). Minor manual editing may be performed to optimise alignment between conserved motifs, as would be apparent to a person skilled in the art. Furthermore, instead of using full-length sequences for the identification of homologues, specific domains (such as the CLE domain) may be used as well. The sequence identity values, which are indicated below as a percentage were determined over the entire nucleic acid or amino acid sequence using the programs mentioned above using the default parameters.

Preferably, the CLE-like polypeptides useful in the methods of the present invention have, in increasing order of preference, at least 30%, 35%, 40%, 45%, 50%, 55%, 60%, 65%, 70%, 75%, 80%, 85%, 90%, 95%, 96%, 97%, 98% or 99% sequence identity to the polypeptide of SEQ ID NO: 233. Alternatively, the sequence identity among homologues may be determined using a specific domain (such as the CLE domain). A CLE domain may be identified and delineated using the databases and tools for protein identification listed above, and/or methods for the alignment of sequences for comparison. In some instances, default parameters may be adjusted to modify the stringency of the search. For example using BLAST, the statistical significance threshold (called “expect” value) for reporting matches against database sequences may be increased to show less stringent matches. In this way, short nearly exact matches may be identified.

The term CLE-like nucleic acid as used herein, refers to any nucleic acid encoding a CLE-like polypeptide as defined above, or the complement thereof. The CLE-like nucleic acid need not be full-length nucleic acids, since performance of the methods of the invention does not rely on the use of full-length nucleic acid sequences. Furthermore, examples of nucleic acids suitable for use in performing the methods of the invention include but are not limited to those represented by any one of: SEQ ID NO: 232, SEQ ID NO: 239, SEQ ID NO: 241, SEQ ID NO: 243, SEQ ID NO: 245 or SEQ ID NO: 247. Nucleic acid variants may also be useful in practising the methods of the invention. Examples of such variants include portions of nucleic acids, hybridising sequences, splice variants, allelic variants either naturally occurring or by DNA manipulation.

Reference herein to a “CLE-like” nucleic acid sequence is taken to mean a sufficient length of substantially contiguous nucleotides from a CLE-like nucleic acid sequence. In order to perform gene silencing, this may be as little as 20, 19, 18, 17, 16, 15, 14, 13, 12, 11, 10 or fewer nucleotides, alternatively this may be as much as the CLE-like nucleic acid sequence (including the 5′ and/or 3′ UTR, either in part or in whole). A nucleic acid sequence encoding a (functional) polypeptide is not a requirement for the various methods discussed above for the substantial reduction of expression of an endogenous CLE-like gene.

The term “portion” or “part” of a CLE-like nucleic acid as used herein refers to a piece of DNA encoding at least part of a CLE-like polypeptide, or the complement thereof, but may also be a part of the 5′ or 3′ untranslated region (UTR) of a CLE polypeptide encoding cDNA, or the complement thereof, or may be the entire 5′ or 3′ UTR, or its complement. The term cDNA as used herein is meant to encompass not only the coding sequences, but also the non-coding sequences that correspond to the 5′ and 3′ UTRs of the mRNA.

The terms “fragment”, “fragment of a sequence” or “part of a sequence” “portion” or “portion thereof” mean a truncated sequence of the original sequence referred to. The truncated sequence (nucleic acid or protein sequence) can vary widely in length; the minimum size being a sequence of sufficient size to provide a sequence with at least a comparable function and/or activity of the original sequence referred to or hybidizing with the nucleic acid molecule of the invention or used in the process of the invention under stringend conditions, while the maximum size is not critical. In some applications, the maximum size usually is not substantially greater than that required to provide the desired activity and/or function(s) of the original sequence. A comparable function means at least 40%, 45% or 50%, preferably at least 60%, 70%, 80% or 90% or more of the original sequence.

A portion may be prepared, for example, by making one or more deletions to a nucleic acid encoding a CLE-like polypeptide as defined hereinabove. The portions may be used in isolated form or they may be fused to other coding (or non-coding) sequences. The portion is typically at least 100 nucleotides in length, preferably at least 200, 250 nucleotides in length, more preferably at least 300, 350 nucleotides in length and most preferably at least 400 or 450 nucleotides in length. Preferably, the portion is a portion of a nucleic acid as represented by any one of SEQ ID NO: 232, SEQ ID NO: 239, SEQ ID NO: 241, SEQ ID NO: 243, SEQ ID NO: 245 or SEQ ID NO: 247. Most preferably the portion is a portion of a nucleic acid as represented by SEQ ID NO: 232. A preferred portion of a CLE-like nucleic acid for use in the methods of the present invention is a portion having high homology to the transcribed sequence of the endogenous target CLE-like gene or to the complement thereof, while having low homology or no homology to transcribed sequences (or the complement sequences thereof) of endogenous non-target CLE-like genes

Another nucleic acid variant useful in the methods of the invention, is a nucleic acid capable of hybridising under reduced stringency conditions, preferably under stringent conditions, with a nucleic acid encoding a CLE-like polypeptide as defined hereinabove, or a with a portion as defined hereinabove.

Hybridising sequences useful in the methods of the invention, encode at least part of a CLE-like polypeptide as defined above, or are capable of hybridising to the 5′ or 3′ UTR of a CLE-like polypeptide encoding mRNA or cDNA. The hybridising sequence is typically at least 100 nucleotides in length, preferably at least 200 nucleotides in length, more preferably at least 300 nucleotides in length and most preferably at least 400 nucleotides nucleotides in length. Preferably, the hybridising sequence is one that is capable of hybridising to any of the nucleic acids represented by (or to probes derived from) SEQ ID NO: 232, SEQ ID NO: 239, SEQ ID NO: 241, SEQ ID NO: 243, SEQ ID NO: 245 or SEQ ID NO: 247, or to a portion of any of the aforementioned sequences, a portion being as defined above. Most preferably the hybridising sequence is capable of hybridising to SEQ ID NO: 232, or to portions (or probes) thereof. Methods for designing probes are well known in the art. Probes are generally less than 500, 400, 300, 200 bp in length, preferably less than 100 bp in length. Commonly, probe lengths for DNA-DNA hybridisations such as Southern blotting, vary between 100 and 500 bp, whereas the hybridising region in probes for DNA-DNA hybridisations such as in PCR amplification generally are shorter than 50 but longer than 10 nucleotides, preferably they are 15, 20, 25, 30, 35, 40, 45 or 50 bp in length.

Another nucleic acid variant useful in the methods of the invention is a splice variant encoding a CLE-like polypeptide as defined hereinabove. Preferred splice variants are splice variants of a nucleic acid encoding the CLE-like polypeptide represented by SEQ ID NO: 233, or splice variants encoding orthologues or paralogues of SEQ ID NO: 233. Further preferred are splice variants of nucleic acids represented by any one of SEQ ID NO: 232, SEQ ID NO: 239, SEQ ID NO: 241, SEQ ID NO: 243, SEQ ID NO: 245 or SEQ ID NO: 247. Most preferred is a splice variant of a nucleic acid as represented by SEQ ID NO: 232.

Another nucleic acid variant useful in performing the methods of the invention is an allelic variant of a nucleic acid encoding a CLE-like protein as defined hereinabove. Allelic variants exist in nature, and encompassed within the methods of the present invention is the use of these natural alleles. The allelic variant may be an allelic variant of a nucleic acid encoding a CLE-like polypeptide represented by SEQ ID NO: 233, or an allelic variant of a nucleic acid encoding orthologues or paralogues of any of the aforementioned SEQ ID NOs. Further preferred are allelic variants of nucleic acids represented by any one of SEQ ID NO: 232, SEQ ID NO: 239, SEQ ID NO: 241, SEQ ID NO: 243, SEQ ID NO: 245 or SEQ ID NO: 247. Most preferred is an allelic variant of a nucleic acid as represented by SEQ ID NO: 232.

According to the present invention, there is provided a method for enhancing yield-related traits in plants, comprising introducing and expressing in a plant portions, hybridising sequences, splice variants, or allelic variants of any one of the nucleic acids given in Table HH of Example 33, or comprising introducing and expressing in a plant portions, hybridising sequences, splice variants, or allelic variants of a nucleic acid encoding an orthologue, paralogue or homologue of any of the amino acid sequences given in Table HH of Example 33.

A further nucleic acid variant useful in the methods of the invention is a nucleic acid variant obtained by gene shuffling. Gene shuffling or directed evolution may also be used to generate variants of nucleic acids encoding CLE-like polypeptides as defined above. Furthermore, nucleic acid variants may also be obtained by site-directed mutagenesis. Several methods are available to achieve site-directed mutagenesis, the most common being PCR based methods (Current Protocols in Molecular Biology. Wiley Eds.).

Also useful in the methods of the invention are nucleic acids encoding homologues of any one of the amino acids represented by SEQ ID NO 233, or orthologues or paralogues of thereof; and nucleic acids encoding derivatives of the polypeptide represented by SEQ ID NO 233 or orthologues or paralogues thereof.

Furthermore, CLE-like nucleic acids useful in the methods of the present invention (at least in their native form) typically, but not necessarily, encode polypeptides having signalling activity. Preferably, CLE-like nucleic acids encode polypeptides which, when overexpressed in plants, cause a wus-like phenotype. Further preferably, a CLE-like polypeptide, when overexpressed in Arabidopsis, results in a Aii phenotype as defined by Strabala et al. (2006). More preferably, a CLE-like nucleic acid, when expressed as an inverted repeat under control of the promoter represented by SEQ ID NO: 236 in rice results in increased seed yield, such as increased total seed weight.

Nucleic acids encoding CLE-like polypeptides may be derived from any natural or artificial source. The nucleic acid may be modified from its native form in composition and/or genomic environment through deliberate human manipulation. Preferably the CLE-like polypeptide-encoding nucleic acid is from a plant, further preferably from a monocotyledonous plant, more preferably from the family of Poaceae, most preferably the nucleic acid is from Saccharum officinarum.

The invention also provides genetic constructs and vectors to facilitate introduction and/or expression of the nucleic acid sequences useful in the methods according to the invention, in a plant.

Therefore, there is provided a gene construct comprising:
(i) a CLE-like nucleic acid as defined hereinabove, or a portion thereof;
(ii) one or more control sequences operably linked to the nucleic acid of (i).

A preferred construct is one comprising an inverted repeat of a CLE-like nucleic acid, preferably capable of forming a hairpin structure, which inverted repeat is under the control of a seed specific promoter.

Constructs useful in the methods according to the present invention may be constructed using recombinant DNA technology well known to persons skilled in the art. The gene constructs may be inserted into vectors, which may be commercially available, suitable for transforming into plants and suitable for transcribing of the gene of interest in the transformed cells. The invention therefore provides use of a gene construct as defined hereinabove in the methods of the invention.

Plants are transformed with a vector comprising the sequence of interest. The skilled artisan is well aware of the genetic elements that must be present on the vector in order to successfully transform, select and propagate host cells containing the sequence of interest. The sequence of interest is operably linked to one or more control sequences (at least to a promoter).

Advantageously, any type of promoter may be used in the methods of the present invention. Preferred promoters are in particular those which bring gene expression in tissues and organs, in seed cells, such as endosperm cells and cells of the developing embryo. Suitable promoters are the oilseed rape napin gene promoter (U.S. Pat. No. 5,608,152), the Vicia faba USP promoter (Baeumlein et al., Mol Gen Genet, 1991, 225 (3): 459-67), the Arabidopsis oleosin promoter (WO 98/45461), the Phaseolus vulgaris phaseolin promoter (U.S. Pat. No. 5,504,200), the Brassica Bce4 promoter (WO 91/13980), the bean arc5 promoter, the carrot DcG3 promoter, or the Legumin B4 promoter (LeB4; Baeumlein et al., 1992, Plant Journal, 2 (2): 233-9), and promoters which bring about the seed-specific expression in monocotyledonous plants such as maize, barley, wheat, rye, rice and the like. Advantageous seed-specific promoters are the sucrose binding protein promoter (WO 00/26388), the phaseolin promoter and the napin promoter. Suitable promoters which must be considered are the barley lpt2 or Ipt1 gene promoter (WO 95/15389 and WO 95/23230), and the promoters described in WO 99/16890 (promoters from the barley hordein gene, the rice glutelin gene, the rice oryzin gene, the rice prolamin gene, the wheat gliadin gene, the wheat glutelin gene, the maize zein gene, the oat glutelin gene, the sorghum kasirin gene and the rye secalin gene). Further suitable promoters are Amy32b, Amy 6-6 and Aleurain [U.S. Pat. No. 5,677,474], Bce4 (oilseed rape) [U.S. Pat. No. 5,530,149], glycinin (soya) [EP 571 741], phosphoenolpyruvate carboxylase (soya) [JP 06/62870], ADR12-2 (soya) [WO 98/08962], isocitrate lyase (oilseed rape) [U.S. Pat. No. 5,689,040] or a amylase (barley) [EP 781 849]. Other promoters which are available for the expression of genes in plants are leaf-specific promoters such as those described in DE-A 19644478 or light-regulated promoters such as, for example, the pea petE promoter.

Preferably, the CLE-like nucleic acid or variant thereof is operably linked to a seed-specific promoter. A seed-specific promoter is transcriptionally active predominantly in seed tissue, but not necessarily exclusively in seed tissue (in cases of leaky expression). The seed-specific promoter may be active during seed development and/or during germination. Seed-specific promoters are well known in the art. Preferably, the seed-specific promoter is an endosperm specific promoter. More preferably, the promoter is a rice Prolamine RP6 or a functionally equivalent promoter. Most preferably, the promoter sequence is as represented by SEQ ID NO: 236. It should be clear that the applicability of the present invention is not restricted to the CLE-like nucleic acid represented by SEQ ID NO: 232, nor is the applicability of the invention restricted to transcription of a CLE-like nucleic acid when driven by a seed-specific promoter. Examples of other seed-specific promoters (including endosperm specific promoters) are listed above.

Optionally, one or more terminator sequences may be used in the construct introduced into a plant. An intron sequence may also be added to the 5′ untranslated region (UTR) or in the coding sequence to increase the amount of the mature message that accumulates in the cytosol. Other control sequences (besides promoter, enhancer, silencer, intron sequences, 3′UTR and/or 5′UTR regions) may be protein and/or RNA stabilizing elements. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

For the detection and/or selection of the successful transfer of the nucleic acid sequences as depicted in the sequence protocol and used in the process of the invention, it is advantageous to use marker genes (=reporter genes). Therefore genetic construct may optionally comprise a selectable marker gene. These marker genes enable the identification of a successful transfer of the nucleic acid molecules via a series of different principles, for example via visual identification with the aid of fluorescence, luminescence or in the wavelength range of light which is discernible for the human eye, by a resistance to herbicides or antibiotics, via what are known as nutritive markers (auxotrophism markers) or antinutritive markers, via enzyme assays or via phytohormones. Examples of such markers which may be mentioned are GFP (=green fluorescent protein); the luciferin/luceferase system, the β-galactosidase with its colored substrates, for example X-Gal, the herbicide resistances to, for example, imidazolinone, glyphosate, phosphinothricin or sulfonylurea, the antibiotic resistances to, for example, bleomycin, hygromycin, streptomycin, kanamycin, tetracyclin, chloramphenicol, ampicillin, gentamycin, geneticin (G418), spectinomycin or blasticidin, to mention only a few, nutritive markers such as the utilization of mannose or xylose, or antinutritive markers such as the resistance to 2-deoxyglucose. Preferred selectable markers in plants comprise those, which confer resistance to an herbicide such as glyphosate or gluphosinate. Other suitable markers are, for example, markers, which encode genes involved in biosynthetic pathways of, for example, sugars or amino acids, such as β galactosidase, ura3 or ilv2. Markers, which encode genes such as luciferase, gfp or other fluorescence genes, are likewise suitable. These markers and the aforementioned markers can be used in mutants in whom these genes are not functional since, for example, they have been deleted by conventional methods. This list is a small number of possible markers. The skilled worker is very familiar with such markers. Different markers are preferred, depending on the organism and the selection method.

In a preferred embodiment of the present invention, modulated expression of a CLE-like protein is decreased expression of a CLE-like protein, preferably decreased expression of an endogenous CLE-like protein.

Reference herein to an “endogenous” CLE-like gene not only refers to a CLE-like gene as found in a plant in its natural form (i.e., without there being any human intervention), but also refers to isolated CLE-like nucleic acid sequences subsequently introduced into a plant. For example, a transgenic plant containing a CLE-like transgene may encounter a substantial reduction of the transgene expression and/or substantial reduction of expression of an endogenous CLE-like gene, according to the methods of the invention.

A preferred method for decreasing expression of a CLE-like protein is by using an expression vector into which a CLE-like nucleic acid sequence encoding CLE-like polypeptide has been cloned as an inverted repeat (in part or completely), separated by a spacer (non-coding DNA).

“Reduction” or “decrease” or “downregulation” of expression or “gene silencing” are used interchangeably herein, and are defined above. Preferably, the decreased expression is not complete elimination of expression. Preferably, reducing the expression of the endogenous CLE-like gene using a CLE-like nucleic acid sequence leads to the appearance of one or more phenotypic traits.

This reduction of endogenous CLE-like gene expression may be achieved by using any one or more of several well-known “gene silencing” methods, as described above.

A preferred method for reducing expression in a plant of an endogenous CLE-like gene via RNA-mediated silencing is by using an inverted repeat of a CLE-like nucleic acid or a part thereof, preferably capable of froming a hairpin structure. The inverted repeat is cloned in an expression vector comprising control sequences. A non-coding DNA nucleic acid sequence (a spacer, for example a matrix attachment region fragment (MAR), an intron, a polylinker, etc) is located between the two inverted CLE-like nucleic acids forming the inverted repeat. After transcription of the inverted repeat, a chimeric RNA with a self-complementary structure is formed (partial or complete). This double-stranded RNA structure is referred to as the hairpin RNA (hpRNA). The hpRNA is processed by the plant into siRNAs that are incorporated into a RISC. The RISC further cleaves the mRNA transcripts encoding a CLE-like polypeptide, thereby substantially reducing the number of mRNA transcripts to be translated into a CLE-like polypeptide. See for example, Grierson et al. (1998) WO 98/53083; Waterhouse et al. (1999) WO 99/53050).

Preferably, a CLE-like nucleic acid sequence from any given plant species is introduced into that same species. For example, a CLE-like nucleic acid sequence from rice is transformed into a rice plant. However, it is not an absolute requirement that the CLE-like nucleic acid sequence to be introduced originates from the same plant species as the plant in which it will be introduced, as shown in the examples section where a sugarcane sequence is introduced into rice to obtain the desired effects. It is sufficient that there is substantial homology between the endogenous CLE-like target gene and the CLE-like nucleic acid to be introduced.

The expression of an endogenous CLE-like gene may also be reduced by introducing a genetic modification, within the locus of the CLE-like gene or elsewhere in the genome. The locus of a gene as defined herein is taken to mean a genomic region, which includes the gene of interest and 10 kb up- or down stream of the coding region.

The genetic modification may be introduced, for example, by any one (or more) of the following methods: T-DNA tagging, TILLING, site-directed mutagenesis, directed evolution, homologous recombination. Following introduction of the genetic modification, there follows a step of selecting for reduced expression of an endogenous CLE-like gene, which reduction in expression gives plants having increased yield compared to control plants.

Site-directed mutagenesis and random mutagenesis may be used to generate variants of CLE-like nucleic acid sequences which variants encode CLE-like polypeptides with reduced activity. Several methods are available to achieve site-directed mutagenesis, the most common being PCR based methods (see for example Current Protocols in Molecular Biology. Wiley Eds).

Directed evolution may also be used to generate variants of CLE-like nucleic acid sequences which variants encode CLE-like polypeptides with reduced activity.

T-DNA tagging, TILLING, site-directed mutagenesis and directed evolution are examples of technologies that enable the generation of novel alleles and variants of CLE-like nucleic acid sequences which variants encode CLE-like polypeptides with reduced activity.

Homologous recombination allows introduction in a genome of a selected nucleic acid at a defined selected position. The nucleic acid to be targeted may be an allele encoding CLE-like polypeptide with reduced activity, used to replace the endogenous gene, and needs to be targeted to the locus of the CLE-like gene.

Alternatively, a screening program may be set up to identify in a plant population natural variants of a CLE-like gene which variants encode CLE-like polypeptides with reduced activity. Such natural variants may also be used for example, to perform homologous recombination.

The methods of the invention are advantageously applicable to any plant. Plants that are particularly useful in the methods of the invention include all plants which belong to the superfamily Viridiplantae, in particular monocotyledonous and dicotyledonous plants including fodder or forage legumes, ornamental plants, food crops, trees or shrubs. According to a preferred embodiment of the present invention, the plant is a crop plant. Examples of crop plants include soybean, sunflower, canola, alfalfa, rapeseed, cotton, tomato, potato and tobacco. Further preferably, the plant is a monocotyledonous plant. Examples of monocotyledonous plants include sugarcane. More preferably the plant is a cereal. Examples of cereals include rice, maize, wheat, barley, millet, rye, triticale, sorghum and oats.

Other advantageous plants are selected from the group consisting of Asteraceae such as the genera Helianthus, Tagetes e.g. the species Helianthus annus [sunflower], Tagetes lucida, Tagetes erecta or Tagetes tenuifolia [Marigold], Brassicaceae such as the genera Brassica, Arabadopsis e.g. the species Brassica napus, Brassica rapa ssp. [canola, oilseed rape, turnip rape] or Arabidopsis thaliana. Fabaceae such as the genera Glycine e.g. the species Glycine max, Soja hispida or Soja max [soybean]. Linaceae such as the genera Linum e.g. the species Linum usitatissimum, [flax, linseed]; Poaceae such as the genera Hordeum, Secale, Avena, Sorghum, Oryza, Zea, Triticum e.g. the species Hordeum vulgare [barley]; Secale cereale [rye], Avena sativa, Avena fatua, Avena byzantina, Avena fatua var. sativa, Avena hybrida [oat], Sorghum bicolor [Sorghum, millet], Oryza sativa, Oryza latifolia [rice], Zea mays [corn, maize] Triticum aestivum, Triticum durum, Triticum turgidum, Triticum hybernum, Triticum macha, Triticum sativum or Triticum vulgare [wheat, bread wheat, common wheat]; Solanaceae such as the genera Solanum, Lycopersicon e.g. the species Solanum tuberosum [potato], Lycopersicon esculentum, Lycopersicon lycopersicum, Lycopersicon pyriforme, Solanum integrifolium or Solanum lycopersicum [tomato].

The present invention also encompasses plants obtainable by the methods according to the present invention. The present invention therefore provides plants, plant parts or plant cells thereof obtainable by the method according to the present invention, which plants or parts or cells thereof comprise a nucleic acid transgene encoding a CLE-like protein as defined above.

The invention furthermore provides a method for the production of transgenic plants having altered yield-related trait relative to control plants, comprising introduction and expression in a plant of a CLE-like nucleic acid as defined hereinabove and useful in a method for downregulating expression as discussed above.

Host plants for the nucleic acids or the vector used in the method according to the invention, the expression cassette or construct or vector are, in principle, advantageously all plants, which are capable of synthesizing the polypeptides used in the inventive method.

More specifically, the present invention provides a method for the production of transgenic plants having altered yield-related traits, which method comprises:
(i) introducing and expressing a CLE-like nucleic acid in a construct for downregulating CLE-like gene expression into a plant cell; and
(ii) cultivating the plant cell under conditions promoting plant growth and development.

The nucleic acid may be introduced directly into a plant cell or into the plant itself (including introduction into a tissue, organ or any other part of a plant). According to a preferred feature of the present invention, the nucleic acid is preferably introduced into a plant by transformation.

Further preferably the construct for downregulating CLE-like gene expression and introduced into the plant cell or plant comprise an inverted repeat of the CLE-like nucleic acid or a part thereof.

The transfer of foreign genes into the genome of a plant is called transformation. In doing this the methods described for the transformation and regeneration of plants from plant tissues or plant cells are utilized for transient or stable transformation. An advantageous transformation method is the transformation in planta. To this end, it is possible, for example, to allow the agrobacteria to act on plant seeds or to inoculate the plant meristem with agrobacteria. It has proved particularly expedient in accordance with the invention to allow a suspension of transformed agrobacteria to act on the intact plant or at least the flower primordia. The plant is subsequently grown on until the seeds of the treated plant are obtained (Clough and Bent, Plant J. (1998) 16, 735-743). To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Further advantageous transformation methods, in particular for plants, are known to the skilled worker and are described herein below

Generally after transformation, plant cells or cell groupings are selected for the presence of one or more markers which are encoded by plant-expressible genes co-transferred with the gene of interest, following which the transformed material is regenerated into a whole plant.

As mentioned Agrobacteria transformed with an expression vector according to the invention may also be used in the manner known per se for the transformation of plants such as experimental plants like Arabidopsis or crop plants, such as, for example, cereals, maize, oats, rye, barley, wheat, soya, rice, cotton, sugarbeet, canola, sunflower, flax, hemp, potato, tobacco, tomato, carrot, bell peppers, oilseed rape, tapioca, cassaya, arrow root, tagetes, alfalfa, lettuce and the various tree, nut, and grapevine species, in particular oil-containing crop plants such as soya, peanut, castor-oil plant, sunflower, maize, cotton, flax, oilseed rape, coconut, oil palm, safflower (Carthamus tinctorius) or cocoa beans, for example by bathing scarified leaves or leaf segments in an agrobacterial solution and subsequently growing them in suitable media.

The genetically modified plant cells can be regenerated via all methods with which the skilled worker is familiar. Suitable methods can be found in the abovementioned publications by S. D. Kung and R. Wu, Potrykus or Höfgen and Willmitzer. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may be evaluated, for instance using Southern analysis, for the presence of the gene of interest, copy number and/or genomic organisation. Alternatively or additionally, downregulation of expression levels of the targeted CLE-like gene may be monitored using Northern and/or Western analysis, or quantitative PCR, all techniques being well known to persons having ordinary skill in the art.

The generated transformed plants may be propagated by a variety of means, such as by clonal propagation or classical breeding techniques. For example, a first generation (or T1) transformed plant may be selfed to give homozygous second generation (or T2) transformants, and the T2 plants further propagated through classical breeding techniques.

The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

The invention also includes host cells containing an isolated CLE-like nucleic acid as defined hereinabove. Preferred host cells according to the invention are plant cells. Host plants for the nucleic acids or the vector used in the method according to the invention, the expression cassette or construct or vector are, in principle, advantageously all plants, which are capable of synthesizing the polypeptides used in the inventive method.

The invention also extends to harvestable parts of a plant such as, but not limited to seeds, leaves, fruits, flowers, stems, roots, rhizomes, tubers and bulbs. The invention furthermore relates to products derived, preferably directly derived, from a harvestable part of such a plant, such as dry pellets or powders, oil, fat and fatty acids, starch or proteins.

Advantageously, performance of the methods according to the present invention results in plants having enhanced yield related traits, particularly increased yield, more particularly increased seed yield and/or increased biomass, relative to control plants.

Performance of the methods of the invention gives plants having enhanced yield-related traits. In particular performance of the methods of the invention gives plants having increased yield, especially increased seed yield relative to control plants. The terms “yield” and “seed yield” are described in more detail in the “definitions” section herein.

Reference herein to enhanced yield-related traits is taken to mean an increase in biomass (weight) of one or more parts of a plant, which may include aboveground (harvestable) parts and/or (harvestable) parts below ground. In particular, such harvestable parts are seeds and/or biomass, and performance of the methods of the invention results in plants having increased seed yield relative to the seed yield of control plants.

Taking corn as an example, a yield increase may be manifested as one or more of the following: increase in the number of plants established per hectare or acre, an increase in the number of ears per plant, an increase in the number of rows, number of kernels per row, kernel weight, thousand kernel weight, ear length/diameter, increase in the seed filling rate (which is the number of filled seeds divided by the total number of seeds and multiplied by 100), among others. Taking rice as an example, a yield increase may manifest itself as an increase in one or more of the following: number of plants per hectare or acre, number of panicles per plant, number of spikelets per panicle, number of flowers (florets) per panicle (which is expressed as a ratio of the number of filled seeds over the number of primary panicles), increase in the seed filling rate (which is the number of filled seeds divided by the total number of seeds and multiplied by 100), increase in thousand kernel weight, among others.

The present invention provides a method for increasing yield, especially seed yield of plants, relative to control plants, which method comprises modulating expression, preferably increasing expression, in a plant of a nucleic acid encoding a CLE-like polypeptide as defined herein.

Since the transgenic plants according to the present invention have increased yield, it is likely that these plants exhibit an increased growth rate (during at least part of their life cycle), relative to the growth rate of control plants at a corresponding stage in their life cycle.

The increased growth rate may be specific to one or more parts of a plant (including seeds), or may be throughout substantially the whole plant. Plants having an increased growth rate may have a shorter life cycle. The life cycle of a plant may be taken to mean the time needed to grow from a dry mature seed up to the stage where the plant has produced dry mature seeds, similar to the starting material. This life cycle may be influenced by factors such as early vigour, growth rate, greenness index, flowering time and speed of seed maturation. The increase in growth rate may take place at one or more stages in the life cycle of a plant or during substantially the whole plant life cycle. Increased growth rate during the early stages in the life cycle of a plant may reflect enhanced vigour. The increase in growth rate may alter the harvest cycle of a plant allowing plants to be sown later and/or harvested sooner than would otherwise be possible (a similar effect may be obtained with earlier flowering time). If the growth rate is sufficiently increased, it may allow for the further sowing of seeds of the same plant species (for example sowing and harvesting of rice plants followed by sowing and harvesting of further rice plants all within one conventional growing period). Similarly, if the growth rate is sufficiently increased, it may allow for the further sowing of seeds of different plants species (for example the sowing and harvesting of corn plants followed by, for example, the sowing and optional harvesting of soybean, potato or any other suitable plant). Harvesting additional times from the same rootstock in the case of some crop plants may also be possible. Altering the harvest cycle of a plant may lead to an increase in annual biomass production per acre (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and T-90 (time taken for plants to reach 90% of their maximal size), amongst others.

According to a preferred feature of the present invention, performance of the methods of the invention gives plants having an increased growth rate relative to control plants. Therefore, according to the present invention, there is provided a method for increasing the growth rate of plants, which method comprises modulating expression, preferably increasing expression, in a plant of a nucleic acid encoding a CLE-like polypeptide as defined herein.

An increase in yield and/or growth rate occurs whether the plant is under non-stress conditions or whether the plant is exposed to various stresses compared to control plants. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35% or 30%, preferably less than 25%, 20% or 15%, more preferably less than 14%, 13%, 12%, 11% or 10% or less in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the everyday biotic and/or abiotic (environmental) stresses to which a plant is exposed. Abiotic stresses may be due to drought or excess water, anaerobic stress, salt stress, chemical toxicity, oxidative stress and hot, cold or freezing temperatures. The abiotic stress may be an osmotic stress caused by a water stress (particularly due to drought), salt stress, oxidative stress or an ionic stress. Biotic stresses are typically those stresses caused by pathogens, such as bacteria, viruses, fungi and insects.

In particular, the methods of the present invention may be performed under non-stress conditions or under conditions of mild drought to give plants having increased yield relative to control plants. As reported in Wang et al. (Planta (2003) 218: 1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani et al. (Plant Physiol (2003) 133: 1755-1767) describes a particularly high degree of “cross talk” between drought stress and high-salinity stress. For example, drought and/or salinisation are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signalling pathways and cellular responses, such as the production of stress proteins, up-regulation of anti-oxidants, accumulation of compatible solutes and growth arrest. The term “non-stress” conditions as used herein are those environmental conditions that allow optimal growth of plants. Persons skilled in the art are aware of normal soil conditions and climatic conditions for a given location.

Performance of the methods of the invention gives plants grown under non-stress conditions or under mild drought conditions increased yield relative to control plants grown under comparable conditions. Therefore, according to the present invention, there is provided a method for increasing yield in plants grown under non-stress conditions or under mild drought conditions, which method comprises increasing expression in a plant of a nucleic acid encoding a CLE-like polypeptide.

Performance of the methods of the invention gives plants grown under conditions of nutrient deficiency, particularly under conditions of nitrogen deficiency, increased yield relative to control plants grown under comparable conditions. Therefore, according to the present invention, there is provided a method for increasing yield in plants grown under conditions of nutrient deficiency, which method comprises increasing expression in a plant of a nucleic acid encoding a CLE-like polypeptide. Nutrient deficiency may result from a lack of nutrients such as nitrogen, phosphates and other phosphorous-containing compounds, potassium, calcium, cadmium, magnesium, manganese, iron and boron, amongst others.

The present invention also encompasses use of CLE-like nucleic acids in altering yield-related traits.

Nucleic acids encoding CLE-like polypeptides may find use in breeding programmes in which a DNA marker is identified which may be genetically linked to a CLE-like gene. The nucleic acids/genes may be used to define a molecular marker. This DNA marker may then be used in breeding programmes to select plants having increased yield as defined hereinabove in the methods of the invention.

Allelic variants of a CLE-like nucleic acid/gene may also find use in marker-assisted breeding programmes. Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give increased yield. Selection is typically carried out by monitoring growth performance of plants containing different allelic variants of the sequence in question. Growth performance may be monitored in a greenhouse or in the field. Further optional steps include crossing plants in which the superior allelic variant was identified with another plant. This could be used, for example, to make a combination of interesting phenotypic features.

A CLE-like nucleic acid may also be used as probes for genetically and physically mapping the genes that they are a part of, and as markers for traits linked to those genes. Such information may be useful in plant breeding in order to develop lines with desired phenotypes. Such use of CLE-like nucleic acids requires only a nucleic acid sequence of at least 15 nucleotides in length. The CLE-like nucleic acids may be used as restriction fragment length polymorphism (RFLP) markers. Southern blots (Sambrook J, Fritsch E F and Maniatis T (1989) Molecular Cloning, A Laboratory Manual) of restriction-digested plant genomic DNA may be probed with the CLE-like nucleic acids. The resulting banding patterns may then be subjected to genetic analyses using computer programs such as MapMaker (Lander et al. (1987) Genomics 1: 174-181) in order to construct a genetic map. In addition, the nucleic acids may be used to probe Southern blots containing restriction endonuclease-treated genomic DNAs of a set of individuals representing parent and progeny of a defined genetic cross. Segregation of the DNA polymorphisms is noted and used to calculate the position of the CLE-like nucleic acid in the genetic map previously obtained using this population (Botstein et al. (1980) Am. J. Hum. Genet. 32:314-331).

The production and use of plant gene-derived probes for use in genetic mapping is described in Bernatzky and Tanksley (Plant Mol. Biol. Reporter 4: 37-41, 1986). Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid probes may be used in direct fluorescence in situ hybridisation (FISH) mapping (Trask (1991) Trends Genet. 7:149-154). Although current methods of FISH mapping favour use of large clones (several kb to several hundred kb; see Laan et al. (1995) Genome Res. 5:13-20), improvements in sensitivity may allow performance of FISH mapping using shorter probes.

A variety of nucleic acid amplification-based methods for genetic and physical mapping may be carried out using the nucleic acids. Examples include allele-specific amplification (Kazazian (1989) J. Lab. Clin. Med 11:95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic Acid Res. 18:3671), Radiation Hybrid Mapping (Walter et al. (1997) Nat. Genet. 7:22-28) and Happy Mapping (Dear and Cook (1989) Nucleic Acid Res. 17:6795-6807). For these methods, the sequence of a nucleic acid is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

The methods according to the present invention result in plants having altered yield-related traits, as described hereinbefore. These traits may also be combined with other economically advantageous traits, such as further yield-enhancing traits, tolerance to other abiotic and biotic stresses, traits modifying various architectural features and/or biochemical and/or physiological features.

### SYR

Surprisingly, it has now been found that modulating expression in a plant of a nucleic acid encoding a SYR polypeptide gives plants, when grown under abiotic stress conditions, having enhanced yield-related traits relative to control plants. According to a first embodiment, the present invention provides a method for enhancing yield-related traits in plants grown under abiotic stress conditions, relative to control plants, comprising modulating expression in a plant of a nucleic acid encoding a SYR polypeptide.

A preferred method for modulating (preferably, increasing) expression of a nucleic acid encoding a SYR polypeptide is by introducing and expressing in a plant a nucleic acid encoding a SYR polypeptide.

Any reference hereinafter to a “protein useful in the methods of the invention” is taken to mean a SYR polypeptide as defined herein. Any reference hereinafter to a “nucleic acid useful in the methods of the invention” is taken to mean a nucleic acid capable of encoding such a SYR polypeptide. The nucleic acid to be introduced into a plant (and therefore useful in performing the methods of the invention) is any nucleic acid encoding the type of protein which will now be described, hereafter also named “SYR nucleic acid” or “SYR gene”.

The term “SYR protein or homologue thereof” as defined herein refers to a polypeptide of about 65 to about 200 amino acids, comprising (i) a leucine rich domain that resembles a leucine zipper in the C-terminal half of the protein, which leucine rich domain is (ii) preceded by a tripeptide with the sequence YFS (conserved motif 1a, SEQ ID NO: 256), or YFT (conserved motif 1b, SEQ ID NO: 257), or YFG (conserved motif 1c, SEQ ID NO: 258) or YLG (conserved motif 1d, SEQ ID NO: 259), and (iii) followed by a conserved motif 2 ((V/A/I) LAFMP (T/S), SEQ ID NO: 260). Preferably, the conserved motif 2 is (A/V) LAFMP (T/S), most preferably, the conserved motif is VLAFMPT. The “SYR protein or homologue thereof” preferably also has a conserved C-terminal peptide ending with the conserved motif 3 (SYL or PYL, SEQ ID NO: 261). The leucine rich domain of the SYR protein or its homologue is about 38 to 48 amino acids long, starting immediately behind the conserved motif 1 and stopping immediately before the conserved motif 2, and comprises at least 30% of leucine. The Leu rich domain preferably has a motif that resembles the Leucine Zipper motif (L-X₆-L-X₆-L-X₆-L, wherein X₆ is a sequence of 6 consecutive amino acids). A preferred example of a SYR protein is represented by SEQ ID NO: 252, an overview of its domains is given in FIG. 24. It should be noted that the term “SYR protein or homologue thereof” does not encompass the ARGOS protein from Arabidopsis thaliana (SEQ ID NO: 276).

Further preferably, SYR proteins have two transmembrane domains, with the N-terminal part and C-terminal part of the protein located inside and the part between the transmembrane domains located outside.

Alternatively, the homologue of a SYR protein has in increasing order of preference at least 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO: 252, provided that the homologous protein comprises the conserved motifs 1 (a, b, c or d), 2 and 3, and the leucine rich domain as outlined above. The overall sequence identity is determined using a global alignment algorithm, such as the Needleman Wunsch algorithm in the program GAP (GCG Wisconsin Package, Accelrys), preferably with default parameters.

The term “domain” and “motif” is defined in the “definitions” section herein. Specialist databases exist for the identification of domains, for example, SMART (Schultz et al. (1998) Proc. Natl. Acad. Sci. USA 95, 5857-5864; Letunic et al. (2002) Nucleic Acids Res 30, 242-244, InterPro (Mulder et al., (2003) Nucl. Acids. Res. 31, 315-318, Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAI Press, Menlo Park; Hulo et al., Nucl. Acids. Res. 32:D134-D137, (2004), or Pfam (Bateman et al., Nucleic Acids Research 30(1): 276-280 (2002). A set of tools for in silico analysis of protein sequences is available on the ExPASy proteomics server (Swiss Institute of Bioinformatics (Gasteiger et al., ExPASy: the proteomics server for in-depth protein knowledge and analysis, Nucleic Acids Res. 31:3784-3788 (2003)). Domains may also be identified using routine techniques, such as by sequence alignment.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e. spanning the complete sequences) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences.). Minor manual editing may be performed to optimise alignment between conserved motifs, as would be apparent to a person skilled in the art. Furthermore, instead of using full-length sequences for the identification of homologues, specific domains may also be used. The sequence identity values may be determined over the entire nucleic acid or amino acid sequence or over selected domains or conserved motif(s), using the programs mentioned above using the default parameters.

Transmembrane domains are about 15 to 30 amino acids long and are usually composed of hydrophobic residues that form an alpha helix. They are usually predicted on the basis of hydrophobicity (for example Klein et al., Biochim. Biophys. Acta 815, 468, 1985; or Sonnhammer et al., In J. Glasgow, T. Littlejohn, F. Major, R. Lathrop, D. Sankoff, and C. Sensen, editors, Proceedings of the Sixth International Conference on Intelligent Systems for Molecular Biology, pages 175-182, Menlo Park, Calif., 1998. AAAI Press.).

Examples of proteins falling under the definition of “SYR polypeptide or a homologue thereof” are given in Table II of the examples section and include sequences from various monocotyledonous plants, such as rice (SEQ ID NO: 252, SEQ ID NO: 262 and SEQ ID NO: 263), corn (SEQ ID NO: 264), wheat (SEQ ID NO: 265), barley (SEQ ID NO: 266), sugarcane (SEQ ID NO: 267 and SEQ ID NO: 268), sorghum (SEQ ID NO: 269); and from dicotyledonous plants such as Arabidopsis (SEQ ID NO: 270 and SEQ ID NO: 271), grape (SEQ ID NO: 272), citrus (SEQ ID NO: 273) or tomato (SEQ ID NO: 274 and SEQ ID NO: 275). It is envisaged that the Leu rich domain is important for the function of the protein, hence proteins with the Leu rich domain but without the conserved motifs 1 or 2 may be useful as well in the methods of the present invention; examples of such proteins are given in SEQ ID NO: 284 and 285.

It is to be understood that the term “SYR polypeptide or a homologue thereof” is not to be limited to the sequence represented by SEQ ID NO: 252 or to the homologues listed as SEQ ID NO: 262 to SEQ ID NO: 275, but that any polypeptide of about 65 to about 200 amino acids meeting the criteria of comprising a leucine rich domain as defined above, preceded by the conserved tripeptide motif 1 (a, b, c or d) and followed by the conserved motif 2 and preferably also by the conserved motif 3; or having at least 38% sequence identity to the sequence of SEQ ID NO: 252, may be suitable for use in the methods of the invention.

The activity of a SYR protein or homologue thereof may be assayed by expressing the SYR protein or homologue thereof under control of a GOS2 promoter in Oryza sativa, which results in plants with increased increased biomass and/or seed yield without a delay in flowering time when grown under conditions of nitrogen deficiency and compared to corresponding wild type plants. This increase in seed yield may be measured in several ways, for example as an increase of total seed weight, number of filled seeds or Thousand Kernel Weight.

The present invention is illustrated by transforming plants with the nucleic acid sequence represented by SEQ ID NO: 251, encoding the polypeptide sequence of SEQ ID NO: 252. However, performance of the invention is not restricted to these sequences; the methods of the invention may advantageously be performed using any SYR-encoding nucleic acid or SYR polypeptide as defined herein.

Examples of nucleic acids encoding SYR polypeptides are given in Table II of Example 38 herein. Such nucleic acids are useful in performing the methods of the invention. The amino acid sequences given in Table II of Example 38 are example sequences of orthologues and paralogues of the SYR polypeptide represented by SEQ ID NO: 252, the terms “orthologues” and “paralogues” being as defined herein. Further orthologues and paralogues may readily be identified by performing a so-called reciprocal blast search. Typically, this involves a first BLAST involving BLASTing a query sequence (for example using any of the sequences listed in Table II of Example 38) against any sequence database, such as the publicly available NCBI database. BLASTN or TBLASTX (using standard default values) are generally used when starting from a nucleotide sequence, and BLASTP or TBLASTN (using standard default values) when starting from a protein sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 251 or SEQ ID NO: 252, the second BLAST would therefore be against rice sequences). The results of the first and second BLASTs are then compared. A paralogue is identified if a high-ranking hit from the first blast is from the same species as from which the query sequence is derived, a BLAST back then ideally results in the query sequence amongst the highest hits; an orthologue is identified if a high-ranking hit in the first BLAST is not from the same species as from which the query sequence is derived, and preferably results upon BLAST back in the query sequence being among the highest hits.

High-ranking hits are those having a low E-value. The lower the E-value, the more significant the score (or in other words the lower the chance that the hit was found by chance). Computation of the E-value is well known in the art. In addition to E-values, comparisons are also scored by percentage identity. Percentage identity refers to the number of identical nucleotides (or amino acids) between the two compared nucleic acid (or polypeptide) sequences over a particular length. In the case of large families, ClustalW may be used, followed by a neighbour joining tree, to help visualize clustering of related genes and to identify orthologues and paralogues.

Nucleic acid variants may also be useful in practising the methods of the invention. Examples of such variants include nucleic acids encoding homologues and derivatives of any one of the amino acid sequences given in Table II of Example 38, the terms “homologue” and “derivative” being as defined herein. Also useful in the methods of the invention are nucleic acids encoding homologues and derivatives of orthologues or paralogues of any one of the amino acid sequences given in Table II of Example 38. Homologues and derivatives useful in the methods of the present invention have substantially the same biological and functional activity as the unmodified protein from which they are derived.

Further nucleic acid variants useful in practising the methods of the invention include portions of nucleic acids encoding SYR polypeptides, nucleic acids hybridising to nucleic acids encoding SYR polypeptides, splice variants of nucleic acids encoding SYR polypeptides, allelic variants of nucleic acids encoding SYR polypeptides and variants of nucleic acids encoding SYR polypeptides obtained by gene shuffling. The terms hybridising sequence, splice variant, allelic variant and gene shuffling are as described herein.

Nucleic acids encoding SYR polypeptides need not be full-length nucleic acids, since performance of the methods of the invention does not rely on the use of full-length nucleic acid sequences. According to the present invention, there is provided a method for enhancing yield-related traits in plants, comprising introducing and expressing in a plant a portion of any one of the nucleic acid sequences given in Table II of Example 38, or a portion of a nucleic acid encoding an orthologue, paralogue or homologue of any of the amino acid sequences given in Table II of Example 38.

A portion of a nucleic acid may be prepared, for example, by making one or more deletions to the nucleic acid. The portions may be used in isolated form or they may be fused to other coding (or non-coding) sequences in order to, for example, produce a protein that combines several activities. When fused to other coding sequences, the resultant polypeptide produced upon translation may be bigger than that predicted for the protein portion.

Portions useful in the methods of the invention, encode a SYR polypeptide as defined herein, and have substantially the same biological activity as the amino acid sequences given in Table II of Example 38. Preferably, the portion is a portion of any one of the nucleic acids given in Table II of Example 38, or is a portion of a nucleic acid encoding an orthologue or paralogue of any one of the amino acid sequences given in Table II of Example 38. Preferably the portion is at least 150, 200, 250, 300, 350, 400, 450, 500, 550, 600 consecutive nucleotides in length, the consecutive nucleotides being of any one of the nucleic acid sequences given in Table II of Example 38, or of a nucleic acid encoding an orthologue or paralogue of any one of the amino acid sequences given in Table II of Example 38. Most preferably the portion is a portion of the nucleic acid of SEQ ID NO: 251. Preferably, the portion encodes encodes a polypeptide of about 65 to about 200 amino acids, comprising a leucine rich domain as defined above, preceded by the conserved tripeptide motif 1 (a, b, c or d) and followed by the conserved motif 2 and preferably also by the conserved motif 3; or having at least 38% sequence identity to the sequence of SEQ ID NO: 252.

Another nucleic acid variant useful in the methods of the invention is a nucleic acid capable of hybridising, under reduced stringency conditions, preferably under stringent conditions, with a nucleic acid encoding a SYR polypeptide as defined herein, or with a portion as defined herein.

According to the present invention, there is provided a method for enhancing yield-related traits in plants, comprising introducing and expressing in a plant a nucleic acid capable of hybridizing to any one of the nucleic acids given in Table II of Example 38, or comprising introducing and expressing in a plant a nucleic acid capable of hybridising to a nucleic acid encoding an orthologue, paralogue or homologue of any of the nucleic acid sequences given in Table II of Example 38.

Hybridising sequences useful in the methods of the invention encode a SYR polypeptide as defined herein, and have substantially the same biological activity as the amino acid sequences given in Table II of Example 38. Preferably, the hybridising sequence is capable of hybridising to any one of the nucleic acids given in Table II of Example 38, or to a portion of any of these sequences, a portion being as defined above, or wherein the hybridising sequence is capable of hybridising to a nucleic acid encoding an orthologue or paralogue of any one of the amino acid sequences given in Table II of Example 38. Most preferably, the hybridising sequence is capable of hybridising to a nucleic acid as represented by SEQ ID NO: 251 or to a portion thereof.

Preferably, the hybridising sequence encodes a polypeptide of about 65 to about 200 amino acids, comprising a leucine rich domain as defined above, preceded by the conserved tripeptide motif 1 (a, b, c or d) and followed by the conserved motif 2 and preferably also by the conserved motif 3; or having at least 38% sequence identity to the sequence of SEQ ID NO: 252.

Another nucleic acid variant useful in the methods of the invention is a splice variant encoding a SYR polypeptide as defined hereinabove, a splice variant being as defined herein.

According to the present invention, there is provided a method for enhancing yield-related traits in plants, comprising introducing and expressing in a plant a splice variant of any one of the nucleic acid sequences given in Table II of Example 38, or a splice variant of a nucleic acid encoding an orthologue, paralogue or homologue of any of the amino acid sequences given in Table II of Example 38.

Preferred splice variants are splice variants of a nucleic acid represented by SEQ ID NO: 251, or a splice variant of a nucleic acid encoding an orthologue or paralogue of SEQ ID NO: 252. Preferably, the amino acid sequence encoded by the splice variant is a polypeptide of about 65 to about 200 amino acids, comprising a leucine rich domain as defined above, preceded by the conserved tripeptide motif 1 (a, b, c or d) and followed by the conserved motif 2 and preferably also by the conserved motif 3; or having at least 38% sequence identity to the sequence of SEQ ID NO: 252.

Another nucleic acid variant useful in performing the methods of the invention is an allelic variant of a nucleic acid encoding a SYR polypeptide as defined hereinabove, an allelic variant being as defined herein.

According to the present invention, there is provided a method for enhancing yield-related traits in plants, comprising introducing and expressing in a plant an allelic variant of any one of the nucleic acids given in Table II of Example 38, or comprising introducing and expressing in a plant an allelic variant of a nucleic acid encoding an orthologue, paralogue or homologue of any of the amino acid sequences given in Table II of Example 38.

The allelic variants useful in the methods of the present invention have substantially the same biological activity as the SYR polypeptide of SEQ ID NO: 252 and any of the amino acids depicted in Table II of Example 38. Allelic variants exist in nature, and encompassed within the methods of the present invention is the use of these natural alleles. Preferably, the allelic variant is an allelic variant of SEQ ID NO: 251 or an allelic variant of a nucleic acid encoding an orthologue or paralogue of SEQ ID NO: 252. Preferably, the amino acid sequence encoded by the allelic variant is a polypeptide of about 65 to about 200 amino acids, comprising a leucine rich domain as defined above, preceded by the conserved tripeptide motif 1 (a, b, c or d) and followed by the conserved motif 2 and preferably also by the conserved motif 3; or having at least 38% sequence identity to the sequence of SEQ ID NO: 252.

Gene shuffling or directed evolution may also be used to generate variants of nucleic acids encoding SYR polypeptides as defined above; the term “gene shuffling” being as defined herein.

According to the present invention, there is provided a method for enhancing yield-related traits in plants, comprising introducing and expressing in a plant a variant of any one of the nucleic acid sequences given in Table II of Example 38, or comprising introducing and expressing in a plant a variant of a nucleic acid encoding an orthologue, paralogue or homologue of any of the amino acid sequences given in Table II of Example 38, which variant nucleic acid is obtained by gene shuffling.

Preferably, the amino acid sequence encoded by the variant nucleic acid obtained by gene shuffling is a polypeptide of about 65 to about 200 amino acids, comprising a leucine rich domain as defined above, preceded by the conserved tripeptide motif 1 (a, b, c or d) and followed by the conserved motif 2 and preferably also by the conserved motif 3; or having at least 38% sequence identity to the sequence of SEQ ID NO: 252.

Furthermore, nucleic acid variants may also be obtained by site-directed mutagenesis. Several methods are available to achieve site-directed mutagenesis, the most common being PCR based methods (Current Protocols in Molecular Biology; Wiley Eds.).

Nucleic acids encoding SYR polypeptides may be derived from any natural or artificial source. The nucleic acid may be modified from its native form in composition and/or genomic environment through deliberate human manipulation. Preferably the SYR polypeptide-encoding nucleic acid is from a plant, further preferably from a monocotyledonous plant, more preferably from the family Poaceae, most preferably the nucleic acid is from Oryza sativa.

Performance of the methods of the invention gives plants having increased abiotic stress resistance (or abiotic stress tolerance, which terms are used interchangeably), effected as enhanced yield-related traits compared to control plants when grown under abiotic stress. In particular, performance of the methods of the invention gives plants having increased yield, especially increased seed yield and increased biomass relative to control plants. The terms “yield” and “seed yield” are described in more detail in the “definitions” section herein.

Reference herein to enhanced yield-related traits is taken to mean an increase in biomass (weight) of one or more parts of a plant, which may include aboveground (harvestable) parts and/or (harvestable) parts below ground. In particular, such harvestable parts are seeds, and performance of the methods of the invention results in plants having increased seed yield relative to the seed yield of control plants.

Taking corn as an example, a yield increase may be manifested as one or more of the following: increase in the number of plants established per hectare or acre, an increase in the number of ears per plant, an increase in the number of rows, number of kernels per row, kernel weight, thousand kernel weight, ear length/diameter, increase in the seed filling rate (which is the number of filled seeds divided by the total number of seeds and multiplied by 100), among others. Taking rice as an example, a yield increase may manifest itself as an increase in one or more of the following: number of plants per hectare or acre, number of panicles per plant, number of spikelets per panicle, number of flowers (florets) per panicle (which is expressed as a ratio of the number of filled seeds over the number of primary panicles), increase in the seed filling rate (which is the number of filled seeds divided by the total number of seeds and multiplied by 100), increase in thousand kernel weight, among others.

The present invention provides a method for increasing abiotic stress resistance of plants, resulting in increased yield, especially seed yield and/or increased biomass of plants, relative to control plants, when grown under conditions of abiotic stress, which method comprises modulating expression, preferably increasing expression, in a plant of a nucleic acid encoding a SYR polypeptide as defined herein.

Since the transgenic plants according to the present invention have increased yield, it is likely that these plants exhibit an increased growth rate (during at least part of their life cycle), relative to the growth rate of control plants at a corresponding stage in their life cycle. Besides the increased yield capacity, an increased efficiency of nutrient uptake may also contribute to the increase in yield. It is observed that the plants according to the present invention show a higher efficiency in nutrient uptake. Increased efficiency of nutrient uptake allows better growth of the plant, when the plant is under stress.

The increased growth rate may be specific to one or more parts of a plant (including seeds), or may be throughout substantially the whole plant. Plants having an increased growth rate may have a shorter life cycle. The life cycle of a plant may be taken to mean the time needed to grow from a dry mature seed up to the stage where the plant has produced dry mature seeds, similar to the starting material. This life cycle may be influenced by factors such as early vigour, growth rate, greenness index, flowering time and speed of seed maturation. The increase in growth rate may take place at one or more stages in the life cycle of a plant or during substantially the whole plant life cycle. Increased growth rate during the early stages in the life cycle of a plant may reflect enhanced vigour. The increase in growth rate may alter the harvest cycle of a plant allowing plants to be sown later and/or harvested sooner than would otherwise be possible (a similar effect may be obtained with earlier flowering time). If the growth rate is sufficiently increased, it may allow for the further sowing of seeds of the same plant species (for example sowing and harvesting of rice plants followed by sowing and harvesting of further rice plants all within one conventional growing period). Similarly, if the growth rate is sufficiently increased, it may allow for the further sowing of seeds of different plants species (for example the sowing and harvesting of corn plants followed by, for example, the sowing and optional harvesting of soybean, potato or any other suitable plant). Harvesting additional times from the same rootstock in the case of some crop plants may also be possible. Altering the harvest cycle of a plant may lead to an increase in annual biomass production per acre (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and T-90 (time taken for plants to reach 90% of their maximal size), amongst others.

According to a preferred feature of the present invention, performance of the methods of the invention gives plants having an increased growth rate relative to control plants when grown under abiotic stress conditions. Therefore, according to the present invention, there is provided a method for increasing the growth rate of plants under abiotic stress conditions, which method comprises modulating expression, preferably increasing expression, in a plant of a nucleic acid encoding a SYR polypeptide as defined herein.

An increase in yield and/or growth rate occurs when the plant is exposed to various abiotic stresses compared to control plants. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35% or 30%, preferably less than 25%, 20% or 15%, more preferably less than 14%, 13%, 12%, 11% or 10% or less in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the everyday biotic and/or abiotic (environmental) stresses to which a plant is exposed. Abiotic stresses may be due to drought or excess water, anaerobic stress, salt stress, chemical toxicity, oxidative stress and hot, cold or freezing temperatures. The abiotic stress may be an osmotic stress caused by a water stress (particularly due to drought), salt stress, oxidative stress or an ionic stress. In a particular embodiment, the abiotic stress is the reduced availability of one or more nutrients that need to be assimilated by the plants for growth and development. Biotic stresses are typically those stresses caused by pathogens, such as bacteria, viruses, fungi and insects.

In particular, the methods of the present invention may be performed under stress conditions, preferably under conditions of reduced availability of one or more nutrients, or under conditions of mild drought to give plants having increased yield relative to control plants. As reported in Wang et al. (Planta (2003) 218: 1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani et al. (Plant Physiol (2003) 133: 1755-1767) describes a particularly high degree of “cross talk” between drought stress and high-salinity stress. For example, drought and/or salinisation are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signalling pathways and cellular responses, such as the production of stress proteins, up-regulation of anti-oxidants, accumulation of compatible solutes and growth arrest. The term “non-stress” conditions as used herein are those environmental conditions that allow optimal growth of plants. Persons skilled in the art are aware of normal soil conditions and climatic conditions for a given location.

Performance of the methods of the invention gives plants grown under abiotic stress conditions or under mild drought conditions increased yield relative to control plants grown under comparable conditions. Therefore, according to the present invention, there is provided a method for increasing yield in plants grown under abiotic stress conditions or under mild drought conditions, which method comprises increasing expression in a plant of a nucleic acid encoding a SYR polypeptide.

Performance of the methods of the invention gives plants grown under conditions of nutrient deficiency, particularly under conditions of nitrogen deficiency, increased yield relative to control plants grown under comparable conditions. Therefore, according to the present invention, there is provided a method for increasing yield in plants grown under conditions of nutrient deficiency, which method comprises increasing expression in a plant of a nucleic acid encoding a SYR polypeptide. Nutrient deficiency may result from a lack of nutrients such as nitrogen, phosphates and other phosphorous-containing compounds, potassium, calcium, cadmium, magnesium, manganese, iron and boron, amongst others.

The present invention encompasses plants or parts thereof (including seeds) obtainable by the methods according to the present invention. The plants or parts thereof comprise a nucleic acid transgene encoding a SYR polypeptide as defined above.

The invention also provides genetic constructs and vectors to facilitate introduction and/or expression in plants of nucleic acids encoding SYR polypeptides. The gene constructs may be inserted into vectors, which may be commercially available, suitable for transforming into plants and suitable for expression of the gene of interest in the transformed cells. The invention also provides use of a gene construct as defined herein in the methods of the invention.

More specifically, the present invention provides a construct comprising:
(a) a nucleic acid encoding a SYR polypeptide as defined above;
(b) one or more control sequences capable of driving expression of the nucleic acid sequence of (a); and optionally (c) a transcription termination sequence.

Preferably, the nucleic acid encoding a SYR polypeptide is as defined above. The term “control sequence” and “termination sequence” are as defined herein.

Plants are transformed with a vector comprising any of the nucleic acids described above. The skilled artisan is well aware of the genetic elements that must be present on the vector in order to successfully transform, select and propagate host cells containing the sequence of interest. The sequence of interest is operably linked to one or more control sequences (at least to a promoter).

Advantageously, any type of promoter, whether natural or synthetic, may be used to drive expression of the nucleic acid sequence. A constitutive promoter is particularly useful in the methods. See the “Definitions” section herein for definitions of the various promoter types.

It should be clear that the applicability of the present invention is not restricted to the SYR polypeptide-encoding nucleic acid represented by SEQ ID NO: 251, nor is the applicability of the invention restricted to expression of a SYR polypeptide-encoding nucleic acid when driven by a constitutive promoter.

The constitutive promoter is preferably a GOS2 promoter, preferably a GOS2 promoter from rice. Further preferably the constitutive promoter is represented by a nucleic acid sequence substantially similar to SEQ ID NO: 255 or SEQ ID NO: 58, most preferably the constitutive promoter is as represented by SEQ ID NO: 255 or SEQ ID NO: 58. See Table 2a in the “Definitions” section herein for further examples of useful constitutive promoters.

Optionally, one or more terminator sequences may be used in the construct introduced into a plant. Additional regulatory elements may include transcriptional as well as translational enhancers. Those skilled in the art will be aware of terminator and enhancer sequences that may be suitable for use in performing the invention. An intron sequence may also be added to the 5′ untranslated region (UTR) or in the coding sequence to increase the amount of the mature message that accumulates in the cytosol, as described in the definitions section. Other control sequences (besides promoter, enhancer, silencer, intron sequences, 3′UTR and/or 5′UTR regions) may be protein and/or RNA stabilizing elements. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

For the detection of the successful transfer of the nucleic acid sequences as used in the methods of the invention and/or selection of transgenic plants comprising these nucleic acids, it is advantageous to use marker genes (or reporter genes). Therefore, the genetic construct may optionally comprise a selectable marker gene. Selectable markers are described in more detail in the “definitions” section herein.

It is known that upon stable or transient integration of nucleic acids into plant cells, only a minority of the cells takes up the foreign DNA and, if desired, integrates it into its genome, depending on the expression vector used and the transfection technique used. To identify and select these integrants, a gene coding for a selectable marker (such as the ones described above) is usually introduced into the host cells together with the gene of interest. These markers can for example be used in mutants in which these genes are not functional by, for example, deletion by conventional methods. Furthermore, nucleic acid molecules encoding a selectable marker can be introduced into a host cell on the same vector that comprises the sequence encoding the polypeptides of the invention or used in the methods of the invention, or else in a separate vector. Cells which have been stably transfected with the introduced nucleic acid can be identified for example by selection (for example, cells which have integrated the selectable marker survive whereas the other cells die). The marker genes may be removed or excised from the transgenic cell once they are no longer needed. Techniques for marker gene removal are known in the art, useful techniques are described above in the definitions section.

The invention also provides a method for the production of transgenic plants having, when grown under abiotic stress conditions, enhanced yield-related traits relative to control plants, comprising introduction and expression in a plant of any nucleic acid encoding a SYR polypeptide as defined hereinabove.

More specifically, the present invention provides a method for the production of transgenic plants having increased enhanced yield-related traits, particularly increased (seed) yield and/or increased biomass, which method comprises:
(i) introducing and expressing in a plant or plant cell a SYR polypeptide-encoding nucleic acid; and
(ii) cultivating the plant cell under conditions promoting plant growth and development.

The nucleic acid of (i) may be any of the nucleic acids capable of encoding a SYR polypeptide as defined herein.

The nucleic acid may be introduced directly into a plant cell or into the plant itself (including introduction into a tissue, organ or any other part of a plant). According to a preferred feature of the present invention, the nucleic acid is preferably introduced into a plant by transformation. The term “transformation” is described in more detail in the “definitions” section herein.

The genetically modified plant cells can be regenerated via all methods with which the skilled worker is familiar. Suitable methods can be found in the abovementioned publications by S. D. Kung and R. Wu, Potrykus or Höfgen and Willmitzer.

Generally after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described above.

Following DNA transfer and regeneration, putatively transformed plants may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

The invention also includes host cells containing an isolated nucleic acid encoding a SYR polypeptide as defined hereinabove. Preferred host cells according to the invention are plant cells. Host plants for the nucleic acids or the vector used in the method according to the invention, the expression cassette or construct or vector are, in principle, advantageously all plants, which are capable of synthesizing the polypeptides used in the inventive method.

The methods of the invention are advantageously applicable to any plant. Plants that are particularly useful in the methods of the invention include all plants which belong to the superfamily Viridiplantae, in particular monocotyledonous and dicotyledonous plants including fodder or forage legumes, ornamental plants, food crops, trees or shrubs. According to a preferred embodiment of the present invention, the plant is a crop plant. Examples of crop plants include soybean, sunflower, canola, alfalfa, rapeseed, cotton, tomato, potato and tobacco. Further preferably, the plant is a monocotyledonous plant. Examples of monocotyledonous plants include sugarcane. More preferably the plant is a cereal. Examples of cereals include rice, maize, wheat, barley, millet, rye, triticale, sorghum and oats.

The invention also extends to harvestable parts of a plant such as, but not limited to seeds, leaves, fruits, flowers, stems, rhizomes, tubers and bulbs. The invention furthermore relates to products derived, preferably directly derived, from a harvestable part of such a plant, such as dry pellets or powders, oil, fat and fatty acids, starch or proteins.

According to a preferred feature of the invention, the modulated expression is increased expression. Methods for increasing expression of nucleic acids or genes, or gene products, are well documented in the art and examples are provided in the definitions section.

As mentioned above, a preferred method for modulating (preferably, increasing) expression of a nucleic acid encoding a SYR polypeptide is by introducing and expressing in a plant a nucleic acid encoding a SYR polypeptide; however the effects of performing the method, i.e. enhancing yield-related traits may also be achieved using other well known techniques, including but not limited to T-DNA activation tagging, TILLING, homologous recombination. A description of these techniques is provided in the definitions section.

The present invention also encompasses use of nucleic acids encoding SYR polypeptides as described herein and use of these SYR polypeptides in enhancing any of the aforementioned yield-related traits in plants when grown under abiotic stress conditions.

Nucleic acids encoding SYR polypeptide described herein, or the SYR polypeptides themselves, may find use in breeding programmes in which a DNA marker is identified which may be genetically linked to a SYR polypeptide-encoding gene. The nucleic acids/genes, or the SYR polypeptides themselves may be used to define a molecular marker. This DNA or protein marker may then be used in breeding programmes to select plants having enhanced yield-related traits as defined hereinabove in the methods of the invention.

Allelic variants of a SYR polypeptide-encoding nucleic acid/gene may also find use in marker-assisted breeding programmes. Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give increased yield. Selection is typically carried out by monitoring growth performance of plants containing different allelic variants of the sequence in question. Growth performance may be monitored in a greenhouse or in the field. Further optional steps include crossing plants in which the superior allelic variant was identified with another plant. This could be used, for example, to make a combination of interesting phenotypic features.

Nucleic acids encoding SYR polypeptides may also be used as probes for genetically and physically mapping the genes that they are a part of, and as markers for traits linked to those genes. Such information may be useful in plant breeding in order to develop lines with desired phenotypes. Such use of SYR polypeptide-encoding nucleic acids requires only a nucleic acid sequence of at least 15 nucleotides in length. The SYR polypeptide-encoding nucleic acids may be used as restriction fragment length polymorphism (RFLP) markers. Southern blots (Sambrook J, Fritsch EF and Maniatis T (1989) Molecular Cloning, A Laboratory Manual) of restriction-digested plant genomic DNA may be probed with the SYR-encoding nucleic acids. The resulting banding patterns may then be subjected to genetic analyses using computer programs such as MapMaker (Lander et al. (1987) Genomics 1: 174-181) in order to construct a genetic map. In addition, the nucleic acids may be used to probe Southern blots containing restriction endonuclease-treated genomic DNAs of a set of individuals representing parent and progeny of a defined genetic cross. Segregation of the DNA polymorphisms is noted and used to calculate the position of the SYR polypeptide-encoding nucleic acid in the genetic map previously obtained using this population (Botstein et al. (1980) Am. J. Hum. Genet. 32:314-331).

The production and use of plant gene-derived probes for use in genetic mapping is described in Bernatzky and Tanksley (1986) Plant Mol. Biol. Reporter 4: 37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid probes may be used in direct fluorescence in situ hybridisation (FISH) mapping (Trask (1991) Trends Genet. 7:149-154). Although current methods of FISH mapping favour use of large clones (several kb to several hundred kb; see Laan et al. (1995) Genome Res. 5:13-20), improvements in sensitivity may allow performance of FISH mapping using shorter probes.

A variety of nucleic acid amplification-based methods for genetic and physical mapping may be carried out using the nucleic acids. Examples include allele-specific amplification (Kazazian (1989) J. Lab. Clin. Med. 11:95-96), polymorphism of PCR-amplified fragments (CAPS; Sheffield et al. (1993) Genomics 16:325-332), allele-specific ligation (Landegren et al. (1988) Science 241:1077-1080), nucleotide extension reactions (Sokolov (1990) Nucleic Acid Res. 18:3671), Radiation Hybrid Mapping (Walter et al. (1997) Nat. Genet. 7:22-28) and Happy Mapping (Dear and Cook (1989) Nucleic Acid Res. 17:6795-6807). For these methods, the sequence of a nucleic acid is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

The methods according to the present invention result in plants having enhanced yield-related traits, as described hereinbefore. These traits may also be combined with other economically advantageous traits, such as further yield-enhancing traits, tolerance to other abiotic and biotic stresses, traits modifying various architectural features and/or biochemical and/or physiological features.

### DESCRIPTION OF FIGURES

### ERLK

FIG. 1 gives an overview of the group of receptor kinase proteins, classified according to their extracellular region (Shiu and Bleecker, 2001). The vertical line marked as TM indicates the transmembrane domain. On the left, locus names or MAtDB names are provided of representative proteins. RLCK stands for receptor-like cytoplasmic kinase, RLK stands for receptor-like kinase. The domain names are given according to the SMART and Pfam databases.

FIG. 2 shows the domain organization of the ERLK protein used in the present invention (SEQ ID NO: 2): indicated in bold: low complexity domain, underlined: transmembrane domain, italics underlined: kinase domain. The analysis was done with SMART.

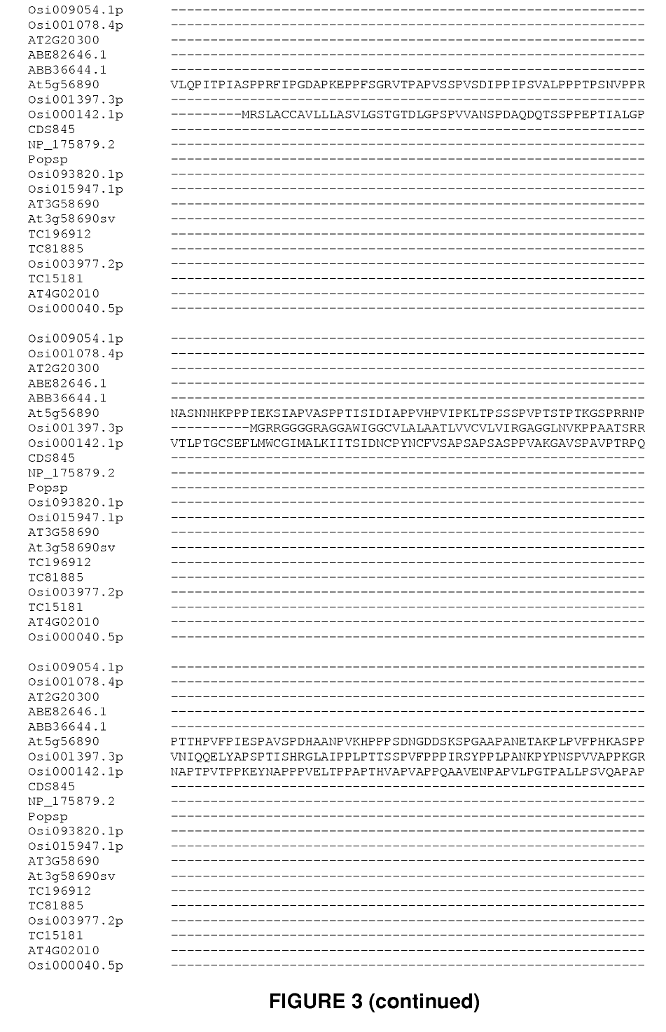

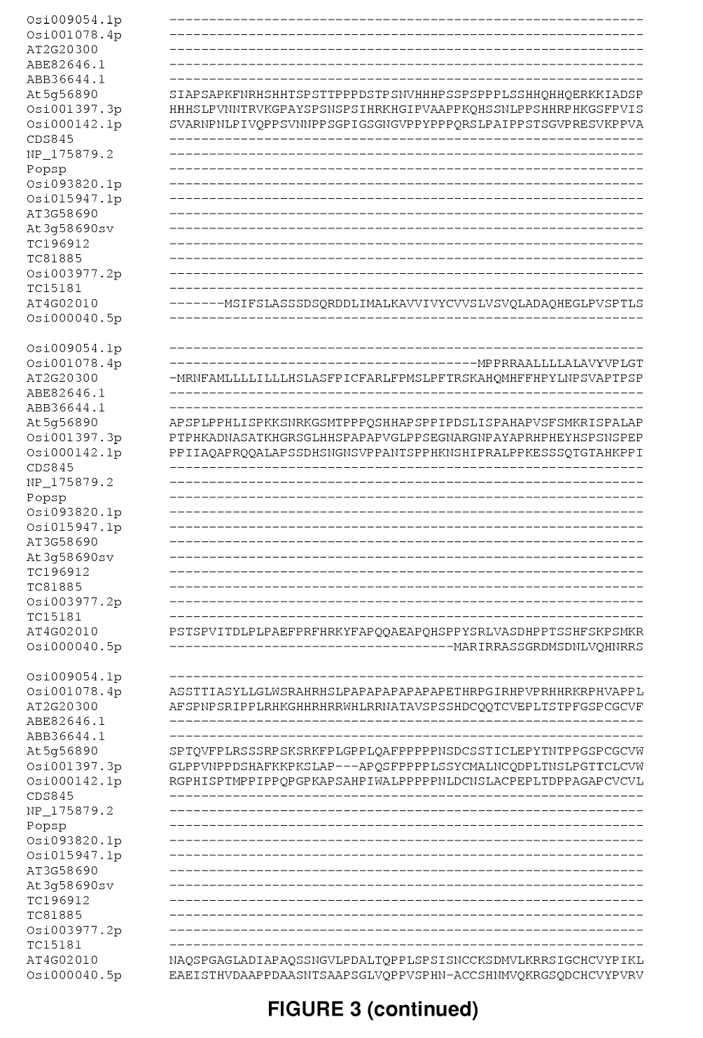

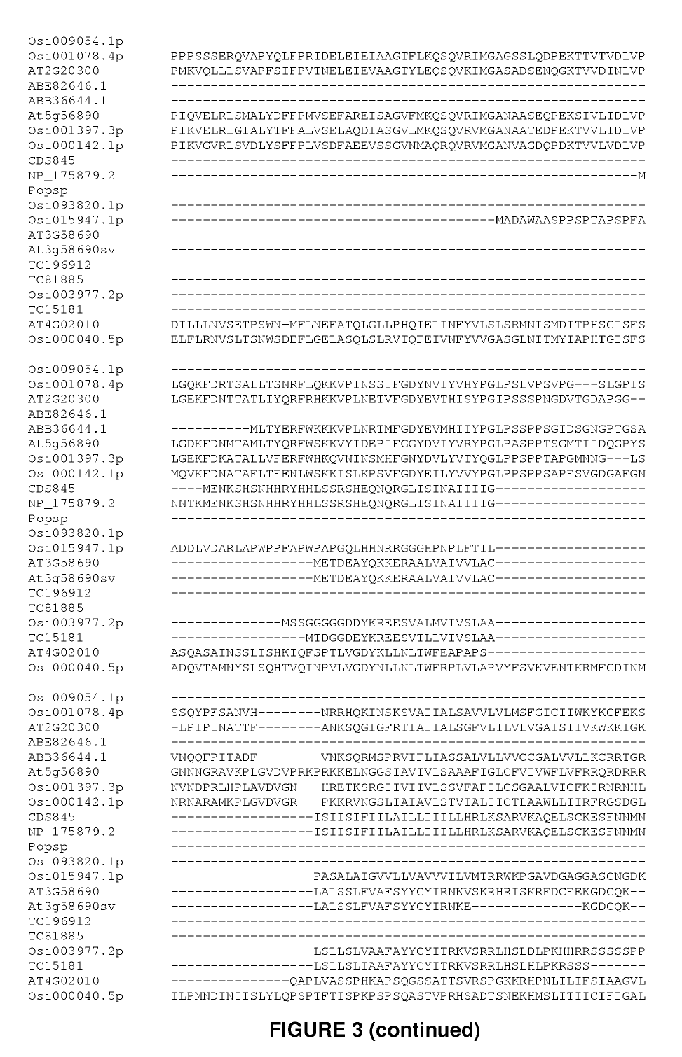

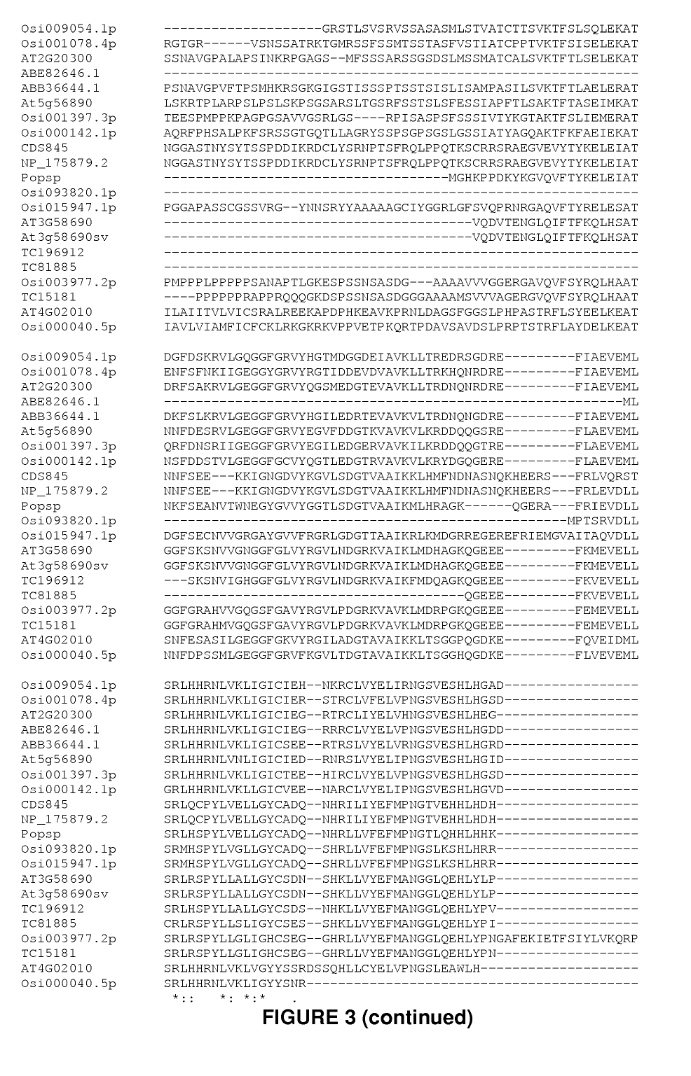

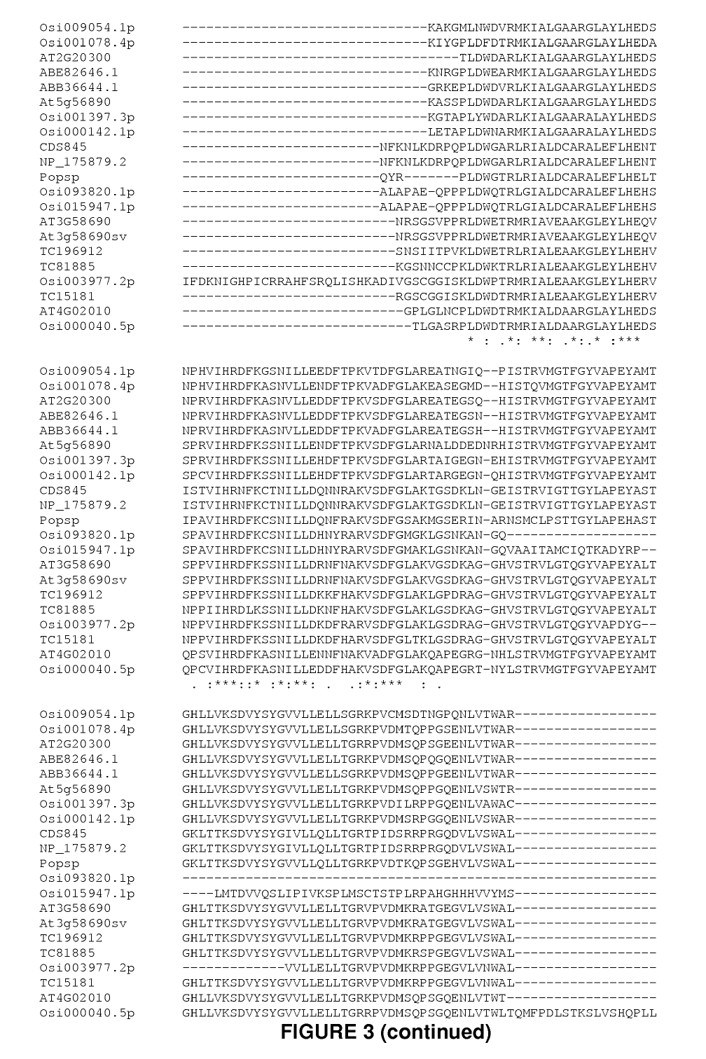

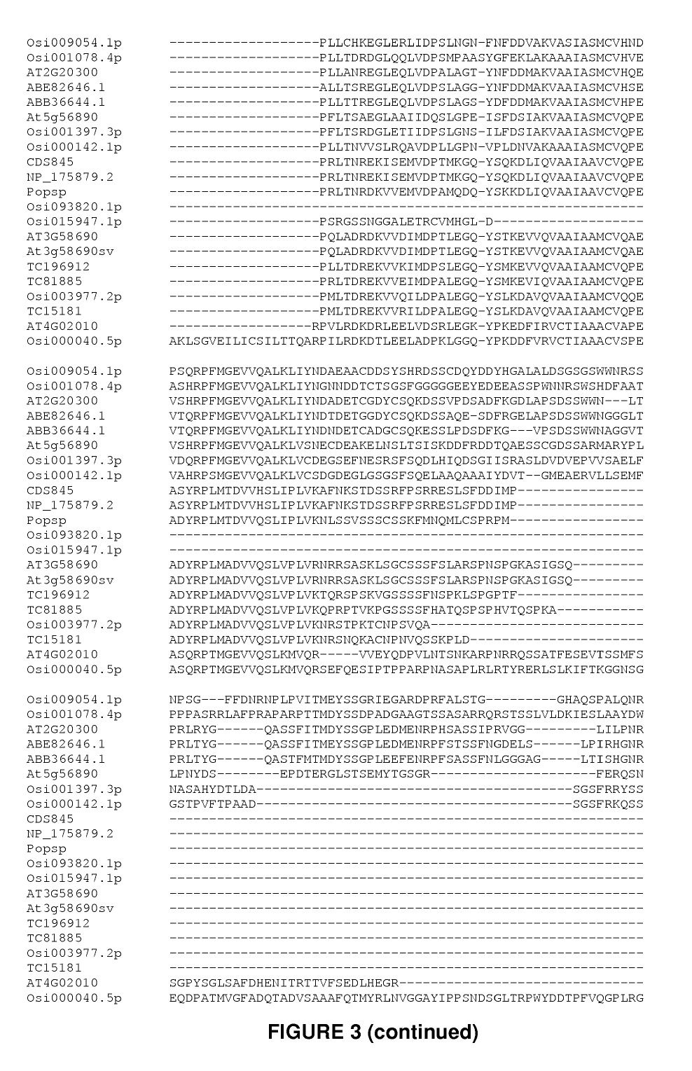

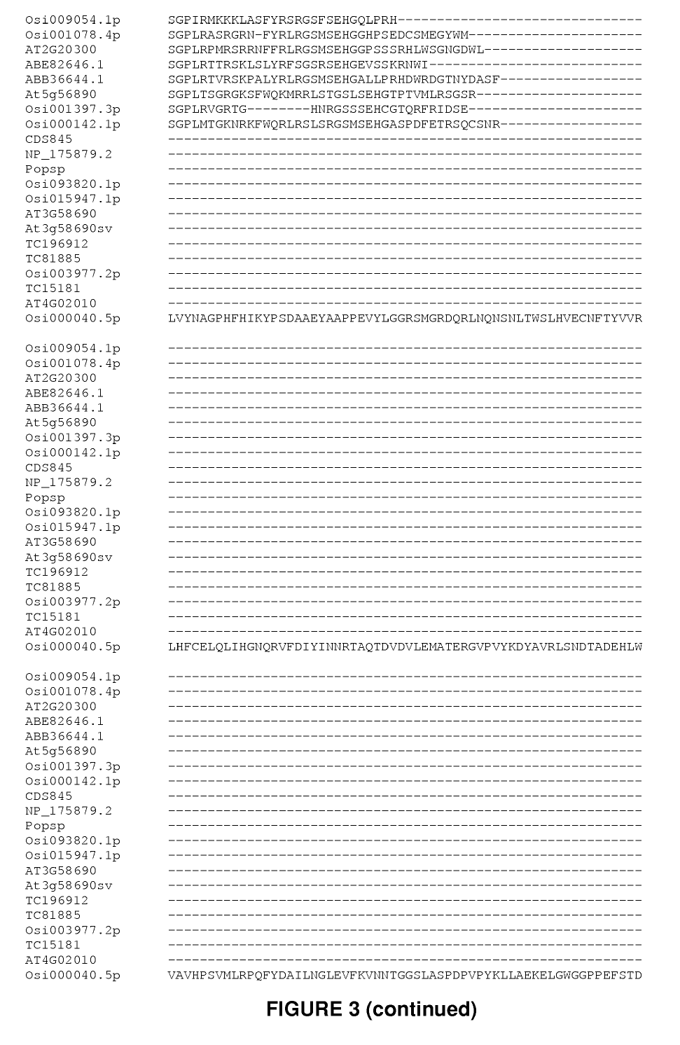

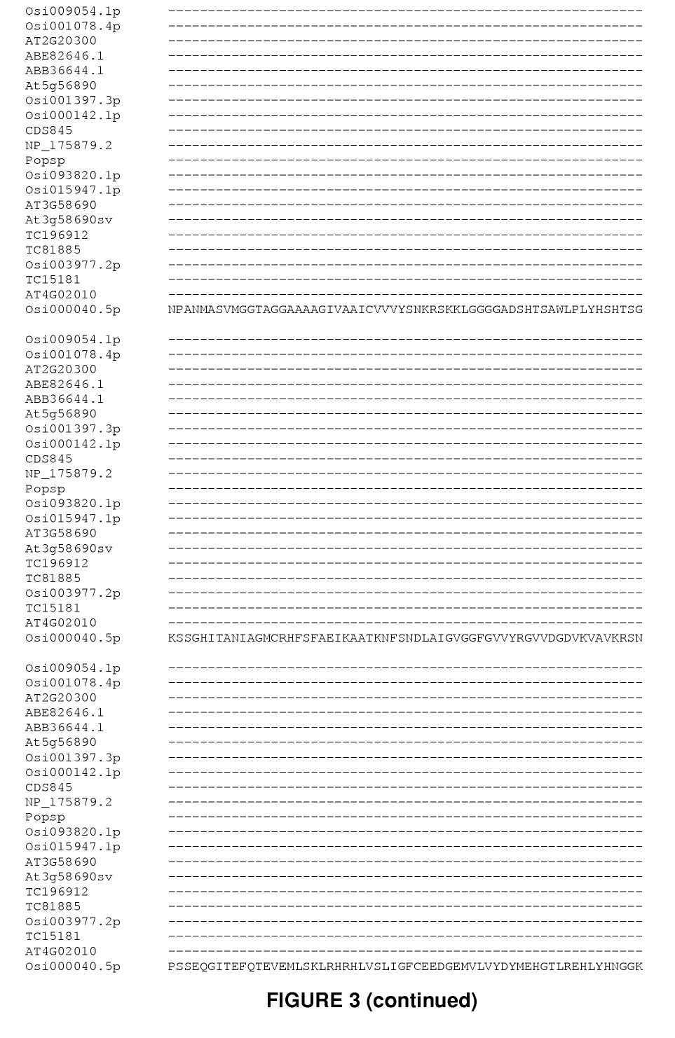

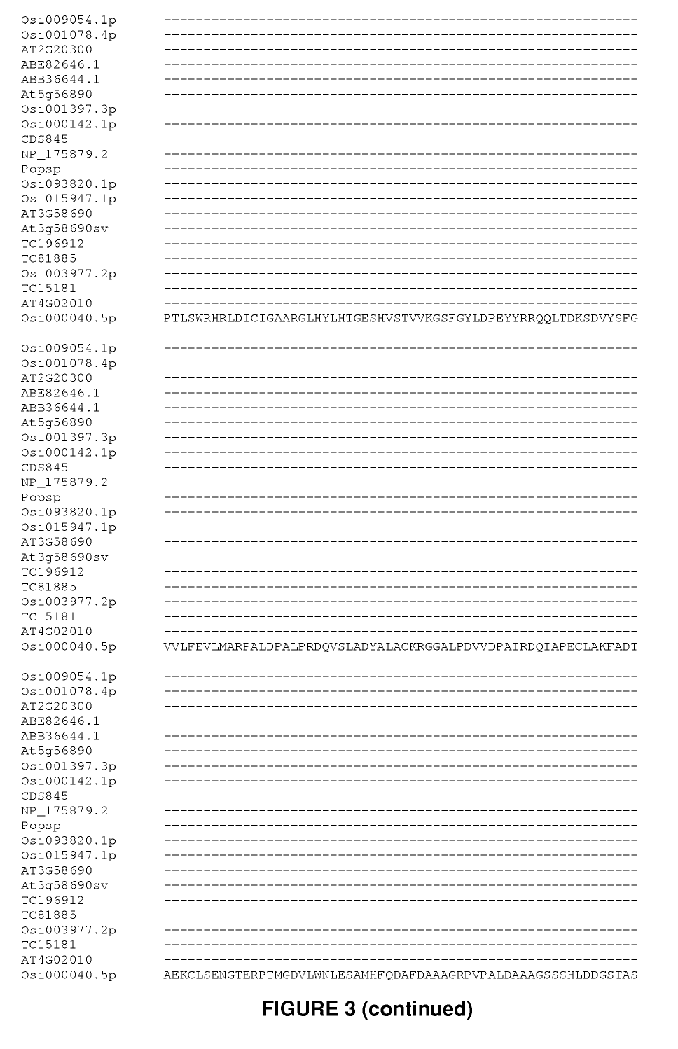

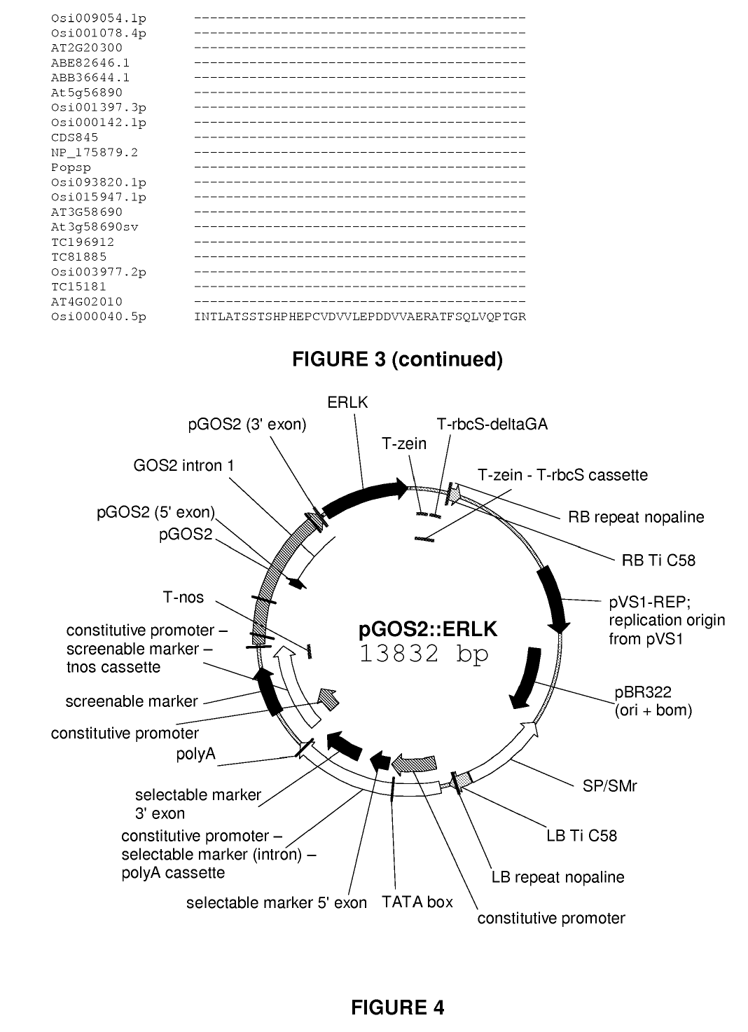

FIG. 3 gives a multiple alignment of the proteins listed as SEQ ID NO: 2, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 34, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO:52, SEQ ID NO: 54 and SEQ ID NO: 56. The alignment was made with CLUSTAL W (1.83), weight matrix: BLOSUM, gap opening penalty: 11, gap extension penalty: 1.

FIG. 4 shows a map of the binary plasmid p030, used for increasing expression in Oryza sativa of an Arabidopsis ERLK-encoding nucleic acid under the control of a GOS2 promoter (SEQ ID NO: 58).

### FBXW

FIG. 5 is a schematic presentation of the structure of FBXW polypeptides in plants. The relative position of the different features is shown: the F-box (PFAM PF00646), the WD40 domain (with seven individual WD40 repeats as in PFAM PF00400), and Motifs 1 to 5 as represented respectively by SEQ ID NO: 97 to 101.

FIG. 6 shows a multiple sequence alignment of plant FBXW polypeptides using CLUSTAL W (1.83) (at GenomeNet service at the Kyoto University Bioinformatics Center), and default values (Blosum 62 as weight matrix, gap open penalty of 10; gap extension penalty of 0.05). The F-box and the WD40 repeats are boxed. Motif 1 and Motif 2 are both marked by a curly bracket. Motifs 3, 4 and 5 are underlined by a black box.

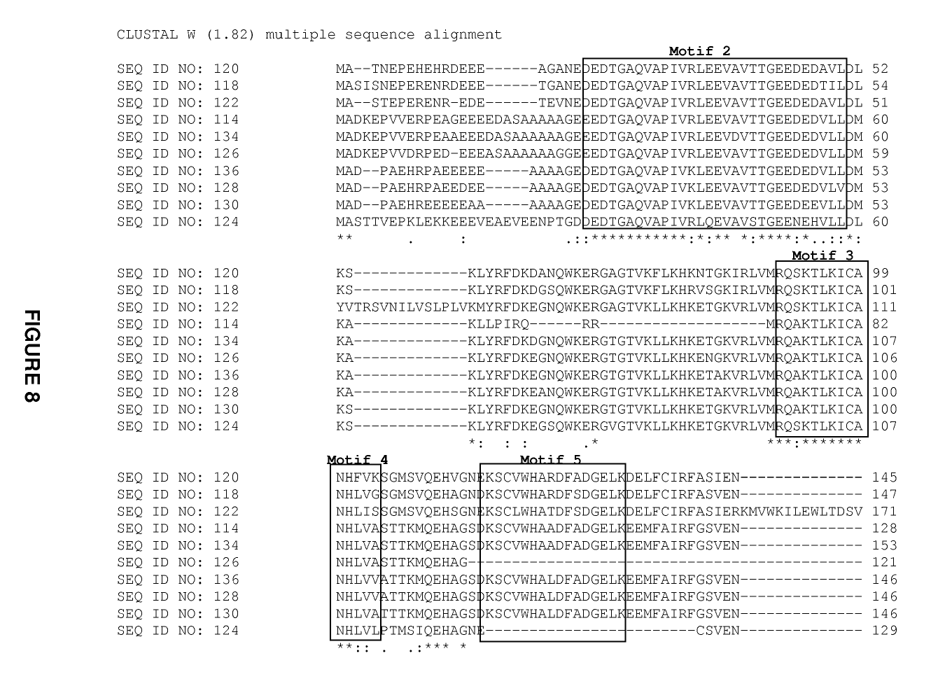

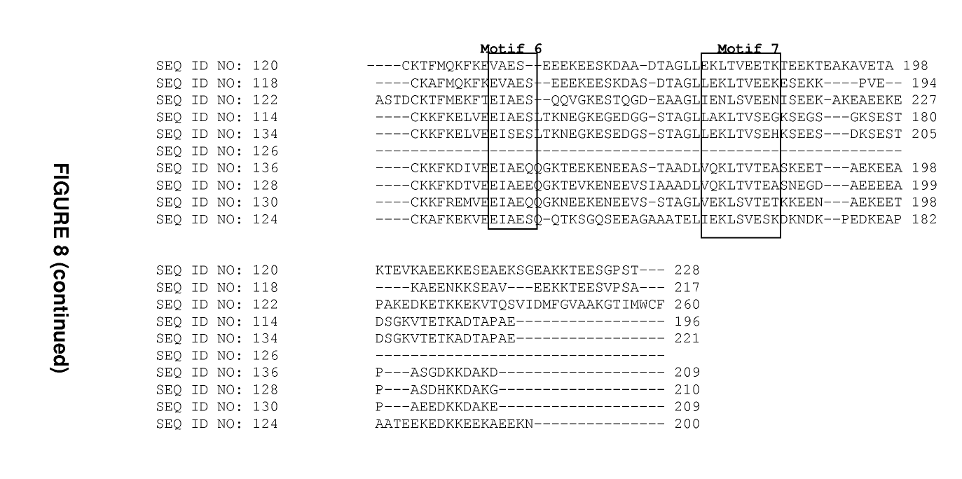

FIG. 7 shows a binary vector p1017, for increased expression in Oryza sativa of an Arabidopsis thaliana nucleic acid encoding an FBXW polypeptide under the control of a GOS2 promoter (SEQ ID NO: 58).

### RANBP

FIG. 8 shows an alignment of RANBP polypeptides as defined hereinabove. The sequences were aligned using AlignX program from Vector NTI suite (InforMax, Bethesda, Md.). Multiple alignment was done with a gap opening penalty of 10 and a gap extension of 0.01. Minor manual editing was also carried out where necessary to better position some conserved regions. Motif II, III, I, IV, V, VI and VII are indicated.

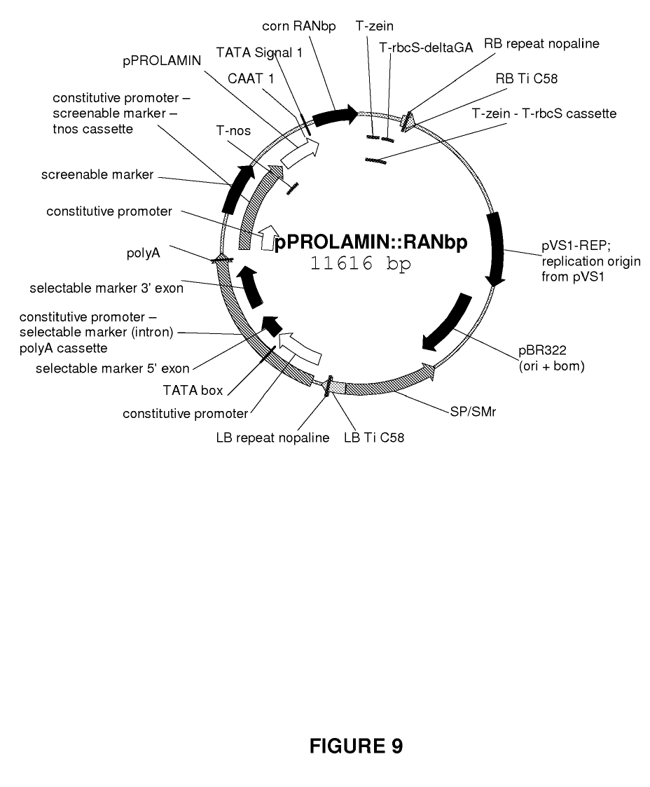

FIG. 9 shows a binary vector p072, for increased expression in Oryza sativa of a Zea Mays RANBP-encoding nucleic acid under the control of a prolamin promoter (internal reference PRO0090).

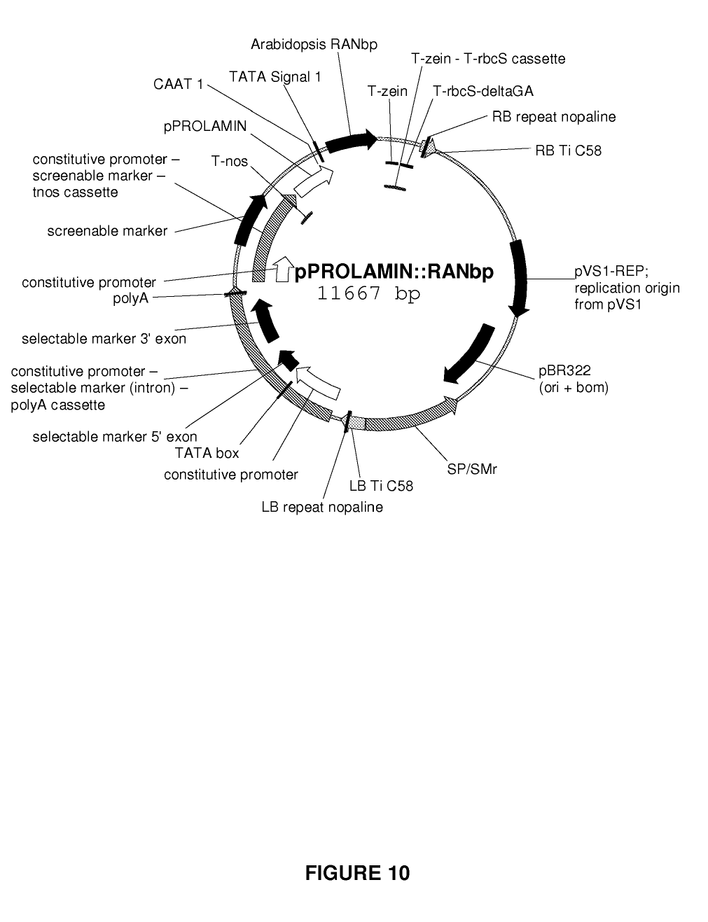

FIG. 10 shows a binary vector p074, for increased expression in Oryza sativa of an Arabidopsis thaliana RANBP-encoding nucleic acid under the control of a prolamin promoter (internal reference PRO0090).

### GLK

FIG. 11 gives a graphical overview of maize and rice GLK genes (Rossini et al., 2001). The horizontal lines represent the untranslated transcribed regions (UTRs). Boxes represent different domains of the coding regions as indicated. NLS is the predicted nuclear localisation signal, DBD is the putative DNA binding domain, which comprises the GARP domain. White triangles designate position of the introns present in all four genes; black triangles designate the position of the intron that is not found in the G2 gene. Note that although OsGLK1 is predicted to have a nuclear localisation, it was not possible to predict with high confidence the presence of a NLS sequence.

FIG. 12 shows the domain organization of the GLK protein used in the present invention (SEQ ID NO: 157). The GARP domain (bold) has some resemblance to the MYB domain (indicated in italics, as identified by SMART); the GCT domain is underlined.

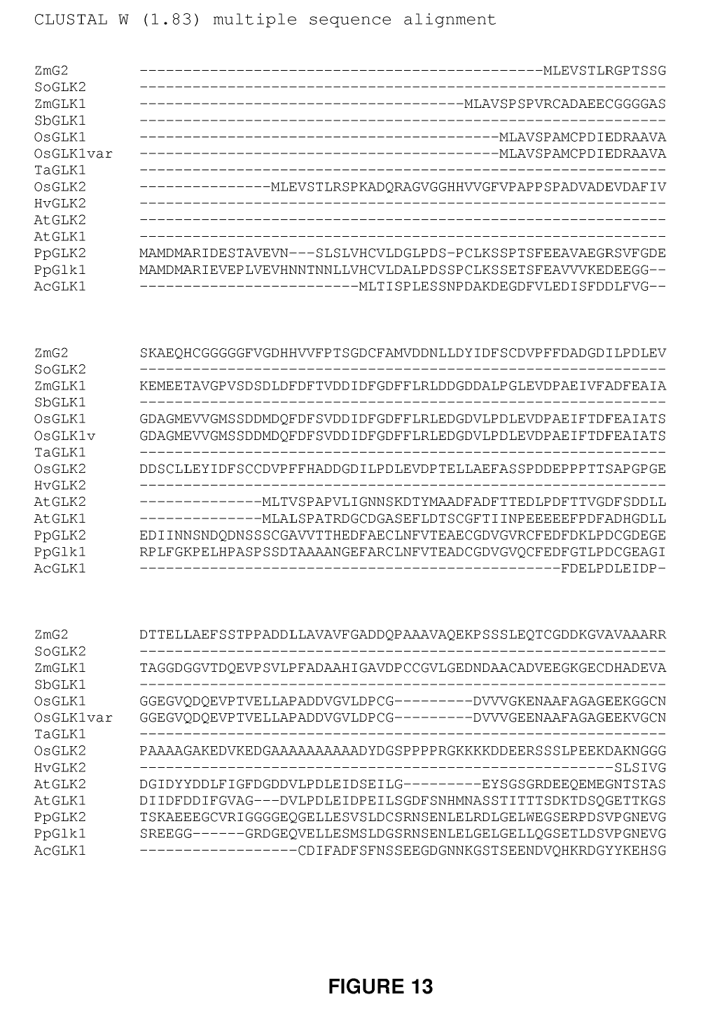

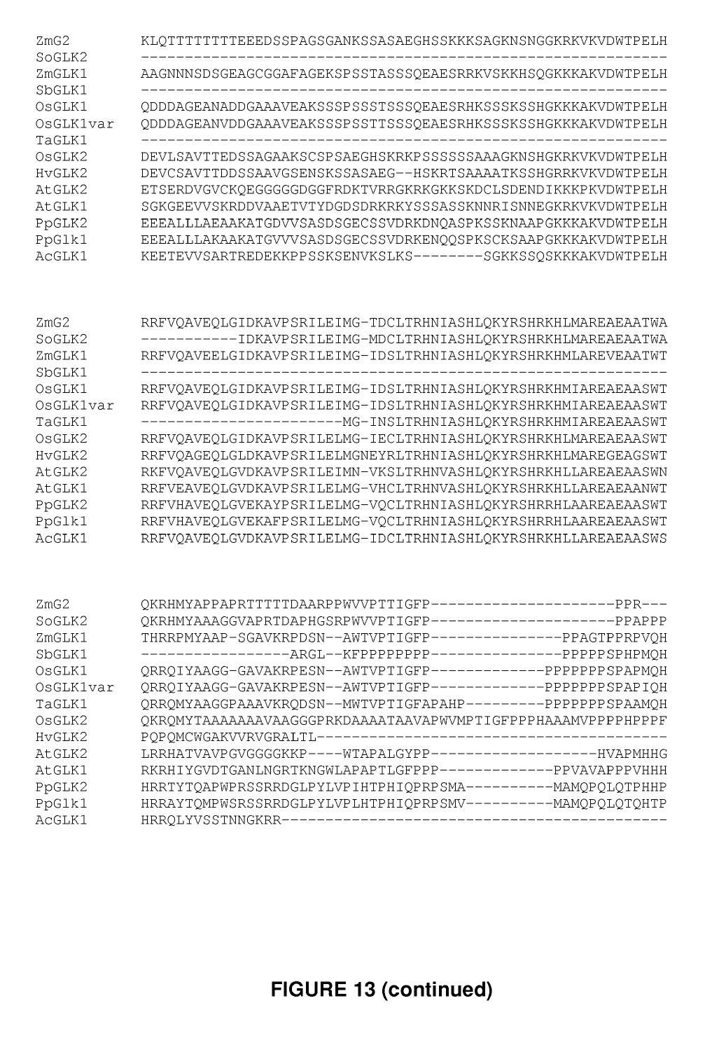

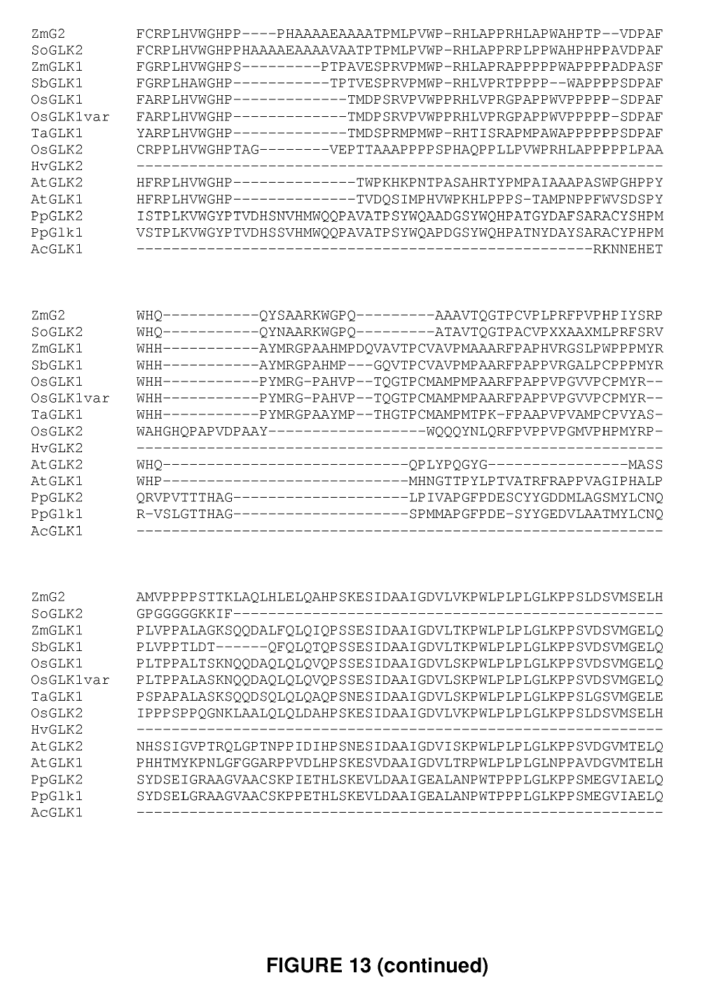

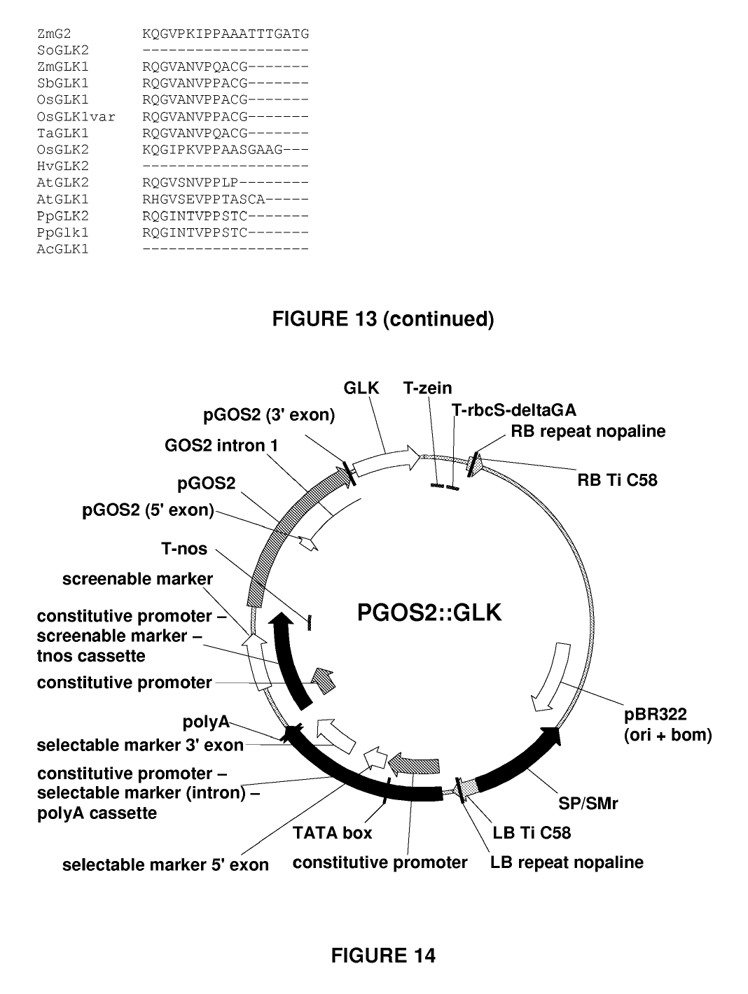

FIG. 13 gives a multiple alignment of the proteins listed as SEQ ID NO: 157 (OsGLK1), SEQ ID NO: 169 (OsGLK2), SEQ ID NO: 171 (AtGLK1), SEQ ID NO: 173 (AtGLK2), SEQ ID NO: 175 (PpGLK1), SEQ ID NO: 177 (PpGLK2), SEQ ID NO: 179 (ZmGLK1), SEQ ID NO: 181 (ZmG2), SEQ ID NO: 183 (TaGLK1), SEQ ID NO: 185 (AcGLK1), SEQ ID NO: 189 (SbGLK1), SEQ ID NO: 193 (OsGLKlvar). The alignment was made with CLUSTAL W (1.83), weight matrix: BLOSUM, gap opening penalty: 10, gap extension penalty: 0.05.

FIG. 14 shows a map of the binary plasmid p045, used for increasing expression in Oryza sativa of an Oryza sativa GLK-encoding nucleic acid under the control of a GOS2 promoter (SEQ ID NO: 58).

### REV ΔHDZip/START

FIG. 15 shows a phylogram of class III HDZip polypeptides. There are two monocot REV polypeptides (Oryza sativa) that cluster up with a single dicot REV polypeptide (Arabidopsis thaliana). The circle indicates the clade of REV polypeptides of which the REV nucleic acid sequences ΔHDZip/START may be useful in performing the methods of the invention. After Floyd et al. (2006) Genetics 173(1): 373-88

FIG. 16. Neighbour-joining tree output after a multiple sequence alignment of all class III HDZip polypeptides from Arabidopsis thaliana (5 in total) and Oryza sativa (5 in total), including examples of REV polypeptide orthologues and paralogues (see Example 27), using CLUSTAL W (1.83) (at GenomeNet service at the Kyoto University Bioinformatics Center), and default values (Blosum 62 as weight matrix, gap open penalty of 10; gap extension penalty of 0.05). The polypeptides of the REV branch are indicated by the curly bracket, and are separated from the other four class III HDZip polypeptides by the bold line. The circle indicates the REV branching out point.

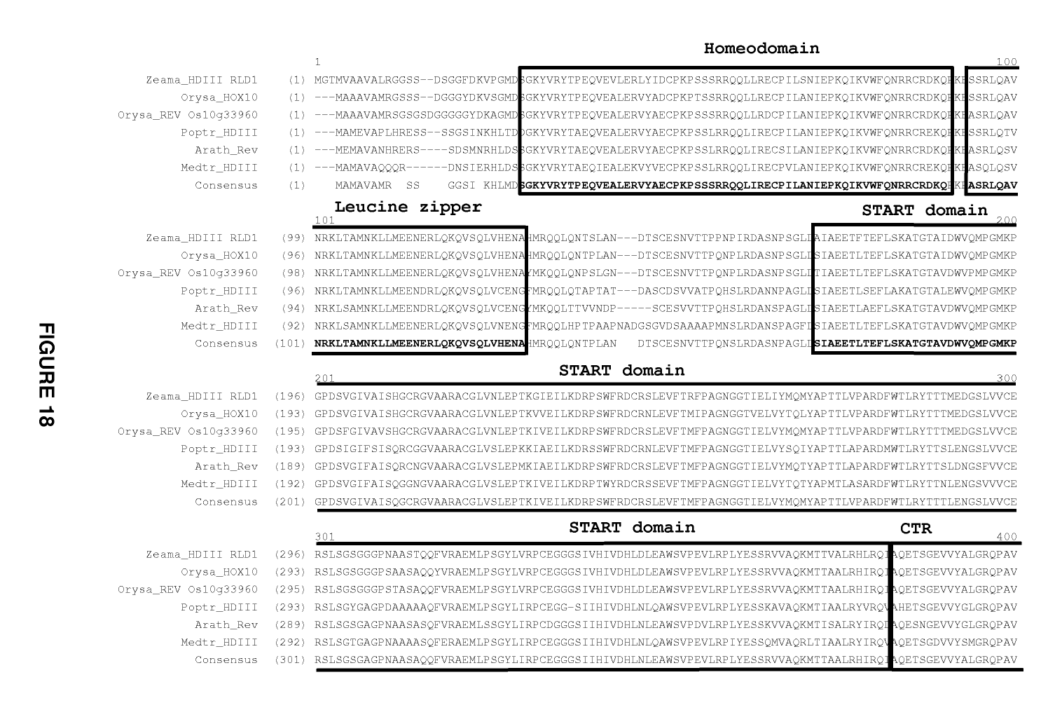

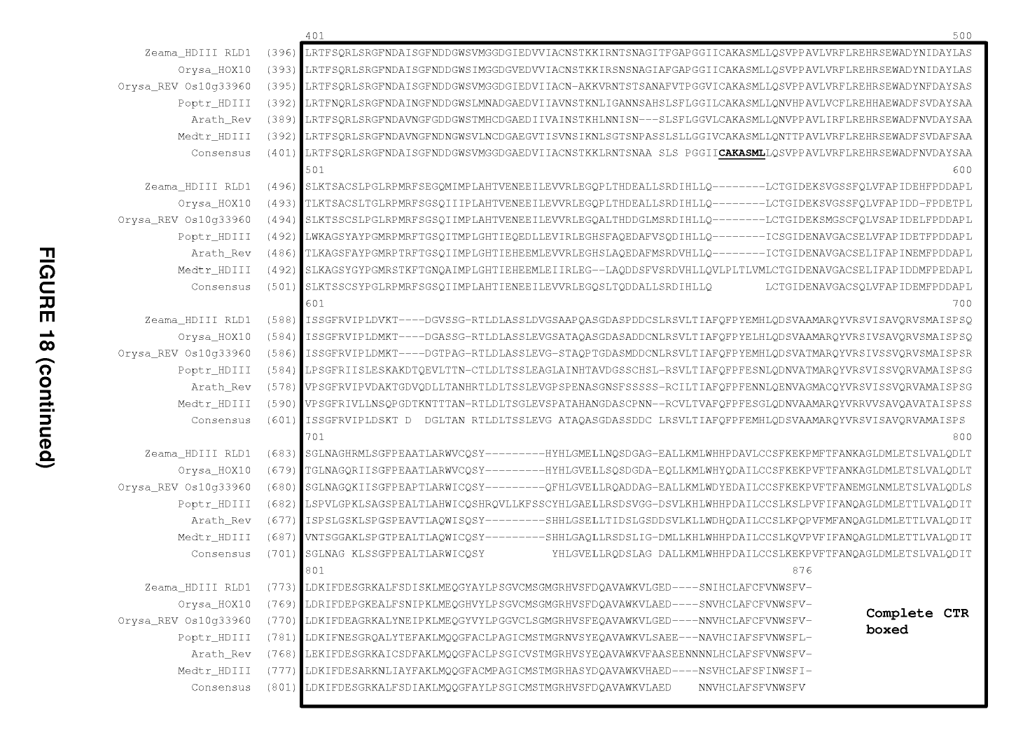

FIG. 17 is a schematic representation of a full-length REV polypeptide. REV polypeptides comprise from N-terminus to C-terminus: (i) a homeodomain (HD) domain, for DNA binding; (ii) a leucine zipper (Zip), for protein-protein interaction; (iii) a START domain for lipid/sterol binding (comprising a miRNA complementary binding site, mir165/166), and (iv) a C-terminal region (CTR), of undefined function. For example, in one REV polypeptide from Oryza sativa as represented by SEQ ID NO: 199, the HD spans amino acids 27 to 87, the leucine zipper amino acids 91 to 127, the START domain amino acids 166 to 376 and the CTR amino acids 377 to 840.

FIG. 18 is a multiple sequence alignment of full length REV polypeptides of which the REV ΔHDZip/START nucleic acid sequences are useful in performing the methods according to the invention, using CLUSTAL W (1.83) (at GenomeNet service at the Kyoto University Bioinformatics Center), and default values (Blosum 62 as weight matrix, gap open penalty of 10; gap extension penalty of 0.05). The homeodomain, the leucine zipper, the START domain and the CTR are heavily boxed.

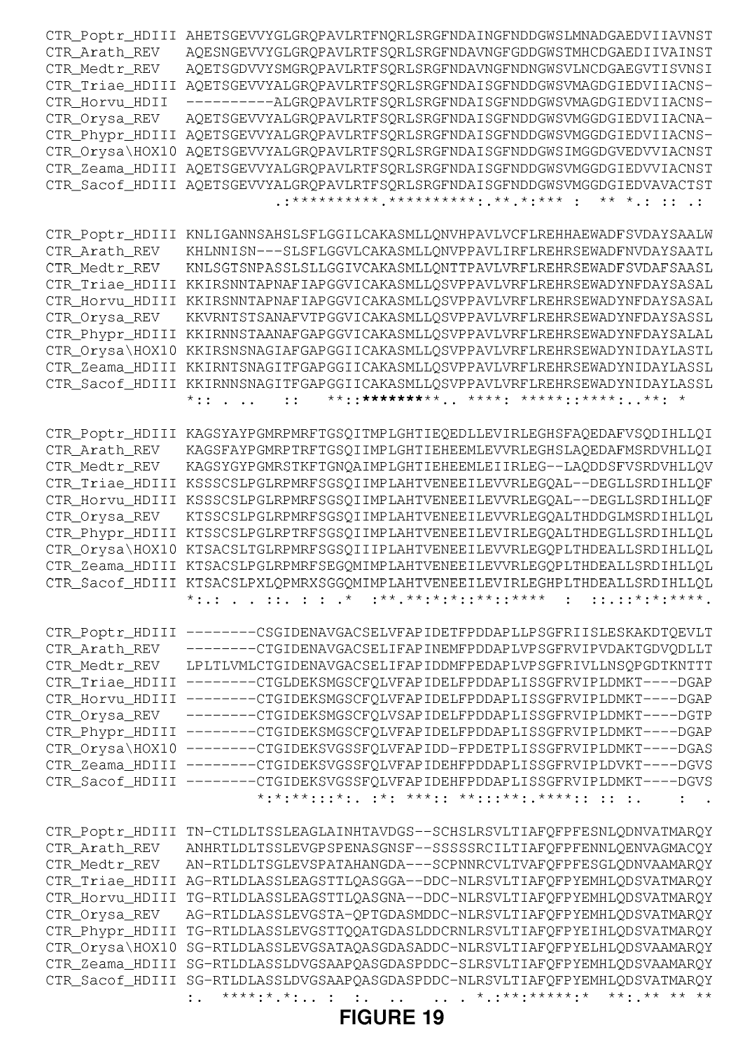

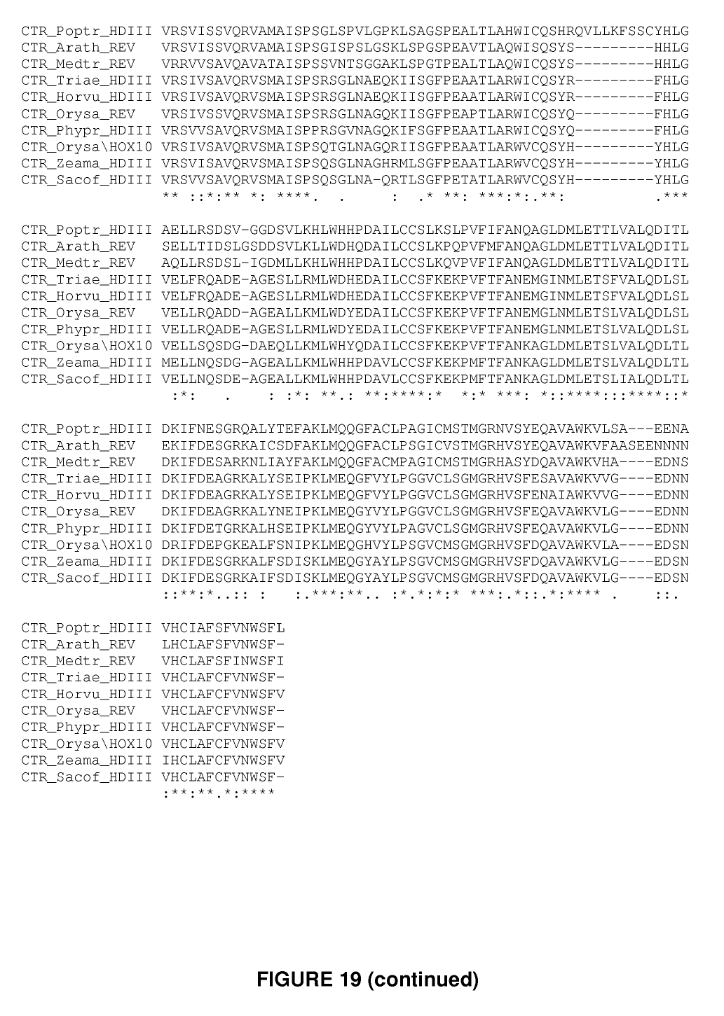

FIG. 19 is a multiple sequence alignment of the CTR of REV polypeptides (both full length and partial polypeptides, as listed in Example 27), of which the REV ΔHDZip/START nucleic acid sequences are useful in performing the methods according to the invention, using CLUSTAL W (1.83) (at GenomeNet service at the Kyoto University Bioinformatics Center), and default values (Blosum 62 as weight matrix, gap open penalty of 10; gap extension penalty of 0.05

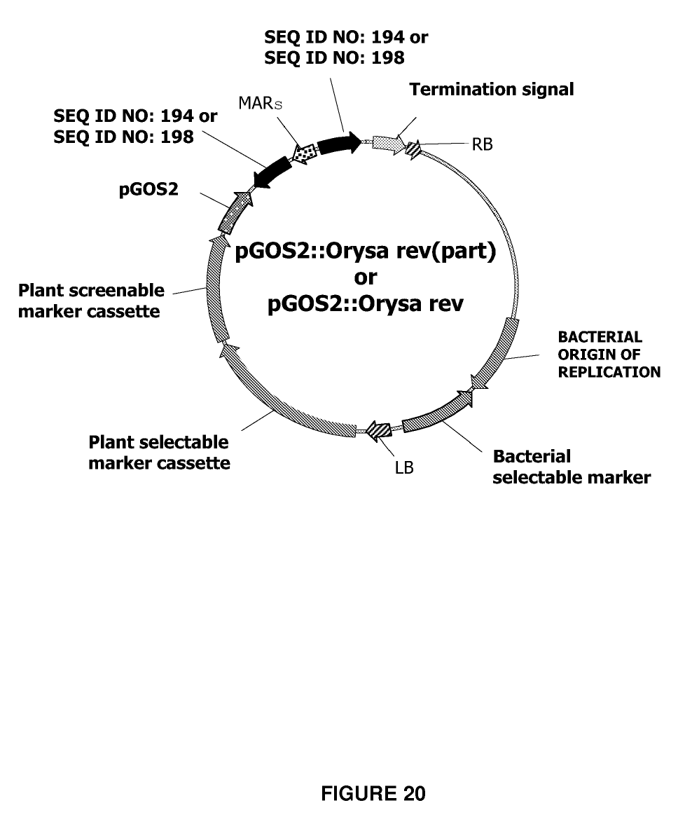

FIG. 20 represents the binary vectors p0443 and p0448 for reduction of an endogenous REV gene expression in Oryza sativa, using respectively the REV ΔHDZip/START nucleic acid sequence as represented by SEQ ID NO: 194 (encoding a partial CTR from a REV polypeptide) and the REV nucleic acid sequence as represented by SEQ ID NO: 198 (encoding the entire REV polypeptide). The nucleic acid sequences are cloned as inverted repeats separated by a non-coding region (here a partial matrix attachment region (MAR) from tobacco) with the aim of obtaining an mRNA with a hairpin conformation. A constitutive promoter (PRO0129) controls the expression of both nucleic acid sequences SEQ ID NO: 194 and SEQ ID NO: 198 in the two different plasmids.

### CLE

FIG. 21 shows the domain organisation of the CLE-like polypeptide of SEQ ID NO: 233: in italics: signal sequence, the conserved Arg residue needed for proteolytic processing is indicated in bold (here Arg73), the CLE domain is underlined.

FIG. 22. gives a multiple alignment of CLE-like polypeptides: a single dot below the sequence alignment indicates a less conserved substitution, a colon indicates a conserved substitution, an asterisk indicates a conserved residue in all sequences.

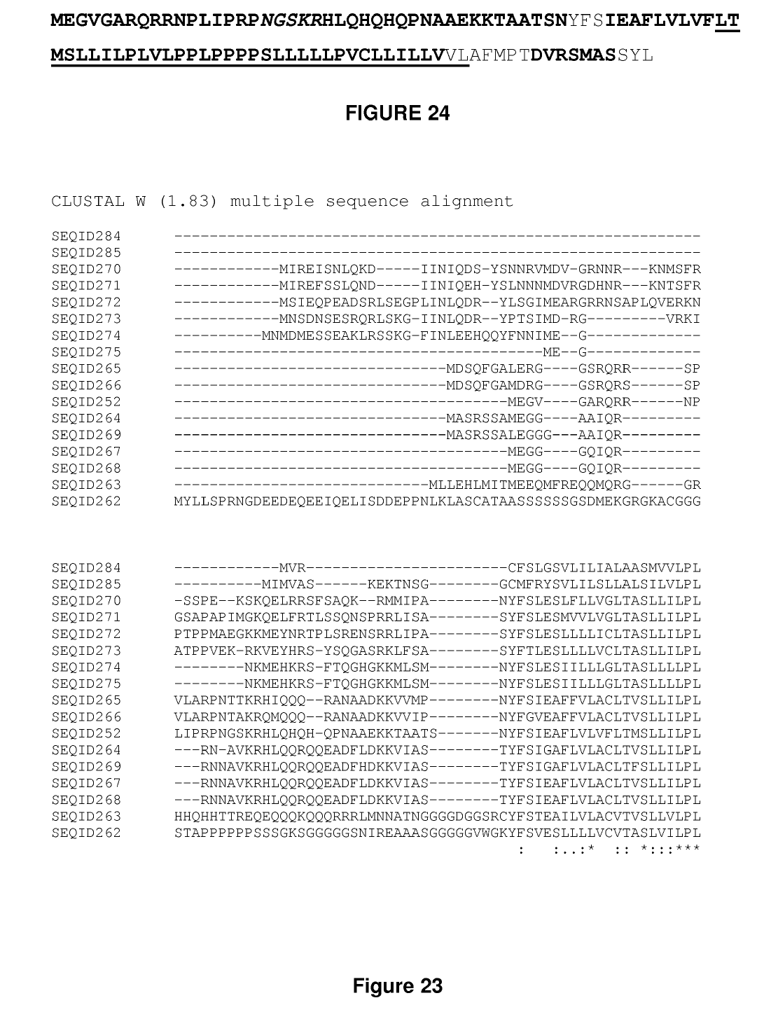

FIG. 23 represents the binary vector p068 for endogenous gene silencing in Oryza sativa, preferentially using the nucleic acid sequence encoding a CLE-like polypeptide, or a part thereof, using a hairpin construct under the control of an endosperm-specific promoter (PRO90).

### SYR NUE

FIG. 24 gives an overview of the conserved motifs present in SEQ ID NO: 252. The leucine rich domain is underlined, the conserved motifs 1, 2 and 3 are indicated in bold and the sequence in italics represents the putative N-glycosylation site with the putative protein kinase C phosphorylation site.

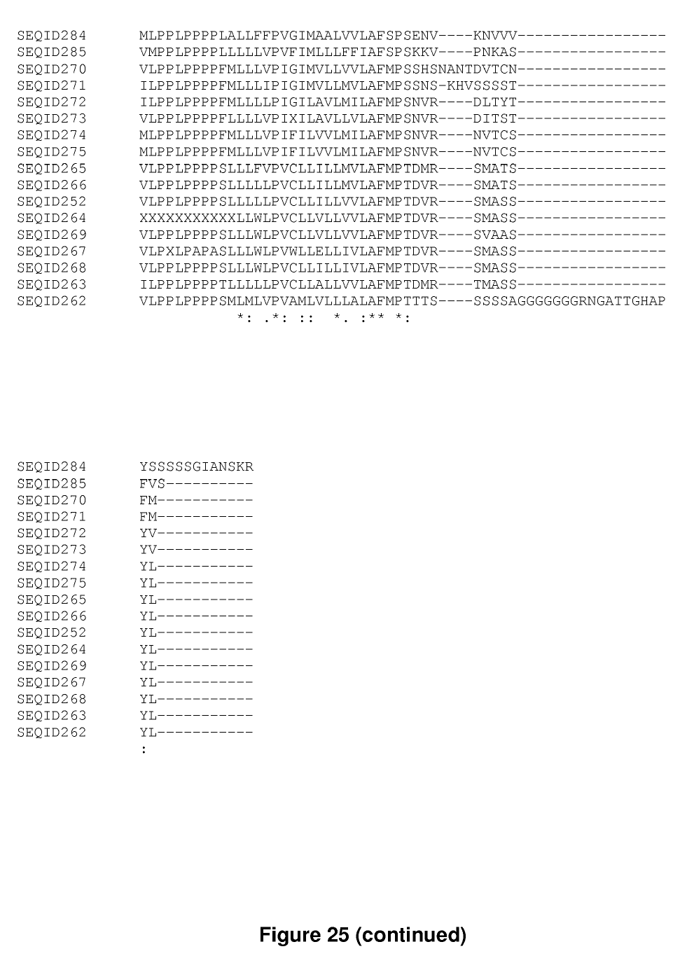

FIG. 25 shows a multiple alignment of various SYR proteins. The asterisks indicate identical amino acid residues, the colons represent highly conserved substitutions and the dots represent less conserved substitutions. With the information from FIG. 1, the various domains and conserved motifs in SEQ ID NO: 252 can be easily identified in the other SYR proteins.

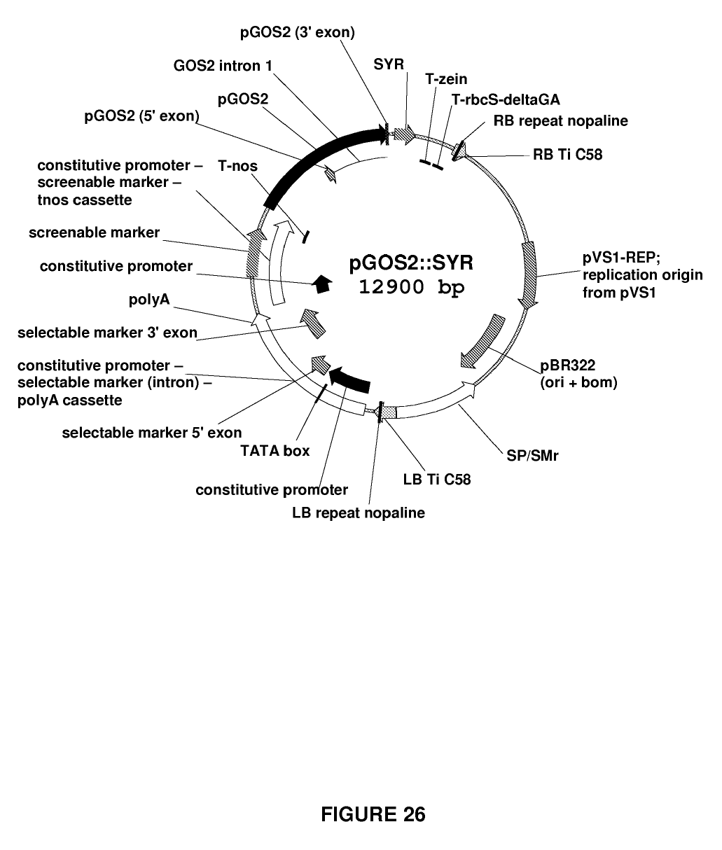

FIG. 26 shows binary vector pGOS2::SYR for transformation and expression in Oryza sativa of an Oryza sativa SYR nucleic acid under the control of a rice GOS2 promoter.

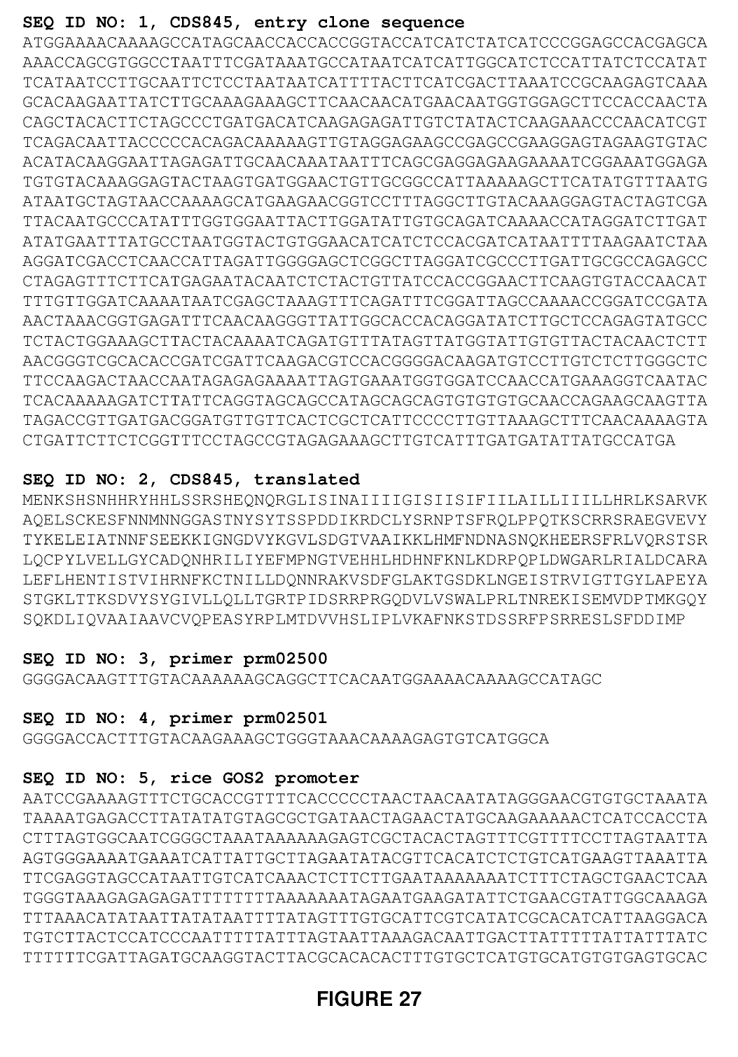

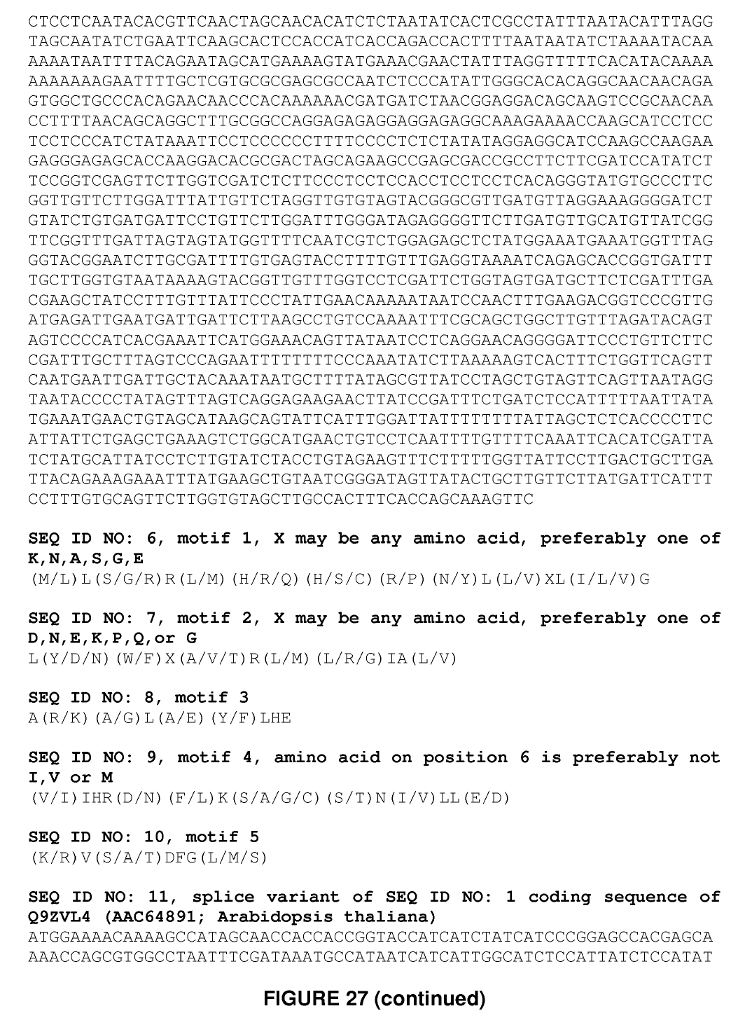

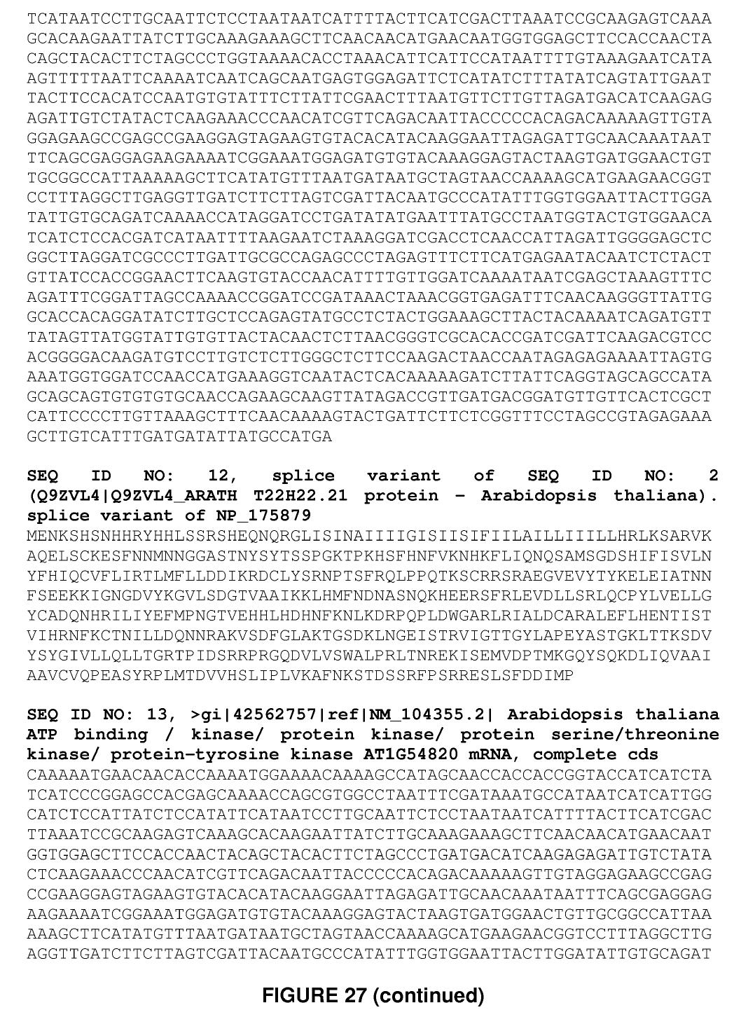

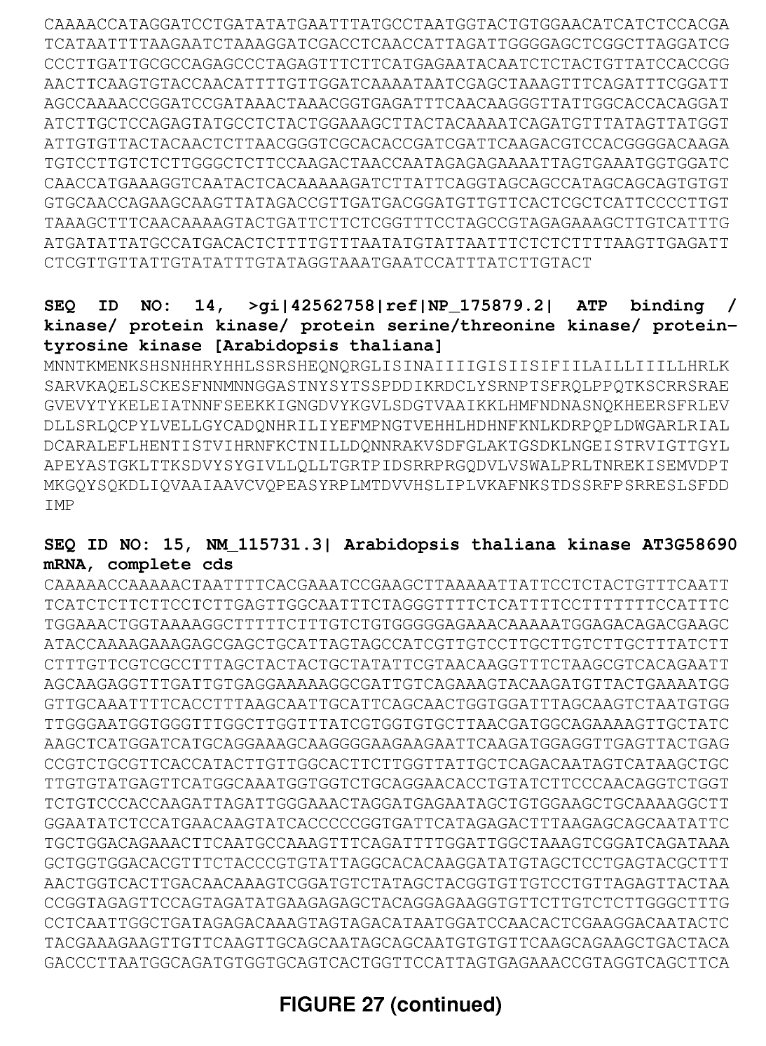

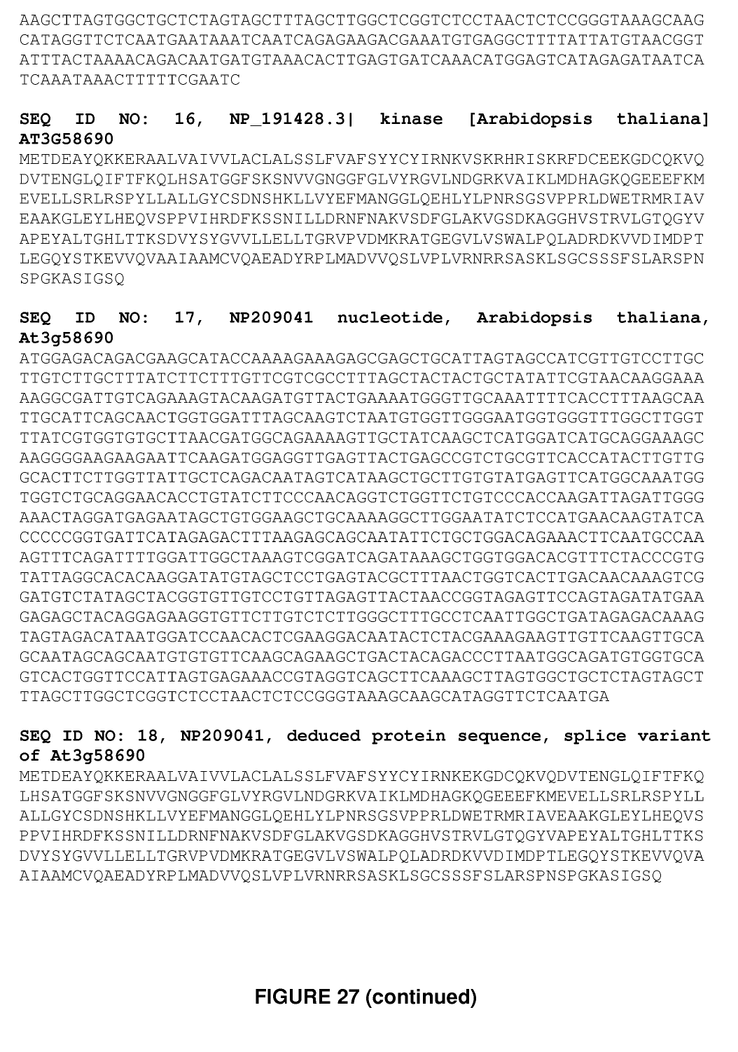

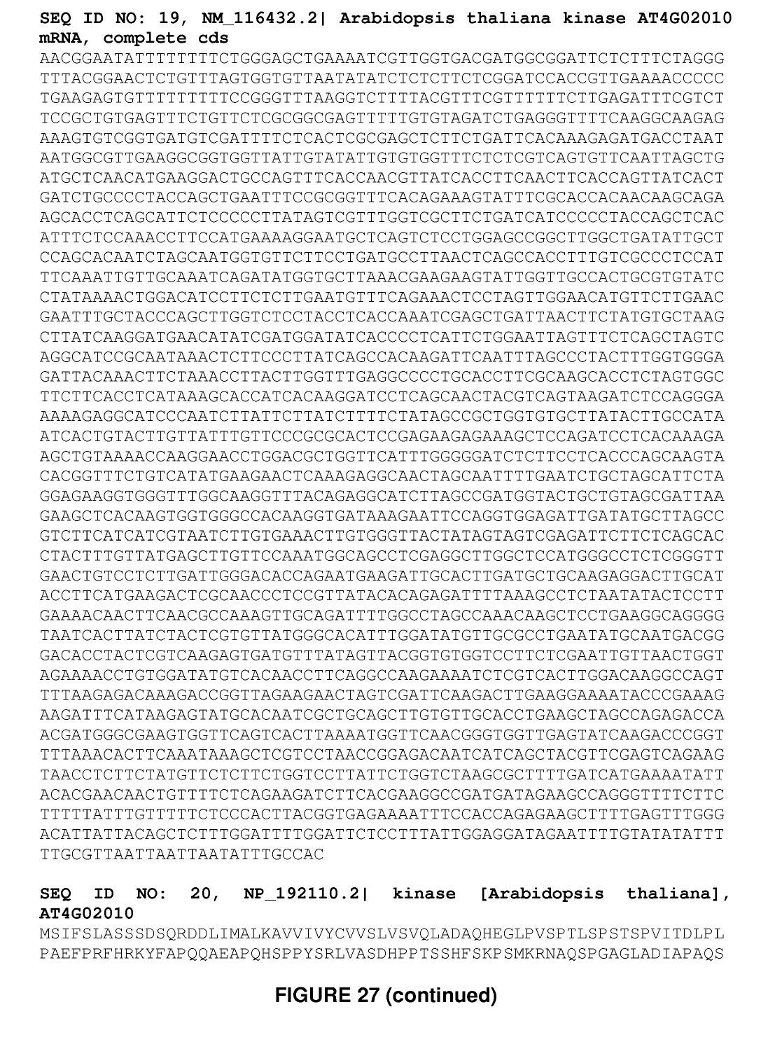

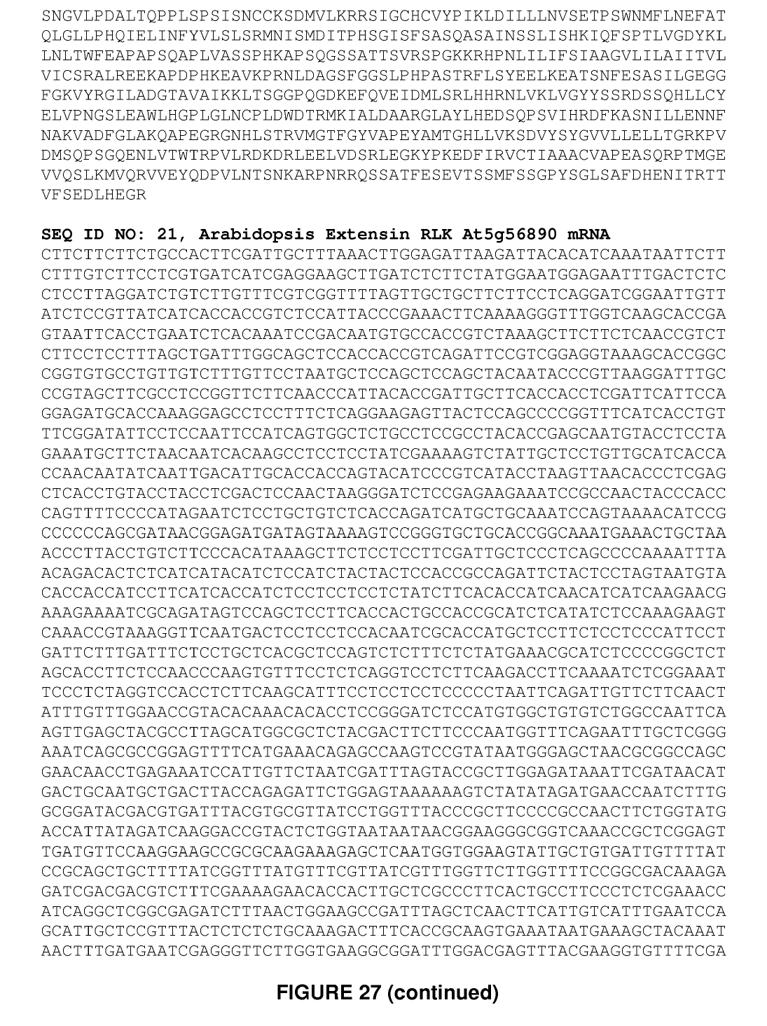

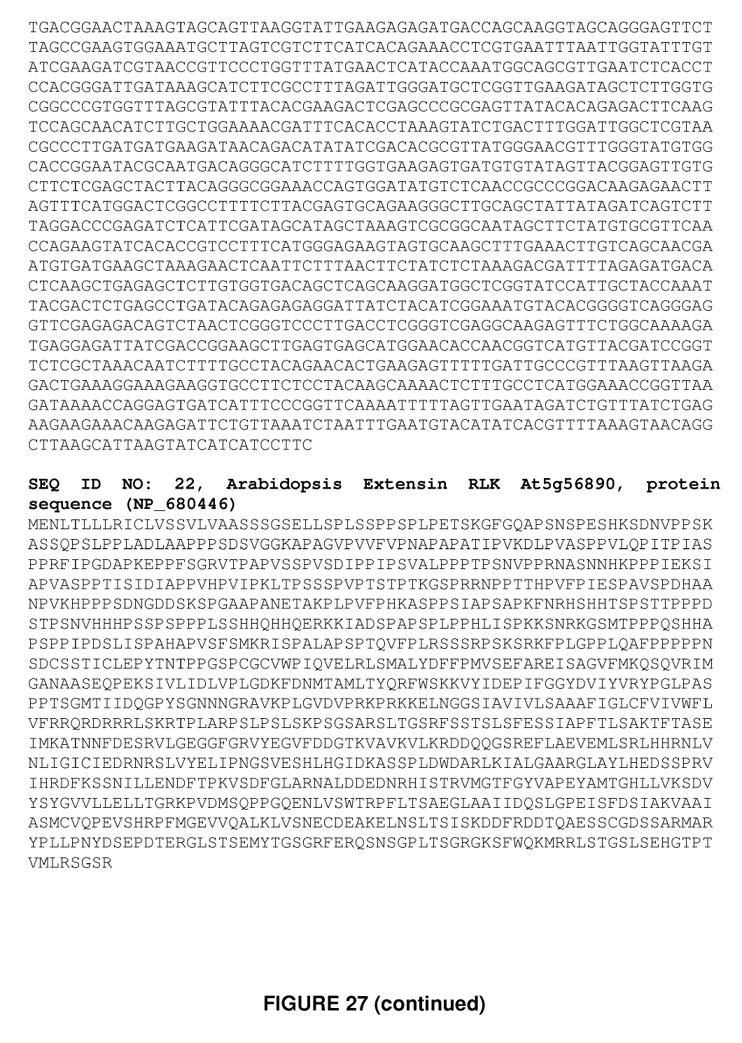

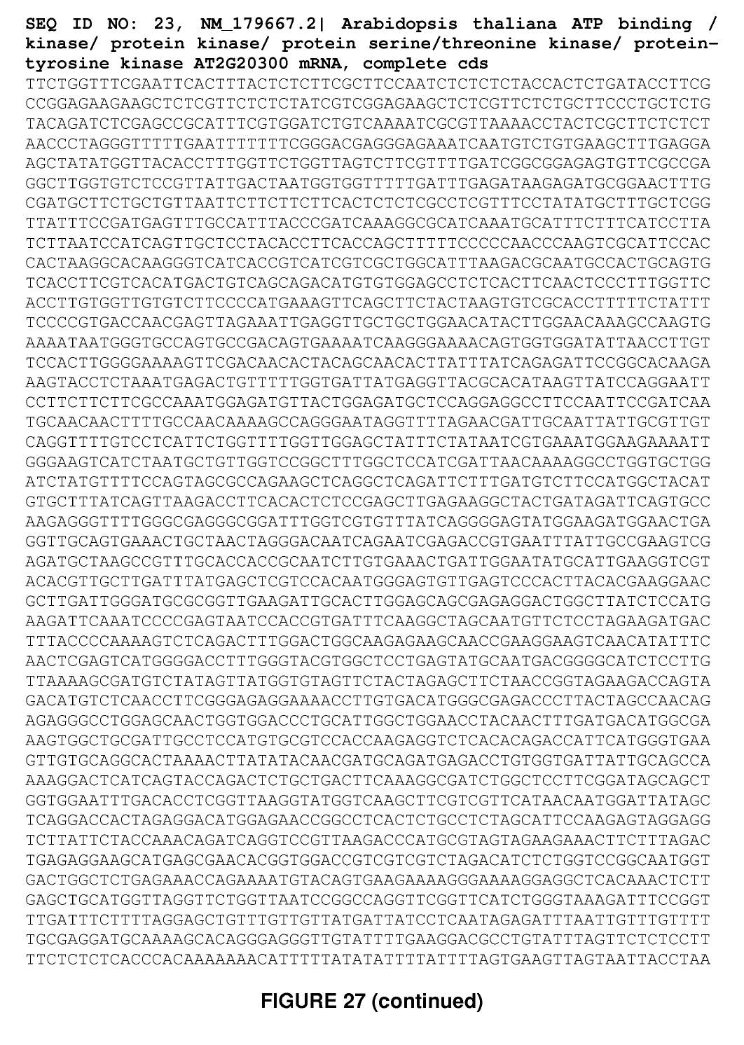

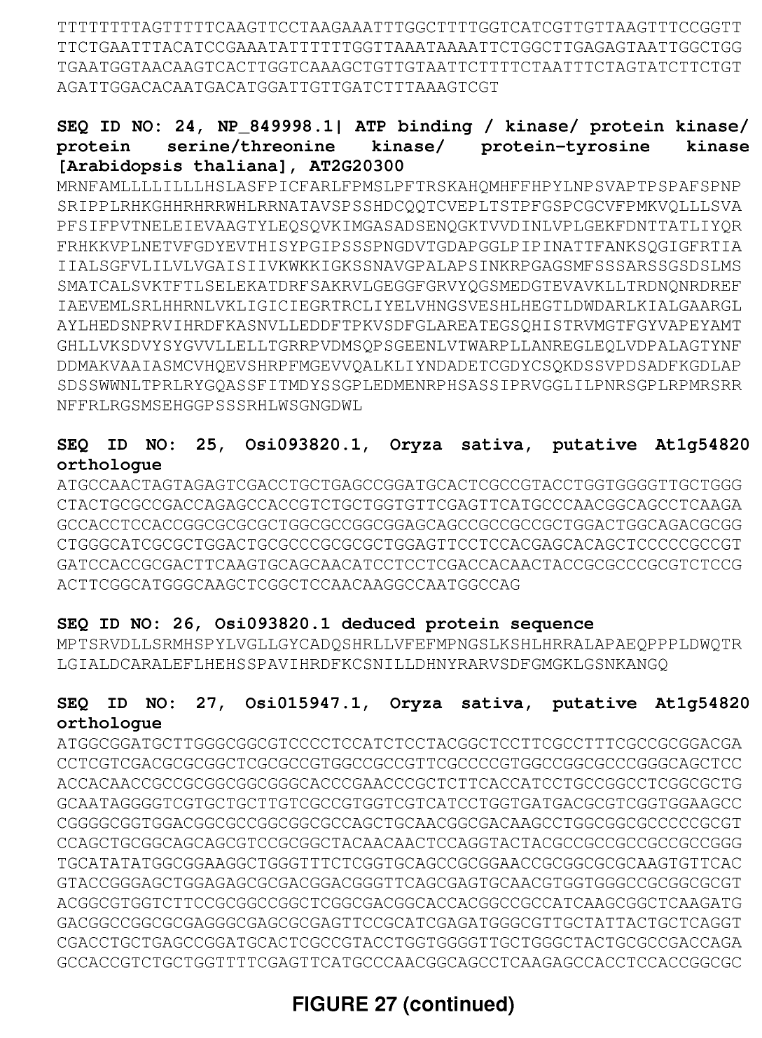

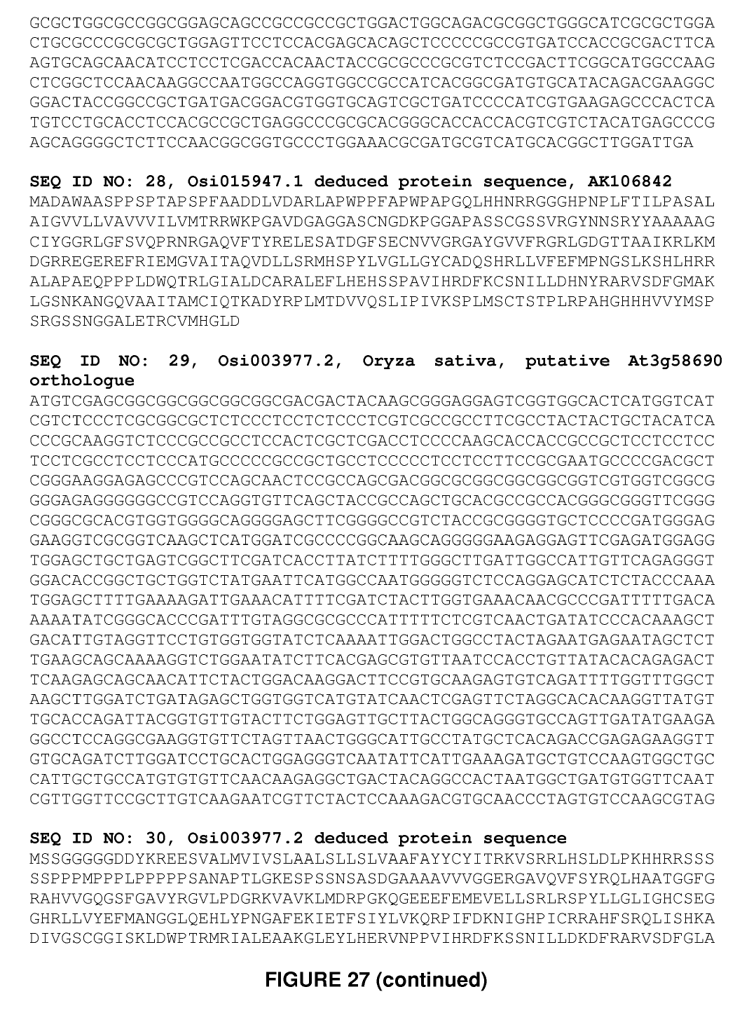

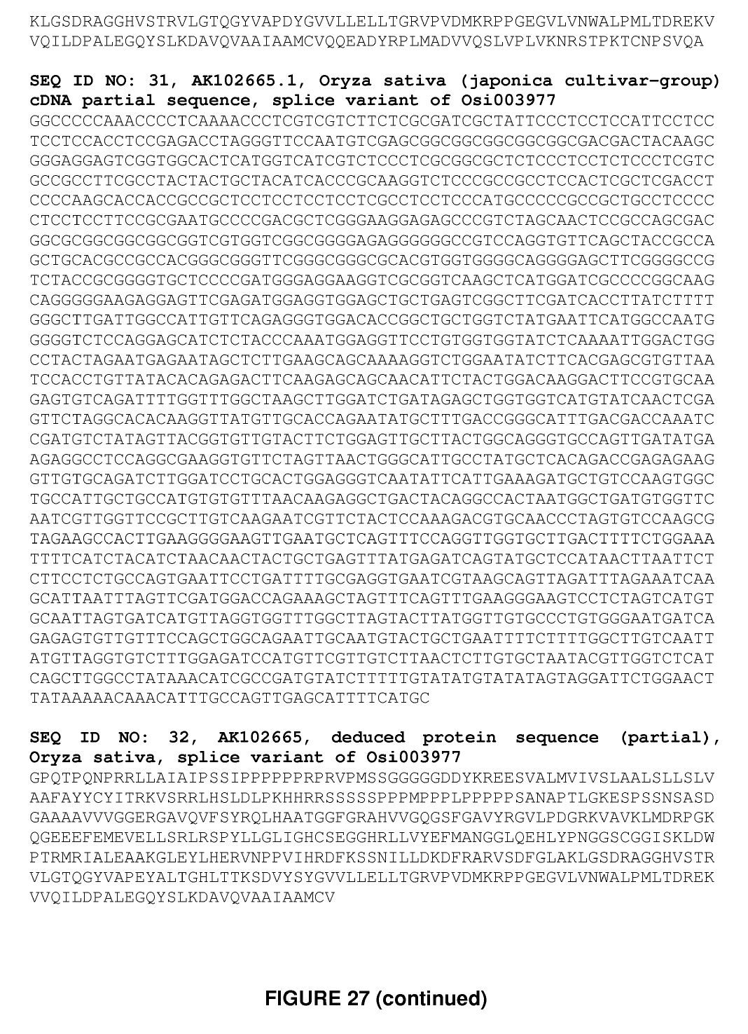

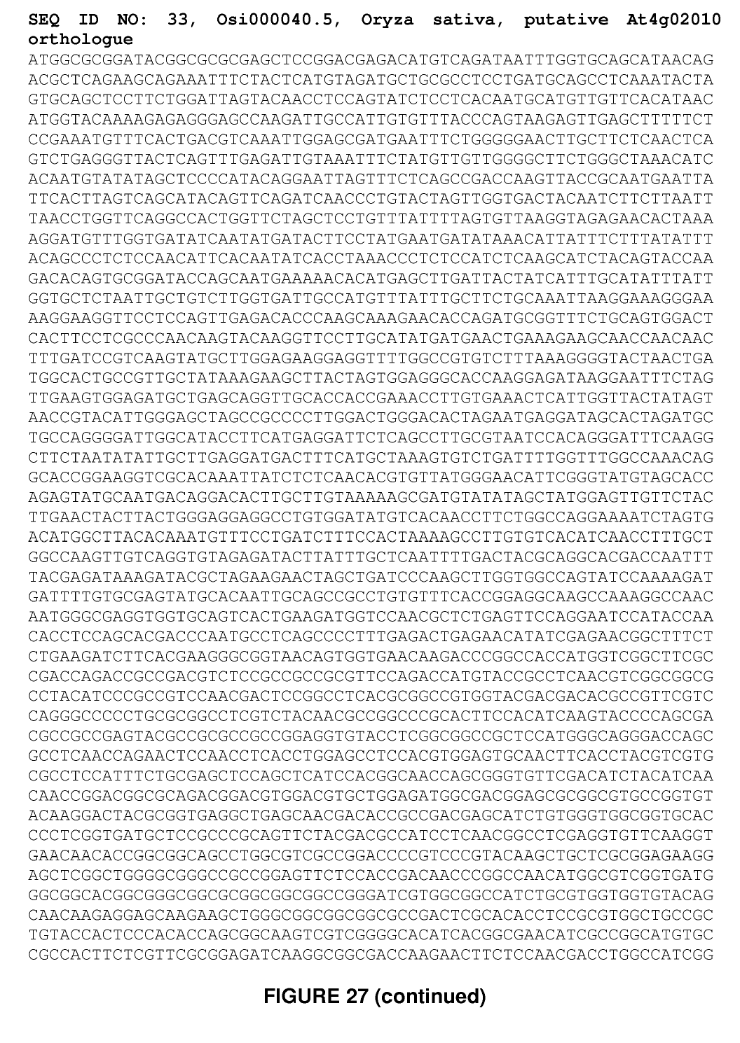

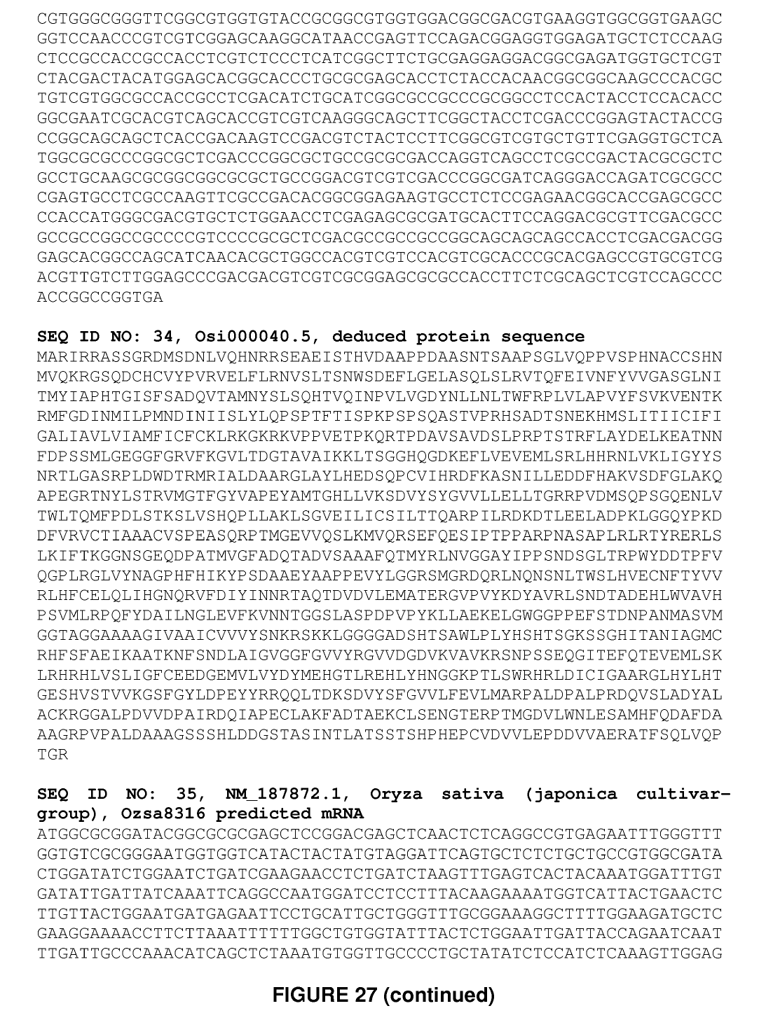

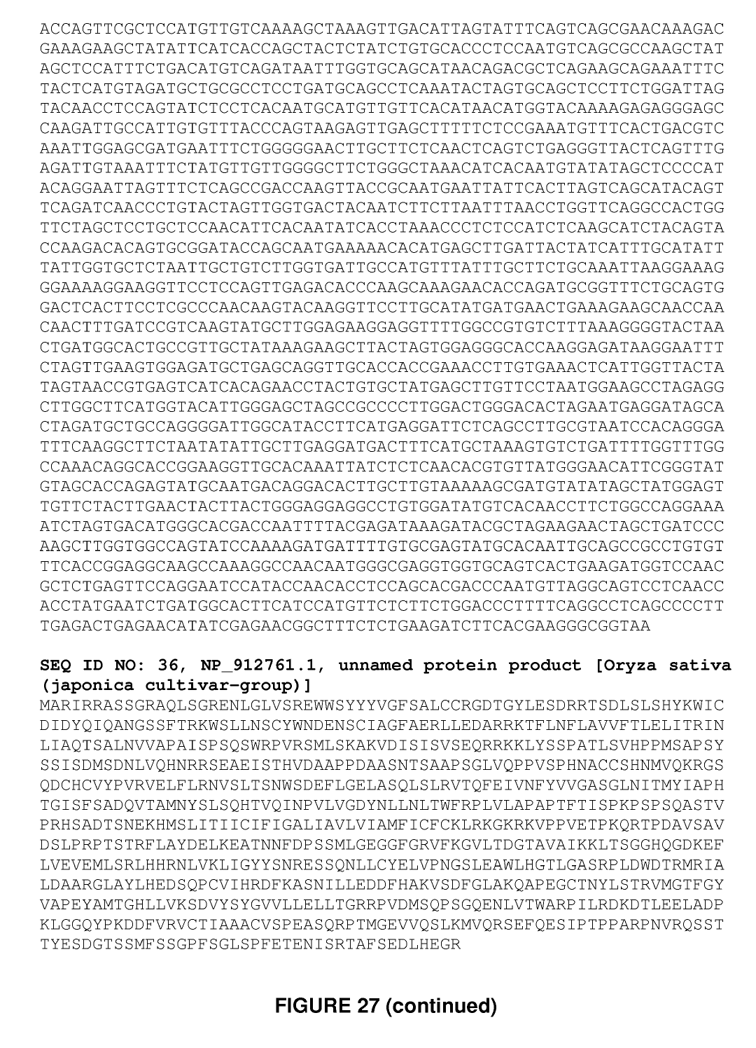

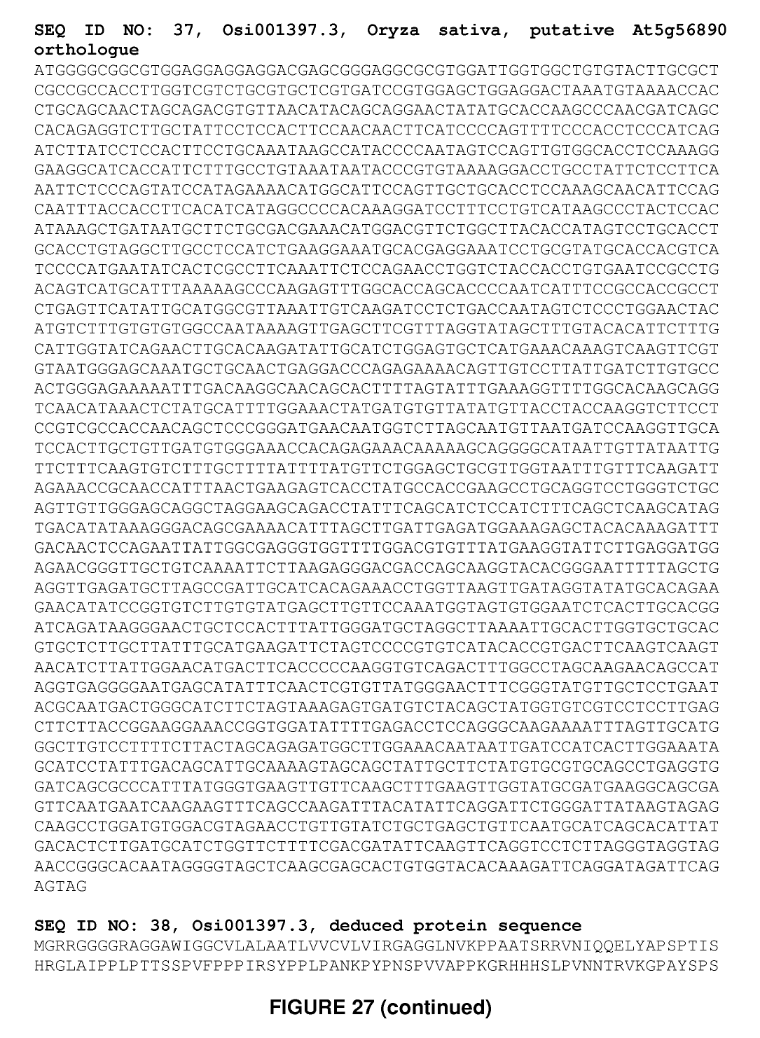

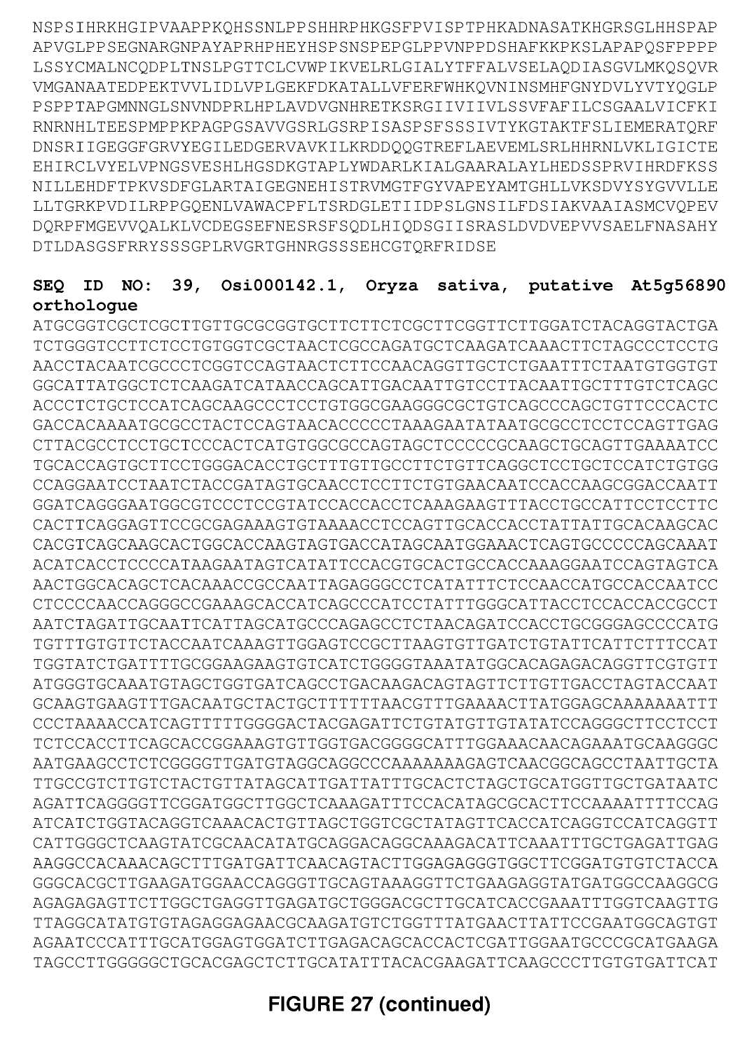

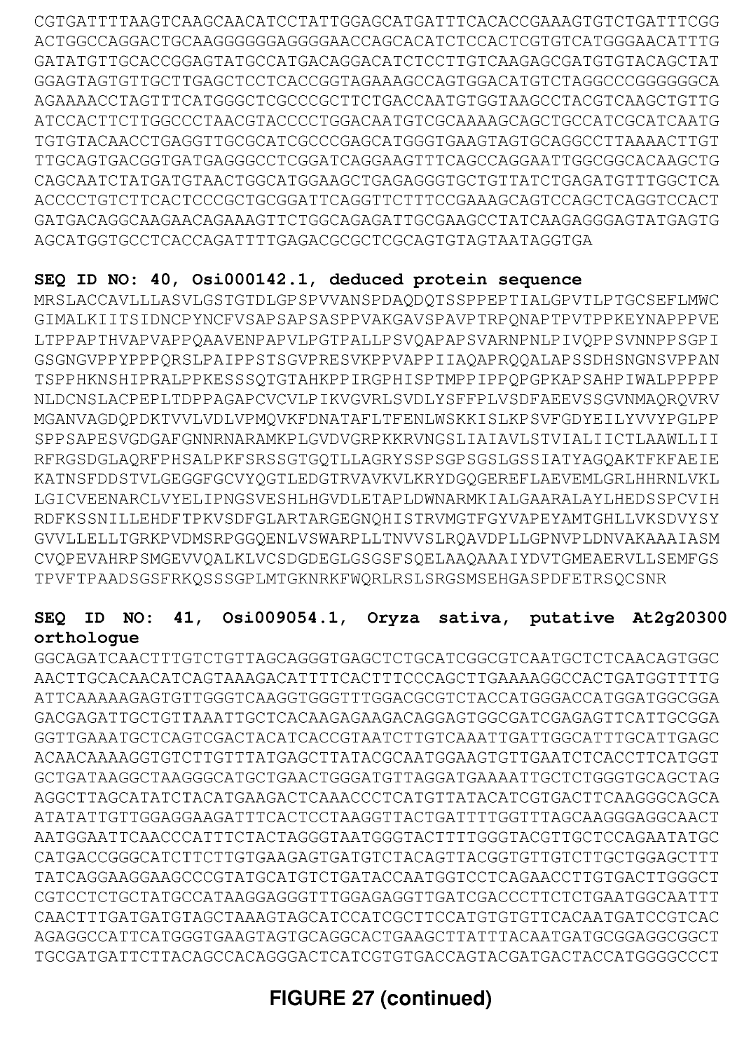

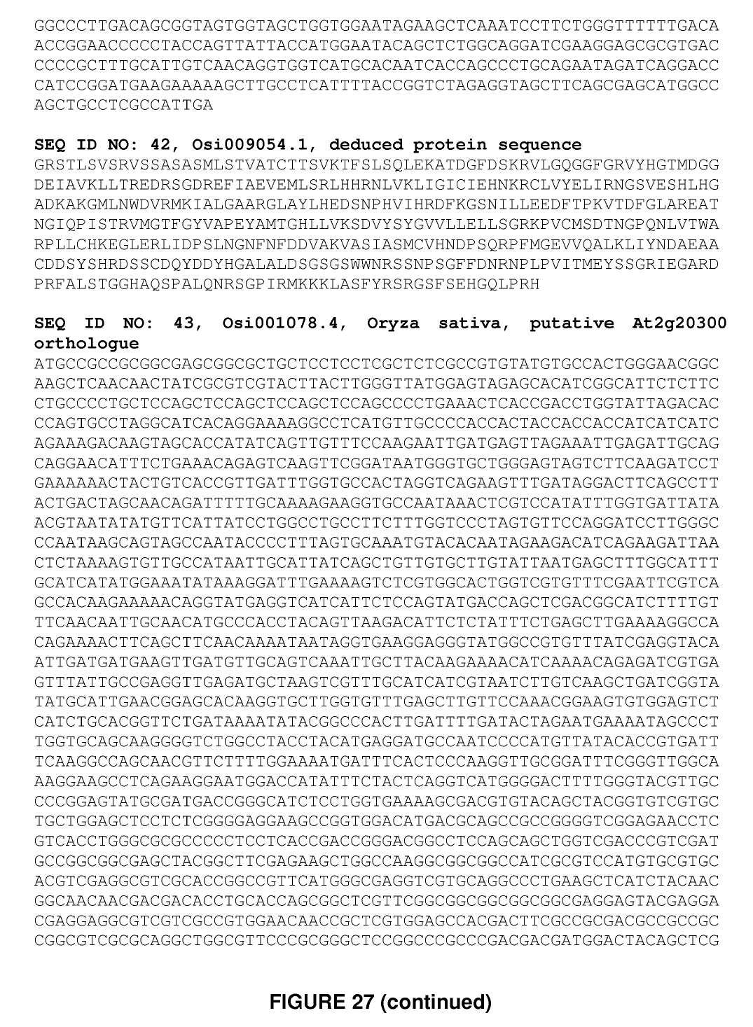

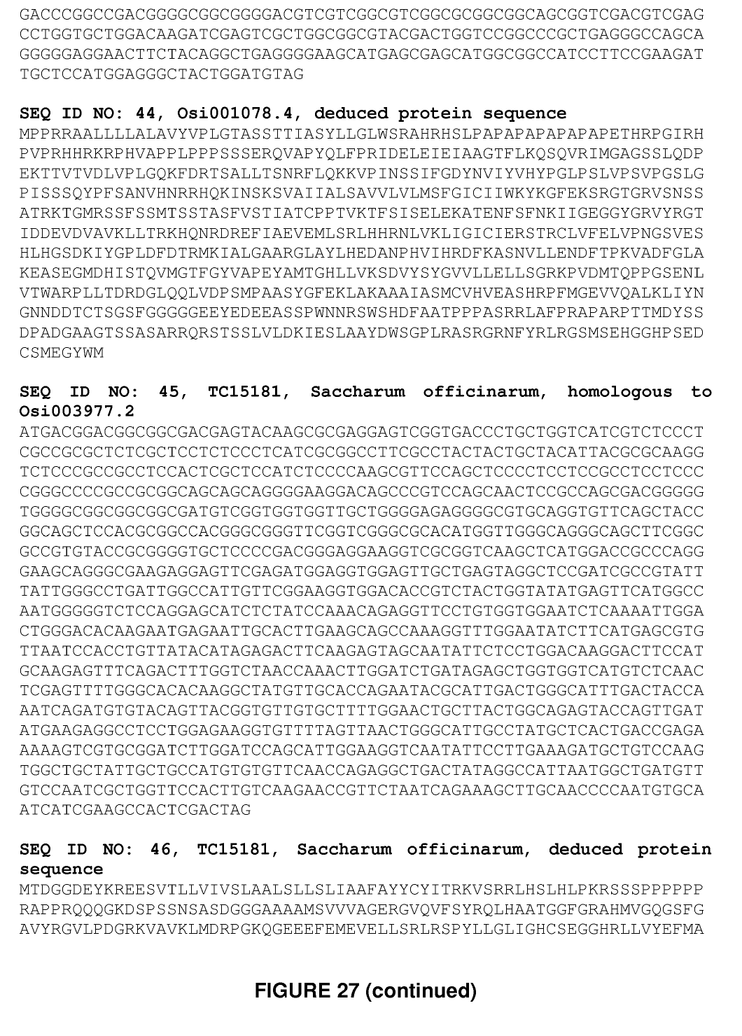

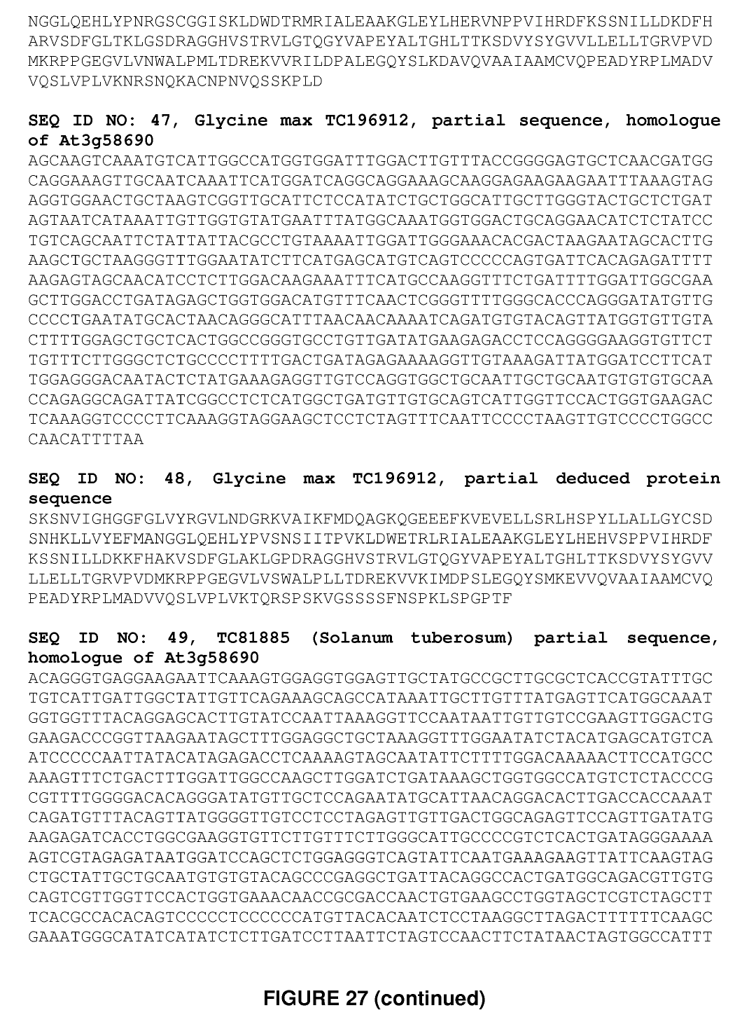

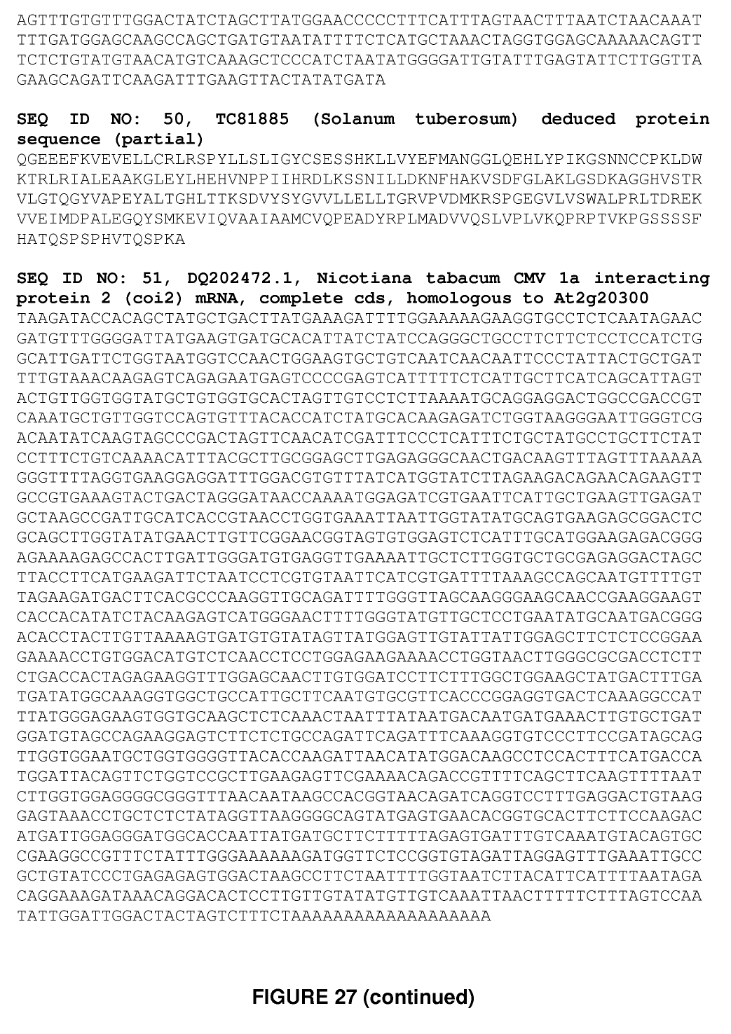

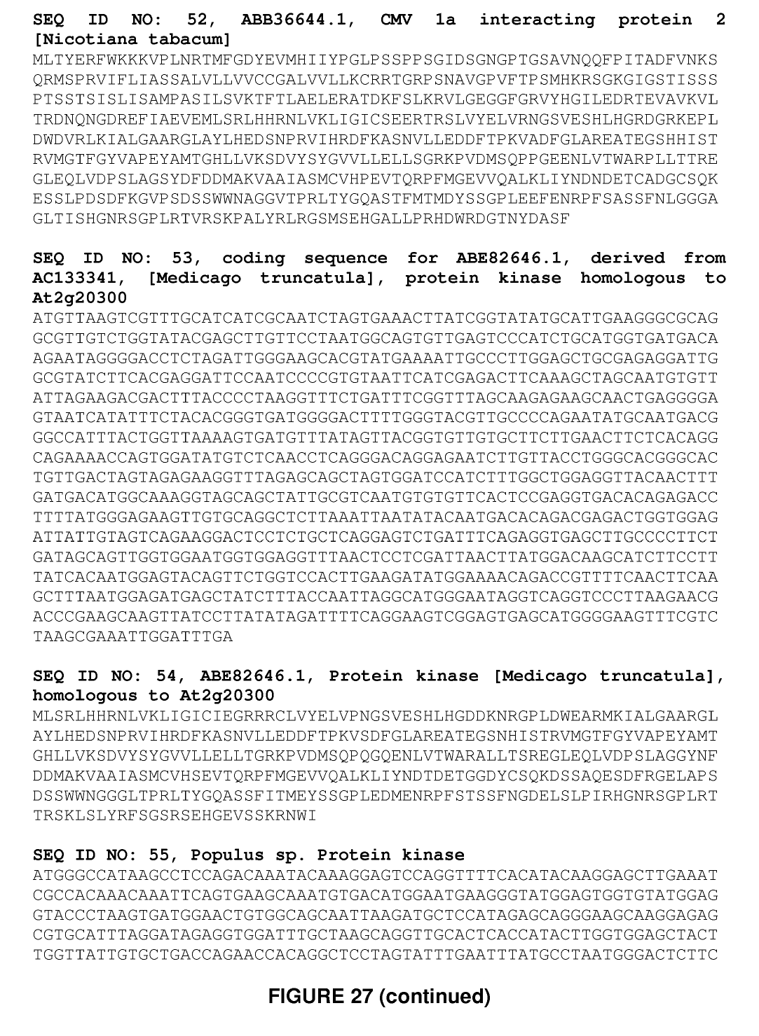

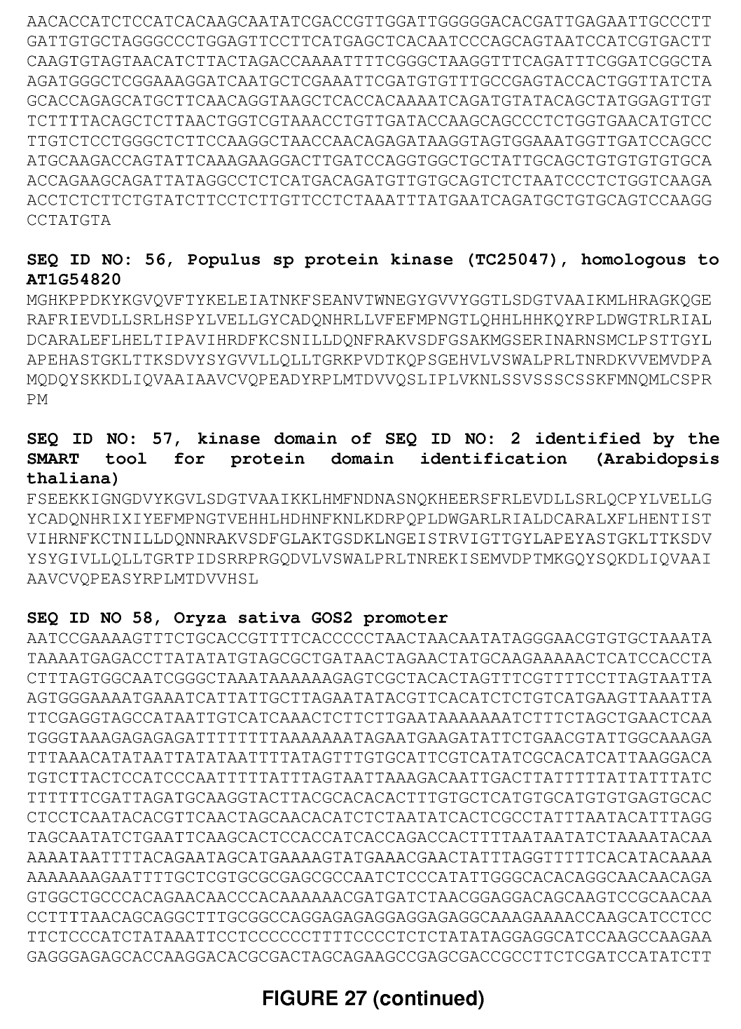

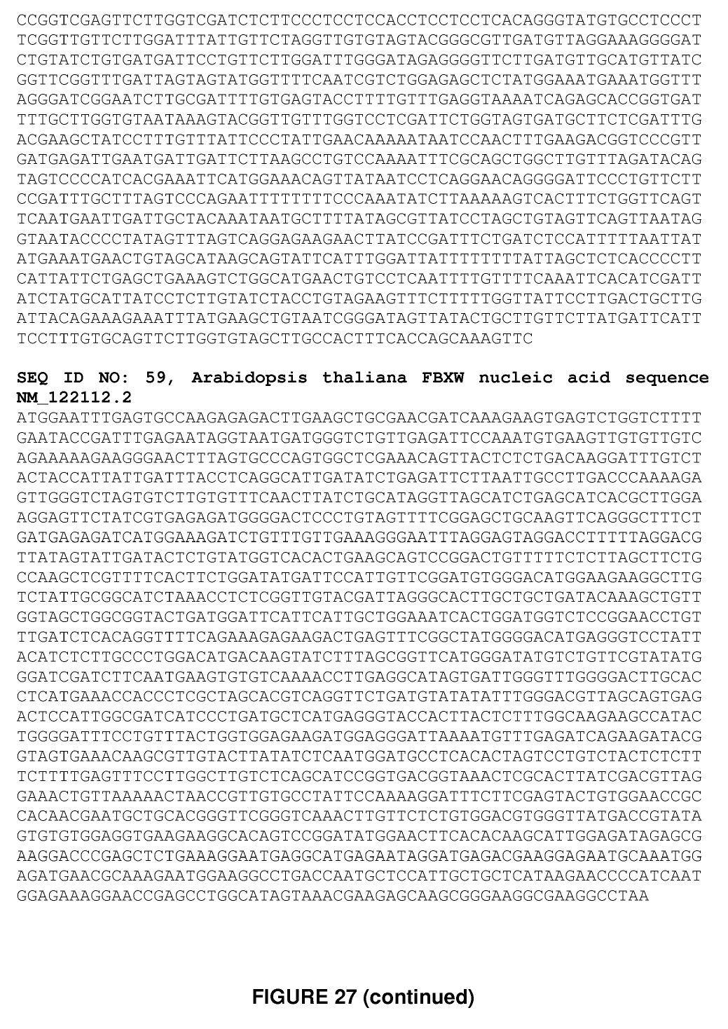

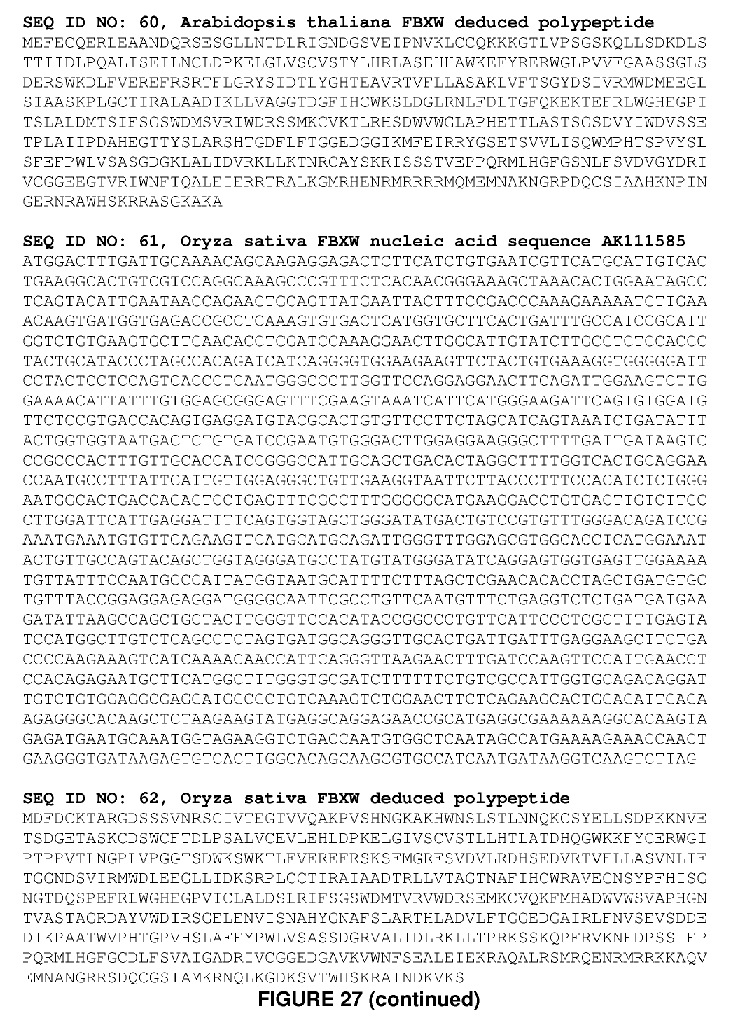

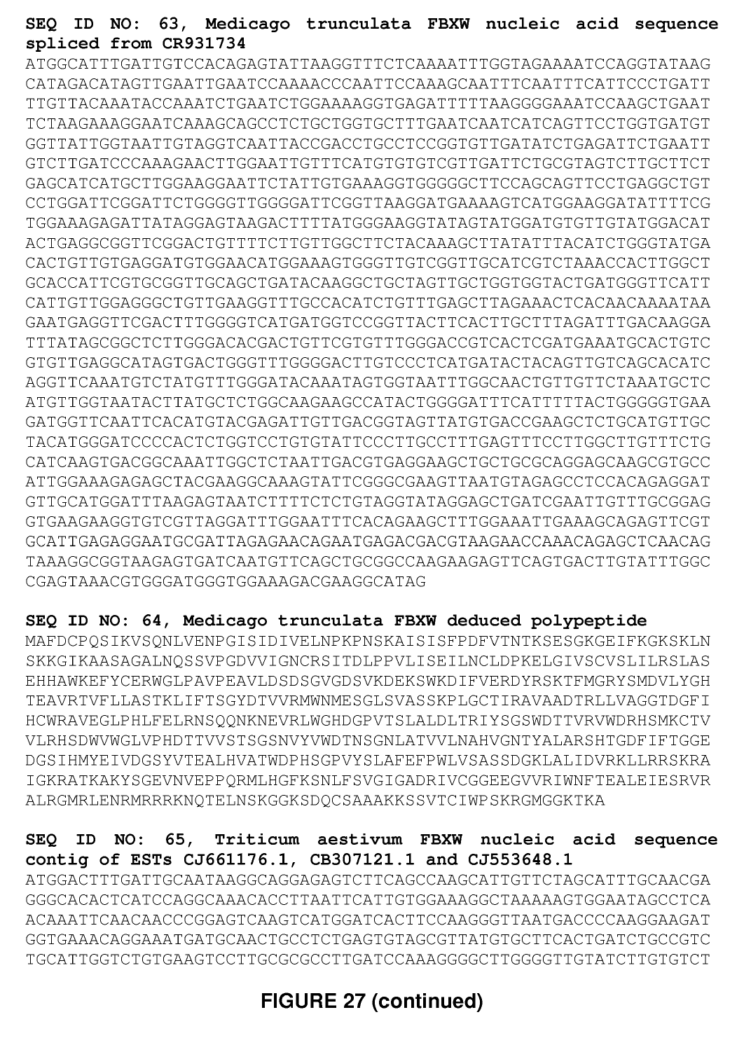

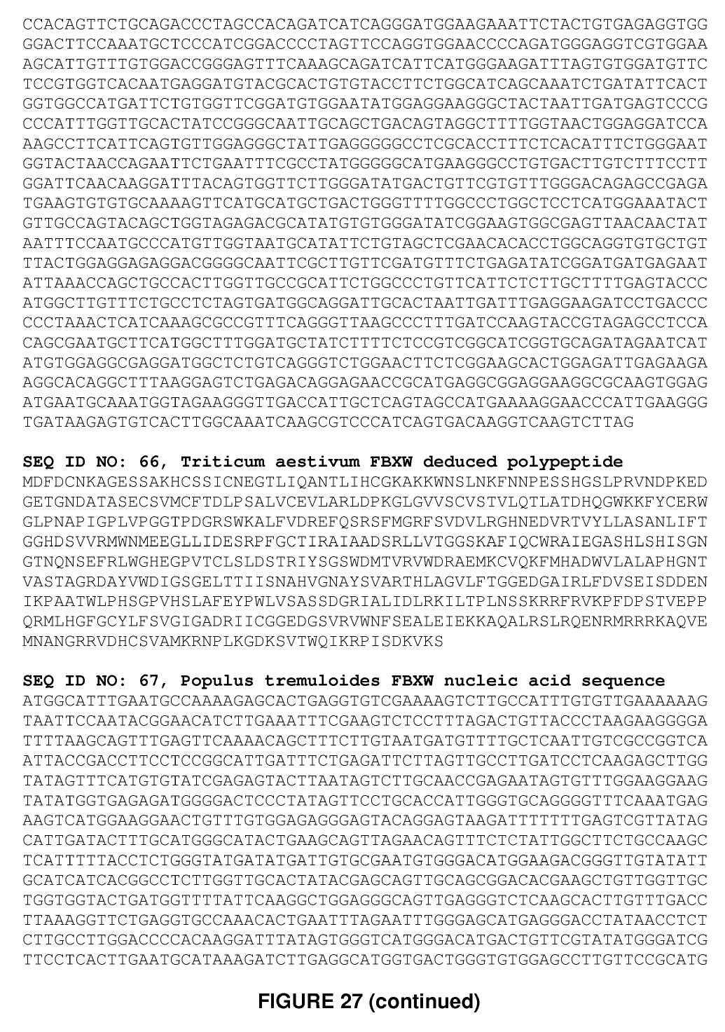

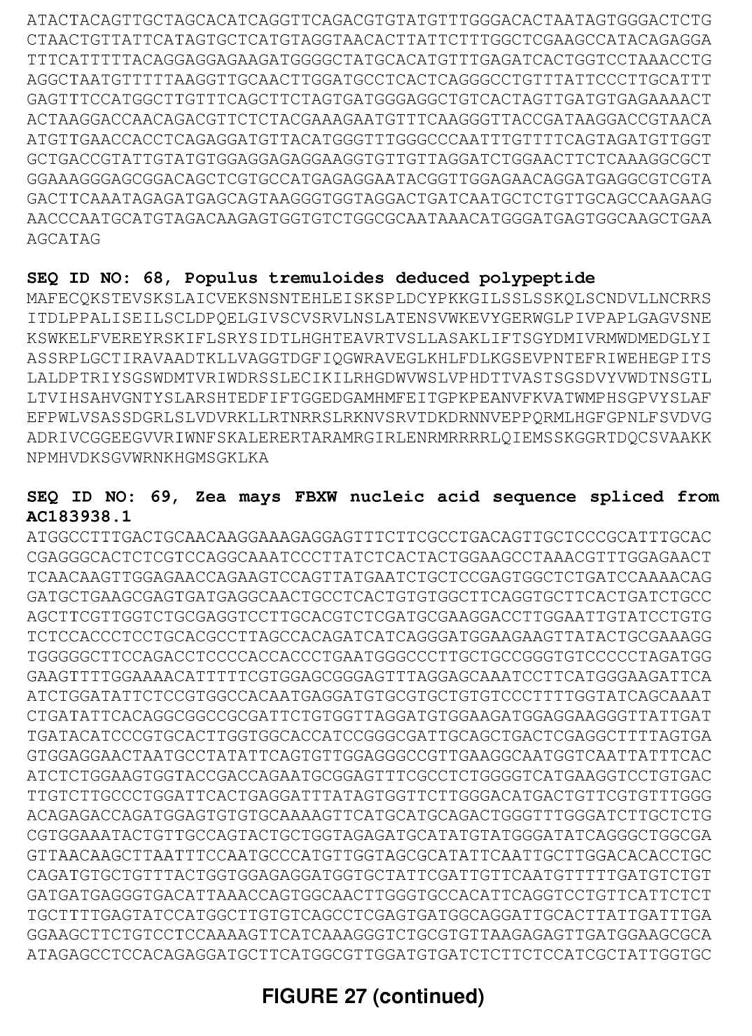

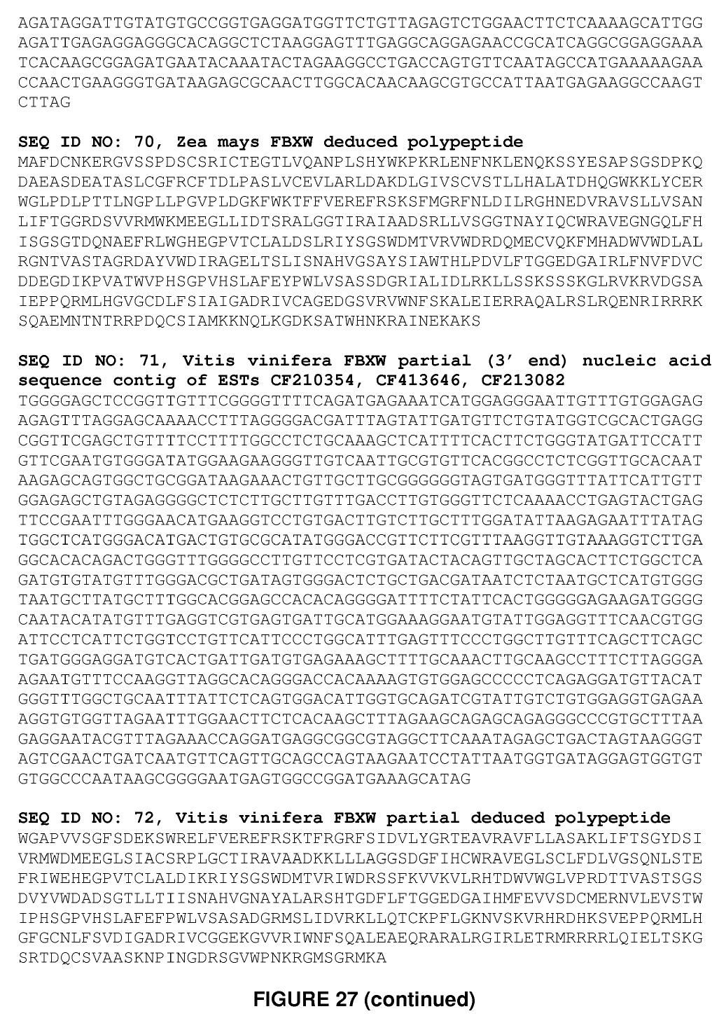

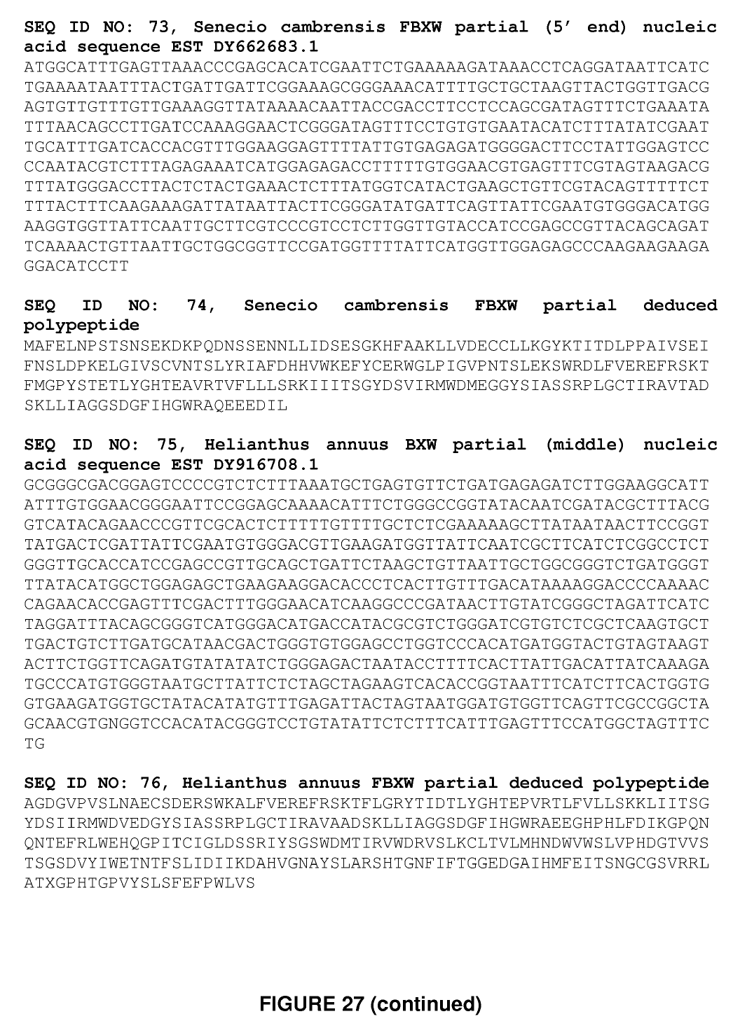

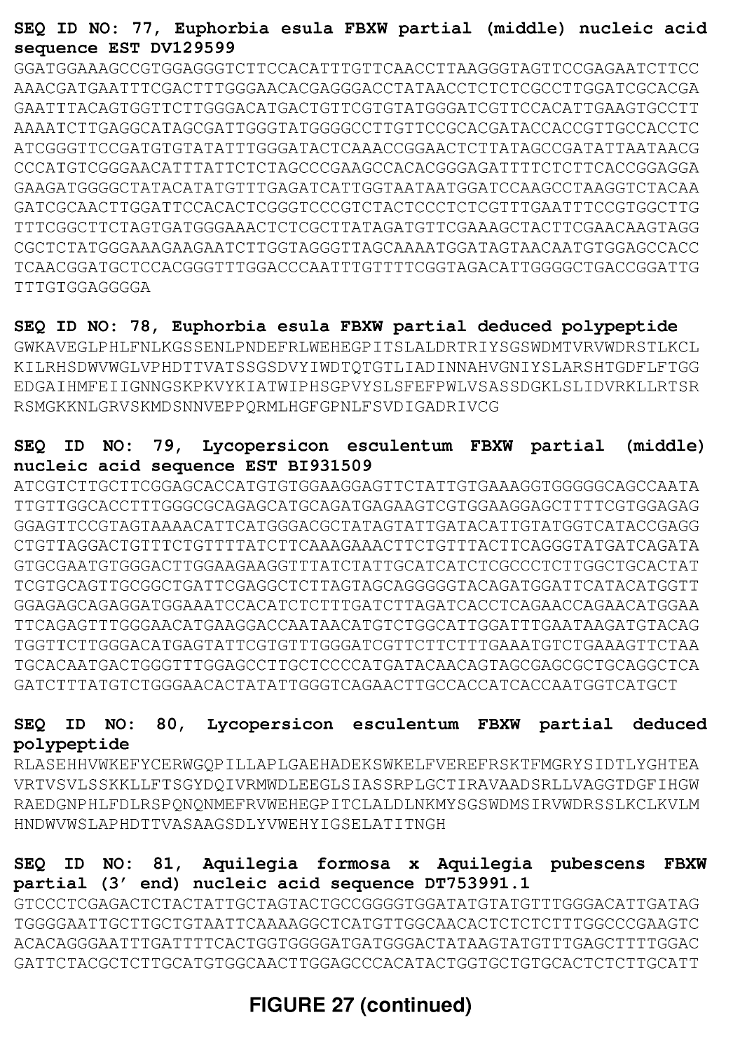

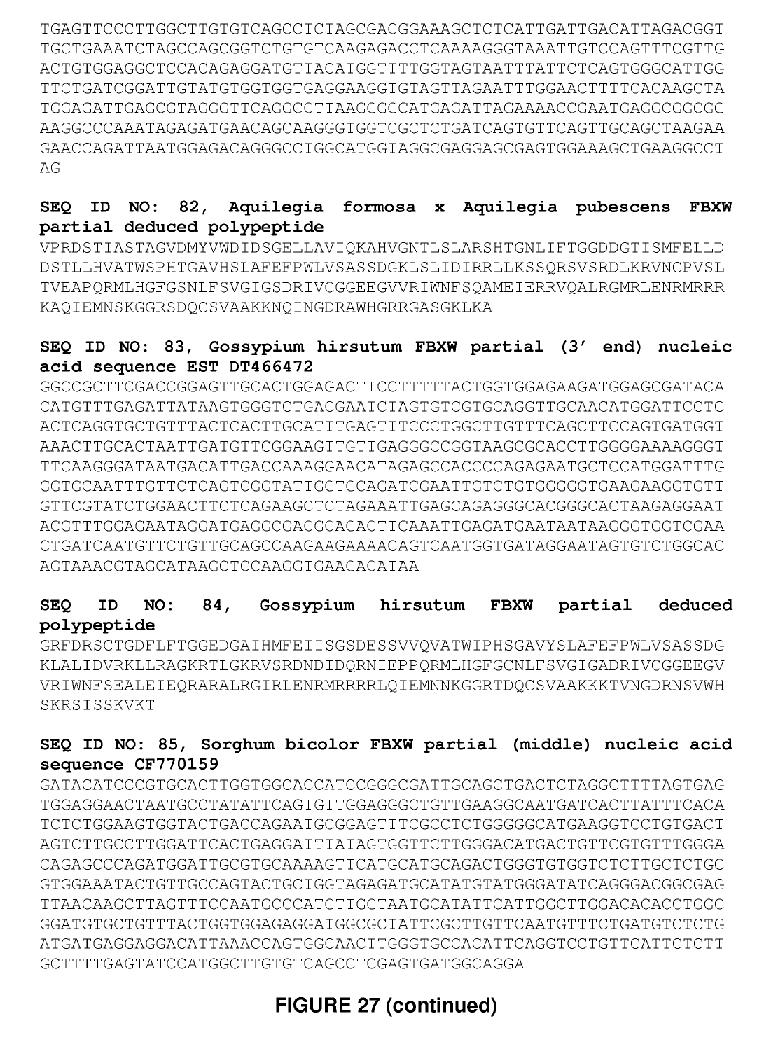

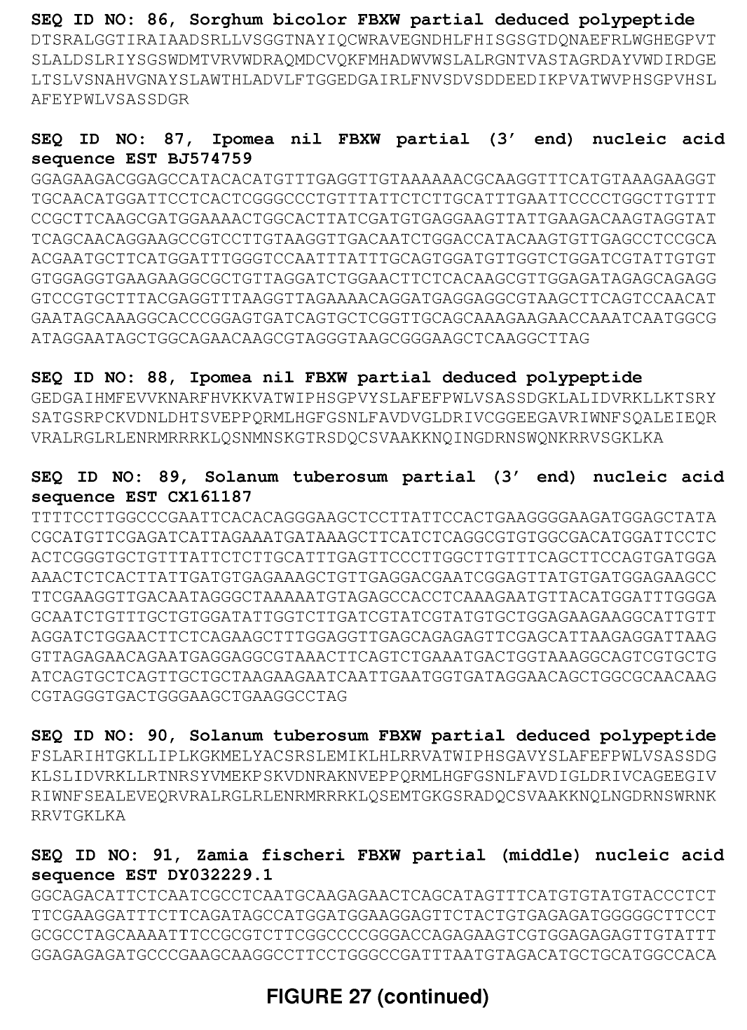

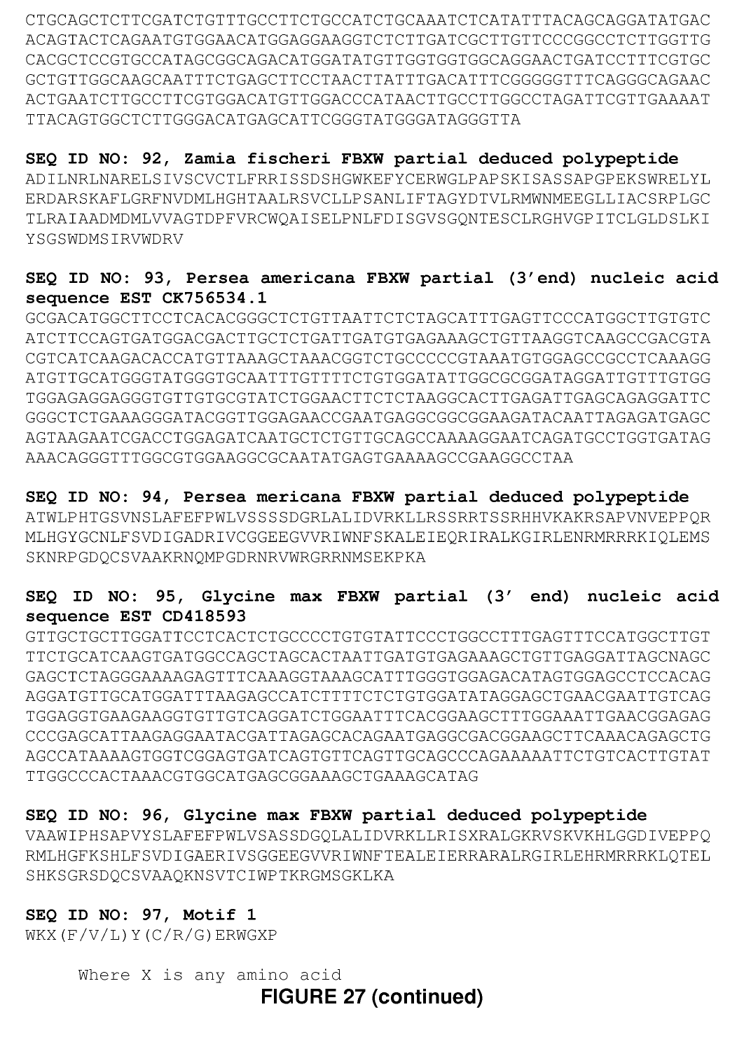

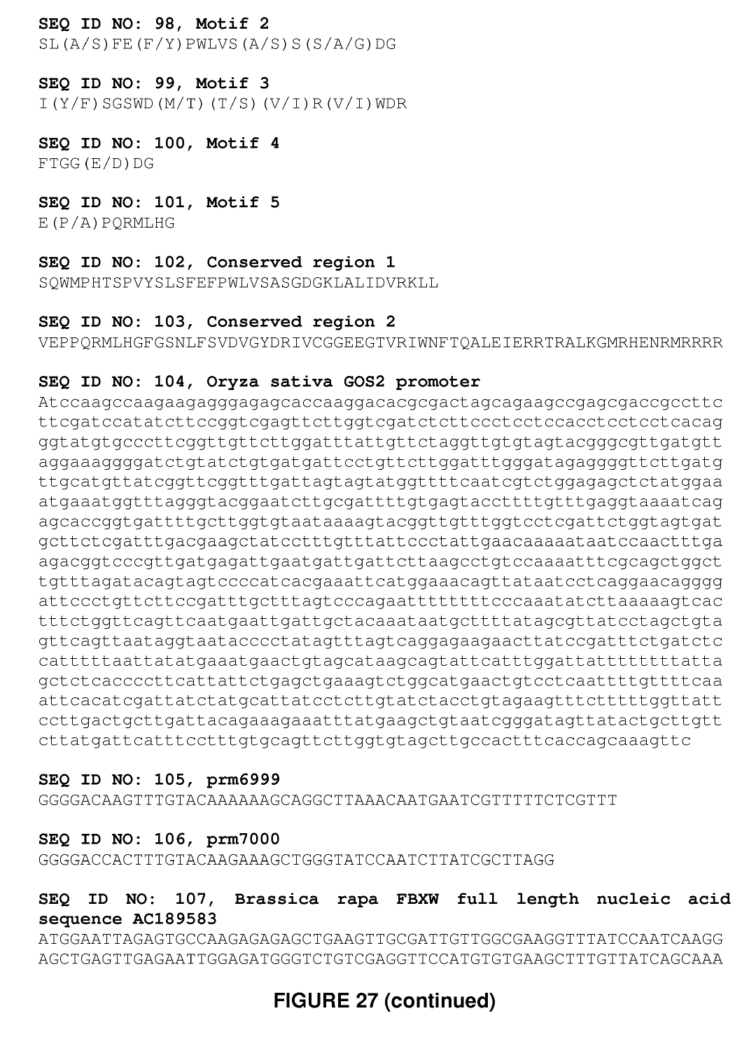

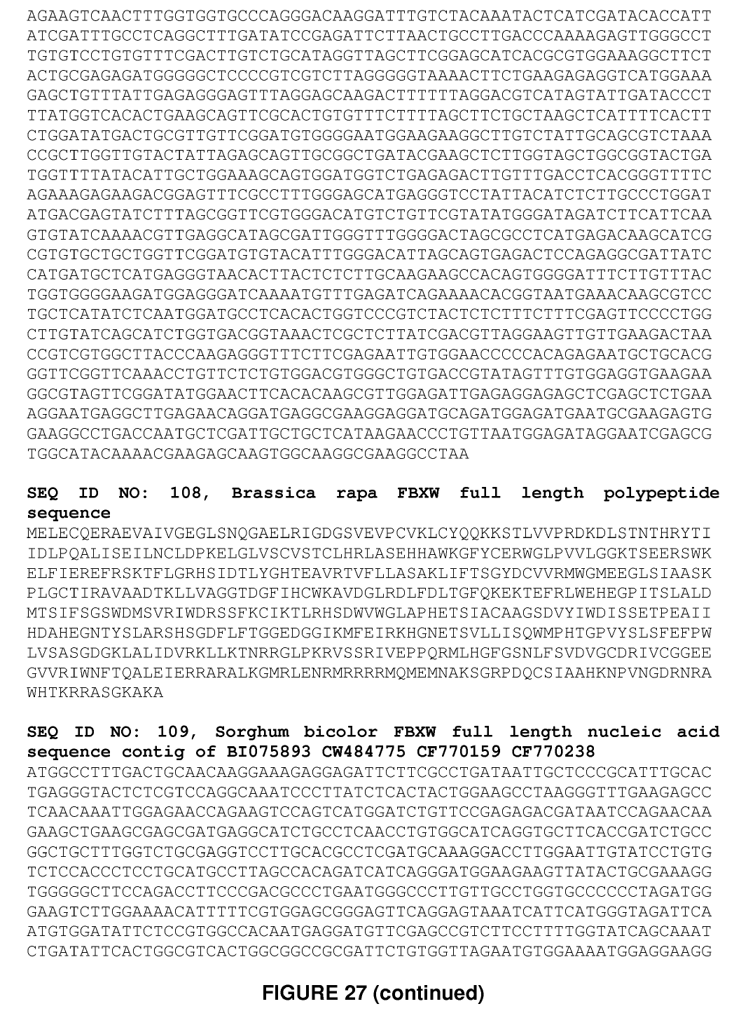

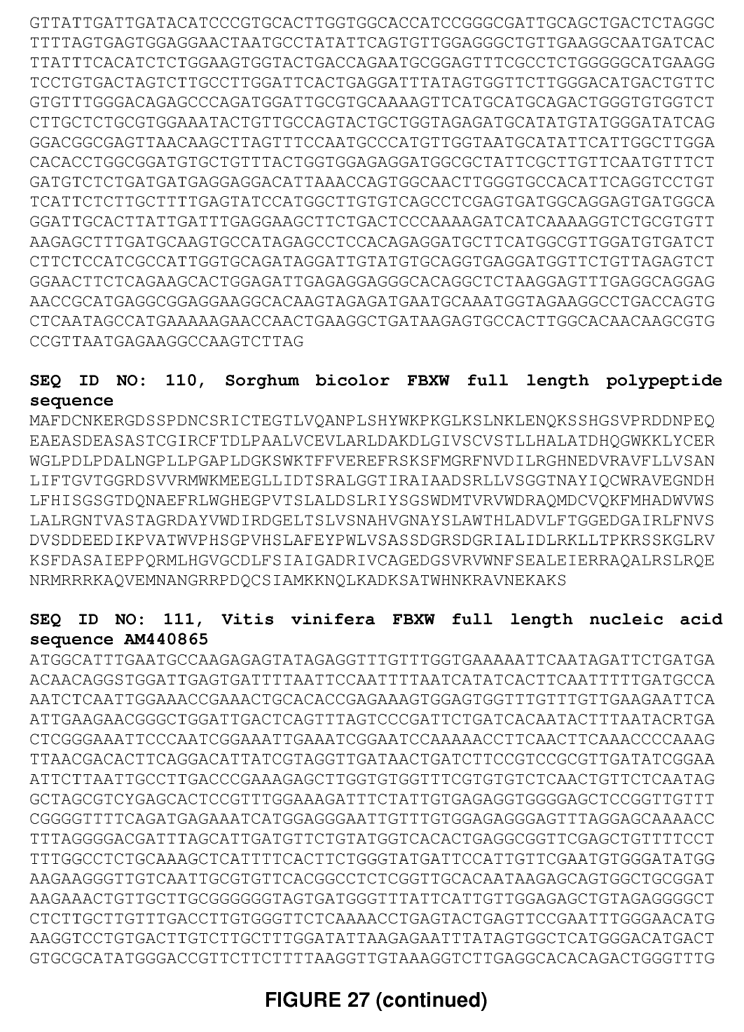

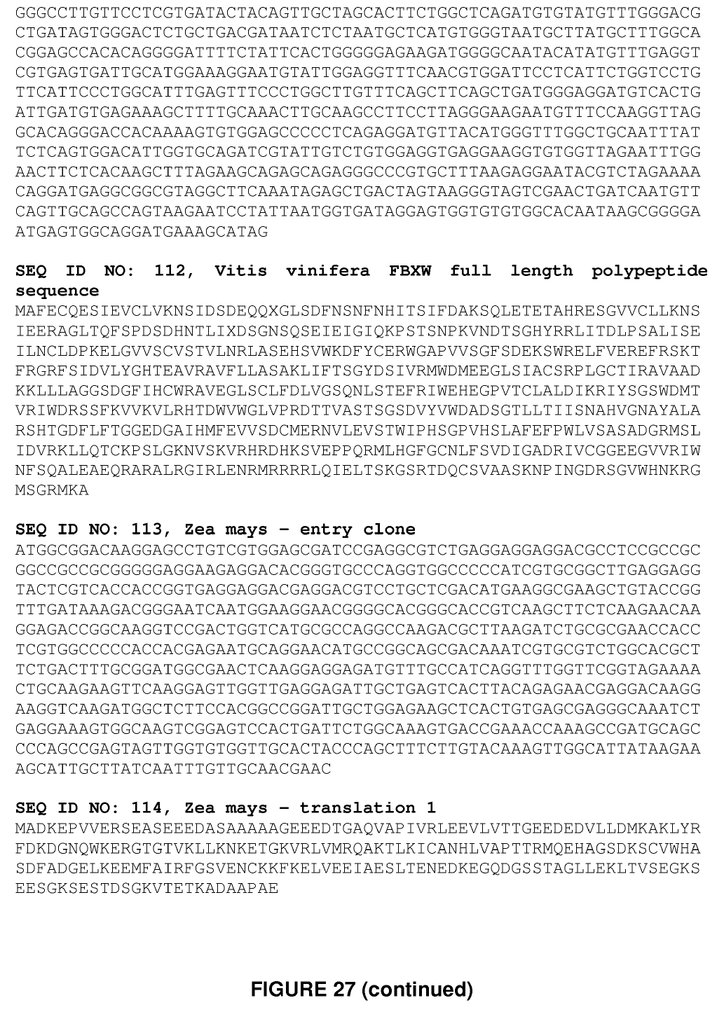

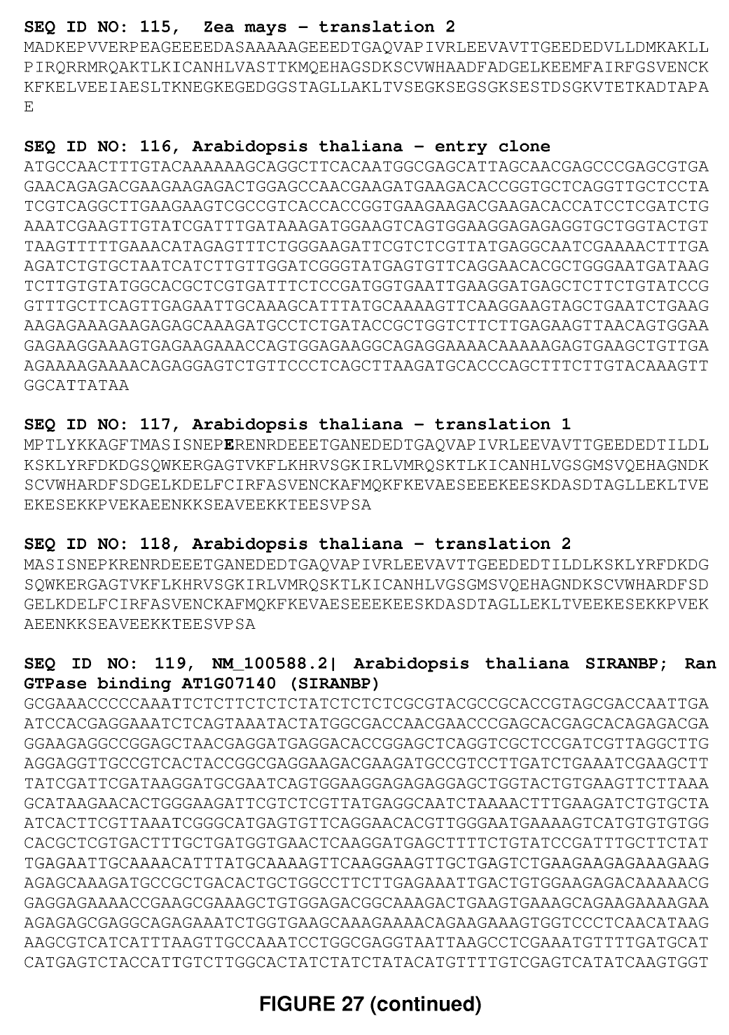

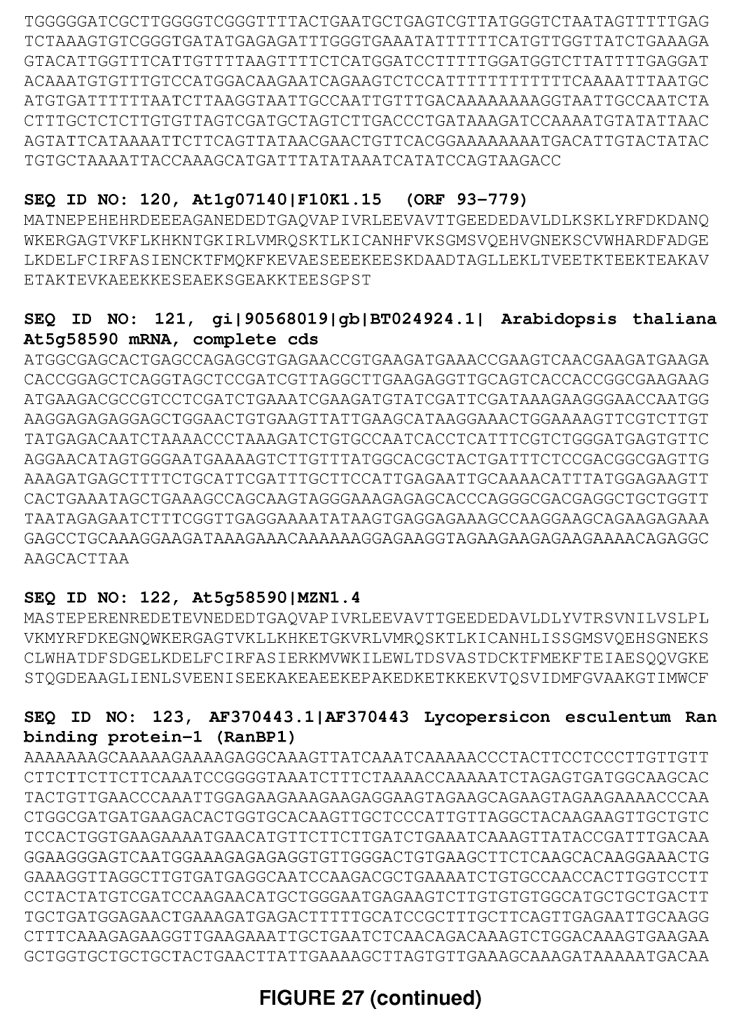

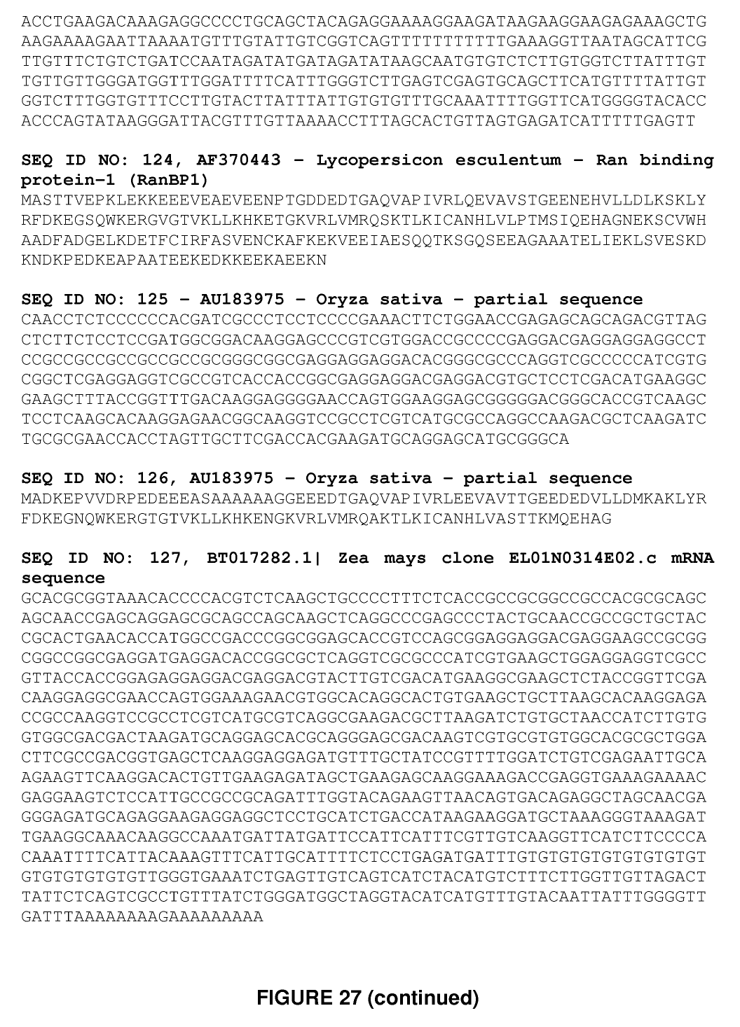

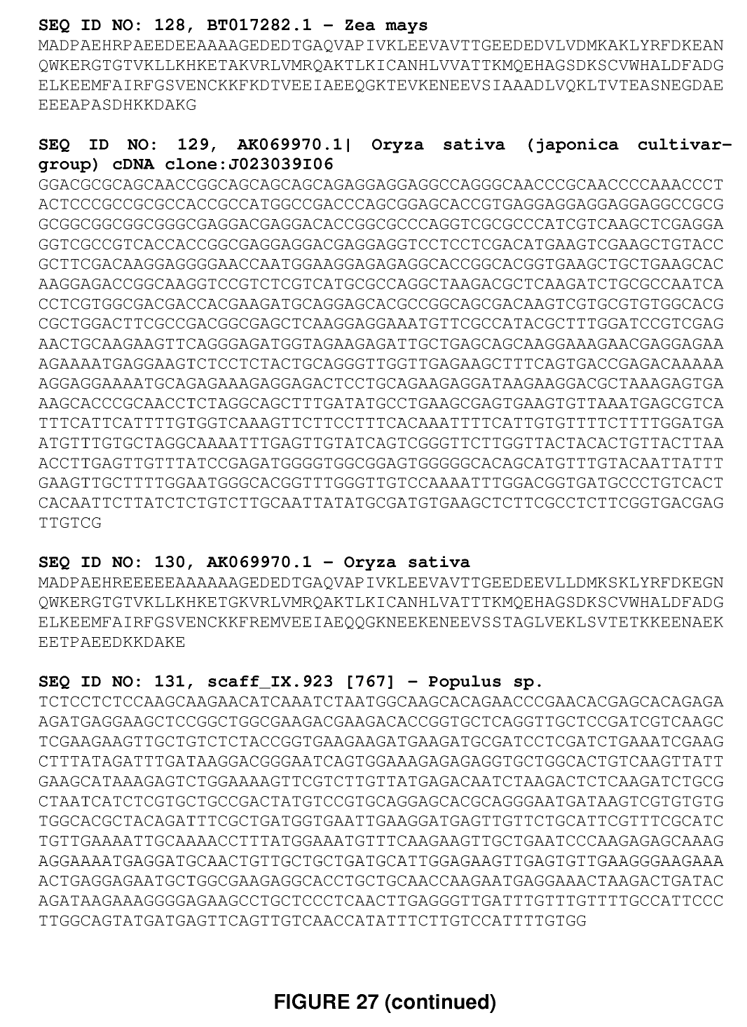

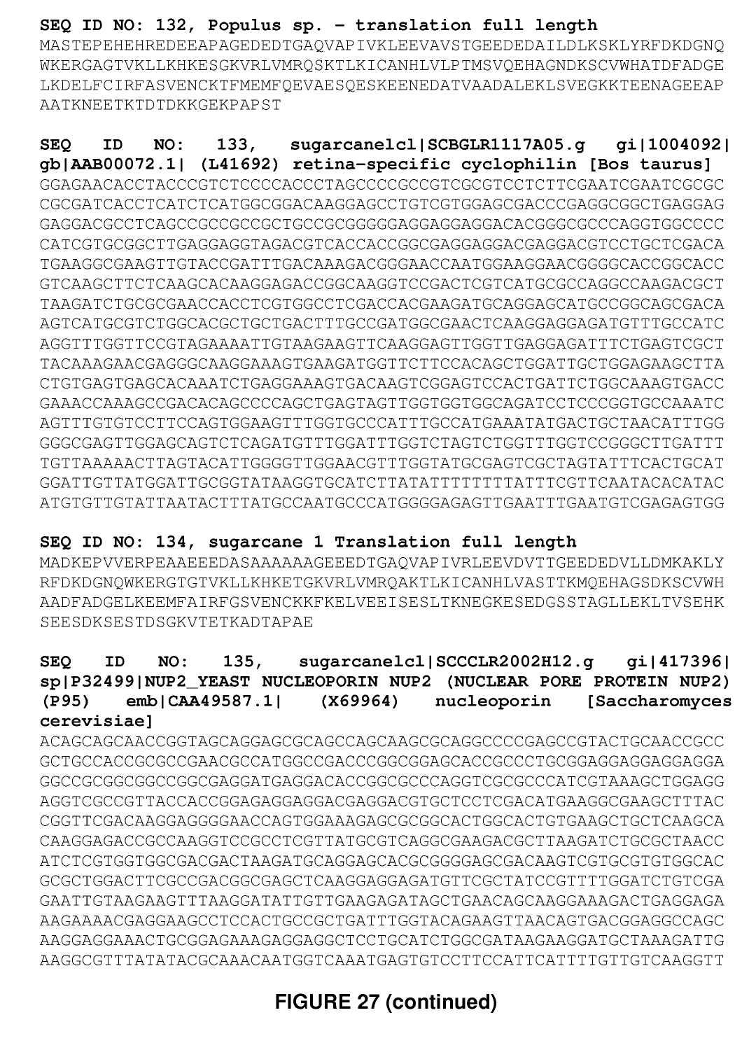

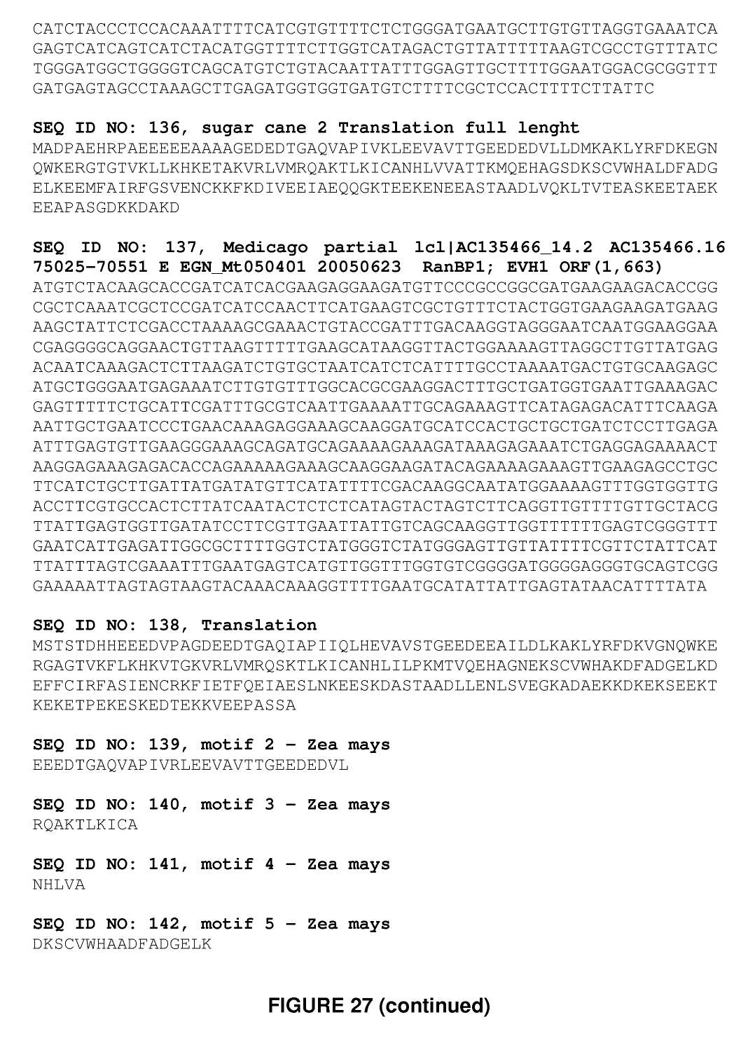

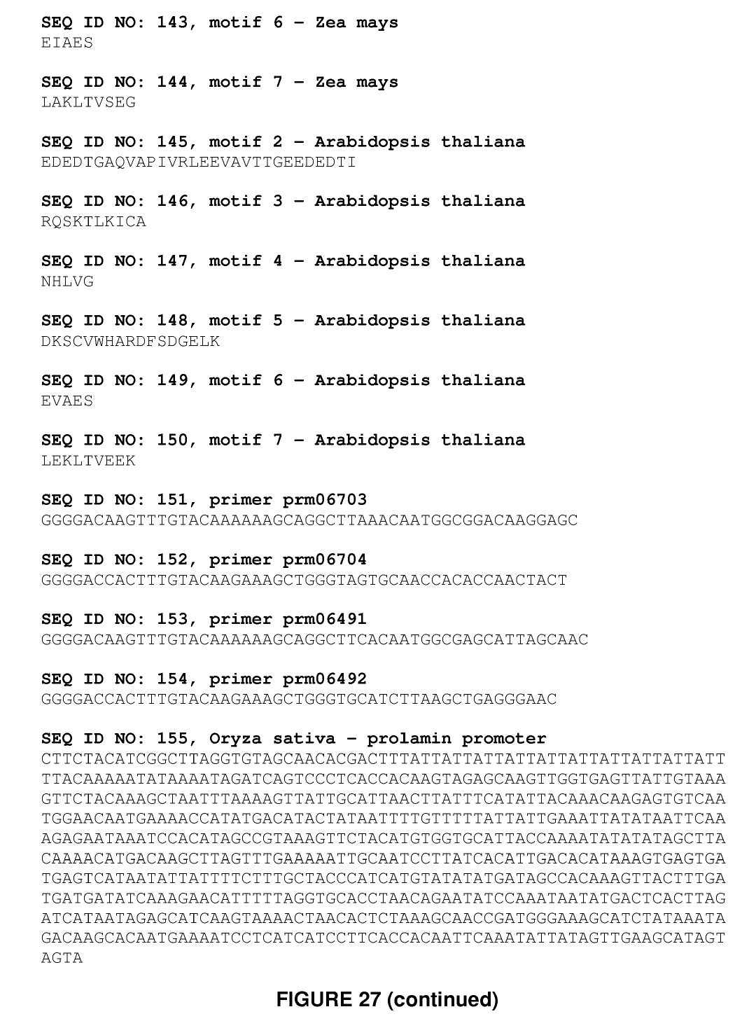

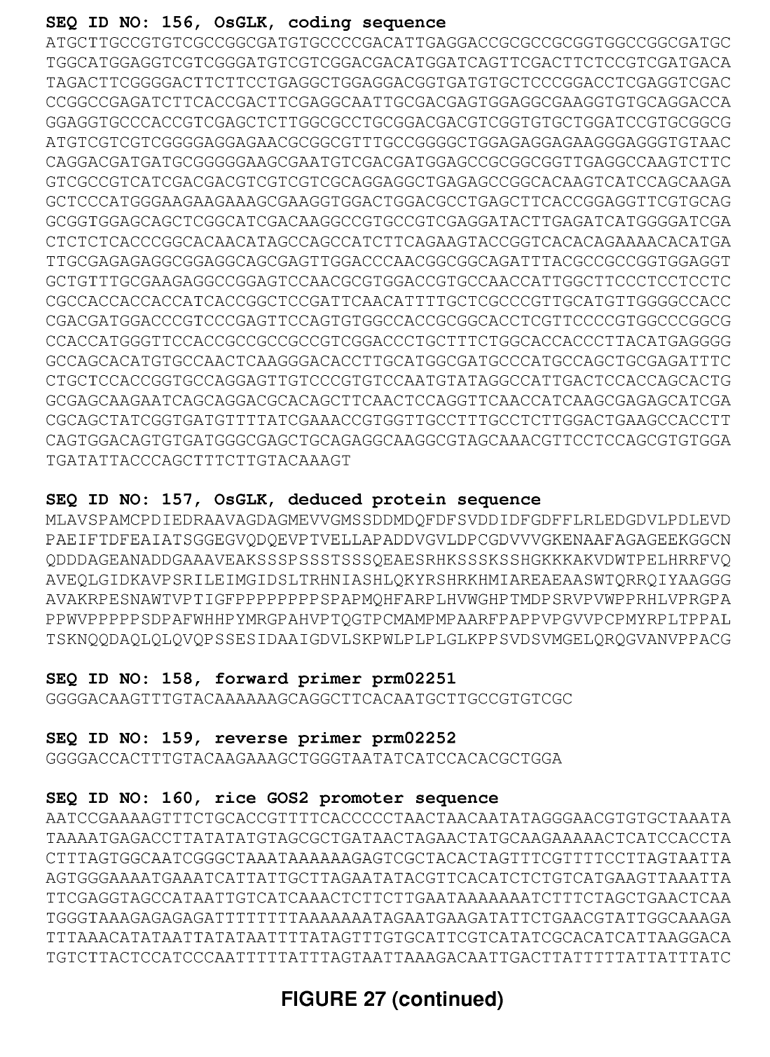

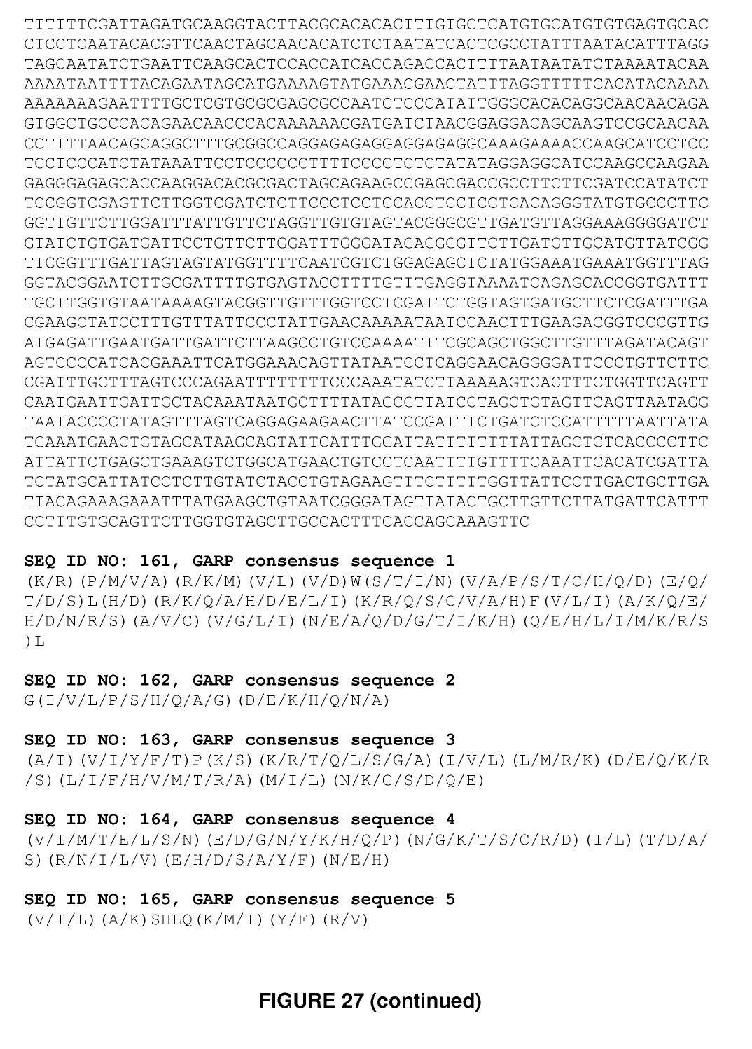

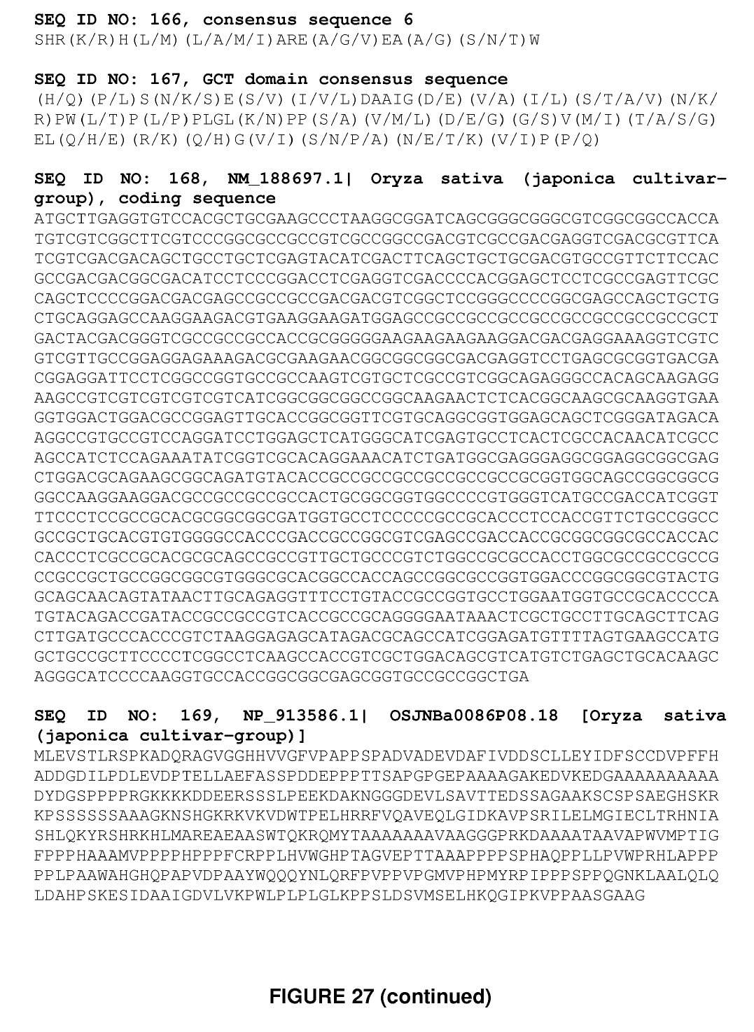

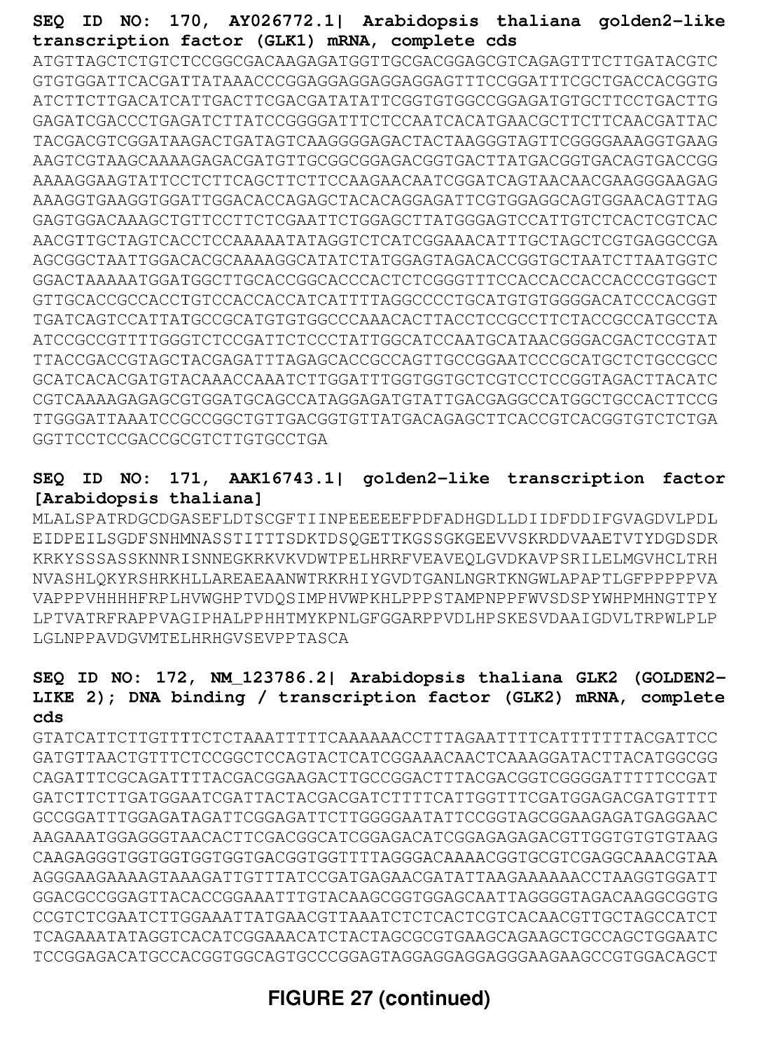

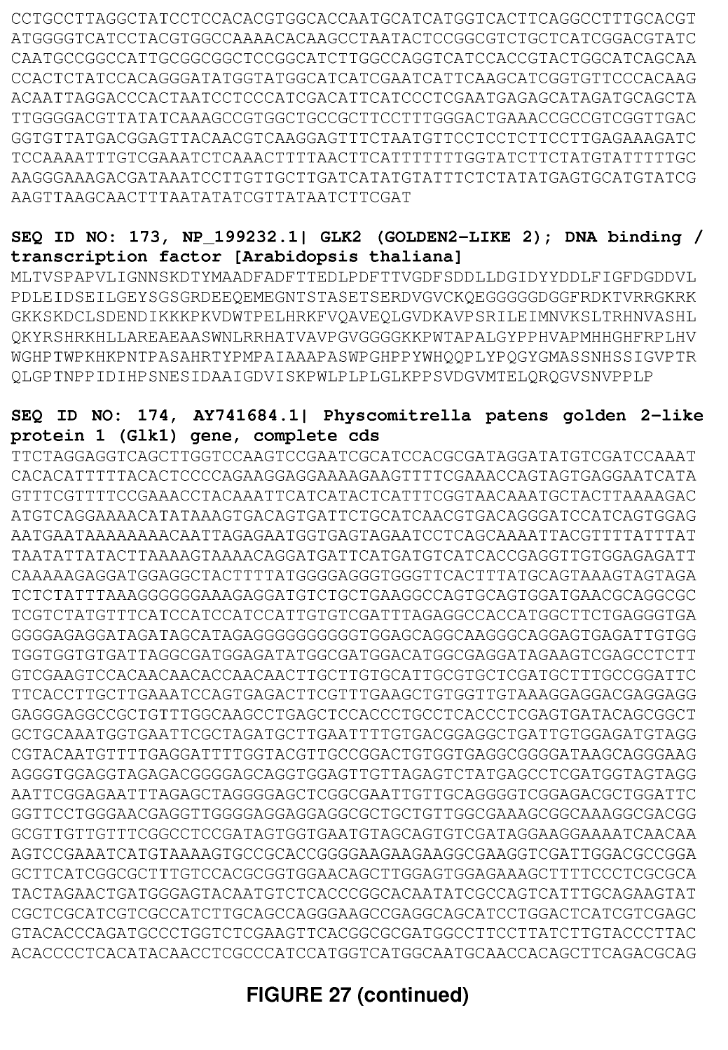

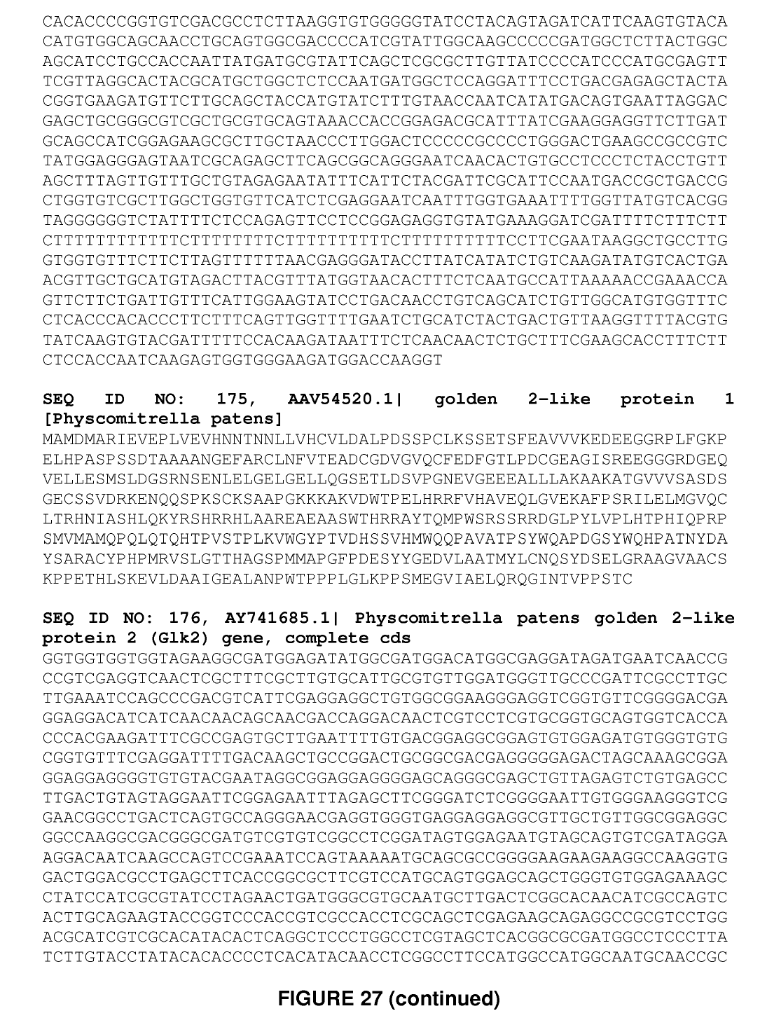

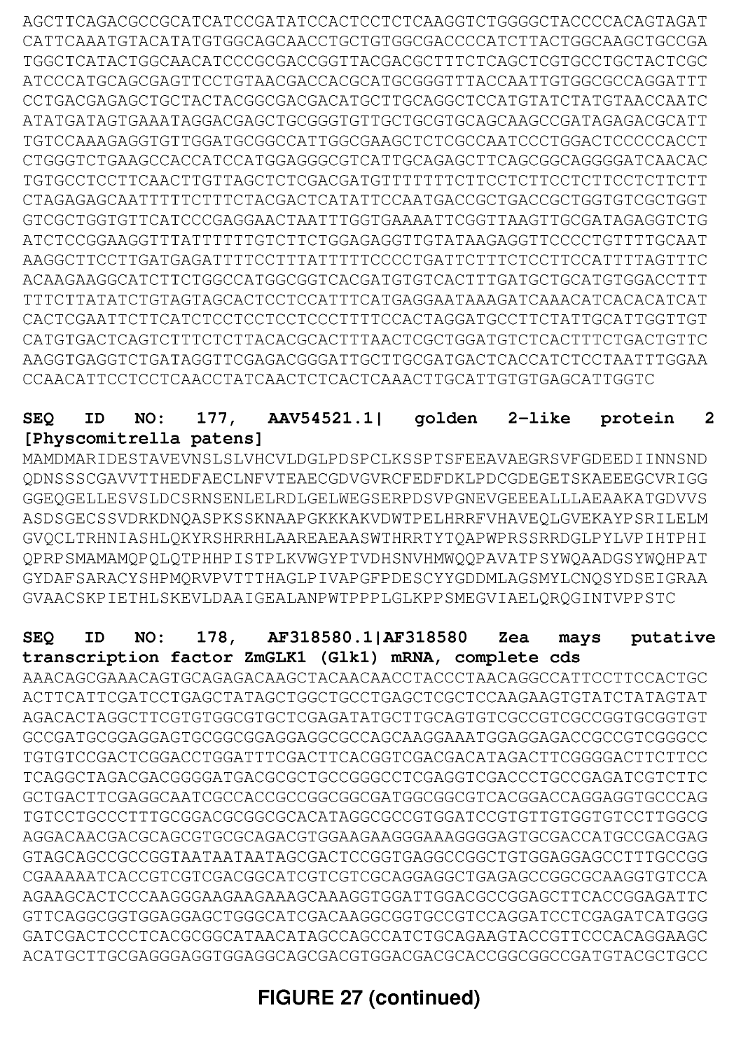

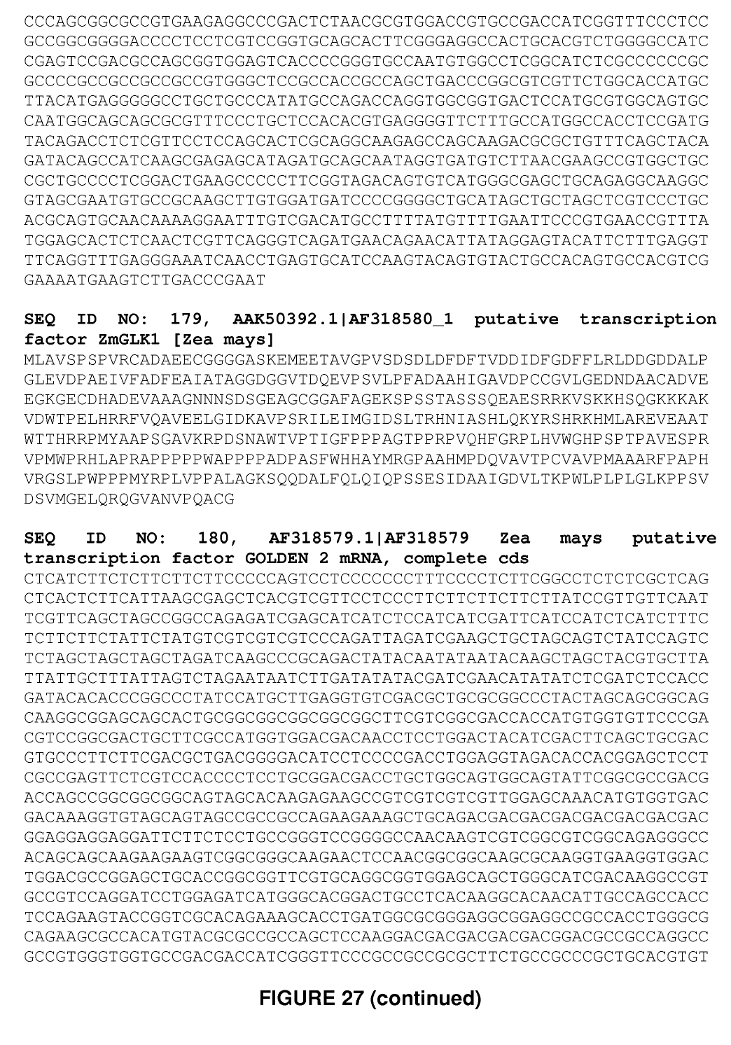

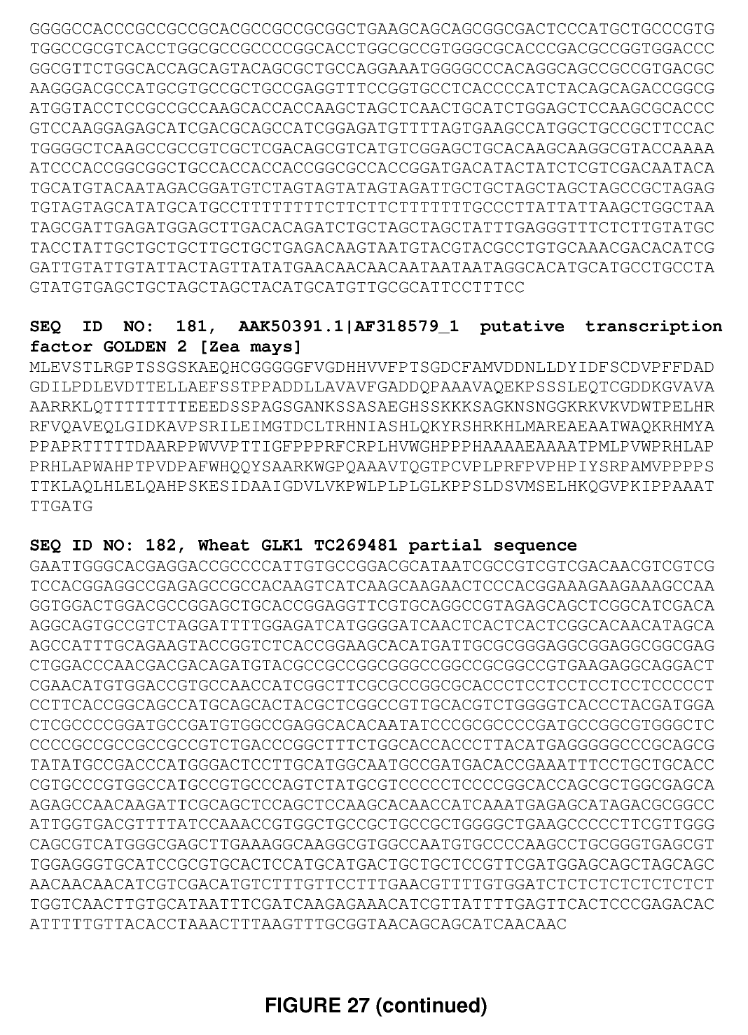

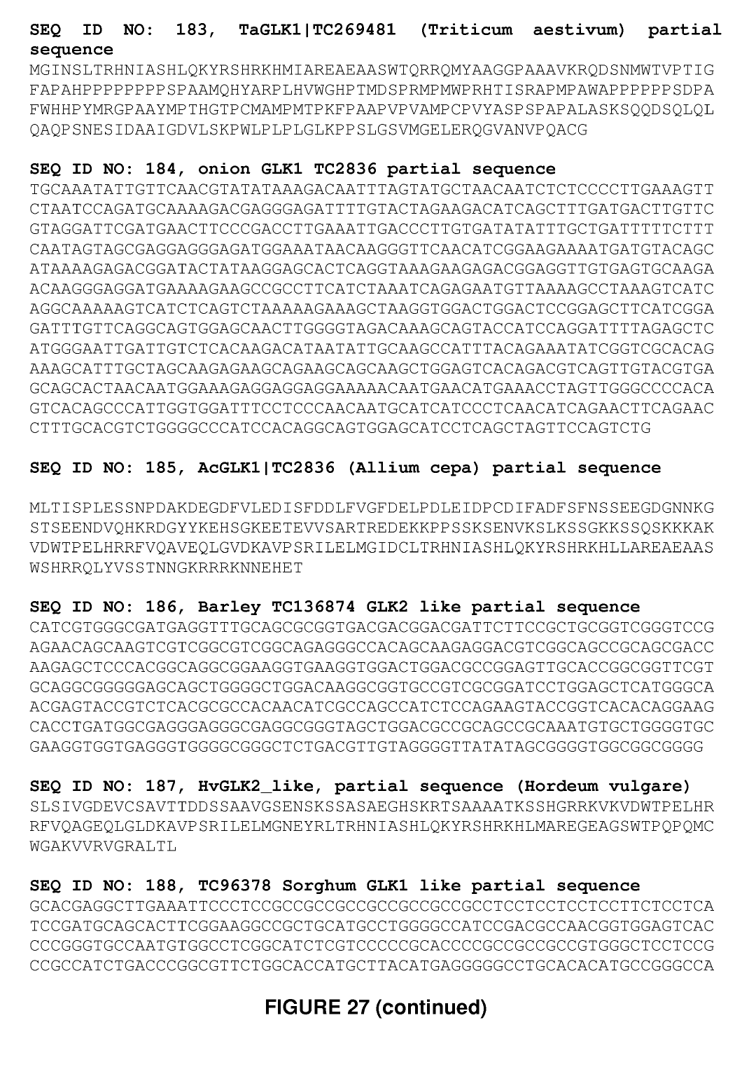

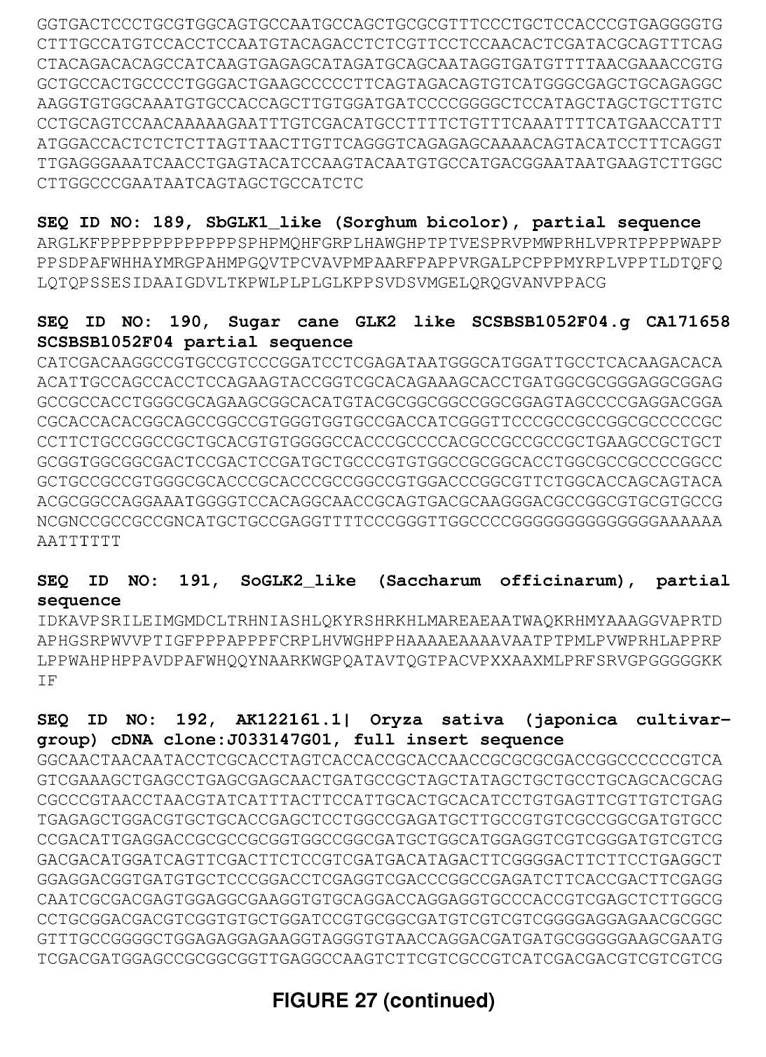

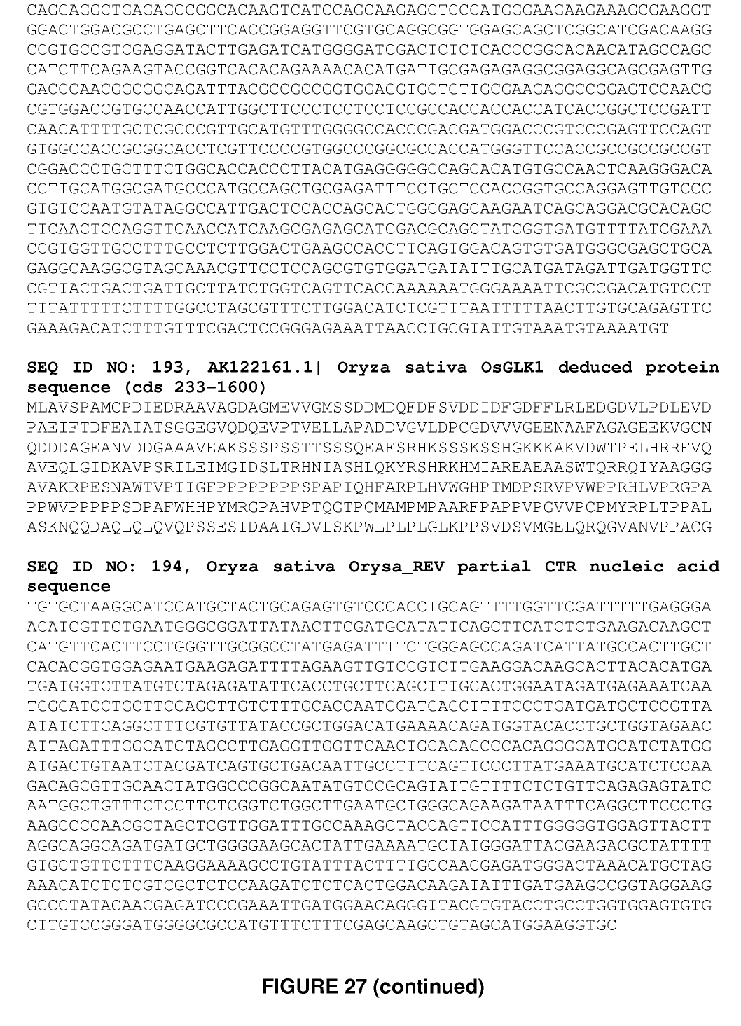

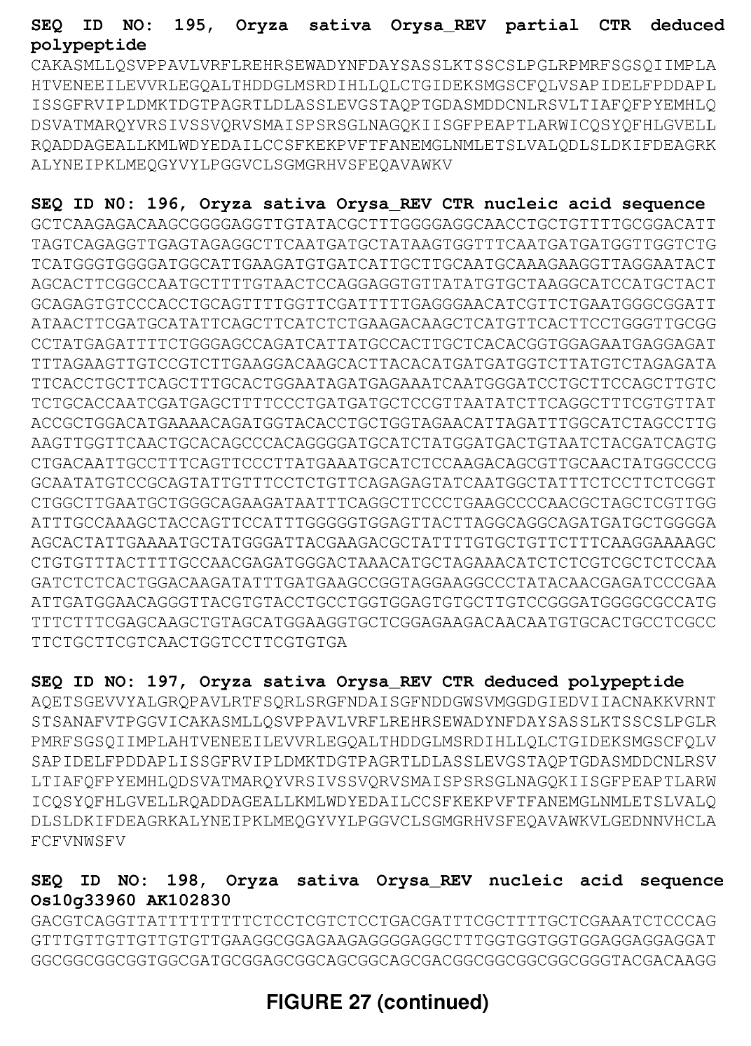

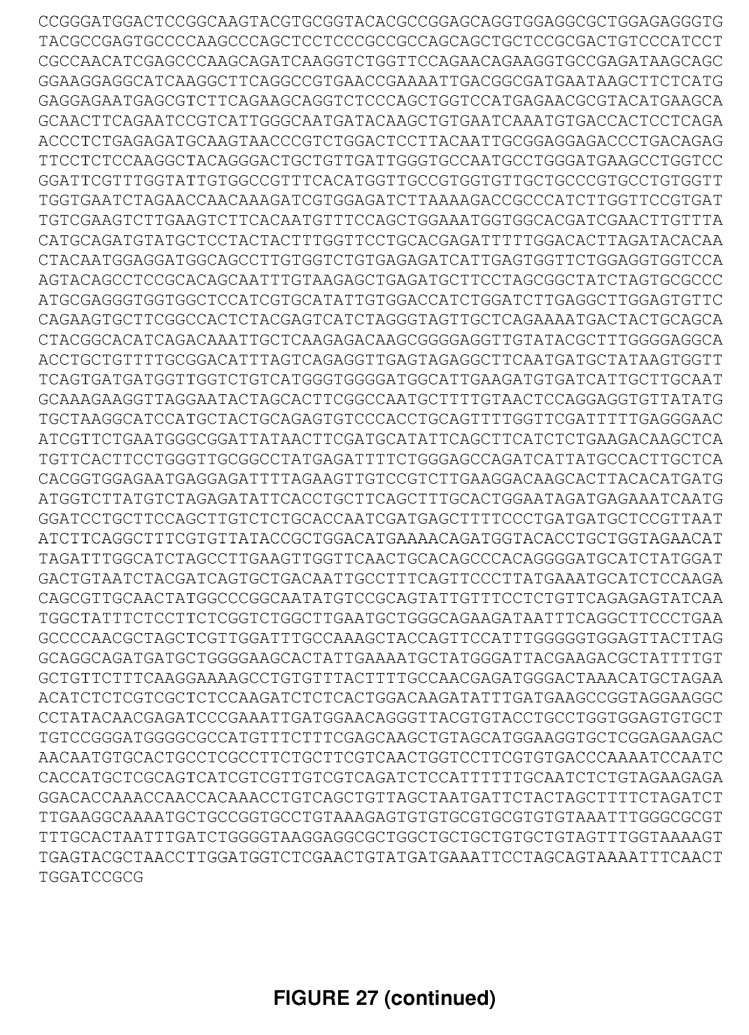

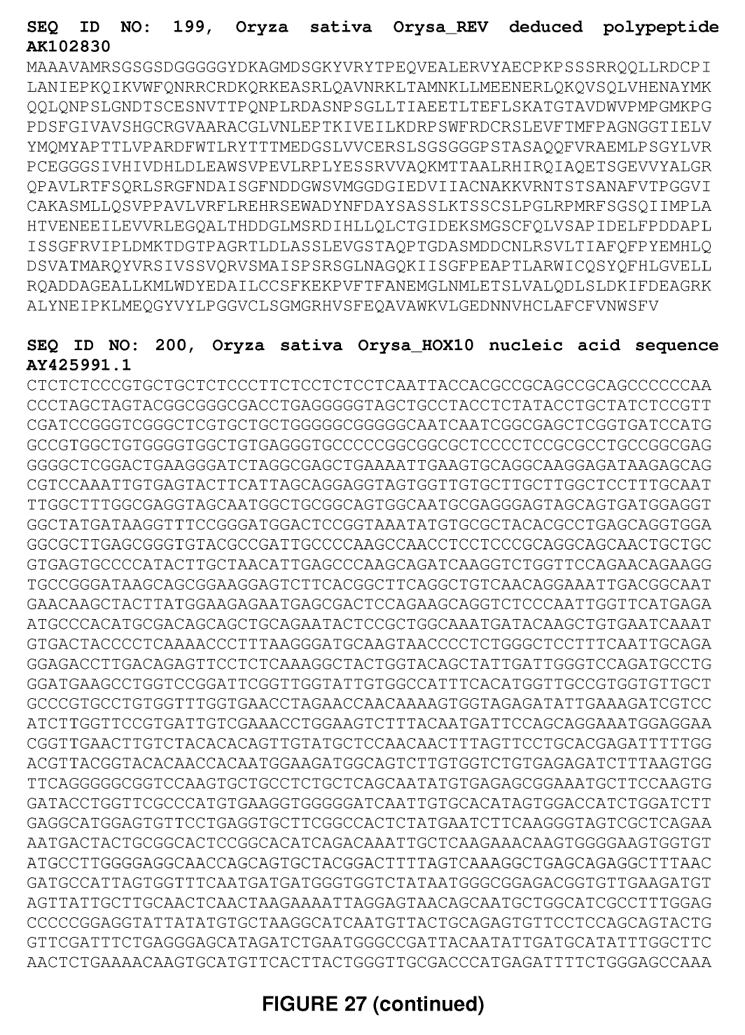

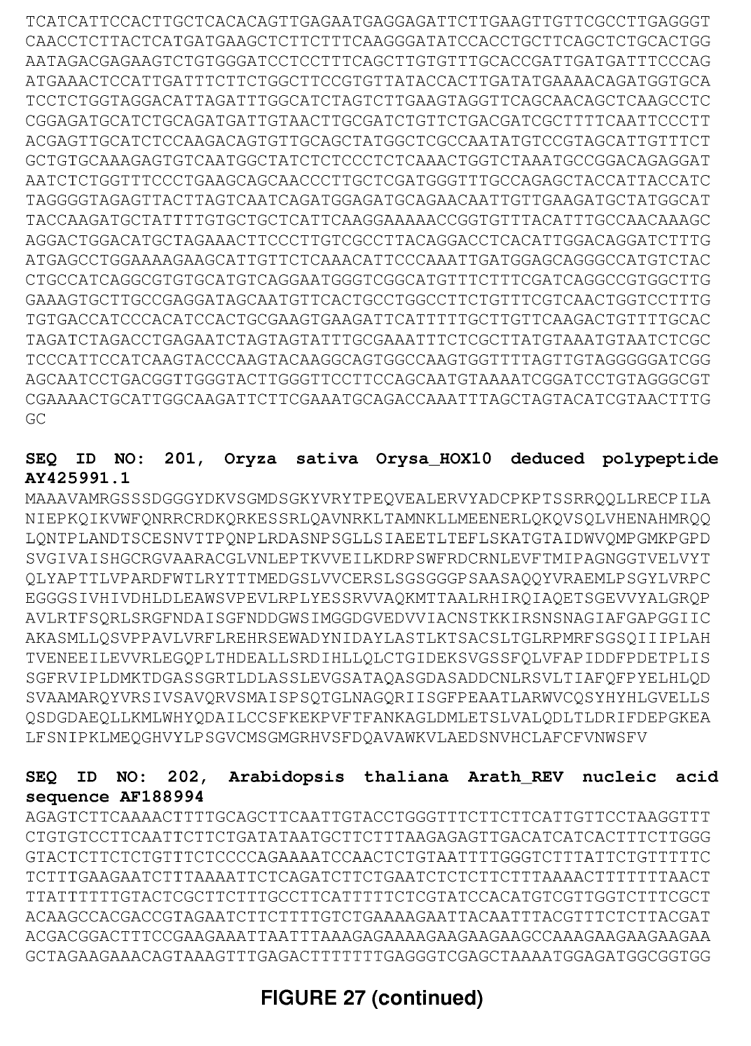

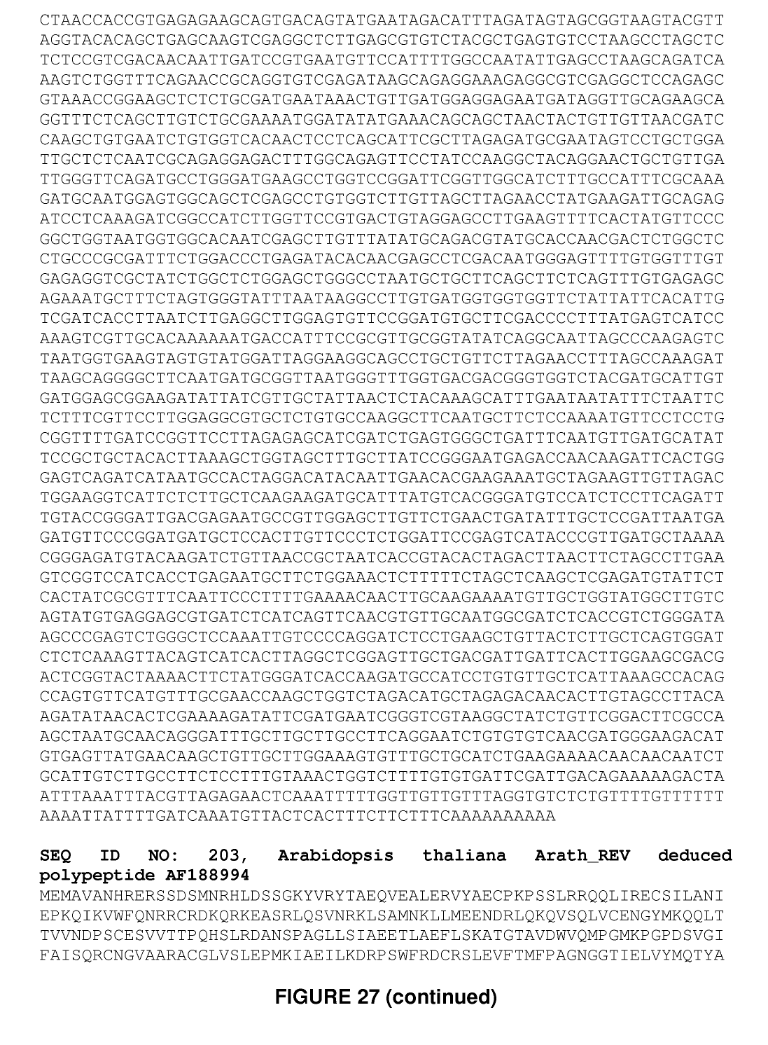

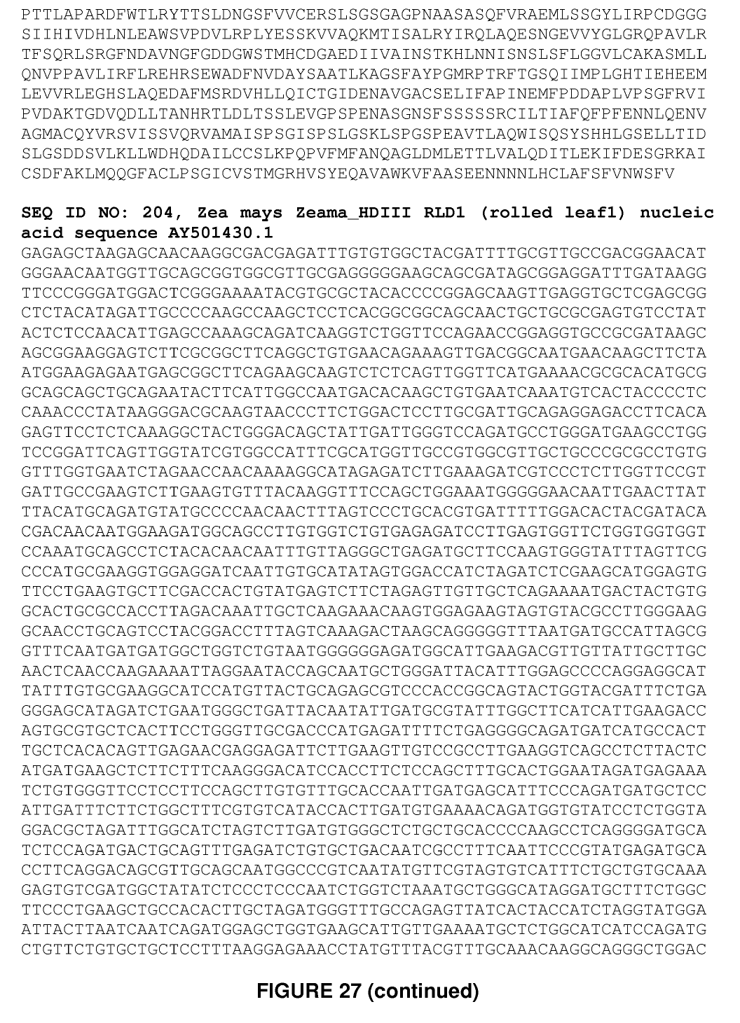

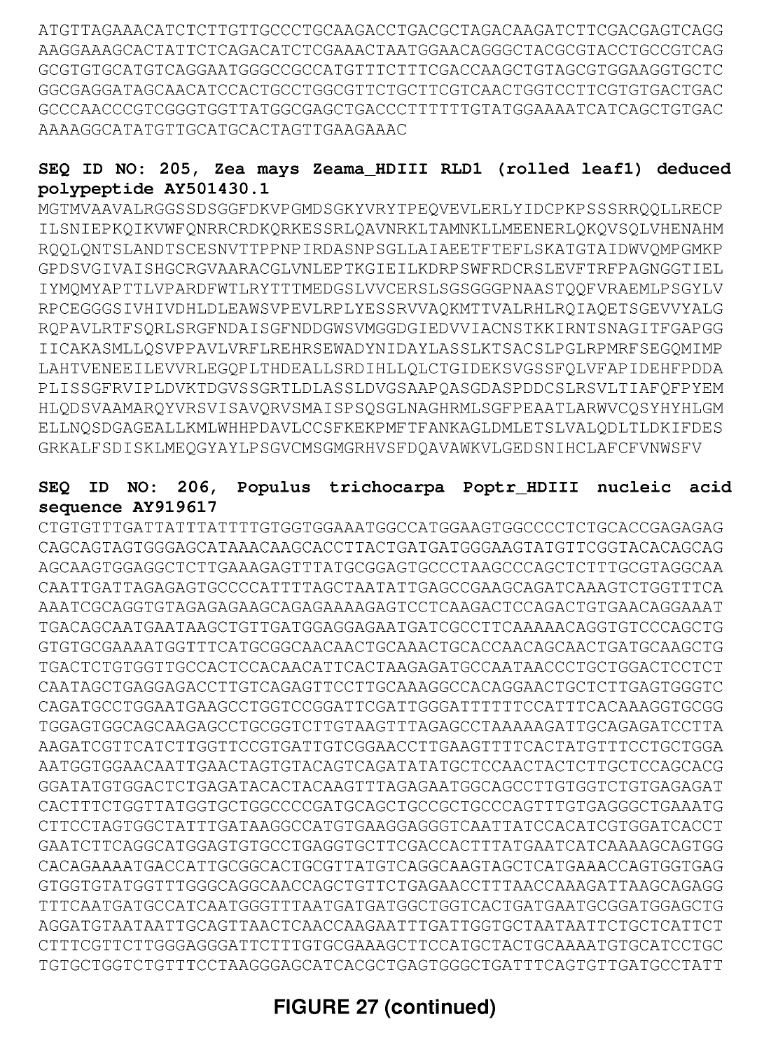

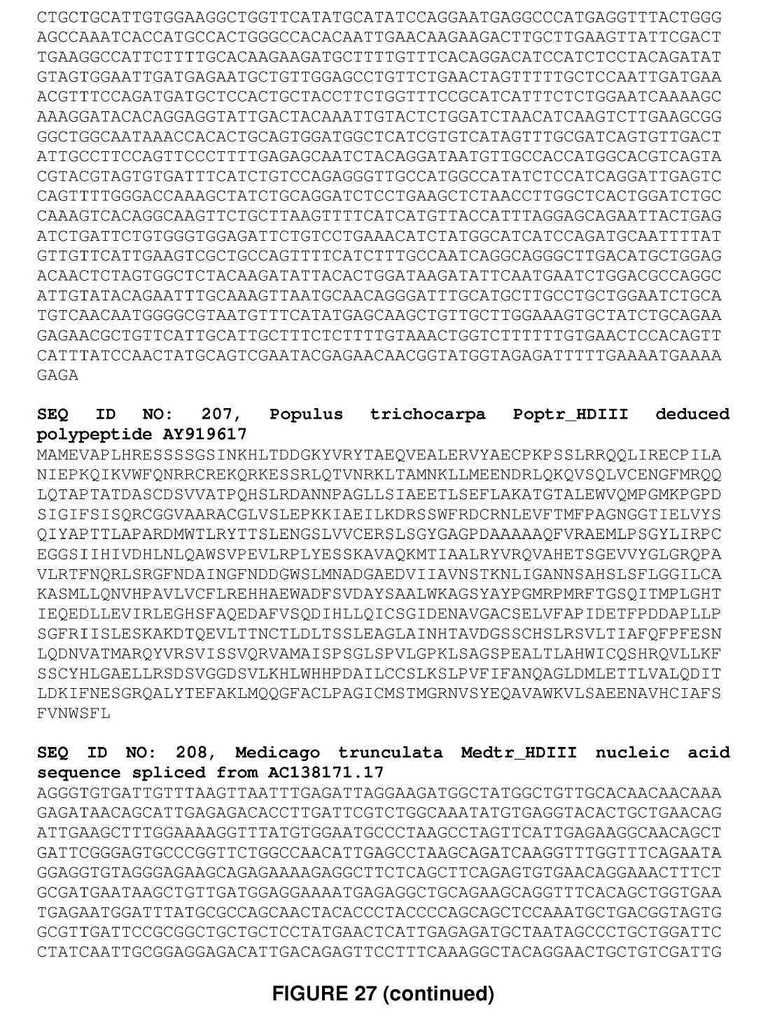

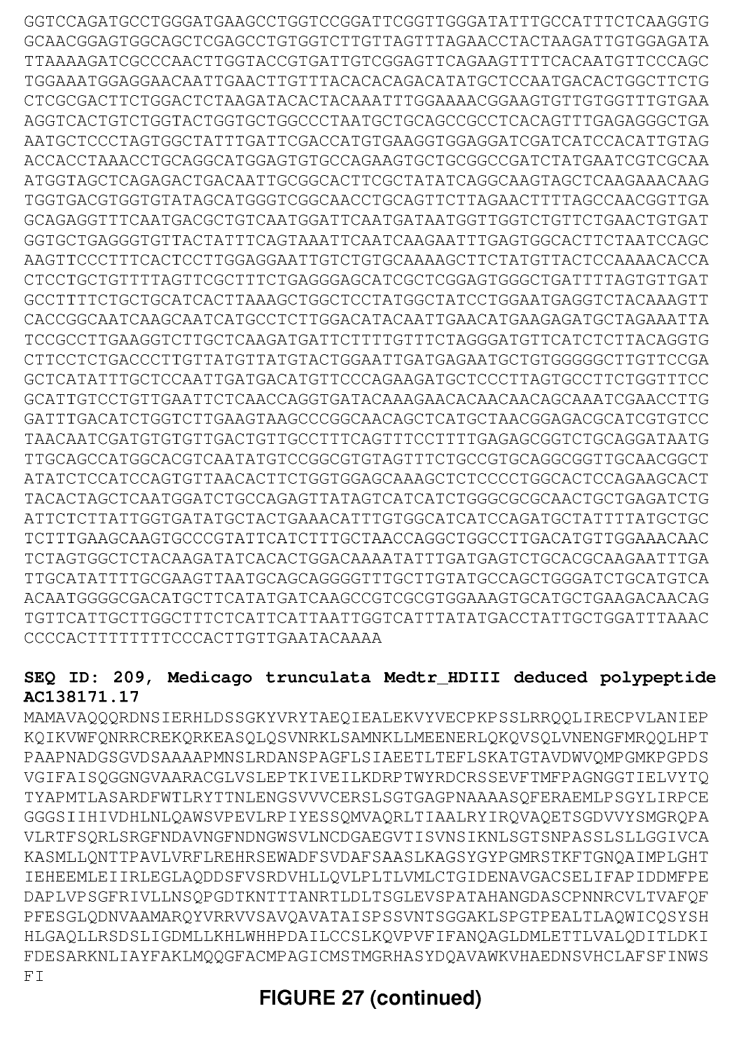

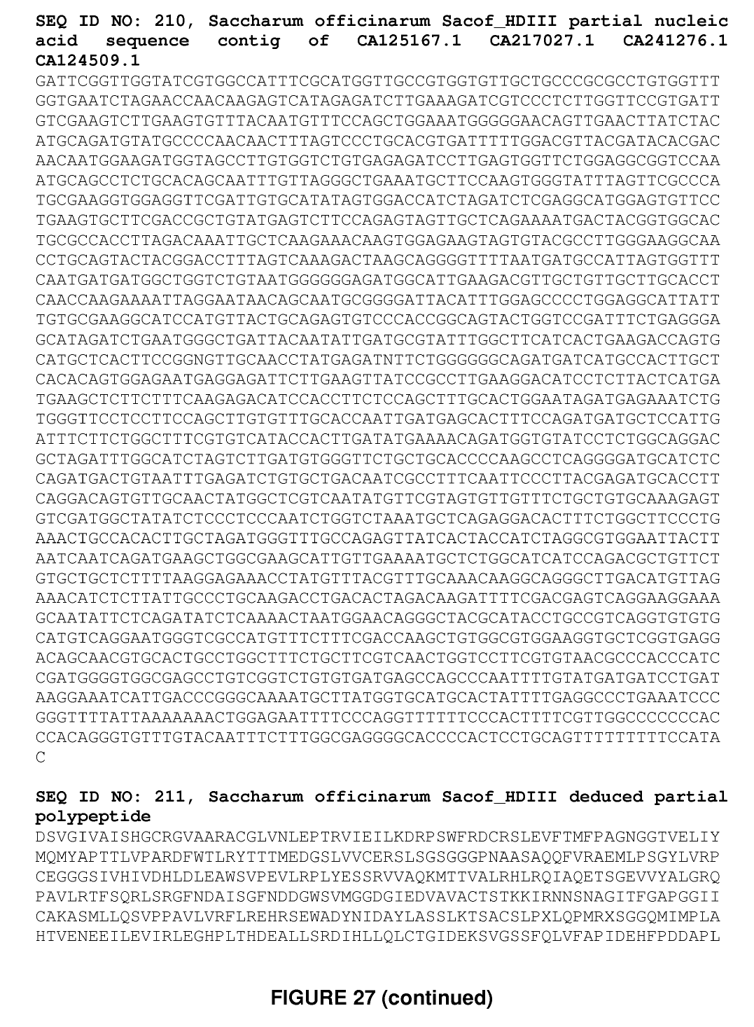

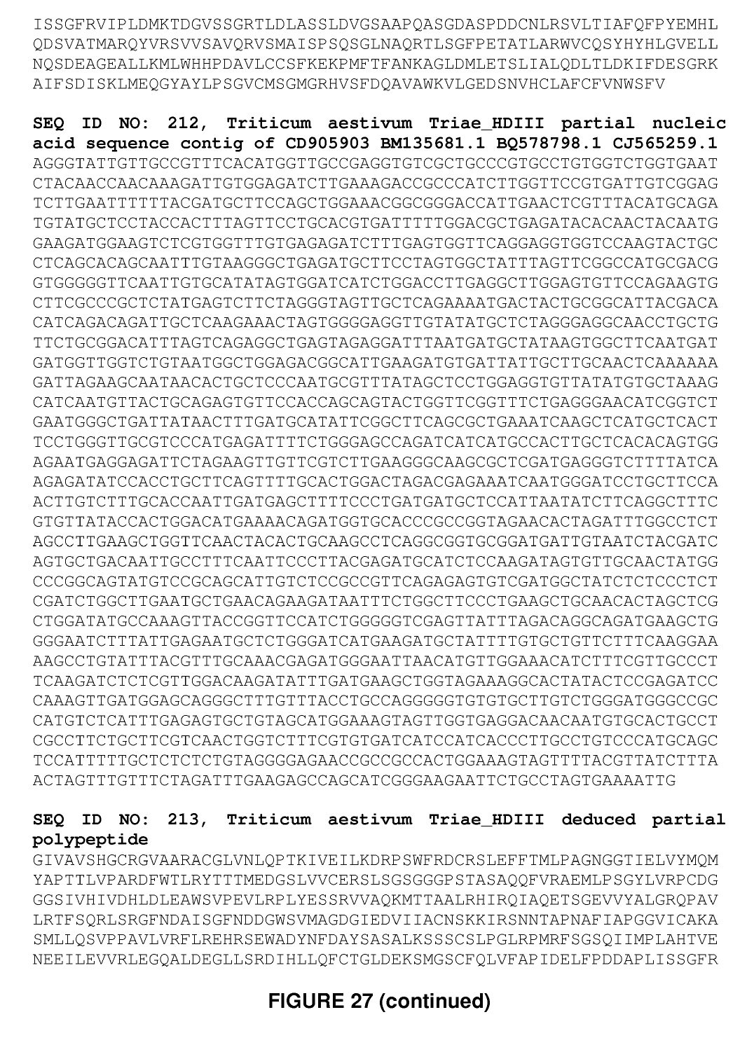

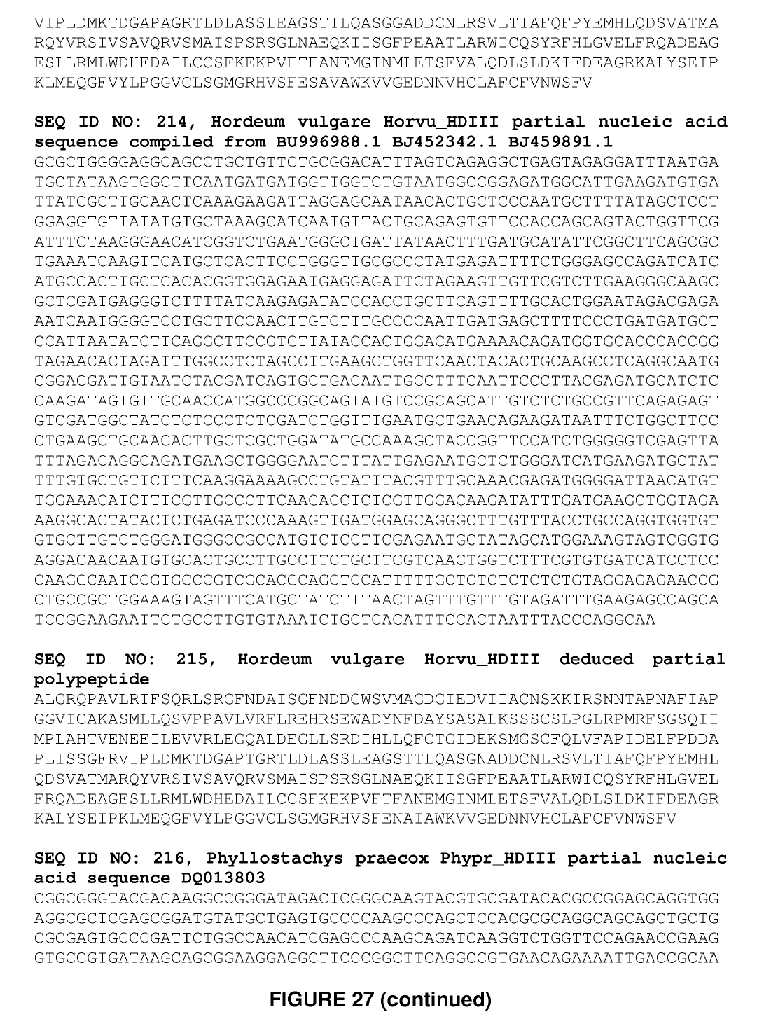

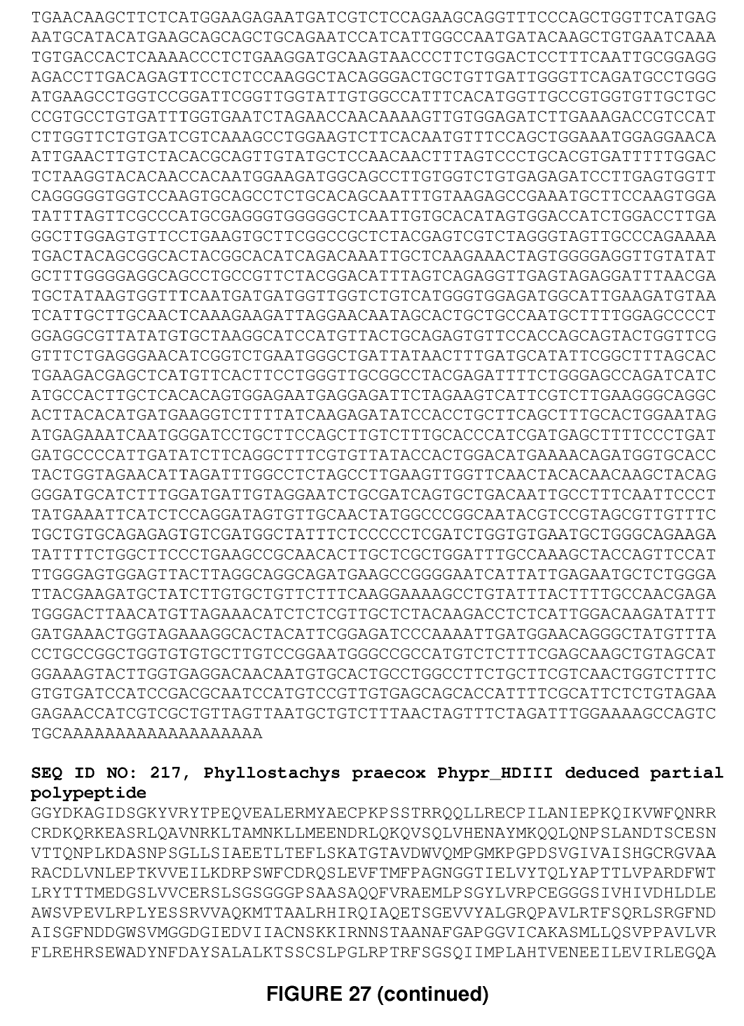

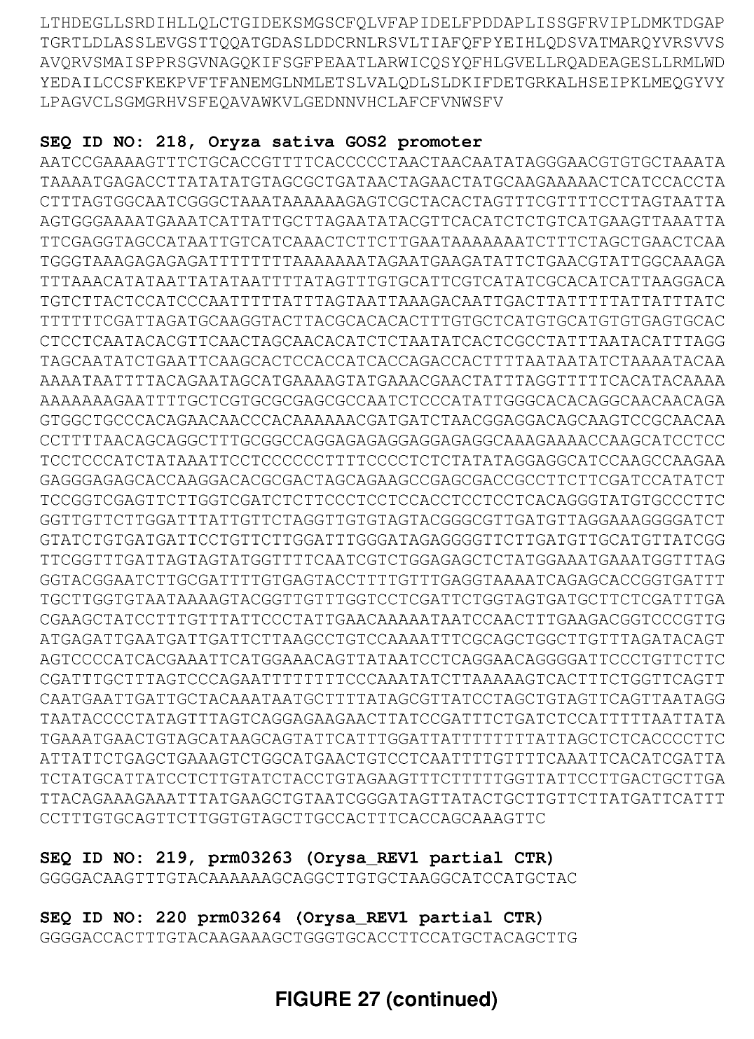

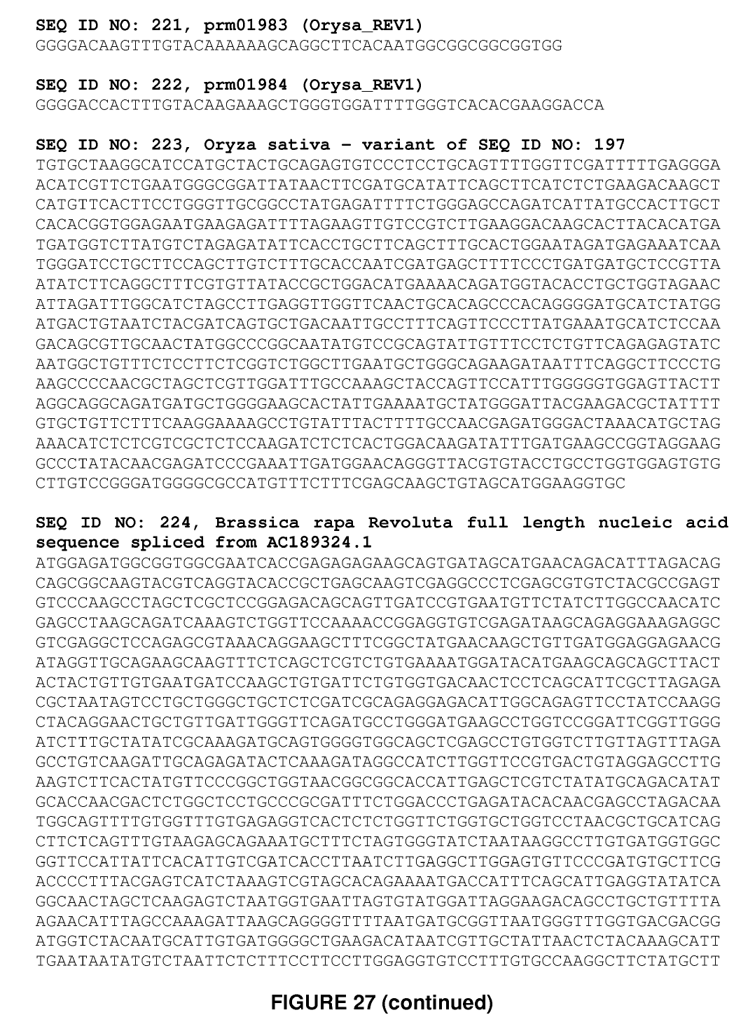

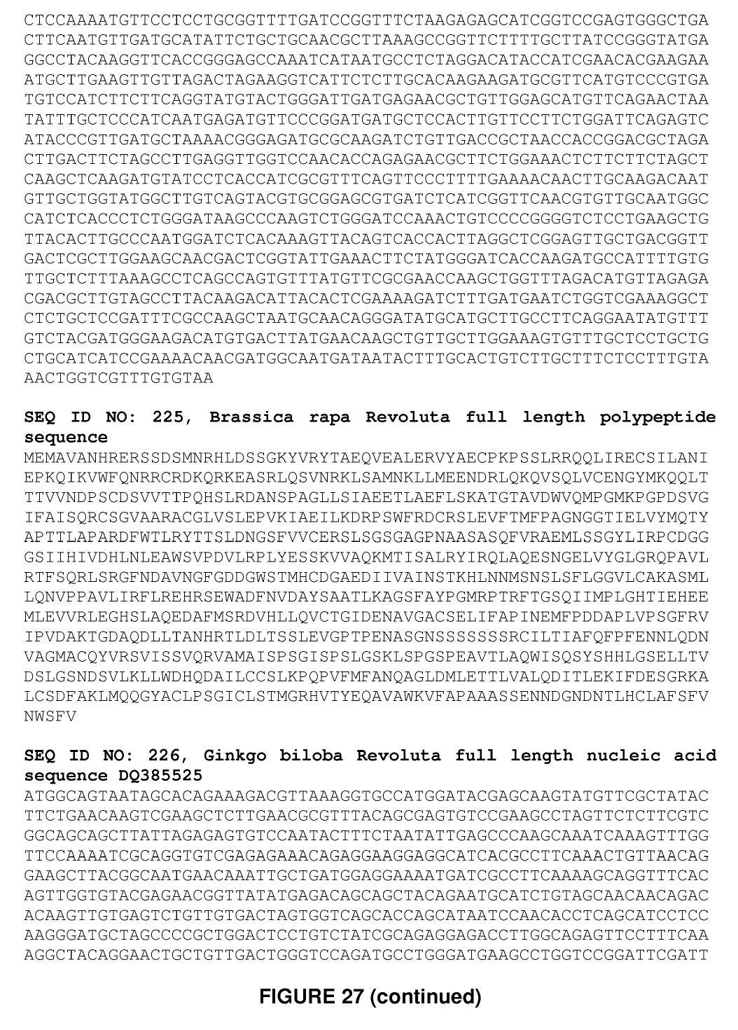

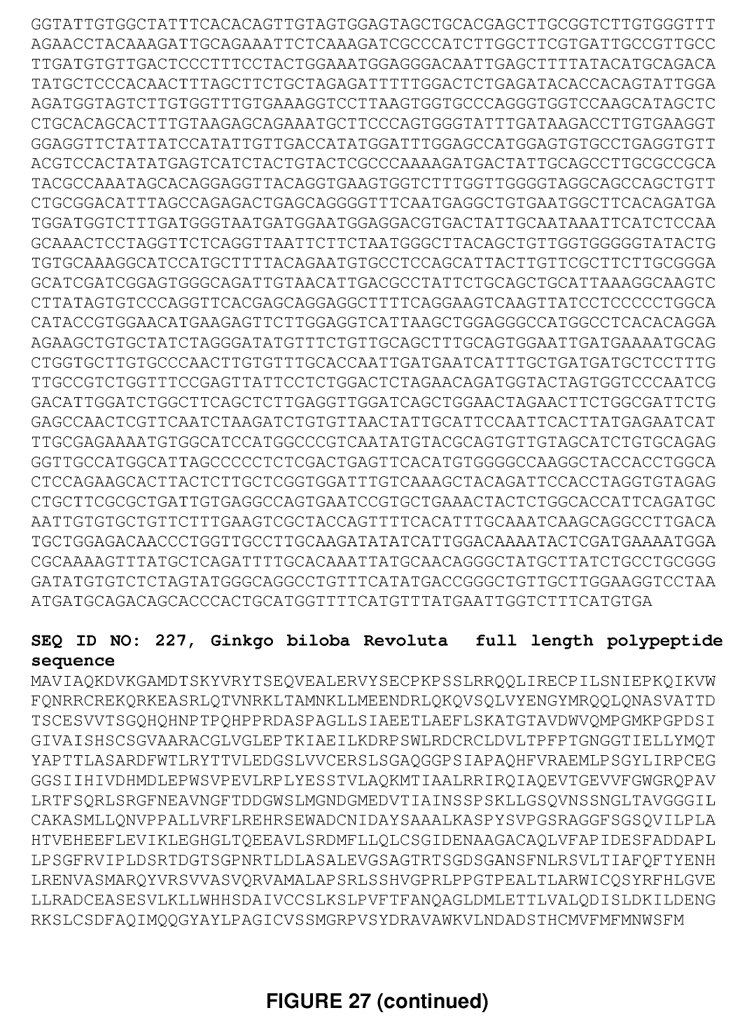

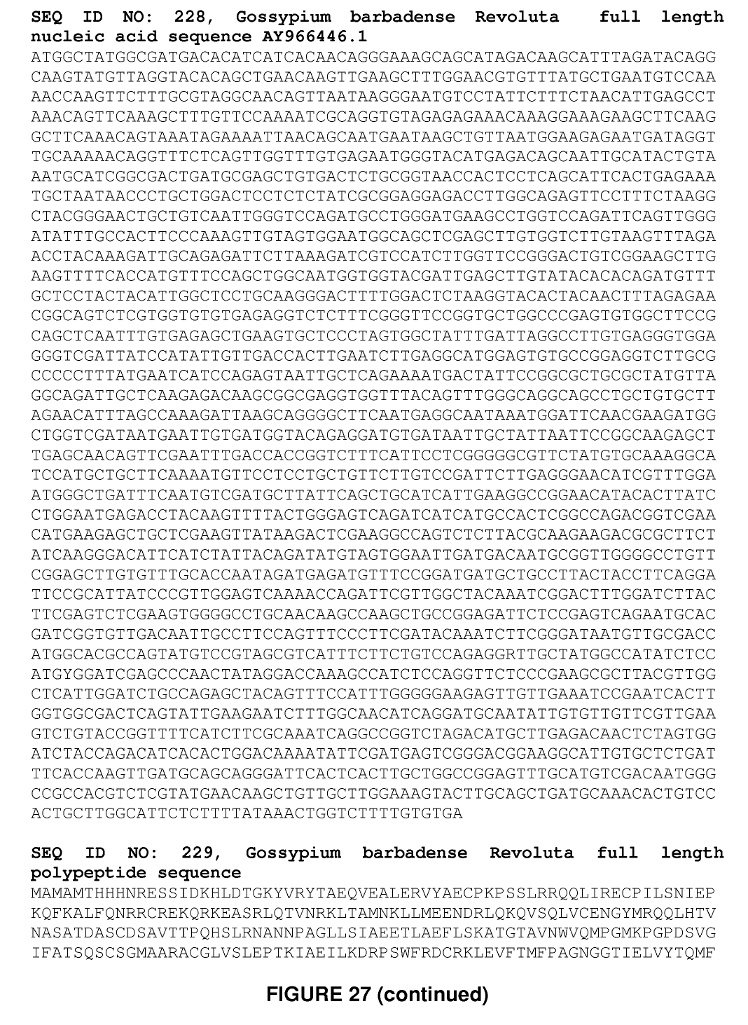

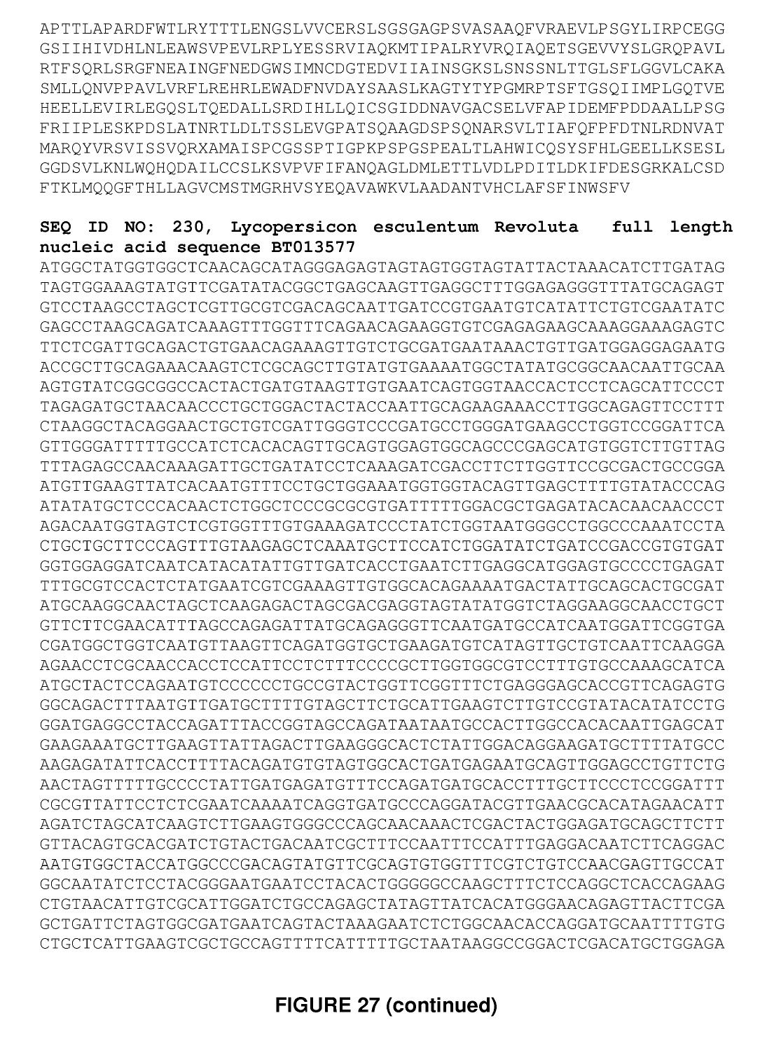

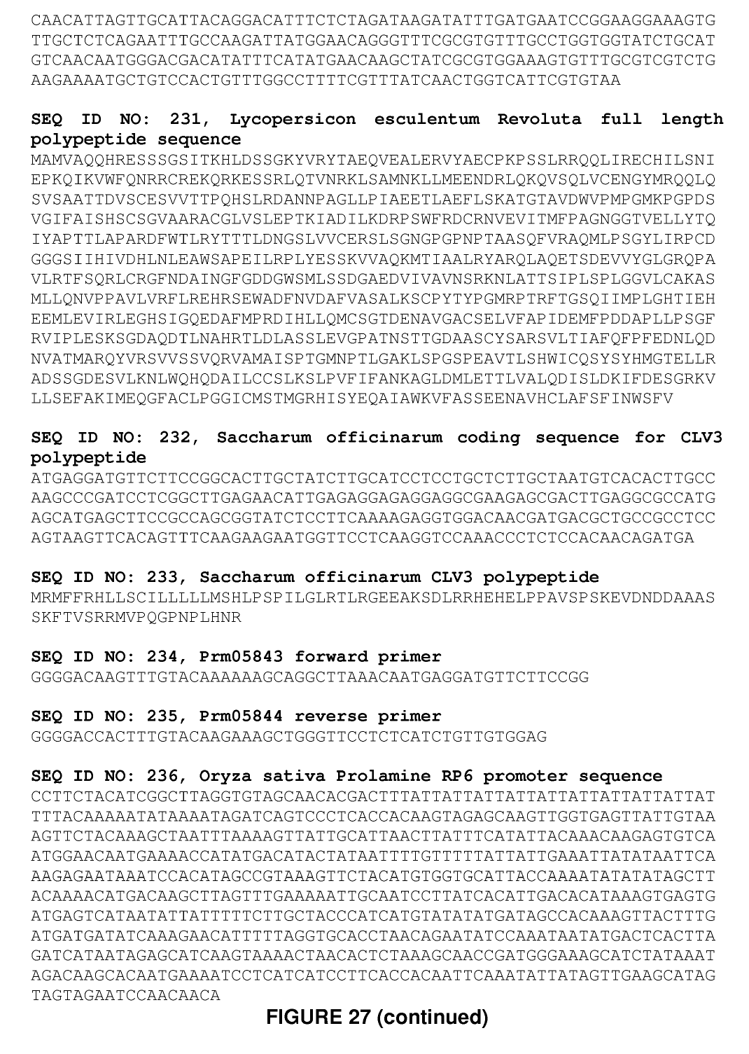

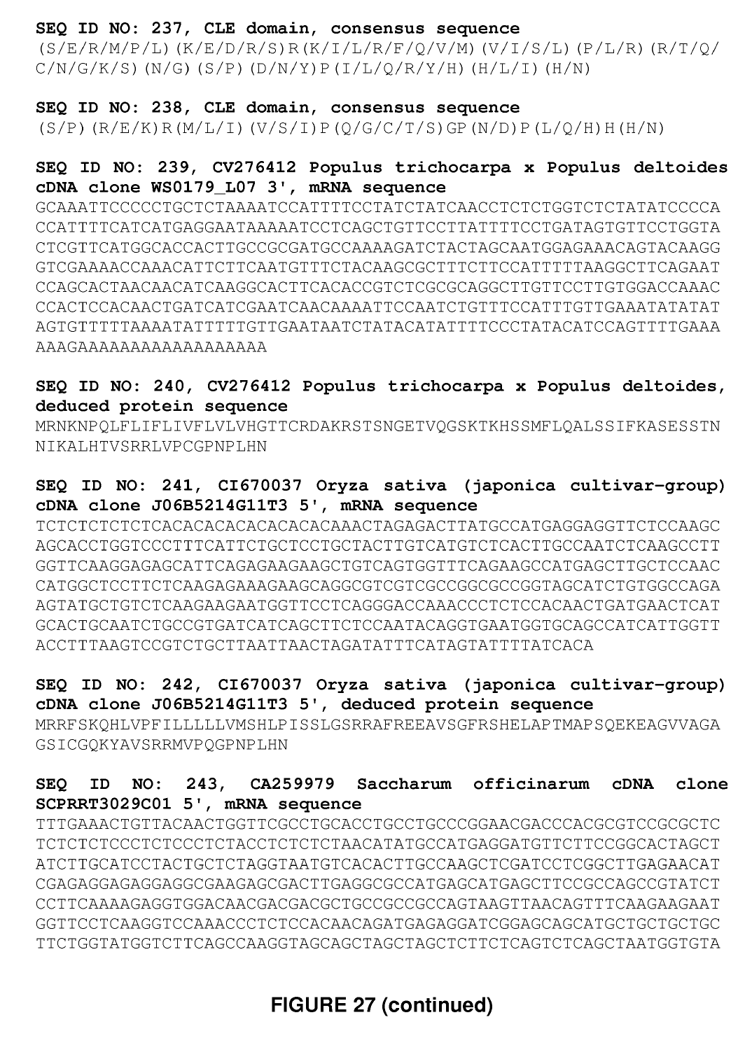

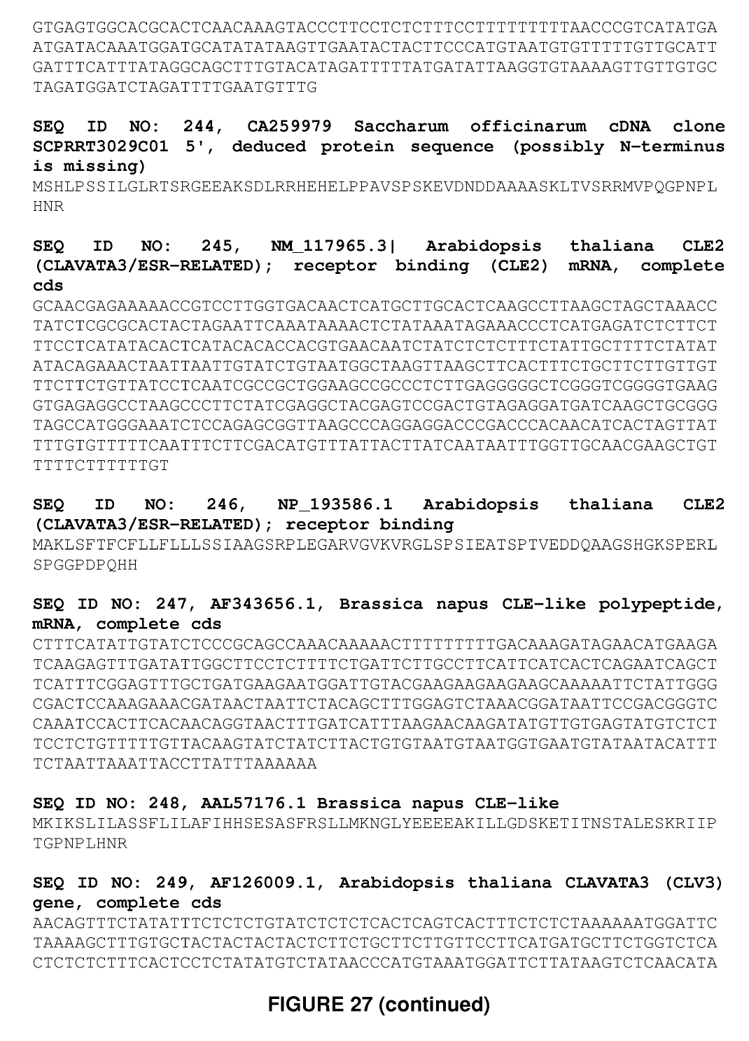

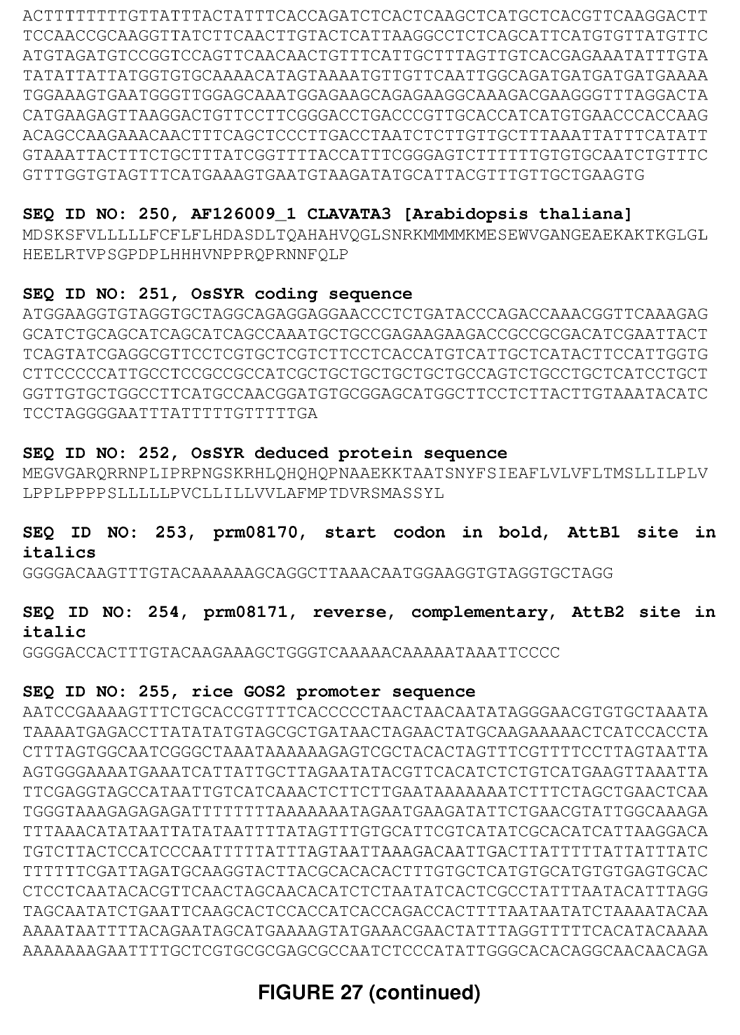

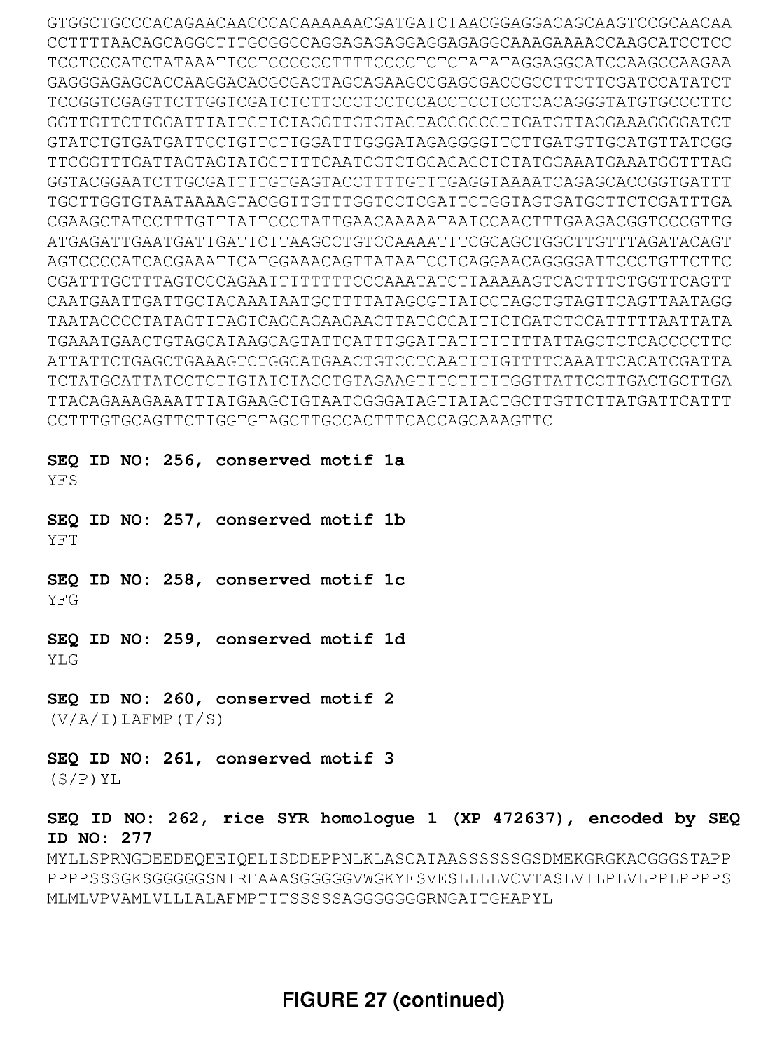

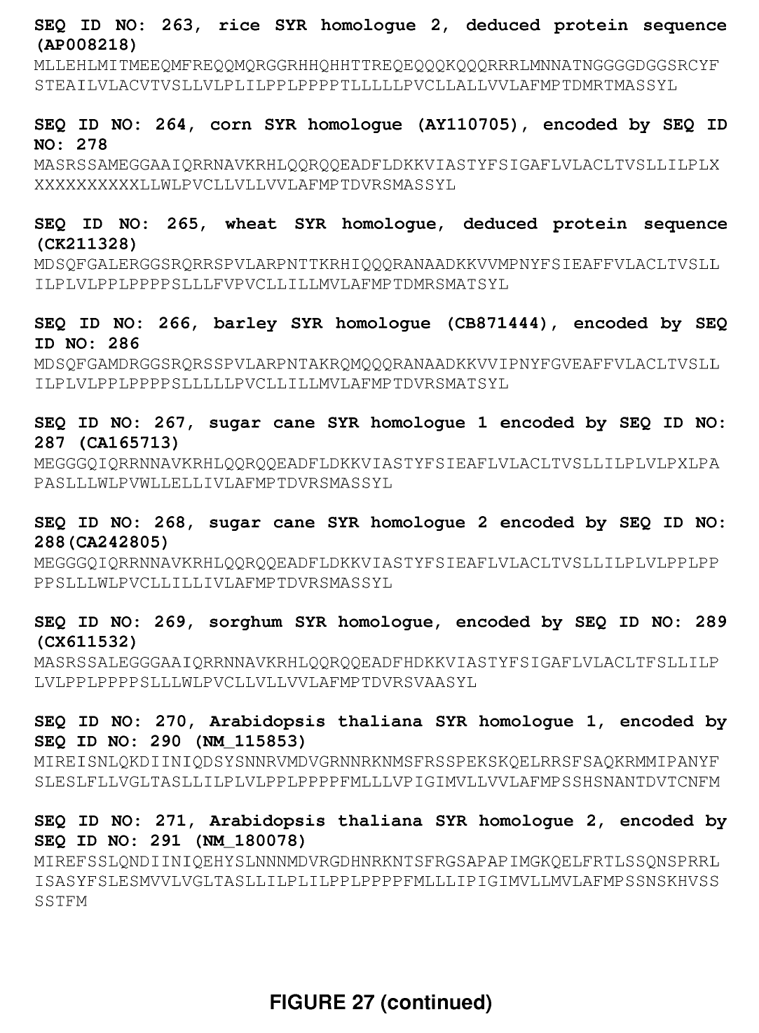

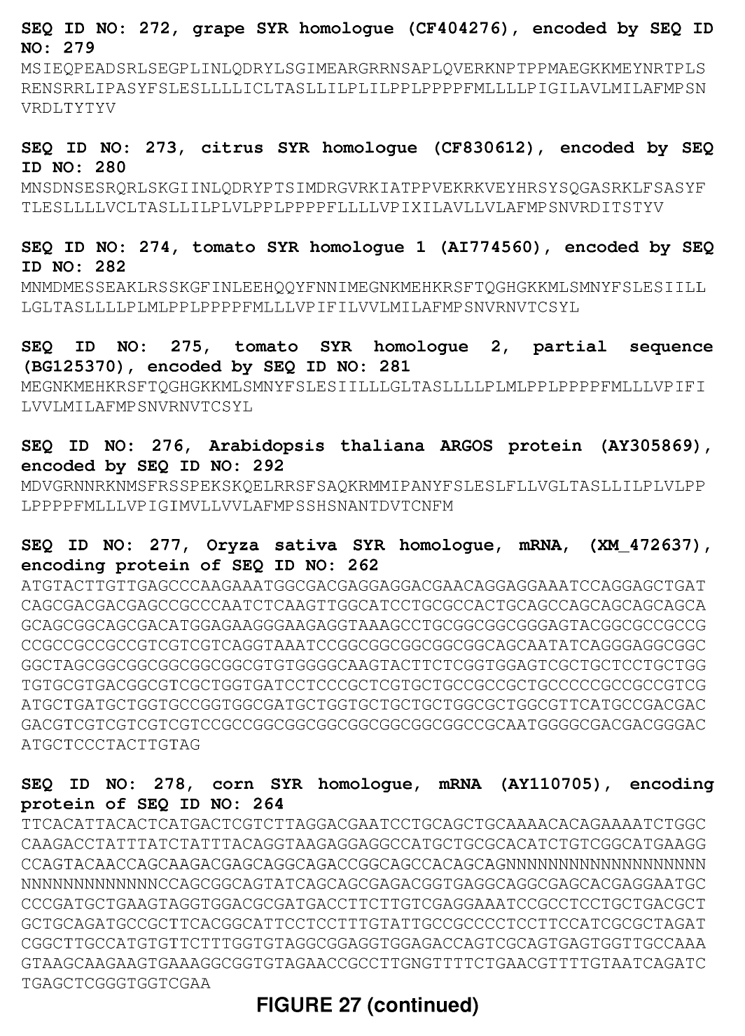

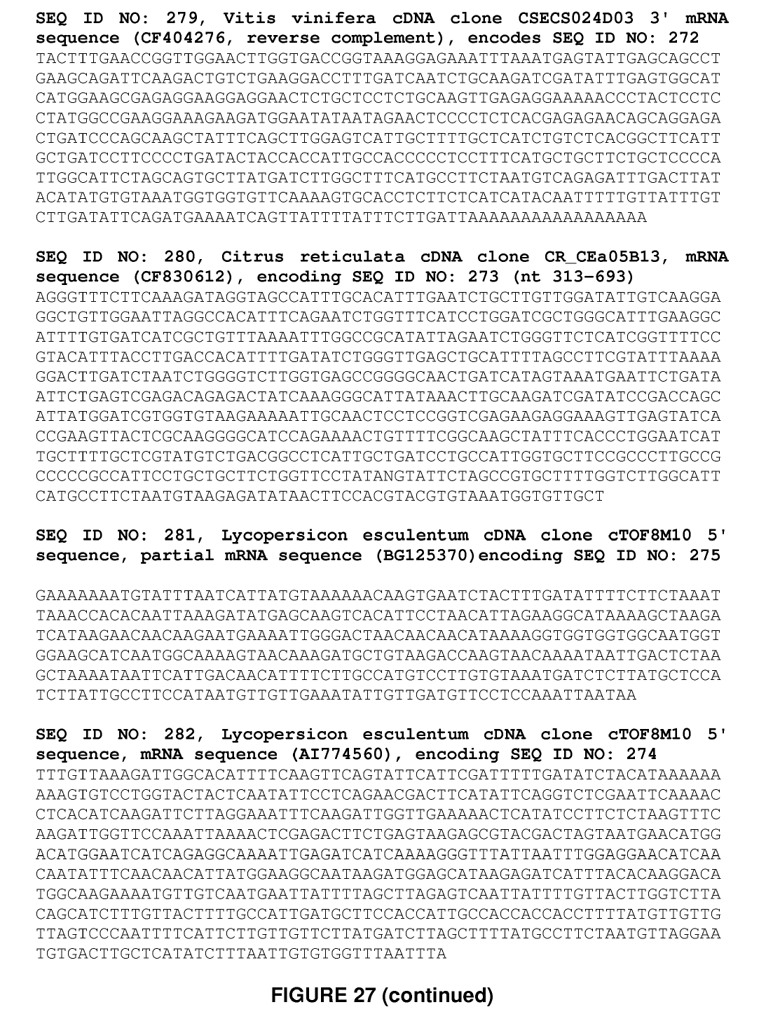

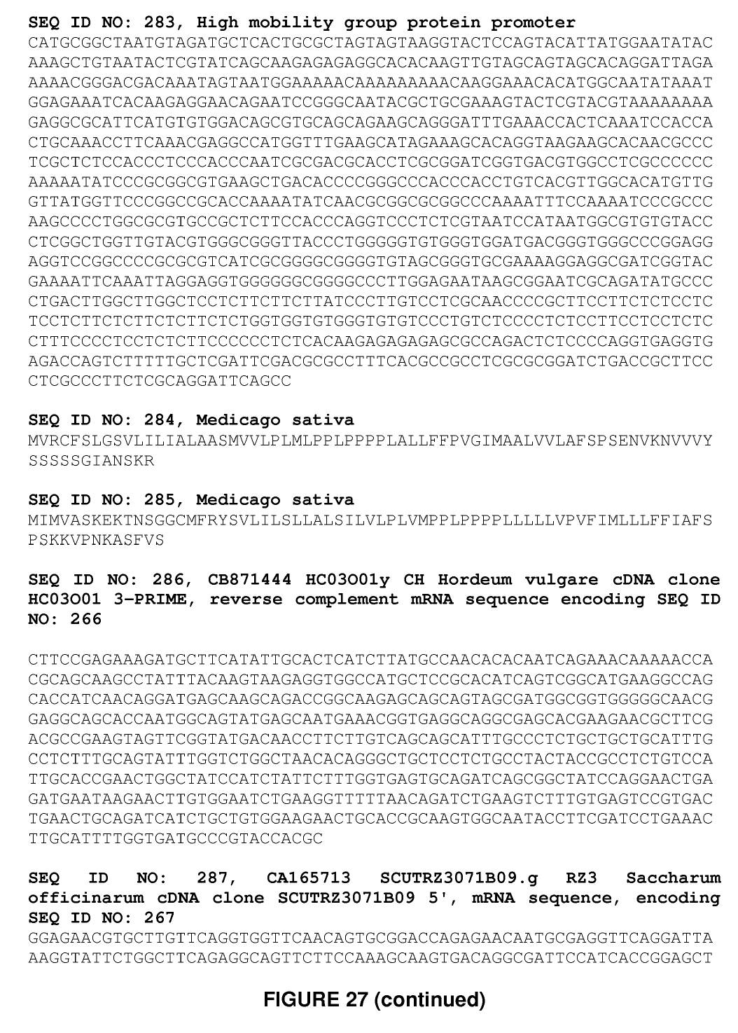

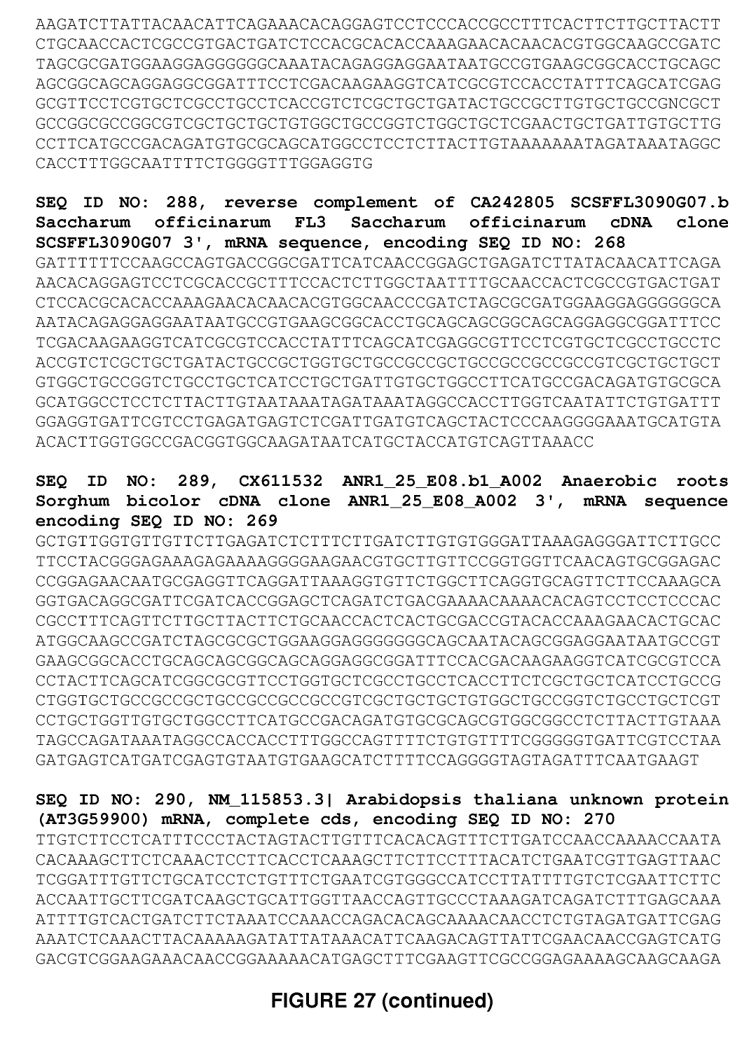

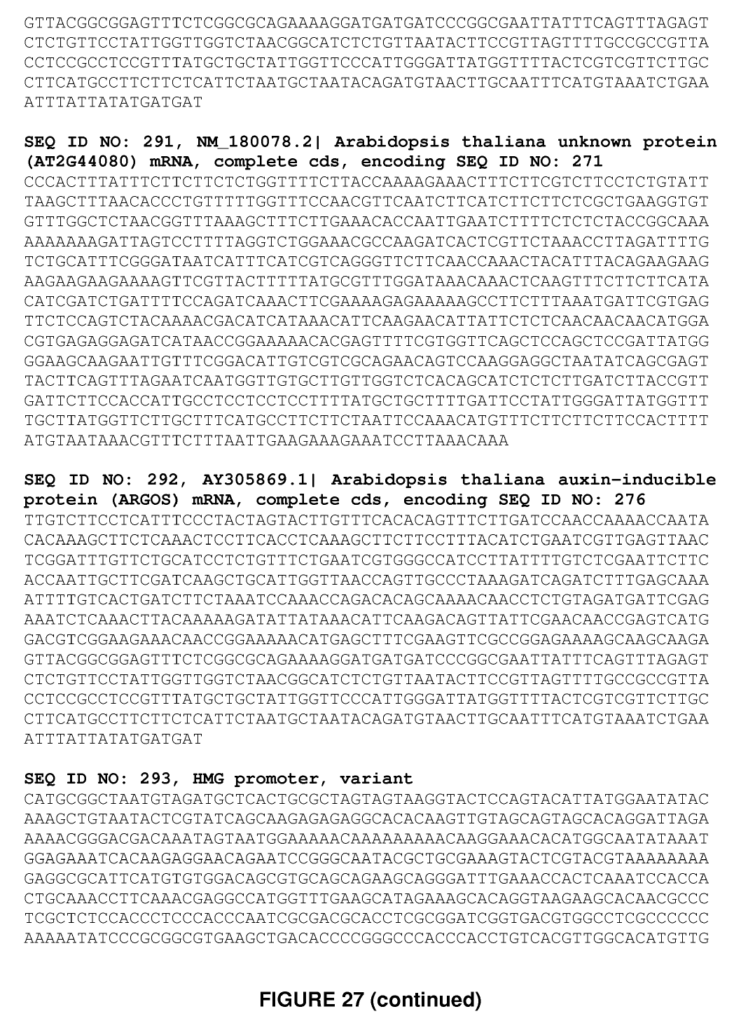

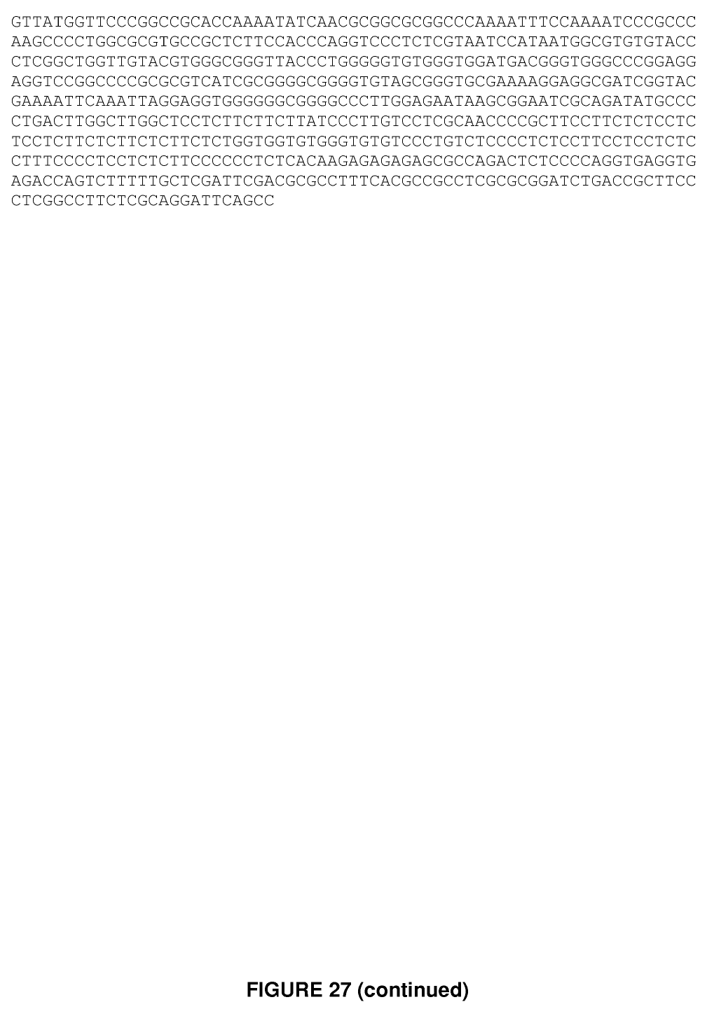

FIG. 27 details examples of sequences useful in performing the methods according to the present invention, or useful in isolating such sequences. Sequences may result from public EST assemblies, with lesser quality sequencing. As a consequence, a few nucleic acid substitutions may be expected. Both 5′ and 3′ UTRs may also be used for the performing the methods of the invention. SEQ ID NO: 1 to SEQ ID NO: 58 relate to ERLK; SEQ ID NO: 58 to SEQ ID NO: 112 relate to FBXWD40; SEQ ID NO: 113 to SEQ ID NO: 155 relate to RANBP; SEQ ID NO: 156 to SEQ ID NO: 193 and SEQ ID NO: 58 relate to GLK; SEQ ID NO: 194 to SEQ ID NO: 231 and SEQ ID NO: 58 relate to REV ΔHDZip/START; SEQ ID NO: 232 to SEQ ID NO: 250 relate to CLE; SEQ ID NO: 58 and SEQ ID NO: 251 to SEQ ID NO: 292 relate to SYR. SEQ ID NO: 276 represents the ARGOS protein sequence (GenBank accession AY305869).

### EXAMPLES

The present invention will now be described with reference to the following examples, which are by way of illustration alone. The following examples are not intended to completely define or otherwise limit the scope of the invention.

DNA manipulation: unless otherwise stated, recombinant DNA techniques are performed according to standard protocols described in (Sambrook (2001) Molecular Cloning: a laboratory manual, 3rd Edition Cold Spring Harbor Laboratory Press, CSH, New York) or in Volumes 1 and 2 of Ausubel et al. (1994), Current Protocols in Molecular Biology, Current Protocols. Standard materials and methods for plant molecular work are described in Plant Molecular Biology Labfax (1993) by R. D. D. Croy, published by BIOS Scientific Publications Ltd (UK) and Blackwell Scientific Publications (UK).

### Example 1

### Identification of Homologues of the ERLK Protein of SEQ ID NO: 2 in Arabidopsis, Rice and Other Plant Species

Sequences (full length cDNA, ESTs or genomic) related to the nucleic acid sequence used in the methods of the present invention were identified amongst those maintained in the Entrez Nucleotides database at the National Center for Biotechnology Information using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). This program is typically used to find regions of local similarity between sequences by comparing nucleic acid or polypeptide sequences to sequence databases and by calculating the statistical significance of matches. The polypeptide encoded by the nucleic acid of the present invention was used with the TBLASTN algorithm, with default settings and the filter for ignoring low complexity sequences was set off. The output of the analysis was viewed by pairwise comparison, and ranked according to the probability score (E-value), where the score reflect the probability that a particular alignment occurs by chance (the lower the E-value, the more significant the hit). In addition to E-values, comparisons were also scored by percentage identity. Percentage identity refers to the number of identical nucleotides (or amino acids) between the two compared nucleic acid (or polypeptide) sequences over a particular length. In some instances, the default parameters may be adjusted to modify the stringency of the search.

Rice sequences and EST sequences from various plant species may also be obtained from other databases, such as KOME (Knowledge-based Oryza Molecular biological Encyclopedia; Kikuchi et al., Science 301, 376-379, 2003), Sputnik (Rudd, S., Nucleic Acids Res., 33: D622-D627, 2005) or the Eukaryotic Gene Orthologs database (EGO, hosted by The Institute for Genomic Research). These databases are searchable with the BLAST tool. SEQ ID NO: 11 to SEQ ID NO: 56 are nucleic acid and protein sequences of homologues of SEQ ID NO: 2 and were obtained from the above-mentioned databases using SEQ ID NO: 2 as a query sequence.

TABLE A
Nucleic acid sequences related to the nucleic acid sequence
(SEQ ID NO: 1) useful in the methods of the present invention,
and the corresponding deduced polypeptides.

| | Nucleic acid | |
|---|---|---|
| Plant Source | SEQ ID NO: | Protein SEQ ID NO: |
| Arabidopsis thaliana ERLK | 1 | 2 |
| Arabidopsis thaliana AAC64891 | 11 | 12 |
| Arabidopsis thaliana NM_104355.2 | 13 | 14 |
| Arabidopsis thaliana At3g58690 | 15 | 16 |
| Arabidopsis thaliana At3g58690 | 17 | 18 |
| Arabidopsis thaliana At4g02010 | 19 | 20 |
| Arabidopsis thaliana At5g56890 | 21 | 22 |
| Arabidopsis thaliana AT2G20300 | 23 | 24 |
| Oryza sativa Osi093820.1 | 25 | 26 |
| Oryza sativa Osi015947.1 | 27 | 28 |
| Oryza sativa Osi003977.2 | 29 | 30 |
| Oryza sativa Osi003977 | 31 | 32 |
| Oryza sativa Osi000040.5 | 33 | 34 |
| Oryza sativa Ozsa8316 | 35 | 36 |
| Oryza sativa Osi001397.3 | 37 | 38 |
| Oryza sativa Osi000142.1 | 39 | 40 |
| Oryza sativa Osi009054.1 | 41 | 42 |
| Oryza sativa Osi001078.4 | 43 | 44 |
| Saccharum officinarum TC15181 | 45 | 46 |
| Glycine max TC196912 | 47 | 48 |
| Solanum tuberosum TC81885 | 49 | 50 |
| Nicotiana tabacum coi2 | 51 | 52 |
| Medicago truncatula ABE82646.1 | 53 | 54 |
| Populus sp TC25047 | 55 | 56 |

### Example 2

### Determination of Global Similarity and Identity Between the Kinase Domains of ERLK Proteins

Percentages of similarity and identity between the kinase domains of ERLK proteins were determined using MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT software generates similarity/identity matrices for DNA or protein sequences without needing pre-alignment of the data. The program performs a series of pair-wise alignments using the Myers and Miller global alignment algorithm (with a gap opening penalty of 12, and a gap extension penalty of 2), calculates similarity and identity using for example Blosum 62 (for polypeptides), and then places the results in a distance matrix. Sequence similarity is shown in the bottom half of the dividing line and sequence identity is shown in the top half of the diagonal dividing line. The sequence of SEQ ID NO: 2 is indicated as number 1 in the matrix.

Results of the software analysis are shown in Table C for the global similarity and identity over the kinase domains of the ERLK polypeptides. The kinase domains were delineated using the SMART tool and the obtained sequences are listed in Table B. Percentage identity is given above the diagonal (in bold) and percentage similarity is given below the diagonal (normal font). Percentage identity between kinase domains of ERLK paralogues and orthologues of CDS845 (SEQ ID NO: 2) ranges between 30% and 68.8%.

TABLE B

sequences of the kinase domains as obtained
upon analysis with SMART and used in the
MATGAT analysis:

| | |
|---|---|
| CDS0845 | FSEEKKIGNGDVYKGVLSDGTVAAIKKLHMFNDNASNQK |
| | HEERSFRLVQRSTSRLQCPYLVELLGYCADQNHRILIYE |
| | FMPNGTVEHHLHDHNFKNLKDRPQPLDWGARLRIALDCA |
| | RALEFLHENTISTVIHRNFKCTNILLDQNNRAKVSDFGL |
| | AKTGSDKLNGEISTRVIGTTGYLAPEYASTGKLTTKSDV |
| | YSYGIVLLQLLTGRTPIDSRRPRGQDVLVSWALPRLTNR |
| | EKISEMVDPTMKGQYSQKDLIQVAAIAAVCVQPEASYRP |
| | LMTDVVHSL |
| NP_175879 | FSEEKKIGNGDVYKGVLSDGTVAAIKKLHMFNDNASNQK |
| | HEERSFRLEVDLLSRLQCPYLVELLGYCADQNHRILIYE |
| | FMPNGTVEHHLHDHNFKNLKDRPQPLDWGARLRIALDCA |
| | RALEFLHENTISTVIHRNFKCTNILLDQNNRAKVSDFGL |
| | AKTGSDKLNGEISTRVIGTTGYLAPEYASTGKLTTKSDV |
| | YSYGIVLLQLLTGRTPIDSRRPRGQDVLVSWALPRLTNR |
| | EKISEMVDPTMKGQYSQKDLIQVAAIAAVCVQPEASYRP |
| | LMTDVVHSL |
| AT3G58690 | FSKSNVVGNGGFGLVYRGVLNDGRKVAIKLMDHAGKQGE |
| | EEFKMEVELLSRLRSPYLLALLGYCSDNSHKLLVYEFMA |
| | NGGLQEHLYLPNRSGSVPPRLDWETRMRIAVEAAKGLEY |
| | LHEQVSPPVIHRDFKSSNILLDRNFNAKVSDFGLAKVGS |
| | DKAGGHVSTRVLGTQGYVAPEYALTGHLTTKSDVYSYGV |
| | VLLELLTGRVPVDMKRATGEGVLVSWALPQLADRDKVVD |
| | IMDPTLEGQYSTKEVVQVAAIAAMCVQAEADYRPLMADV |
| | VQSL |
| At3g58690 | FSKSNVVGNGGFGLVYRGVLNDGRKVAIKLMDHAGKQGE |
| | EEFKMEVELLSRLRSPYLLALLGYCSSDNSHKLLVYEFM |
| | ANGGLQEHLYLPNRSGSVPPRLDWETRMRIAVEAAKGLE |
| | YLHEQVSPPVIHRDFKSSNILLDRNFNAKVSDFGLAKVG |
| | SDKAGGHVSTRVLGTQGYVAPEYALTGHLTTKSDVYSYG |
| | VVLLELLTGRVPVDMKRATGEGVLVSWALPQLADRDKVV |
| | DIMDPTLEGQYSTKEVVQVAAIAAMCVQAEADYRPLMAD |
| | VVQSL |
| AT4G02010 | FESASILGEGGFGKVYRGILADGTAVAIKKLTSGGPQGD |
| | KEFQVEIDMLSRLHHRNLVKLVGYYSSRDSSQHLLCYEL |
| | VPNGSLEAWLHGPLGLNCPLDWDTRMKIALDAARGLAYL |
| | HEDSQPSVIHRDEKASNILLENNFNAKVADFGLAKQAPE |
| | GRGNHLSTRVMGTFGYVAPEYAMTGHLLVKSDVYSYGVV |
| | LLELLTGRKPVDMSQPSGQENLVTWTRPVLRDKDRLEEL |
| | VDSRLEGKYPKEDFIRVCTIAAACVAPEASQRPTMGEVV |
| | QSL |
| At5g56890 | FDESRVLGEGGFGRVYEGVFDDGTKVAVKVLKRDDQQGS |
| | REFLAEVEMLSRLHHRNLVNLIGICIEDRNRSLVYELIP |
| | NGSVESHLHGIDKASSPLDWDARLKIALGAARGLAYLHE |
| | DSSPRVIHRDFKSSNILLENDFTPKVSDFGLARNALDDE |
| | DNRHISTRVMGTFGYVAPEYAMTGHLLVKSDVYSYGVVL |
| | LELLTGRKPVDMSQPPGQENLVSWTRPFLTSAEGLAAII |
| | DQSLGPEISFDSIAKVAAIASMCVQPEVSHRPFMGEVVQ |
| | AL |
| AT2G20300 | FSAKRVLGEGGFGRVYQGSMEDGTEVAVKLLTRDNQNRD |
| | REFIAEVEMLSRLHHRNLVKLIGICIEGRTRCLIYELVH |
| | NGSVESHLHEGTLDWDARLKIALGAARGLAYLHEDSNPR |
| | VIHRDFKASNVLLEDDFTPKVSDFGLAREATEGSQHIST |
| | RVMGTFGYVAPEYAMTGHLLVKSDVYSYGVVLLELLTGR |
| | RPVDMSQPSGEENLVTWARPLLANREGLEQLVDPALAGT |
| | YNFDDMAKVAAIASMCVHQEVSHRPFMGEVVQAL |
| Osi015947 | FSECNVVGRGAYGVVFRGRLGDGTTAAIKRLKMDGRREG |
| | EREFRIEMGVAITAQVDLLSRMHSPYLVGLLGYCADQSH |
| | RLLVFEFMPNGSLKSHLHRRALAPAEQPPPLDWQTRLGI |
| | ALDCARALEFLHEHSSPAVIHRDFKCSNILLDHNYRARV |
| | SDFGMAKLGSNKANGQVAAITAMOIQIKADYRELMIDVV |
| | QSL |
| Osi003977 | FGRAHVVGQGSFGAVYRGVLPDGRKVAVKLMDRPGKQGE |
| | EEFEMEVELLSRLRSPYLLGLIGHCSEGGHRLLVYEFMA |
| | NGGLQEHLYPNGAFEKIETFSIYLVKQRPIFDKNIGHPI |
| | CRRAHFSRQLISHKADIVGSCGGISKLDWPTRMRIALEA |
| | AKGLEYLHERVNPPVIHRDFKSSNILLDKDFRARVSDFG |
| | LAKLGSDRAGGHVSTRVLGTQGYVAPDYGVVLLELLTGR |
| | VPVDMKRPPGEGVLVNWALPMLTDREKVVQILDPALEGQ |
| | YSLKDAVQVAAIAAMCVQQEADYRPLMADVVQSL |
| Osi000040a | FDPSSMLGEGGFGRVFKGVLTDGTAVAIKKLTSGGHQGD |
| | KEFLVEVEMLSRLHHRNLVKLIGYYSNRTLGASRPLDWD |
| | TRMRIALDAARGLAYLHEDSQPCVIHRDFKASNILLEDD |
| | FHAKVSDFGLAKQAPEGRTNYLSTRVMGTFGYVAPEYAM |
| | TGHLLVKSDVYSYGVVLLELLTGRRPVDMSQPSGQENLV |
| | TWLTQMFPDLSTKSLVSHQPLLAKLSGVEILICSILTTQ |
| | ARPILRDKDTLEELADPKLGGQYPKDDFVRVCTIAAACV |
| | SPEASQRPTMGEVVQSL |
| Osi000040b | FSNDLAIGVGGFGVVYRGVVDGDVKVAVKRSNPSSEQGI |
| | TEFQTEVEMLSKLRHRHLVSLIGFCEEDGEMVLVYDYME |
| | HGTLREHLYHNGGKPTLSWRHRLDICIGAARGLHYLHTG |
| | ESHVSTVVKGSFGYLDPEYYRRQQLTDKSDVYSFGVVLF |
| | EVLMARPALDPALPRDQVSLADYALACKRGGALPDVVDP |
| | AIRDQIAPECLAKFADTAEKCLSENGTERPTMGDVLWNL |
| Osi001397 | FDNSRIIGEGGFGRVYEGILEDGERVAVKILKRDDQQGT |
| | REFLAEVEMLSRLHHRNLVKLIGICTEEHIRCLVYELVP |
| | NGSVESHLHGSDKGTAPLYWDARLKIALGAARALAYLHE |
| | DSSPRVIHRDFKSSNILLEHDFTPKVSDFGLARTAIGEG |
| | NEHISTRVMGTFGYVAPEYAMTGHLLVKSDVYSYGVVLL |
| | ELLTGRKPVDILRPPGQENLVAWACPFLTSRDGLETIID |
| | PSLGNSILFDSIAKVAAIASMCVQPEVDQRPFMGEVVQA |
| | L |
| Osi000142 | FDDSTVLGEGGFGCVYQGTLEDGTRVAVKVLKRYDGQGE |
| | REFLAEVEMLGRLHHRNLVKLLGICVEENARCLVYELIP |
| | NGSVESHLHGVDLETAPLDWNARMKIALGAARALAYLHE |
| | DSSPCVIHRDFKSSNILLEHDFTPKVSDFGLARTARGEG |
| | NQHISTRVMGTFGYVAPEYAMTGHLLVKSDVYSYGVVLL |
| | ELLTGRKPVDMSRPGGQENLVSWARPLLTNVVSLRQAVD |
| | PLLGPNVPLDNVAKAAAIASMCVQPEVAHRPSMGEVVQA |
| | L |
| Osi009054 | FDSKRVLGQGGFGRVYHGTMDGGDEIAVKLLTREDRSGD |
| | REFIAEVEMLSRLHHRNLVKLIGICIEHNKRCLVYELIR |
| | NGSVESHLHGADKAKGMLNWDVRMKIALGAARGLAYLHE |
| | DSNPHVIHRDFKGSNILLEEDFTPKVTDFGLAREATNGI |
| | QPISTRVMGTFGYVAPEYAMTGHLLVKSDVYSYGVVLLE |
| | LLSGRKPVCMSDTNGPQNLVTWARPLLCHKEGLERLIDP |
| | SLNGNFNFDDVAKVASIASMCVHNDPSQRPFMGEVVQAL |
| Osi001078 | ESENKIIGEGGYGRVYRGTIDDEVDVAVKLLTRKHQNRD |
| | REFIAEVEMLSRLHHRNLVKLIGICIERSTRCLVFELVP |
| | NGSVESHLHGSDKIYGPLDFDTRMKIALGAARGLAYLHE |
| | DANPHVIHRDFKASNVLLENDFTPKVADFGLAKEASEGM |
| | DHISTQVMGTFGYVAPEYAMTGHLLVKSDVYSYGVVLLE |
| | LLSGRKPVDMTQPPGSENLVTWARPLLTDRDGLQQLVDP |
| | SMPAASYGFEKLAKAAAIASMCVHVEASHRPFMGEVVQA |
| | L |
| TC15181 | FGRAHMVGQGSFGAVYRGVLPDGRKVAVKLMDRPGKQGE |
| | EEFEMEVELLSRLRSPYLLGLIGHCSEGGHRLLVYEFMA |
| | NGGLQEHLYPNRGSCGGISKLDWDTRMRIALEAAKGLEY |
| | LHERVNPPVIHRDFKSSNILLDKDFHARVSDFGLTKLGS |
| | DRAGGHVSTRVLGTQGYVAPEYALTGHLTTKSDVYSYGV |
| | VLLELLTGRVPVDMKRPPGEGVLVNWALPMLTDREKVVR |
| | ILDPALEGQYSLKDAVQVAAIAAMCVQPEADYRPLMADV |
| | VQSL |
| TC196912 | SKSNVIGHGGFGLVYRGVLNDGRKVAIKFMDQAGKQGEE |
| | EFKVEVELLSRLHSPYLLALLGYCSDSNHKLLVYEFMAN |
| | GGLQEHLYPVSNSIITPVKLDWETRLRIALEAAKGLEYL |
| | HEHVSPPVIHRDEKSSNILLDKKFHAKVSDFGLAKLGPD |
| | RAGGHVSTRVLGTQGYVAPEYALTGHLTTKSDVYSYGVV |
| | LLELLTGRVPVDMKRPPGEGVLVSWALPLLTDREKVVKI |
| | MDPSLEGQYSMKEVVQVAAIAAMCVQPEADYRPLMADVV |
| | QSL |
| TC81885 | EEEFKVEVELLCRLRSPYLLSLIGYCSESSHKLLVYEFM |
| | ANGGLQEHLYPIKGSNNCCPKLDWKTRLRIALEAAKGLE |
| | YLHEHVNPPIIHRDLKSSNILLDKNFHAKVSDFGLAKLG |
| | SDKAGGHVSTRVLGTQGYVAPEYALTGHLTTKSDVYSYG |
| | VVLLELLTGRVPVDMKRSPGEGVLVSWALPRLTDREKVV |
| | EIMDPALEGQYSMKEVIQVAAIAAMCVQPEADYRPLMAD |
| | VVQSL |
| ABB36644 | FSLKRVLGEGGFGRVYHGILEDRTEVAVKVLTRDNQNGD |
| | REFIAEVEMLSRLHHRNLVKLIGICSEERIRSLVYELVR |
| | NGSVESHLHGRDGRKEPLDWDVRLKIALGAARGLAYLHE |
| | DSNPRVIHRDFKASNVLLEDDFTPKVADFGLAREATEGS |
| | HHISTRVMGTFGYVAPEYAMTGHLLVKSDVYSYGVVLLE |
| | LLSGRKPVDMSQPPGEENLVTWARPLLTTREGLEQLVDP |
| | SLAGSYDFDDMAKVAAIASMCVHPEVTQRPFMGEVVQAL |
| ABE82646 | MLSRLHHRNLVKLIGICIEGRRRCLVYELVPNGSVESHL |
| | HGDDKNRGPLDWEARMKIALGAARGLAYLHEDSNPRVIH |
| | RDFKASNVLLEDDFTPKVSDFGLAREATEGSNHISTRVM |
| | GTFGYVAPEYAMTGHLLVKSDVYSYGVVLLELLTGRKPV |
| | DMSQPQGQENLVTWARALLTSREGLEQLVDPSLAGGYNF |
| | DDMAKVAAIASMCVHSEVTQRPFMGEVVQAL |
| Popsp | VTWNEGYGVVYGGTLSDGTVAAIKMLHRAGKQGERAFRI |
| | EVDLLSRLHSPYLVELLGYCADQNHRLLVFEFMPNGTLQ |
| | HHLHHKQYRPLDWGTRLRIALDCARALEFLHELTIPAVI |
| | HRDFKCSNILLDQNFRAKVSDFGSAKMGSERINARNSMC |
| | LPSTTGYLAPEHASTGKLTTKSDVYSYGVVLLQLLTGRK |
| | PVDTKQPSGEHVLVSWALPRLTNRDKVVEMVDPAMQDQY |
| | SKKDLIQVAAIAAVCVQPEADYRPLMTDVVQSL |

TABLE C

| | Ident | | | | | | | | | | |
|---|---|---|---|---|---|---|---|---|---|---|---|
| Sim | 1 | 2 | 3 | 4 | 5 | 6 | 7 | 8 | 9 | 10 | 11 |
| 1. CDS0845 | | 98.2 | 57.5 | 57.5 | 47.7 | 48.3 | 47.7 | 39.2 | 44.8 | 40.7 | 30.0 |
| 2. NP_175879 | 98.2 | | 58.9 | 58.9 | 48.8 | 49.3 | 48.8 | 41.0 | 46.0 | 41.6 | 31.0 |
| 3. At3g58690 | 74.8 | 76.6 | | 100.0 | 56.3 | 54.0 | 52.3 | 40.8 | 65.6 | 47.5 | 35.4 |
| 4. At3g58690s | 74.8 | 76.6 | 100.0 | | 56.3 | 54.0 | 52.3 | 40.8 | 65.6 | 47.5 | 35.4 |
| 5. At4g02010 | 66.0 | 67.7 | 73.6 | 73.6 | | 64.0 | 64.9 | 33.2 | 42.6 | 68.4 | 32.6 |
| 6. At5g56890 | 64.5 | 66.3 | 70.8 | 70.8 | 75.7 | | 76.4 | 33.7 | 44.9 | 56.1 | 37.2 |
| 7. At2g20300 | 64.9 | 66.7 | 70.8 | 70.8 | 76.4 | 84.7 | | 30.4 | 44.1 | 58.2 | 37.7 |
| 8. Osi015947 | 50.4 | 52.1 | 53.4 | 53.4 | 47.8 | 48.0 | 47.4 | | 35.8 | 26.9 | 27.3 |
| 9. Osi003977 | 63.5 | 65.1 | 76.9 | 76.9 | 60.3 | 59.0 | 59.3 | 48.2 | | 38.7 | 28.9 |
| 10. Osi000040a | 58.6 | 60.3 | 64.1 | 64.1 | 82.4 | 67.9 | 70.0 | 39.3 | 56.7 | | 29.4 |
| 11. Osi000040b | 46.1 | 47.9 | 51.6 | 51.6 | 48.6 | 49.8 | 51.5 | 43.2 | 42.0 | 42.8 | |
| 12. Osi001397 | 64.2 | 66.0 | 69.7 | 69.7 | 75.0 | 89.5 | 84.7 | 46.7 | 58.3 | 66.2 | 50.4 |
| 13. Osi000142 | 64.5 | 66.3 | 67.1 | 67.1 | 76.1 | 86.5 | 82.5 | 48.2 | 57.7 | 68.3 | 48.2 |
| 14. Osi009054 | 62.1 | 63.8 | 69.0 | 69.0 | 73.9 | 80.7 | 88.6 | 44.7 | 57.7 | 67.9 | 50.5 |
| 15. Osi001078 | 65.6 | 67.4 | 70.4 | 70.4 | 76.4 | 82.2 | 89.4 | 46.0 | 59.3 | 69.3 | 49.3 |
| 16. TC15181 | 71.3 | 73.0 | 91.0 | 91.0 | 71.8 | 69.7 | 70.0 | 50.9 | 83.7 | 63.8 | 49.1 |
| 17. TC196912 | 74.1 | 75.9 | 92.4 | 92.4 | 75.0 | 71.4 | 70.7 | 53.3 | 77.5 | 64.8 | 50.4 |
| 18. TC81885 | 66.0 | 67.7 | 80.9 | 80.9 | 63.8 | 60.7 | 63.4 | 49.8 | 67.1 | 54.5 | 48.1 |
| 19. ABB36644 | 66.0 | 67.7 | 70.0 | 70.0 | 77.9 | 85.8 | 91.6 | 46.2 | 59.3 | 71.0 | 51.6 |
| 20. ABE82646 | 59.2 | 59.2 | 59.2 | 59.2 | 65.9 | 72.0 | 78.4 | 43.4 | 50.2 | 58.6 | 48.7 |
| 21. Popsp | 78.7 | 80.5 | 76.5 | 76.5 | 67.4 | 65.1 | 68.3 | 55.4 | 63.8 | 59.0 | 49.4 |

| | Ident | | | | | | | | | |
|---|---|---|---|---|---|---|---|---|---|---|
| Sim | 12 | 13 | 14 | 15 | 16 | 17 | 18 | 19 | 20 | 21 |
| 1. CDS0845 | 47.7 | 48.8 | 42.0 | 45.7 | 54.7 | 58.6 | 51.8 | 47.0 | 42.9 | 68.8 |
| 2. NP_175879 | 48.8 | 49.8 | 43.0 | 46.7 | 56.1 | 60.0 | 53.2 | 48.1 | 43.3 | 70.6 |
| 3. At3g58690 | 53.8 | 54.2 | 48.7 | 51.1 | 81.0 | 87.0 | 73.6 | 52.7 | 44.0 | 61.6 |
| 4. At3g58690s | 53.8 | 54.2 | 48.7 | 51.1 | 81.0 | 87.0 | 73.6 | 52.7 | 44.0 | 61.6 |
| 5. At4g02010 | 63.1 | 63.2 | 61.2 | 65.0 | 54.5 | 56.1 | 49.1 | 67.4 | 58.0 | 52.5 |
| 6. At5g56890 | 83.3 | 78.9 | 73.1 | 71.7 | 56.5 | 56.8 | 48.2 | 78.2 | 66.9 | 50.2 |
| 7. At2g20300 | 75.5 | 74.1 | 77.7 | 78.1 | 54.9 | 53.1 | 47.3 | 87.5 | 74.0 | 49.8 |
| 8. Osi015947 | 33.8 | 34.0 | 29.7 | 30.3 | 37.6 | 40.1 | 33.1 | 30.8 | 24.5 | 46.0 |
| 9. Osi003977 | 44.1 | 43.8 | 41.9 | 42.7 | 78.9 | 66.1 | 59.0 | 44.4 | 38.2 | 49.1 |
| 10. Osi000040a | 53.7 | 54.3 | 53.7 | 52.9 | 48.4 | 48.1 | 40.8 | 57.2 | 48.9 | 45.0 |
| 11. Osi000040b | 38.0 | 35.3 | 36.0 | 37.7 | 36.5 | 35.0 | 29.6 | 38.0 | 29.9 | 30.8 |
| 12. Osi001397 | | 80.7 | 71.5 | 72.7 | 56.3 | 56.3 | 48.0 | 78.5 | 67.2 | 50.7 |
| 13. Osi000142 | 87.6 | | 71.2 | 69.8 | 54.5 | 56.0 | 46.9 | 74.8 | 65.0 | 51.8 |
| 14. Osi009054 | 82.1 | 79.6 | | 72.6 | 52.0 | 50.2 | 44.0 | 78.8 | 67.0 | 44.9 |
| 15. Osi001078 | 83.2 | 83.2 | 86.1 | | 53.6 | 52.5 | 46.4 | 79.6 | 68.2 | 50.5 |
| 16. TC15181 | 69.7 | 68.6 | 67.5 | 69.7 | | 82.3 | 74.4 | 56.0 | 47.5 | 60.3 |
| 17. TC196912 | 70.3 | 68.5 | 68.8 | 72.1 | 91.0 | | 77.2 | 54.9 | 46.4 | 63.8 |
| 18. TC81885 | 60.6 | 59.5 | 60.4 | 62.8 | 79.8 | 81.5 | | 48.0 | 51.9 | 57.0 |
| 19. ABB36644 | 85.8 | 81.8 | 87.9 | 88.3 | 70.8 | 70.7 | 61.9 | | 74.4 | 50.2 |
| 20. ABE82646 | 71.5 | 69.3 | 74.0 | 74.8 | 59.6 | 59.4 | 67.8 | 77.7 | | 43.9 |
| 21. Popsp | 66.4 | 65.7 | 64.8 | 68.6 | 74.0 | 77.5 | 70.8 | 67.4 | 59.2 | |

### Example 3

### Cloning of the Nucleic Acid Sequence Used in the Methods of the Invention

DNA manipulation: unless otherwise stated, recombinant DNA techniques are performed according to standard protocols described in (Sambrook (2001) Molecular Cloning: a laboratory manual, 3rd Edition Cold Spring Harbor Laboratory Press, CSH, New York) or in Volumes 1 and 2 of Ausubel et al. (1994), Current Protocols in Molecular Biology, Current Protocols. Standard materials and methods for plant molecular work are described in Plant Molecular Biology Labfax (1993) by R. D. D. Croy, published by BIOS Scientific Publications Ltd (UK) and Blackwell Scientific Publications (UK).

The nucleic acid sequence used in the methods of the invention was amplified by PCR using as template a custom-made Arabidopsis thaliana seedlings cDNA library (in pCMV Sport 6.0; Invitrogen, Paisley, UK). PCR was performed using Hifi Taq DNA polymerase in standard conditions, using 200 ng of template in a 50 μl PCR mix. The primers used were prm2500 (SEQ ID NO: 3; sense, start codon in bold:

5′ ggggacaagtttgtacaaaaaagcaggcttcacaatggaaaacaaaa
gccatagc 3′)

and prm2501 (SEQ ID NO: 4; reverse, complementary,:

5′ ggggaccactttgtacaagaaagctgggtaaacaaaagagtgtcatg
gca 3′),

which include the AttB sites for Gateway recombination. The amplified PCR fragment was purified also using standard methods. The first step of the Gateway procedure, the BP reaction, was then performed, during which the PCR fragment recombines in vivo with the pDONR201 plasmid to produce, according to the Gateway terminology, an “entry clone”, p031. Plasmid pDONR201 was purchased from Invitrogen, as part of the Gateway® technology.

### Example 4

### Expression Vector Construction

The entry clone p031 was subsequently used in an LR reaction with p05050, a destination vector used for Oryza sativa transformation. This vector contains as functional elements within the T-DNA borders: a plant selectable marker; a screenable marker expression cassette; and a Gateway cassette intended for LR in vivo recombination with the nucleic acid sequence of interest already cloned in the entry clone. A rice non-viral constitutive promoter, the GOS2 promoter (SEQ ID NO: 58) was located upstream of this Gateway cassette.

After the LR recombination step, the resulting expression vector p030 (FIG. 4) was transformed into Agrobacterium strain LBA4044 and subsequently to Oryza sativa plants.

### Example 5

### Plant Transformation

### Rice Transformation

The Agrobacterium containing the expression vector was used to transform Oryza sativa plants. Mature dry seeds of the rice japonica cultivar Nipponbare were dehusked. Sterilization was carried out by incubating for one minute in 70% ethanol, followed by 30 minutes in 0.2% HgCl₂, followed by a 6 times 15 minutes wash with sterile distilled water. The sterile seeds were then germinated on a medium containing 2,4-D (callus induction medium). After incubation in the dark for four weeks, embryogenic, scutellum-derived calli were excised and propagated on the same medium. After two weeks, the calli were multiplied or propagated by subculture on the same medium for another 2 weeks. Embryogenic callus pieces were sub-cultured on fresh medium 3 days before co-cultivation (to boost cell division activity).

Agrobacterium strain LBA4404 containing the expression vector was used for co-cultivation. Agrobacterium was inoculated on AB medium with the appropriate antibiotics and cultured for 3 days at 28° C. The bacteria were then collected and suspended in liquid co-cultivation medium to a density (OD₆₀₀) of about 1. The suspension was then transferred to a Petri dish and the calli immersed in the suspension for 15 minutes. The callus tissues were then blotted dry on a filter paper and transferred to solidified, co-cultivation medium and incubated for 3 days in the dark at 25° C. Co-cultivated calli were grown on 2,4-D-containing medium for 4 weeks in the dark at 28° C. in the presence of a selection agent. During this period, rapidly growing resistant callus islands developed. After transfer of this material to a regeneration medium and incubation in the light, the embryogenic potential was released and shoots developed in the next four to five weeks. Shoots were excised from the calli and incubated for 2 to 3 weeks on an auxin-containing medium from which they were transferred to soil. Hardened shoots were grown under high humidity and short days in a greenhouse.

Approximately 35 independent T0 rice transformants were generated for one construct. The primary transformants were transferred from a tissue culture chamber to a greenhouse. After a quantitative PCR analysis to verify copy number of the T-DNA insert, only single copy transgenic plants that exhibit tolerance to the selection agent were kept for harvest of T1 seed. Seeds were then harvested three to five months after transplanting. The method yielded single locus transformants at a rate of over 50% (Aldemita and Hodgesl996, Chan et al. 1993, Hiei et al. 1994).

### Corn Transformation

Transformation of maize (Zea mays) is performed with a modification of the method described by Ishida et al. (1996) Nature Biotech 14(6): 745-50. Transformation is genotype-dependent in corn and only specific genotypes are amenable to transformation and regeneration. The inbred line A188 (University of Minnesota) or hybrids with A188 as a parent are good sources of donor material for transformation, but other genotypes can be used successfully as well. Ears are harvested from corn plant approximately 11 days after pollination (DAP) when the length of the immature embryo is about 1 to 1.2 mm. Immature embryos are cocultivated with Agrobacterium tumefaciens containing the expression vector, and transgenic plants are recovered through organogenesis. Excised embryos are grown on callus induction medium, then maize regeneration medium, containing the selection agent (for example imidazolinone but various selection markers can be used). The Petri plates are incubated in the light at 25° C. for 2-3 weeks, or until shoots develop. The green shoots are transferred from each embryo to maize rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Wheat Transformation

Transformation of wheat is performed with the method described by Ishida et al. (1996) Nature Biotech 14(6): 745-50. The cultivar Bobwhite (available from CIMMYT, Mexico) is commonly used in transformation. Immature embryos are co-cultivated with Agrobacterium tumefaciens containing the expression vector, and transgenic plants are recovered through organogenesis. After incubation with Agrobacterium, the embryos are grown in vitro on callus induction medium, then regeneration medium, containing the selection agent (for example imidazolinone but various selection markers can be used). The Petri plates are incubated in the light at 25° C. for 2-3 weeks, or until shoots develop. The green shoots are transferred from each embryo to rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Soybean Transformation

Soybean is transformed according to a modification of the method described in the Texas A&M U.S. Pat. No. 5,164,310. Several commercial soybean varieties are amenable to transformation by this method. The cultivar Jack (available from the Illinois Seed foundation) is commonly used for transformation. Soybean seeds are sterilised for in vitro sowing. The hypocotyl, the radicle and one cotyledon are excised from seven-day old young seedlings. The epicotyl and the remaining cotyledon are further grown to develop axillary nodes. These axillary nodes are excised and incubated with Agrobacterium tumefaciens containing the expression vector. After the cocultivation treatment, the explants are washed and transferred to selection media. Regenerated shoots are excised and placed on a shoot elongation medium. Shoots no longer than 1 cm are placed on rooting medium until roots develop. The rooted shoots are transplanted to soil in the greenhouse. T1 seeds are produced from plants that exhibit tolerance to the selection agent and that contain a single copy of the T-DNA insert.

### Rapeseed/Canola Transformation

Cotyledonary petioles and hypocotyls of 5-6 day old young seedling are used as explants for tissue culture and transformed according to Babic et al. (1998, Plant Cell Rep 17: 183-188). The commercial cultivar Westar (Agriculture Canada) is the standard variety used for transformation, but other varieties can also be used. Canola seeds are surface-sterilized for in vitro sowing. The cotyledon petiole explants with the cotyledon attached are excised from the in vitro seedlings, and inoculated with Agrobacterium (containing the expression vector) by dipping the cut end of the petiole explant into the bacterial suspension. The explants are then cultured for 2 days on MSBAP-3 medium containing 3 mg/l BAP, 3% sucrose, 0.7% Phytagar at 23° C., 16 hr light. After two days of co-cultivation with Agrobacterium, the petiole explants are transferred to MSBAP-3 medium containing 3 mg/l BAP, cefotaxime, carbenicillin, or timentin (300 mg/l) for 7 days, and then cultured on MSBAP-3 medium with cefotaxime, carbenicillin, or timentin and selection agent until shoot regeneration. When the shoots are 5-10 mm in length, they are cut and transferred to shoot elongation medium (MSBAP-0.5, containing 0.5 mg/l BAP). Shoots of about 2 cm in length are transferred to the rooting medium (MSO) for root induction. The rooted shoots are transplanted to soil in the greenhouse. T1 seeds are produced from plants that exhibit tolerance to the selection agent and that contain a single copy of the T-DNA insert.

### Alfalfa Transformation

A regenerating clone of alfalfa (Medicago sativa) is transformed using the method of (McKersie et al., 1999 Plant Physiol 119: 839-847). Regeneration and transformation of alfalfa is genotype dependent and therefore a regenerating plant is required. Methods to obtain regenerating plants have been described. For example, these can be selected from the cultivar Rangelander (Agriculture Canada) or any other commercial alfalfa variety as described by Brown DCW and A Atanassov (1985. Plant Cell Tissue Organ Culture 4: 111-112). Alternatively, the RA3 variety (University of Wisconsin) has been selected for use in tissue culture (Walker et al., 1978 μm J Bot 65:654-659). Petiole explants are cocultivated with an overnight culture of Agrobacterium tumefaciens C58C1 pMP90 (McKersie et al., 1999 Plant Physiol 119: 839-847) or LBA4404 containing the expression vector. The explants are cocultivated for 3 d in the dark on SH induction medium containing 288 mg/L Pro, 53 mg/L thioproline, 4.35 g/L K2SO4, and 100 μm acetosyringinone. The explants are washed in half-strength Murashige-Skoog medium (Murashige and Skoog, 1962) and plated on the same SH induction medium without acetosyringinone but with a suitable selection agent and suitable antibiotic to inhibit Agrobacterium growth. After several weeks, somatic embryos are transferred to BOi2Y development medium containing no growth regulators, no antibiotics, and 50 g/L sucrose. Somatic embryos are subsequently germinated on half-strength Murashige-Skoog medium. Rooted seedlings were transplanted into pots and grown in a greenhouse. T1 seeds are produced from plants that exhibit tolerance to the selection agent and that contain a single copy of the T-DNA insert.

### Example 6

### Evaluation Procedure

### 6.1 Evaluation Setup

Approximately 30 independent TO rice transformants were generated. The primary transformants were transferred from a tissue culture chamber to a greenhouse for growing and harvest of T1 seed. Seven events, of which the T1 progeny segregated 3:1 for presence/absence of the transgene, were retained. For each of these events, approximately 10 T1 seedlings containing the transgene (hetero- and homo-zygotes) and approximately 10 T1 seedlings lacking the transgene (nullizygotes) were selected by monitoring visual marker expression. The transgenic plants and the corresponding nullizygotes were grown side-by-side at random positions. Greenhouse conditions were of shorts days (12 hours light), 28° C. in the light and 22° C. in the dark, and a relative humidity of 70%. Four T1 events were further evaluated in the T2 generation following the same evaluation procedure as for the T1 generation but with more individuals per event. From the stage of sowing until the stage of maturity the plants were passed several times through a digital imaging cabinet. At each time point digital images (2048×1536 pixels, 16 million colours) were taken of each plant from at least 6 different angles.

6.2 Statistical Analysis: t-Test and F-Test

A two factor ANOVA (analysis of variants) was used as a statistical model for the overall evaluation of plant phenotypic characteristics. An F-test was carried out on all the parameters measured of all the plants of all the events transformed with the gene of the present invention. The F-test was carried out to check for an effect of the gene over all the transformation events and to verify for an overall effect of the gene, also known as a global gene effect. The threshold for significance for a true global gene effect was set at a 5% probability level for the F-test. A significant F-test value points to a gene effect, meaning that it is not only the mere presence or position of the gene that is causing the differences in phenotype.

### Example 7

### Evaluation Results

The plant aboveground area (or leafy biomass) was determined by counting the total number of pixels on the digital images from aboveground plant parts discriminated from the background. This value was averaged for the pictures taken on the same time point from the different angles and was converted to a physical surface value expressed in square mm by calibration. Experiments show that the aboveground plant area measured this way correlates with the biomass of plant parts above ground. The above ground area is the time point at which the plant had reached its maximal leafy biomass.

The mature primary panicles were harvested, counted, bagged, barcode-labeled and then dried for three days in an oven at 37° C. The panicles were then threshed and all the seeds were collected and counted. The filled husks were separated from the empty ones using an air-blowing device. The empty husks were discarded and the remaining fraction was counted again. The filled husks were weighed on an analytical balance. The number of filled seeds was determined by counting the number of filled husks that remained after the separation step. The total seed yield was measured by weighing all filled husks harvested from a plant.

As presented in Tables D to F, the aboveground biomass, the seed yield, the number of filled seeds are increased in the transgenic plants with increased expression of a nucleic acid encoding an ERLK protein, compared to suitable control plants. Results from the T1 and the T2 generations are shown.

Table D shows the increase in aboveground biomass in percent, as well as the statistical relevance of this increase according to the F-test, in the T1 and T2 generation of transgenic rice with increased expression of a nucleic acid encoding an ERLK protein.

TABLE D
Aboveground biomass

| | % Difference | P value of F test |
|---|---|---|
| T1 generation | 11 | 0.0137 |
| T2 generation | 12 | 0.0229 |

Table E shows the increase in total seed yield (total seed weight) in percent, as well as the statistical relevance of this increase according to the F-test, in the T1 and T2 generation of transgenic rice with increased expression of a nucleic acid encoding an ERLK protein.

TABLE E
Seed yield

| | % Difference | P value of F test |
|---|---|---|
| T1 generation | 18 | 0.0253 |
| T2 generation | 22 | 0.0062 |

Table F shows the increase in the number of filled seeds in percent, as well as the statistical relevance of this increase according to the F-test, in the T1 and T2 generation of transgenic rice with increased expression of a nucleic acid encoding an ERLK protein.

TABLE F
Number of filled seeds

| | % Difference | P value of F test |
|---|---|---|
| T1 generation | 15 | 0.0042 |
| T2 generation | 18 | 0.0019 |

### FBXW

### Example 8

### Identification of Sequences Related to the Nucleic Acid Sequence Used in the Methods of the Invention

Sequences (full length cDNA, ESTs or genomic) related to the nucleic acid sequence used in the methods of the present invention were identified amongst those maintained in the Entrez Nucleotides database at the National Center for Biotechnology Information using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). The program is used to find regions of local similarity between sequences by comparing nucleic acid or polypeptide sequences to sequence databases and by calculating the statistical significance of matches. The polypeptide encoded by the nucleic acid of the present invention was used for the TBLASTN algorithm, with default settings and the filter to ignore low complexity sequences set off. The output of the analysis was viewed by pairwise comparison, and ranked according to the probability score (E-value), where the score reflect the probability that a particular alignment occurs by chance (the lower the E-value, the more significant the hit). In addition to E-values, comparisons were also scored by percentage identity. Percentage identity refers to the number of identical nucleotides (or amino acids) between the two compared nucleic acid (or polypeptide) sequences over a particular length. In some instances, the default parameters may be adjusted to modify the stringency of the search The Table below provides a list of nucleic acid sequences related to the nucleic acid sequence useful in performing the methods of the present invention.

TABLE G
nucleic acid sequences related to the nucleic acid sequence (SEQ ID NO: 59) used
in the methods of the present invention, and the corresponding deduced polypeptides

| | | | | NCBI | |
|---|---|---|---|---|---|
| | Nucleic acid | Polypeptide | Sequence | accession | Source |
| Name | SEQ ID NO | SEQ ID NO | length | number | organism |
| Arath_FBXW | 59 | 60 | Full length | NM_122112.2 | Arabidopsis |
| (At5g21040) | | | | | thaliana |
| Orysa_FBXW | 61 | 62 | Full length | AK111585 | Oryza sativa |
| Medtr_FBXW | 63 | 64 | Full length | CR931734 | Medicago |
| | | | | (spliced out) | trunculata |
| Triae_FBXW | 65 | 66 | Full length | CJ661176.1 | Triticum |
| | | | | CB307121.1 | aestivum |
| | | | | CJ553648.1 | |
| Poptr_FBXW | 67 | 68 | Full length | Proprietary | Populus |
| | | | | | tremuloides |
| Zeama_FBXW | 69 | 70 | Full length | AC183938.1 | Zea mays |
| | | | | (spliced out) | |
| Vitvi_FBXW | 71 | 72 | Partial (3′) | CF210354 | Vitis vinifera |
| | | | | CF413646 | |
| | | | | CF213082 | |
| Senca_FBXW | 73 | 74 | Partial (5′) | DY662683.1 | Senecio |
| | | | | | cambrensis |
| Helan_FBXW | 75 | 76 | Partial | DY916708 | Helianthus |
| | | | (middle) | | annuus |
| Eupes_FBXW | 77 | 78 | Partial | DV129599 | Euphorbia |
| | | | (middle) | | esula |
| Lyces_FBXW | 79 | 80 | partial | BI931509 | Lycopersicon |
| | | | (middle) | | esculentum |
| Aqufo_FBXW | 81 | 82 | Partial | DT753991.1 | Aquilegia |
| | | | (middle) | | formosa x |
| | | | | | Aquilegia |
| | | | | | pubescens |
| Goshi_FBXW | 83 | 84 | Partial (3′) | DT466472 | Gossypium |
| | | | | | hirsutum |
| Sorbi_FBXW | 85 | 86 | Partial | CF770159 | Sorghum |
| | | | (middle) | | bicolor |
| Iponi_FBXW | 87 | 88 | Partial (3′) | BJ574759.1 | Ipomea nil |
| Soltu_FBXW | 89 | 90 | Partial (3′) | CX161187 | Solanum |
| | | | | | tuberosum |
| Zamfi_FBXW | 91 | 92 | Partial | DY032229 | Zamia |
| | | | (middle) | | fischeri |
| Peram_FBXW | 93 | 94 | Partial (3′) | CK756534 | Persea |
| | | | | | americana |
| Glyma_FBXW | 95 | 96 | Partial (3′) | CD418593.1 | Glycine max |
| Brara_FBXW | 107 | 108 | full length | AC189583 | Brassica |
| | | | | | rapa |
| Sorbi_FBXW | 109 | 110 | full length | contig of | Sorghum |
| | | | | BI075893 | bicolor |
| | | | | CW484775 | |
| | | | | CF770159 | |
| | | | | CF770238 | |
| Vitvi_FBXW | 111 | 112 | full length | AM440865 | Vitis vinifera |

### Example 9

### Determination of Global Similarity and Identity Between FBXW Polypeptides, and Their Conserved Regions as Represented by SEQ ID NO: 102 and SEQ ID NO: 103 (Both Comprised in SEQ ID NO: 60)

Global percentages of similarity and identity between full length FBXW polypeptides were determined using one of the methods available in the art, the MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT software generates similarity/identity matrices for DNA or protein sequences without needing pre-alignment of the data. The program performs a series of pair-wise alignments using the Myers and Miller global alignment algorithm (with a gap opening penalty of 12, and a gap extension penalty of 2), calculates similarity and identity using for example Blosum 62 (for polypeptides), and then places the results in a distance matrix. Sequence similarity is shown in the bottom half of the dividing line and sequence identity is shown in the top half of the diagonal dividing line. The sequence of SEQ ID NO: 60 is from Arabidopsis thaliana (code Arath_FBXW).

Parameters used in the comparison were:
Scoring matrix: Blosum62
First Gap: 12
Extending gap: 2

Results of the software analysis are shown in Table H for the global similarity and identity over the full length of the FBXW polypeptides. Percentage identity is given above the diagonal and percentage similarity is given below the diagonal. Percentage identity between the FBXW paralogues and orthologues ranges between 45 and 80%, reflecting the relatively low sequence identity conservation between them.

TABLE H
MatGAT results for global similarity and identity over the
full length of the FBXW polypeptides.

| Global similarity | | | | | | |
|---|---|---|---|---|---|---|
| and identity over the | | | | | | |
| full length of the | | | | | | |
| FBXW polypeptides | 1 | 2 | 3 | 4 | 5 | 6 |
| 1. Arath_FBXW | | 57.9 | 51.1 | 63.5 | 50.4 | 49.6 |
| 2. Medtr_FBXW | 72 | | 51.2 | 61.7 | 50.2 | 49.1 |
| 3. Orysa_FBXW | 68.7 | 66.6 | | 51.6 | 76.5 | 74.4 |
| 4. Poptr_FBXW | 77.7 | 75.1 | 69.9 | | 52.8 | 52.3 |
| 5. Triae_FBXW | 67.7 | 65.7 | 87.7 | 69.6 | | 72.4 |
| 6. Zeama_FBXW | 67.2 | 65.2 | 83.7 | 67.2 | 82.6 | |

Results of the software analysis are shown in Tables I and J for the global similarity and identity over the conserved regions 1 (as represented by SEQ ID NO: 102 comprised in SEQ ID NO: 60) and 2 (SEQ ID NO: 103 comprised in SEQ ID NO: 60) of the FBXW polypeptides. Percentage identity is given above the diagonal and percentage similarity is given below the diagonal. Percentage identity of FBXW paralogues and orthologues within the conserved region 1 (as in SEQ ID NO: 102) and within the conserved region 2 (as in SEQ ID NO: 103) ranges between 65% and 100% (similarity between 85 and 100%).

TABLE I
MatGAT results for global similarity and identity over the conserved region 1 (as in
SEQ ID NO: 102) of the FBXW polypeptides.

| Global similarity and identity | | | | | | | | | | | | | |
|---|---|---|---|---|---|---|---|---|---|---|---|---|---|
| over the conserved region 1 of | | | | | | | | | | | | | |
| the FBXW polypeptides | 1 | 2 | 3 | 4 | 5 | 6 | 7 | 8 | 9 | 10 | 11 | 12 | 13 |
| 1. Cons1_Iponi_FBXW | | 94.7 | 84.2 | 92.1 | 89.5 | 84.2 | 81.6 | 97.4 | 97.4 | 81.6 | 81.6 | 81.6 | 84.2 |
| 2. Cons1_Soltu_FBXW | 97.4 | | 81.6 | 86.8 | 89.5 | 84.2 | 86.8 | 97.4 | 92.1 | 76.3 | 76.3 | 76.3 | 78.9 |
| 3. Cons1_Peram_FBXW | 94.7 | 97.4 | | 78.9 | 81.6 | 76.3 | 78.9 | 84.2 | 84.2 | 81.6 | 78.9 | 73.7 | 78.9 |
| 4. Cons1_Glyma_FBXW | 94.7 | 92.1 | 89.5 | | 84.2 | 78.9 | 73.7 | 89.5 | 89.5 | 76.3 | 76.3 | 78.9 | 78.9 |
| 5. Cons1_Poptr_FBXW | 100 | 97.4 | 94.7 | 94.7 | | 84.2 | 78.9 | 86.8 | 89.5 | 78.9 | 78.9 | 76.3 | 81.6 |
| 6. Cons1_Vitvi_FBXW | 100 | 97.4 | 97.4 | 94.7 | 100 | | 76.3 | 81.6 | 81.6 | 78.9 | 78.9 | 73.7 | 81.6 |
| 7. Cons1_Aqufo_FBXW | 94.7 | 97.4 | 97.4 | 89.5 | 94.7 | 94.7 | | 84.2 | 81.6 | 78.9 | 73.7 | 71.1 | 76.3 |
| 8. Cons1_Goshi_FBXW | 97.4 | 100 | 97.4 | 92.1 | 97.4 | 97.4 | 97.4 | | 94.7 | 78.9 | 78.9 | 78.9 | 81.6 |
| 9. Cons1_Medtr_FBXW | 97.4 | 94.7 | 92.1 | 92.1 | 97.4 | 97.4 | 94.7 | 94.7 | | 81.6 | 81.6 | 81.6 | 84.2 |
| 10. Cons1_Orysa_FBXW | 100 | 97.4 | 97.4 | 94.7 | 100 | 100 | 94.7 | 97.4 | 97.4 | | 89.5 | 71.1 | 94.7 |
| 11. Cons1_Triae_FBXW | 100 | 97.4 | 97.4 | 94.7 | 100 | 100 | 94.7 | 97.4 | 97.4 | 100 | | 65.8 | 94.7 |
| 12. Cons1_Arath_FBXW | 92.1 | 89.5 | 86.8 | 94.7 | 92.1 | 92.1 | 86.8 | 89.5 | 89.5 | 92.1 | 92.1 | | 68.4 |
| 13. Cons1_Zeama_FBXW | 100 | 97.4 | 97.4 | 94.7 | 100 | 100 | 94.7 | 97.4 | 97.4 | 100 | 100 | 92.1 | |

TABLE J
MatGAT results for global similarity and identity over the conserved region 2 (as in
SEQ ID NO: 103) of the FBXW polypeptides.

| Global similarity and identity | | | | | | | | | | | | | |
|---|---|---|---|---|---|---|---|---|---|---|---|---|---|
| over the conserved region 2 of | | | | | | | | | | | | | |
| the FBXW polypeptides | 1 | 2 | 3 | 4 | 5 | 6 | 7 | 8 | 9 | 10 | 11 | 12 | 13 |
| 1. Cons2_Ipono_FBXW | | 92.3 | 84.6 | 78.5 | 81.5 | 83.1 | 84.6 | 83.1 | 84.6 | 72.3 | 73.8 | 84.6 | 69.2 |
| 2. Cons2_Soltu_FBXW | 96.9 | | 83.1 | 78.5 | 78.5 | 81.5 | 81.5 | 83.1 | 84.6 | 70.8 | 73.8 | 78.5 | 70.8 |
| 3. Cons2_Peram_FBXW | 95.4 | 95.4 | | 81.5 | 84.6 | 87.7 | 81.5 | 89.2 | 84.6 | 73.8 | 75.4 | 81.5 | 73.8 |
| 4. Cons2_Glyma_FBXW | 92.3 | 92.3 | 93.8 | | 80 | 81.5 | 78.5 | 83.1 | 87.7 | 70.8 | 72.3 | 78.5 | 67.7 |
| 5. Cons2_Poptr_FBXW | 90.8 | 90.8 | 93.8 | 92.3 | | 86.2 | 78.5 | 86.2 | 80 | 69.2 | 72.3 | 81.5 | 69.2 |
| 6. Cons2_Vitvi_FBXW | 90.8 | 90.8 | 95.4 | 92.3 | 93.8 | | 80 | 90.8 | 81.5 | 73.8 | 75.4 | 80 | 70.8 |
| 7. Cons2_Aqufo_FBXW | 93.8 | 93.8 | 95.4 | 92.3 | 90.8 | 90.8 | | 83.1 | 87.7 | 73.8 | 75.4 | 81.5 | 70.8 |
| 8. Cons2_Goshi_FBXW | 92.3 | 92.3 | 96.9 | 93.8 | 93.8 | 95.4 | 95.4 | | 87.7 | 80 | 80 | 80 | 75.4 |
| 9. Cons2_Medtr_FBXW | 92.3 | 92.3 | 93.8 | 93.8 | 89.2 | 89.2 | 95.4 | 93.8 | | 75.4 | 76.9 | 83.1 | 69.2 |
| 10. Cons2_Orysa_FBXW | 90.8 | 87.7 | 92.3 | 87.7 | 89.2 | 89.2 | 89.2 | 93.8 | 87.7 | | 86.2 | 69.2 | 84.6 |
| 11. Cons2_Triae_FBXW | 89.2 | 86.2 | 90.8 | 89.2 | 87.7 | 87.7 | 89.2 | 93.8 | 87.7 | 96.9 | | 69.2 | 83.1 |
| 12. Cons2_Arath_FBXW | 93.8 | 92.3 | 92.3 | 90.8 | 89.2 | 89.2 | 90.8 | 90.8 | 89.2 | 89.2 | 89.2 | | 66.2 |
| 13. Cons2_Zeama_FBXW | 87.7 | 87.7 | 89.2 | 84.6 | 86.2 | 86.2 | 86.2 | 90.8 | 84.6 | 96.9 | 93.8 | 87.7 | |

### Example 10

### Cloning of the Nucleic Acid Sequence Used in the Methods of the Invention

DNA manipulation: unless otherwise stated, recombinant DNA techniques are performed according to standard protocols described in (Sambrook (2001) Molecular Cloning: a laboratory manual, 3rd Edition Cold Spring Harbor Laboratory Press, CSH, New York) or in Volumes 1 and 2 of Ausubel et al. (1994), Current Protocols in Molecular Biology, Current Protocols. Standard materials and methods for plant molecular work are described in Plant Molecular Biology Labfax (1993) by R. D. D. Croy, published by BIOS Scientific Publications Ltd (UK) and Blackwell Scientific Publications (UK).

The nucleic acid sequence used in the methods of the invention was amplified by PCR using as template a custom-made Arabidopsis thaliana mixed tissues cDNA library (in pCMV Sport 6.0; Invitrogen, Paisley, UK). PCR was performed using Hifi Taq DNA polymerase in standard conditions, using 200 ng of template in a 50 μl PCR mix. The primers used were prmO6999 (SEQ ID NO: 105; sense, AttB1 site in lower case:

5′-ggggacaagtttgtacaaaaaagcaggcttaaacaATGAATCGTTTT
TCTCGTTT 3′)

and prmO7000 (SEQ ID NO: 106; reverse, complementary, AttB2 site in lower case:

5′ ggggaccactttgtacaagaaagctgggtATCCAATCTTATCGCTTA
GG3′),

which include the AttB sites for Gateway recombination. The amplified PCR fragment was purified also using standard methods. The first step of the Gateway procedure, the BP reaction, was then performed, during which the PCR fragment recombines in vivo with the pDONR201 plasmid to produce, according to the Gateway terminology, an “entry clone”, p08433. Plasmid pDONR201 was purchased from Invitrogen, as part of the Gateway® technology.

### Example 11

### Expression Vector Construction

The entry clone p08433 was subsequently used in an LR reaction with p00640, a destination vector used for Oryza sativa transformation. This vector contains as functional elements within the T-DNA borders: a plant selectable marker; a screenable marker expression cassette; and a Gateway cassette intended for LR in vivo recombination with the nucleic acid sequence of interest already cloned in the entry clone. A rice GOS2 promoter (SEQ ID NO: 58) for constitutive expression (PRO0129) was located upstream of this Gateway cassette.

After the LR recombination step, the resulting expression vector p15973 (FIG. 7) was transformed into Agrobacterium strain LBA4044 and subsequently to Oryza sativa plants. Transformed rice plants were allowed to grow and were then examined for the parameters described below.

### Example 12

### Evaluation Procedure

### 12.1 Evaluation Setup

Approximately 35 independent T0 rice transformants were generated. The primary transformants were transferred from a tissue culture chamber to a greenhouse for growing and harvest of T1 seed. Seven events, of which the T1 progeny segregated 3:1 for presence/absence of the transgene, were retained. For each of these events, approximately 10 T1 seedlings containing the transgene (hetero- and homo-zygotes) and approximately 10 T1 seedlings lacking the transgene (nullizygotes) were selected by monitoring visual marker expression. The transgenic plants and the corresponding nullizygotes were grown side-by-side at random positions. Greenhouse conditions were of shorts days (12 hours light), 28° C. in the light and 22° C. in the dark, and a relative humidity of 70%.

Four T1 events were further evaluated in the T2 generation following the same evaluation procedure as for the T1 generation but with more individuals per event. From the stage of sowing until the stage of maturity the plants were passed several times through a digital imaging cabinet. At each time point digital images (2048×1536 pixels, 16 million colours) were taken of each plant from at least 6 different angles.

### 12.2 Statistical Analysis: F-Test

A two factor ANOVA (analysis of variants) was used as a statistical model for the overall evaluation of plant phenotypic characteristics. An F-test was carried out on all the parameters measured of all the plants of all the events transformed with the gene of the present invention. The F-test was carried out to check for an effect of the gene over all the transformation events and to verify for an overall effect of the gene, also known as a global gene effect. The threshold for significance for a true global gene effect was set at a 5% probability level for the F-test. A significant F-test value points to a gene effect, meaning that it is not only the mere presence or position of the gene that is causing the differences in phenotype.

### Example 13

### Evaluation Results

The plant aboveground area (or leafy biomass) was determined by counting the total number of pixels on the digital images from aboveground plant parts discriminated from the background. This value was averaged for the pictures taken on the same time point from the different angles and was converted to a physical surface value expressed in square mm by calibration. Experiments show that the aboveground plant area measured this way correlates with the biomass of plant parts above ground. The above ground area is the time point at which the plant had reached its maximal leafy biomass.

The mature primary panicles were harvested, counted, bagged, barcode-labeled and then dried for three days in an oven at 37° C. The panicles were then threshed and all the seeds were collected and counted. The filled husks were separated from the empty ones using an air-blowing device. The empty husks were discarded and the remaining fraction was counted again. The filled husks were weighed on an analytical balance. The number of filled seeds was determined by counting the number of filled husks that remained after the separation step. The total seed yield was measured by weighing all filled husks harvested from a plant. Total seed number per plant was measured by counting the number of husks harvested from a plant. Thousand kernel weight (TKW) is extrapolated from the number of filled seeds counted and their total weight. The harvest index (HI) in the present invention is defined as the ratio between the total seed yield and the above ground area (mm²), multiplied by a factor 10⁶. The total number of flowers per panicle as defined in the present invention is the ratio between the total number of seeds and the number of mature primary panicles. The seed fill rate as defined in the present invention is the proportion (expressed as a %) of the number of filled seeds over the total number of seeds (or florets).

As presented in Tables K to O, the aboveground biomass, the number of flowers per panicle, the seed yield, the total number of seeds, the number of filled seeds, the thousand kernel weight (TKW) and harvest index are increased in the transgenic plants with increased expression a nucleic acid encoding a FBXW polypeptide, compared to suitable control plants. Results from the T1 and the T2 generations are shown.

Table K shows the number of transgenic events with an increase in total seed yield (total seed weight), the percentage of this increase, as well as the statistical relevance of this increase according to the F-test.

TABLE K
Number of transgenic events with an increase in total seed yield,
the percentage of the increase, and P value of the F-test in
T1 and T2 generation of transgenic rice with
increased expression of a nucleic acid encoding an FBXW polypeptide.
Total seed yield

| | Number of events showing | | P value |
|---|---|---|---|
| | an increase | % Difference | of F test |
| T1 generation | 6 out of 7 | 21 | 0.001 |
| T2 generation | 3 out of 4 | 17 | 0.0002 |

Table L shows the number of transgenic events with an increase in the number of filled seeds, the percentage of this increase, as well as the statistical relevance of this increase according to the F-test.

TABLE L
Number of transgenic events with an increase in number
of filled seeds, the percentage of the increase, and P value
of the F-test in T1 and T2 generation of transgenic rice with
increased expression of a nucleic acid encoding an FBXW polypeptide.
Number of filled seeds

| | | |
|---|---|---|
| Number of events showing | | P value |
| an increase | % Difference | of F test |

| | | | |
|---|---|---|---|
| T1 generation | 6 out of 7 | 20 | 0.0017 |
| T2 generation | 3 out of 4 | 17 | 0.0002 |

Table M shows the number of transgenic events with an increase in harvest index, the percentage of this increase, as well as the statistical relevance of this increase according to the F-test.

TABLE M
Number of transgenic events with an increase in harvest index, the
percentage of the increase, and P value of the F-test in T1 and T2
generation of transgenic rice with increased
expression of a nucleic acid encoding an FBXW polypeptide.
Harvest index

| | Number of events showing | | P value |
|---|---|---|---|
| | an increase | % Difference | of F test |
| T1 generation | 6 out of 7 | 17 | 0.0009 |
| T2 generation | 3 out of 4 | 15 | <0.0001 |

Table N shows the number of transgenic events with an increase in the thousand kernel weight (TKW), the percentage of this increase, as well as the statistical relevance of this increase according to the F-test.

TABLE N
Number of transgenic events with an increase in thousand
kernel weight (TKW), the percentage of the increase, and
P value of the F-test in T1 and T2 generation of transgenic rice with
increased expression of a nucleic acid encoding an FBXW polypeptide.
TKW

| | Number of events showing | | P value |
|---|---|---|---|
| | an increase | % Difference | of F test |
| T1 generation | 5 out of 7 | 2 | 0.0187 |
| T2 generation | 2 out of 4 | 1 | 0.0022 |

Table O shows the number of transgenic events with an increase in the seed fill rate, the percentage of this increase, as well as the statistical relevance of this increase according to the F-test.

TABLE O
Number of transgenic events with an increase in fill rate,
the percentage of the increase, and P value of the F-test
in T1 and T2 generation of transgenic rice with increased
expression of a nucleic acid encoding an FBXW polypeptide.
Seed fill rate

| | Number of events showing | | P value |
|---|---|---|---|
| | an increase | % Difference | of F test |
| T1 generation | 6 out of 7 | 15 | 0.0005 |
| T2 generation | 3 out of 4 | 9 | <0.0001 |

### RANBP

### Example 14

### Identification of Sequences Related to the Nucleic Acid Sequence Used in the Methods of the Invention

Sequences (full length cDNA, ESTs or genomic) related to the nucleic acid sequence used in the methods of the present invention were identified amongst those maintained in the Entrez Nucleotides database at the National Center for Biotechnology Information (NCBI) using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). The program is used to find regions of local similarity between sequences by comparing nucleic acid or polypeptide sequences to sequence databases and by calculating the statistical significance of matches. For example, the polypeptide encoded by the nucleic acid used in the present invention was used for the TBLASTN algorithm, with default settings and the filter to ignore low complexity sequences set off. The output of the analysis was viewed by pairwise comparison, and ranked according to the probability score (E-value), where the score reflect the probability that a particular alignment occurs by chance (the lower the E-value, the more significant the hit). In addition to E-values, comparisons were also scored by percentage identity. Percentage identity refers to the number of identical nucleotides (or amino acids) between the two compared nucleic acid (or polypeptide) sequences over a particular length. In some instances, the default parameters may be adjusted to modify the stringency of the search. For example the E-value may be increased to show less stringent matches. This way, short nearly exact matches may be identified.

Table P provides a list of nucleic acid sequences related to the nucleic acid sequence used in the methods of the present invention.

TABLE P
Nucleic acid sequences related to the nucleic acid sequence
(SEQ ID NO: 113) useful in the methods of the present invention,
and the corresponding deduced polypeptides.

| | Nucleic acid | Protein |
|---|---|---|
| Plant Source | SEQ ID NO: | SEQ ID NO: |
| Zea mays | 113 | 114 |
| Zea mays | 113 | 115 |
| Arabidopsis thaliana | 116 | 117 |
| Arabidopsis thaliana | 116 | 118 |
| Arabidopsis thaliana | 119 | 120 |
| Arabidopsis thaliana | 121 | 122 |
| Lycopersicon esculentum | 123 | 124 |
| Oryza sativa | 125 | 126 |
| Zea mays | 127 | 128 |
| Oryza sativa | 129 | 130 |
| Populus sp | 131 | 132 |
| Saccharum officinarum | 133 | 134 |
| Saccharum officinarum | 135 | 136 |
| Medicago | 137 | 138 |

In some instances, related sequences have tentatively been assembled and publicly disclosed by research institutions, such as The Institute for Genomic Research (TIGR). The Eukaryotic Gene Orthologs (EGO) database may be used to identify such related sequences, either by keyword search or by using the BLAST algorithm with the nucleic acid or polypeptide sequence of interest.

### Example 15

### Cloning of a Zea Mays RANBP-Encoding Nucleic Acid Sequence Used in the Methods of the Invention

The nucleic acid sequence used in the methods of the invention was amplified by PCR using as template a corn endosperm cDNA library. PCR was performed using Hifi Taq DNA polymerase in standard conditions, using 200 ng of template in a 50 μl PCR mix. The primers used were prmO6703 (SEQ ID NO: 151; sense, start codon in bold, AttB1 site in italics:

5′-ggggacaagtttgtacaaaaaagcaggcttaaacaatggcggacaag
gagc-3′)

and prm06704 (SEQ ID NO: 152; reverse, complementary, AttB2 site in italics:

5′ ggggaccactttgtacaagaaagctgggtagtgcaacc
acaccaactact 3′)

which include the AttB sites for Gateway recombination. The amplified PCR fragment was purified also using standard methods. The first step of the Gateway procedure, the BP reaction, was then performed, during which the PCR fragment recombines in vivo with the pDONR201 plasmid to produce, according to the Gateway terminology, an “entry clone”. Plasmid pDONR201 was purchased from Invitrogen, as part of the Gateway® technology.

### Example 16

### Expression Vector Construction

The entry clone was subsequently used in an LR reaction with a destination vector used for Oryza sativa transformation. This vector contains as functional elements within the T-DNA borders: a plant selectable marker; a screenable marker expression cassette; and a Gateway cassette intended for LR in vivo recombination with the nucleic acid sequence of interest already cloned in the entry clone. A prolamin promoter (SEQ ID NO: 155) for embryo-specific expression (internal reference PR090) was located upstream of this Gateway cassette.

After the LR recombination step, the resulting expression vector p072 (FIG. 9) was transformed into Agrobacterium strain LBA4044 and subsequently to Oryza sativa plants. Transformed rice plants were allowed to grow and were then examined for the parameters described below.

### Example 17

### Cloning of the Arabidopsis thaliana RANBP-Encoding Nucleic Acid Sequence Used in the Methods of the Invention

The nucleic acid sequence used in the methods of the invention was amplified by PCR using as template an Arabidopsis thaliana cDNA library (in pCMV Sport 6.0; Invitrogen, Paisley, UK). PCR was performed using Hifi Taq DNA polymerase in standard conditions, using 200 ng of template in a 50 μl PCR mix. The primers used were 6491 (SEQ ID NO: 153; sense, start codon in bold, AttB1 site in italic:

5′- ggggacaagtttgtacaaaaaagcaggcttcacaatggcgagcatt
agcaac 3′)

and 6492 (SEQ ID NO: 154; reverse, complementary, AttB2 site in italic:

5′ ggggaccactttgtacaagaaagctgggtgcatcttaagctgaggga
ac 3′),

which include the AttB sites for Gateway recombination. The amplified PCR fragment was purified also using standard methods. The first step of the Gateway procedure, the BP reaction, was then performed, during which the PCR fragment recombines in vivo with the pDONR201 plasmid to produce, according to the Gateway terminology, an “entry clone”. Plasmid pDONR201 was purchased from Invitrogen, as part of the Gatewaye technology.

### Example 18

### Expression Vector Construction

The entry clone was subsequently used in an LR reaction with a destination vector used for Oryza sativa transformation. This vector contains as functional elements within the T-DNA borders: a plant selectable marker; a screenable marker expression cassette; and a Gateway cassette intended for LR in vivo recombination with the nucleic acid sequence of interest already cloned in the entry clone. A prolamin promoter (SEQ ID NO: 155) for embryo-specific expression (internal reference PRO90) was located upstream of this Gateway cassette.

After the LR recombination step, the resulting expression vector p074 (FIG. 10) was transformed into Agrobacterium strain LBA4044 and subsequently to Oryza sativa plants. Transformed rice plants were allowed to grow and were then examined for the parameters described below.

### Example 19

### Evaluation Procedure

### 19.1 Evaluation Setup

Approximately 30 independent T0 rice transformants were generated. The primary transformants were transferred from a tissue culture chamber to a greenhouse for growing and harvest of T1 seed. Seven events, of which the T1 progeny segregated 3:1 for presence/absence of the transgene, were retained. For each of these events, approximately 10 T1 seedlings containing the transgene (hetero- and homo-zygotes) and approximately 10 T1 seedlings lacking the transgene (nullizygotes) were selected by monitoring visual marker expression. The transgenic plants and the corresponding nullizygotes were grown side-by-side at random positions. Greenhouse conditions were of shorts days (12 hours light), 28° C. in the light and 22° C. in the dark, and a relative humidity of 70%.

Four T1 events were further evaluated in the T2 generation following the same evaluation procedure as for the T1 generation but with more individuals per event. From the stage of sowing until the stage of maturity the plants were passed several times through a digital imaging cabinet. At each time point digital images (2048×1536 pixels, 16 million colours) were taken of each plant from at least 6 different angles.

19.2 Statistical Analysis: t-Test and F-Test

A two factor ANOVA (analysis of variants) was used as a statistical model for the overall evaluation of plant phenotypic characteristics. An F-test was carried out on all the parameters measured of all the plants of all the events transformed with the gene of the present invention. The F-test was carried out to check for an effect of the gene over all the transformation events and to verify for an overall effect of the gene, also known as a global gene effect. The threshold for significance for a true global gene effect was set at a 5% probability level for the F-test. A significant F-test value points to a gene effect, meaning that it is not only the mere presence or position of the gene that is causing the differences in phenotype.

To check for an effect of the genes within an event, i.e., for a line-specific effect, a t-test was performed within each event using data sets from the transgenic plants and the corresponding null plants. “Null plants” or “null segregants” or “nullizygotes” are the plants treated in the same way as the transgenic plant, but from which the transgene has segregated. Null plants can also be described as the homozygous negative transformed plants. The threshold for significance for the t-test is set at 10% probability level. The results for some events can be above or below this threshold. This is based on the hypothesis that a gene might only have an effect in certain positions in the genome, and that the occurrence of this position-dependent effect is not uncommon. This kind of gene effect is also named herein a “line effect of the gene”. The p-value is obtained by comparing the t-value to the t-distribution or alternatively, by comparing the F-value to the F-distribution. The p-value then gives the probability that the null hypothesis (i.e., that there is no effect of the transgene) is correct.

### Example 20

### Evaluation Results

Transgenic rice plants expressing a corn RANBP under the control of a prolamin promoter gave an increase in average seed weight, number of filled seeds, harvest index, biomass, fill rate, thousand kernel weight (TKW), average seed area, average seed length and average seed width, each relative to control plants. In particular, TKW was increased in the T1 generation and this increase was confirmed in T2 generation plants. The increase was found to be statistically significant with a p-value from the F-test of >0.00001. Also noteworthy was the increase in fill rate compared to control plants, with the increase in the T1 generation being confirmed in T2 generation plants. The increase was also found to be statistically significant with a p-value from the F-test of 0.001.

### Comparative Data

Transgenic rice plants expressing a corn RANBP under the control of a constitutive GOS2 promoter gave no real difference in yield compared to control plants. There was no increase in average seed weight, number of filled seeds, harvest index, biomass, fill rate or thousand kernel weight (TKW) in transgenic plants compared to control plants.

Transgenic rice plants expressing an Arabidopsis thaliana RANBP under the control of a prolamin promoter gave an increase in biomass, average seed weight, number of filled seeds, number of flowers per panicle, harvest index, fill rate and thousand kernel weight, each relative to control plants.

### GLK

### Example 21

### Identification of Homologues of the GLK Protein of SEQ ID NO: 157 in Arabidopsis, Rice and Other Plant Species

Sequences (full length cDNA, ESTs or genomic) related to the nucleic acid sequence used in the methods of the present invention were identified amongst those maintained in the Entrez Nucleotides database at the National Center for Biotechnology Information using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). This program is typically used to find regions of local similarity between sequences by comparing nucleic acid or polypeptide sequences to sequence databases and by calculating the statistical significance of matches. The polypeptide encoded by the nucleic acid of the present invention was used with the TBLASTN algorithm, with default settings and the filter for ignoring low complexity sequences was set off. The output of the analysis was viewed by pairwise comparison, and ranked according to the probability score (E-value), where the score reflect the probability that a particular alignment occurs by chance (the lower the E-value, the more significant the hit). In addition to E-values, comparisons were also scored by percentage identity. Percentage identity refers to the number of identical nucleotides (or amino acids) between the two compared nucleic acid (or polypeptide) sequences over a particular length. In some instances, the default parameters may be adjusted to modify the stringency of the search.

Rice sequences and EST sequences from various plant species may also be obtained from other databases, such as KOME (Knowledge-based Oryza Molecular biological Encyclopedia; Kikuchi et al., Science 301, 376-379, 2003), Sputnik (Rudd, S., Nucleic Acids Res., 33: D622-D627, 2005) or the Eukaryotic Gene Orthologs database (EGO, hosted by The Institute for Genomic Research). These databases are searchable with the BLAST tool. SEQ ID NO: 168 to SEQ ID NO: 193 are nucleic acid and protein sequences of homologues of SEQ ID NO: 157 and were obtained from the above-mentioned databases using SEQ ID NO: 157 as a query sequence.

TABLE Q
Nucleic acid sequences related to the nucleic acid sequence
(SEQ ID NO: 156) useful in the methods of the present invention,
and the corresponding deduced polypeptides.

| | Nucleic acid | Protein |
|---|---|---|
| Plant Source | SEQ ID NO: | SEQ ID NO: |
| OsGLK | 156 | 157 |
| OSJNBa0086P08.18 | 168 | 169 |
| Arabidopsis thaliana GLK1 | 170 | 171 |
| Arabidopsis thaliana GLK2 | 172 | 173 |
| Physcomitrella patens Glk1 | 174 | 175 |
| Physcomitrella patens Glk2 | 176 | 177 |
| Zea mays ZmGLK1 | 178 | 179 |
| Zea mays ZmGLK2 | 180 | 181 |
| Triticum aestivum TaGLK1 | 182 | 183 |
| Allium cepa AcGLK1 | 184 | 185 |
| Hordeum vulgare HvGLK2 | 186 | 187 |
| Sorghum bicolor SbGLK1 | 188 | 189 |
| Saccharum officinarum oGLK2 | 190 | 191 |
| Oryza sativa OsGLK1 | 192 | 193 |

### Example 22

### Determination of Global Similarity and Identity Between Glk Proteins

Percentages of similarity and identity between the full length GLK protein sequences and between the GARP or GCT domains of GLK proteins were determined using MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT software generates similarity/identity matrices for DNA or protein sequences without needing pre-alignment of the data. The program performs a series of pair-wise alignments using the Myers and Miller global alignment algorithm (with a gap opening penalty of 12, and a gap extension penalty of 2), calculates similarity and identity using for example Blosum 62 (for polypeptides), and then places the results in a distance matrix. Sequence similarity is shown in the bottom half of the dividing line and sequence identity is shown in the top half of the diagonal dividing line.

The GARP and GCT domains were delineated using a multiple alignment and the obtained sequences are listed in Tables R and S. Results of the software analysis are shown in Tables T to V for the similarity and identity over the full-length protein sequences and for the GARP or GCT domains of the GLK polypeptides. The sequence of SEQ ID NO: 157 is indicated as number 6 (OsGLK1) in the matrices. Percentage identity is given above the diagonal (in bold) and percentage similarity is given below the diagonal (normal font). Percentage identity between full-length sequences of GLK paralogues and orthologues of SEQ ID NO: 157 ranges between 30% and 98.7%. These percentages are considerably higher when the sequence of the GARP domain is used instead of the full-length sequence.

TABLE R

sequences of the GARP domains as obtained upon alignment of
the proteins sequences and used in the MATGAT analysis:

| | |
|---|---|
| ZmG2 | KVKVDWTPELHRRFVQAVEQLGIDKAVPSRILEIMGTDCLTRHNIASHLQKYRSHR |
| ZmGLK1 | KAKVDWTPELHRRFVQAVEELGIDKAVPSRILEIMGIDSLTRHNIASHLQKYRSHR |
| OsGLK2 | KVKVDWTPELHRRFVQAVEQLGIDKAVPSRILELMGIECLTRHNIASHLQKYRSHR |
| PpGLK2 | KAKVDWTPELHRRFVHAVEQLGVEKAYPSRILELMGVQCLTRHNIASHLQKYRSHR |
| PpGlk1 | KAKVDWTPELHRRFVHAVEQLGVEKAFPSRILELMGVQCLTRHNIASHLQKYRSHR |
| OsGLK1 | KAKVDWTPELHRRFVQAVEQLGIDKAVPSRILEIMGIDSLTRHNIASHLQKYRSHR |
| AtGLK2 | KPKVDWTPELHRKFVQAVEQLGVDKAVPSRILEIMNVKSLTRHNVASHLQKYRSHR |
| AtGLK1 | KVKVDWTPELHRRFVEAVEQLGVDKAVPSRILELMGVHCLTRHNVASHLQKYRSHR |
| AcGLK1 | KAKVDWTPELHRRFVQAVEQLGVDKAVPSRILELMGIDCLTRHNIASHLQKYRSHR |

TABLE S

sequences of the GCT domains as obtained upon alignment of the
proteins sequences and used in the MATGAT analysis:

| | |
|---|---|
| ZmG2 | KHLMAREAEAATWAQKRHMYAPPAPRTTTTTDAARPPWVVPTTIGFPPPRFCRPLHVWGHPP |
| | PHAAAAEAAAATPMLPVWPRHLAPPRHLAPWAHPTPVDPAFWHQQYSAARKWGPQAAAVTQG |
| | TPCVPLPRFPVPHPIYSRPAMVPPPPSTTKLAQLHLELQAHPSKESIDAAIGDVLVKPWLPL |
| | PLGLKPPSLDSVMSELHKQGVPKIPPAAATTTGATG |
| ZmGLK1 | KHMLAREVEAATWTTHRRPMYAAPSGAVKRPDSNAWTVPTIGFPPPAGTPPRPVQHFGRPLH |
| | VWGHPSPTPAVESPRVPMWPRHLAPRAPPPPPWAPPPPADPASFWHHAYMR PAAHMPDQVA |
| | VTPCVAVPMAAARFPAPHVRGSLPWPPPMYRPLVPPALAGKSQQDALFQLQIQPSSESIDAA |
| | IGDVLTKPWLPLPLGLKPPSVDSVMGELQRQGVANVPQACG |
| OsGLK2 | KHLMAREAEAASWTQKRQMYTAAAAAAAVAAGGGPRKDAAAATAAVAPWVMPTIGFPPPHAA |
| | AMVPPPPHPPPFCRPPLHVWGHPTAGVEPTTAAAPPPPSPHAQPPLLPVWPRHLAPPPPPLP |
| | AAWAHGHQPAPVDPAAYWQQQYNLQRFPVPPVPGMVPHPMYRPIPPPSPPQGNKLAALQLQL |
| | DAHPSKESIDAAIGDVLVKPWLPLPLGLKPPSLDSVMSELHKQGIPKVPPAASGAAG |
| PpGLK2 | RHLAAREAEAASWTHRRTYTQAPWPRSSRRDGLPYLVPIHTPHIQPRPSMAMAMQPQLQTPH |
| | HPISTPLKVWGYPTVDHSNVHMWQQPAVATPSYWQAADGSYWQHPATGYDAFSARACYSHPM |
| | QRVPVTTTHAGLPIVAPGFPDESCYYGDDMLAGSMYLCNQSYDSEIGRAAGVAACSKPIETH |
| | LSKEVLDAAIGEALANPWTPPPLGLKPPSMEGVIAELQRQGINTVPPSTC |
| PpGlk1 | RHLAAREAEAASWTHRRAYTQMPWSRSSRRDGLPYLVPLHTPHIQPRPSMVMAMQPQLQTQH |
| | TPVSTPLKVWGYPTVDHSSVHMWQQPAVATPSYWQAPDGSYWQHPATNYDAYSARACYPHPM |
| | RVSLGTTHAGSPMMAPGFPDESYYGEDVLAATMYLCNQSYDSELGRAAGVAACSKPPETHLS |
| | KEVLDAAIGEALANPWTPPPLGLKPPSMEGVIAELQRQGINTVPPSTC |
| OsGLK1 | KHMIAREAEAASWTQRRQIYAAGGGAVAKRPESNAWTVPTIGFPPPPPPPPSPAPMQHFARP |
| | LHVWGHPTMDPSRVPVWPPRHLVPRGPAPPWVPPPPPSDPAFWHHPYMRGPAHVPTQGTPCM |
| | AMPMPAARFPAPPVPGVVPCPMYRPLTPPALTSKNQQDAQLQLQVQPSSESIDAAIGDVLSK |
| | PWLPLPLGLKPPSVDSVMGELQRQGVANVPPACG |
| AtGLK2 | KHLLAREAEAASWNLRRHATVAVPGVGGGGKKPWTAPALGYPPHVAPMHHGHFRPLHVWGHP |
| | TWPKHKPNTPASAHRTYPMPAIAAAPASWPGHPPYWHQQPLYPQGYGMASSNHSSIGVPTRQ |
| | LGPTNPPIDIHPSNESIDAAIGDVISKPWLPLPLGLKPPSVDGVMTELQRQGVSNVPPLP |
| AtGLK1 | KHLLAREAEAANWTRKRHIYGVDTGANLNGRTKNGWLAPAPTLGFPPPPPVAVAPPPVHHHH |
| | FRPLHVWGHPTVDQSIMPHVWPKHLPPPSTAMPNPPFWVSDSPYWHPMHNGTTPYLPTVATR |
| | FRAPPVAGIPHALPPHHTMYKPNLGFGGARPPVDLHPSKESVDAAIGDVLTRPWLPLPLGLN |
| | PPAVDGVMTELHRHGVSEVPPTASCA |

TABLE T
MATGAT matrix of full length sequences

| | 1 | 2 | 3 | 4 | 5 | 6 | 7 | 8 | 9 |
|---|---|---|---|---|---|---|---|---|---|
| 1. ZmG2 | | 43.9 | 53.6 | 29.4 | 29.7 | 45.0 | 38.8 | 40.0 | 44.8 |
| 2. ZmGLK1 | 54.3 | | 42.1 | 32.0 | 33.2 | 68.3 | 39.5 | 40.9 | 68.5 |
| 3. OsGLK2 | 63.2 | 54.8 | | 32.9 | 31.3 | 46.9 | 37.6 | 40.5 | 46.9 |
| 4. PpGLK2 | 43.5 | 45.1 | 46.8 | | 78.7 | 33.3 | 31.3 | 34.3 | 33.5 |
| 5. PpGIk1 | 42.9 | 44.4 | 43.5 | 85.1 | | 33.6 | 30.9 | 32.9 | 34.2 |
| 6. OsGLK1 | 57.7 | 75.6 | 56.5 | 45.5 | 46.4 | | 40.7 | 45.1 | 98.7 |
| 7. AtGLK2 | 49.5 | 49.9 | 47.0 | 42.2 | 41.9 | 50.8 | | 46.1 | 40.3 |
| 8. AtGLK1 | 51.8 | 52.2 | 52.0 | 45.8 | 43.6 | 58.7 | 58.6 | | 45.1 |
| 9. OsGLK1var | 57.9 | 75.6 | 56.7 | 44.5 | 46.8 | 99.3 | 50.5 | 58.7 | |

TABLE U
MATGAT matrix of GARP domains

| | 1 | 2 | 3 | 4 | 5 | 6 | 7 | 8 | 9 |
|---|---|---|---|---|---|---|---|---|---|
| 1. ZmG2 | | 92.9 | 94.6 | 85.7 | 85.7 | 94.6 | 85.7 | 89.3 | 92.9 |
| 2. ZmGLK1 | 94.6 | | 91.1 | 83.9 | 83.9 | 98.2 | 85.7 | 83.9 | 92.9 |
| 3. OsGLK2 | 98.2 | 96.4 | | 87.5 | 87.5 | 92.9 | 83.9 | 91.1 | 94.6 |
| 4. PpGLK2 | 91.1 | 92.9 | 94.6 | | 98.2 | 85.7 | 82.1 | 89.3 | 91.1 |
| 5. PpGlk1 | 91.1 | 92.9 | 94.6 | 100.0 | | 85.7 | 82.1 | 89.3 | 91.1 |
| 6. OsGLK1 | 94.6 | 100.0 | 96.4 | 92.9 | 92.9 | | 87.5 | 85.7 | 94.6 |
| 7. AtGLK2 | 91.1 | 94.6 | 94.6 | 91.1 | 91.1 | 94.6 | | 87.5 | 85.7 |
| 8. AtGLK1 | 96.4 | 94.6 | 98.2 | 92.9 | 92.9 | 94.6 | 92.9 | | 91.1 |
| 9. AcGLK1 | 96.4 | 98.2 | 98.2 | 94.6 | 94.6 | 98.2 | 92.9 | 96.4 | |

TABLE V
MATGAT matrix of GCT domains

| | 1 | 2 | 3 | 4 | 5 | 6 | 7 | 8 |
|---|---|---|---|---|---|---|---|---|
| 1. ZmG2 | | 48.2 | 56.3 | 26.7 | 28.5 | 47.6 | 38.0 | 37.0 |
| 2. ZmGLK1 | 58.6 | | 44.0 | 32.4 | 33.9 | 72.7 | 41.0 | 44.3 |
| 3. OsGLK2 | 65.0 | 57.6 | | 33.6 | 32.0 | 51.6 | 40.6 | 42.5 |
| 4. PpGLK2 | 41.9 | 47.0 | 44.0 | | 88.6 | 32.4 | 33.8 | 35.0 |
| 5. PpGlk1 | 42.3 | 45.7 | 42.4 | 93.2 | | 35.2 | 32.6 | 34.5 |
| 6. OsGLK1 | 61.3 | 81.1 | 60.5 | 44.1 | 49.6 | | 44.2 | 48.9 |
| 7. AtGLK2 | 48.6 | 48.5 | 48.6 | 43.2 | 43.2 | 52.3 | | 47.3 |
| 8. AtGLK1 | 50.9 | 55.5 | 54.7 | 49.2 | 46.6 | 61.8 | 57.1 | |

### Example 23

### Cloning of the Nucleic Acid Sequence Used in the Methods of the Invention

DNA manipulation: unless otherwise stated, recombinant DNA techniques are performed according to standard protocols described in (Sambrook (2001) Molecular Cloning: a laboratory manual, 3rd Edition Cold Spring Harbor Laboratory Press, CSH, New York) or in Volumes 1 and 2 of Ausubel et al. (1994), Current Protocols in Molecular Biology, Current Protocols. Standard materials and methods for plant molecular work are described in Plant Molecular Biology Labfax (1993) by R. D. D. Croy, published by BIOS Scientific Publications Ltd (UK) and Blackwell Scientific Publications (UK).

The nucleic acid sequence used in the methods of the invention was amplified by PCR using as template a custom-made Oryza sativa seedlings cDNA library (in pCMV Sport 6.0; Invitrogen, Paisley, UK). PCR was performed using Hifi Taq DNA polymerase in standard conditions, using 200 ng of template in a 50 μl PCR mix. The primers used were prm2251 (SEQ ID NO: 158; sense, start codon in bold:

5′ ggggacaagtttgtacaaaaaagcaggcttcacaatgcttgccgtgt
cgc 3′)

and prm2252 (SEQ ID NO: 159; reverse, complementary:

5′ ggggaccactttgtacaagaaagctgggtaatatcatccacacgctg
ga 3′),

which include the AttB sites for Gateway recombination. The amplified PCR fragment was purified also using standard methods. The first step of the Gateway procedure, the BP reaction, was then performed, during which the PCR fragment recombines in vivo with the pDONR201 plasmid to produce, according to the Gateway terminology, an “entry clone”, p034. Plasmid pDONR201 was purchased from Invitrogen, as part of the Gateway® technology.

### Example 24

### Expression Vector Construction

The entry clone p031 was subsequently used in an LR reaction with p00640, a destination vector used for Oryza sativa transformation. This vector contains as functional elements within the T-DNA borders: a plant selectable marker; a screenable marker expression cassette; and a Gateway cassette intended for LR in vivo recombination with the nucleic acid sequence of interest already cloned in the entry clone. A rice non-viral constitutive promoter, the GOS2 promoter (SEQ ID NO: 58) (PRO0129) was located upstream of this Gateway cassette.

After the LR recombination step, the resulting expression vector p045 (FIG. 14) was transformed into Agrobacterium strain LBA4044 and subsequently to Oryza sativa plants. Transformed rice plants were allowed to grow and were then examined for the parameters described below.

### Example 25

### Evaluation Procedure

### 25.1 Evaluation Setup

Approximately 30 independent T0 rice transformants were generated. The primary transformants were transferred from a tissue culture chamber to a greenhouse for growing and harvest of T1 seed. Four events, of which the T1 progeny segregated 3:1 for presence/absence of the transgene, were retained. For each of these events, approximately 10 T1 seedlings containing the transgene (hetero- and homo-zygotes) and approximately 10 T1 seedlings lacking the transgene (nullizygotes) were selected by monitoring visual marker expression. The transgenic plants and the corresponding nullizygotes were grown side-by-side at random positions. Greenhouse conditions were of shorts days (12 hours light), 28° C. in the light and 22° C. in the dark, and a relative humidity of 70%.

The four T1 events were further evaluated in the T2 generation following the same evaluation procedure as for the T1 generation but with more individuals per event. From the stage of sowing until the stage of maturity the plants were passed several times through a digital imaging cabinet. At each time point digital images (2048×1536 pixels, 16 million colours) were taken of each plant from at least 6 different angles.

25.2 Statistical Analysis: t-Test and F-Test

A two factor ANOVA (analysis of variants) was used as a statistical model for the overall evaluation of plant phenotypic characteristics. An F-test was carried out on all the parameters measured of all the plants of all the events transformed with the gene of the present invention. The F-test was carried out to check for an effect of the gene over all the transformation events and to verify for an overall effect of the gene, also known as a global gene effect. The threshold for significance for a true global gene effect was set at a 5% probability level for the F-test. A significant F-test value points to a gene effect, meaning that it is not only the mere presence or position of the gene that is causing the differences in phenotype.

### Example 26

### Evaluation Results

The plant aboveground area (or leafy biomass) was determined by counting the total number of pixels on the digital images from aboveground plant parts discriminated from the background. This value was averaged for the pictures taken on the same time point from the different angles and was converted to a physical surface value expressed in square mm by calibration. Experiments show that the aboveground plant area measured this way correlates with the biomass of plant parts above ground. The above ground area is the time point at which the plant had reached its maximal leafy biomass.

The mature primary panicles were harvested, counted, bagged, barcode-labeled and then dried for three days in an oven at 37° C. The panicles were then threshed and all the seeds were collected and counted. The filled husks were separated from the empty ones using an air-blowing device. The empty husks were discarded and the remaining fraction was counted again. The filled husks were weighed on an analytical balance. The number of filled seeds was determined by counting the number of filled husks that remained after the separation step. The total seed yield was measured by weighing all filled husks harvested from a plant.

As presented in Tables W to Y, the aboveground biomass, the seed yield and the number of filled seeds are increased in the transgenic plants with increased expression of a nucleic acid encoding a GLK protein, compared to suitable control plants. Results from the T1 and the T2 generations are shown.

Table W shows the increase in aboveground biomass in percent, as well as the statistical relevance of this increase according to the F-test, in the T1 and T2 generation of transgenic rice with increased expression of a nucleic acid encoding a GLK protein.

TABLE W
Aboveground biomass

| | % Difference | P value of F test |
|---|---|---|
| T1 generation | 12 | 0.0018 |
| T2 generation | 27 | 0.0000 |

Table X shows the increase in total seed yield (total seed weight) in percent, as well as the statistical relevance of this increase according to the F-test, in the T1 and T2 generation of transgenic rice with increased expression of a nucleic acid encoding a GLK protein.

TABLE X
Seed yield

| | % Difference | P value of F test |
|---|---|---|
| T1 generation | 20 | 0.0048 |
| T2 generation | 20 | 0.0160 |

Table Y shows the increase in the number of filled seeds in percent, as well as the statistical relevance of this increase according to the F-test, in the T1 and T2 generation of transgenic rice with increased expression of a nucleic acid encoding a GLK protein.

TABLE Y
Number of filled seeds

| | % Difference | P value of F test |
|---|---|---|
| T1 generation | 22 | 0.0021 |
| T2 generation | 22 | 0.0065 |

### REV ΔHDZip/START

### Example 27

### Identification of Sequences Related to the Nucleic Acid Sequence Used in the Methods of the Invention

Sequences (full length cDNA, ESTs or genomic) related to the nucleic acid sequence used in the methods of the present invention were identified amongst those maintained in the Entrez Nucleotides database at the National Center for Biotechnology Information using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). The program is used to find regions of local similarity between sequences by comparing nucleic acid or polypeptide sequences to sequence databases and by calculating the statistical significance of matches. The polypeptide encoded by the nucleic acid sequence of the present invention was used for the TBLASTN algorithm, with default settings and the filter to ignore low complexity sequences set off. The output of the analysis was viewed by pairwise comparison, and ranked according to the probability score (E-value), where the score reflects the probability that a particular alignment occurs by chance (the lower the E-value, the more significant the hit). In addition to E-values, comparisons were also scored by percentage identity. Percentage identity refers to the number of identical nucleotides (or amino acids) between the two compared nucleic acid (or polypeptide) sequences over a particular length. In some instances, the default parameters may be adjusted to modify the stringency of the search

Table Z provides a list of nucleic acid sequences related to the nucleic acid sequence used in the methods of the present invention.

TABLE Z
Nucleic acid sequences related to the nucleic acid sequence
(SEQ ID NO: 194) used in the methods of the present invention,
and the corresponding deduced polypeptides.

| | | Nucleic | | |
|---|---|---|---|---|
| | | acid | | database |
| | source | SEQ | Polypeptide | accession |
| Name | organism | ID NO: | SEQ ID NO: | number |
| Orysa_REV | Oryza sativa | 194 | 195 | Part of |
| partial | | | | AK102830 |
| CTR | | | | (Os10g33960) |
| Orysa_REV | Oryza sativa | 196 | 197 | Part of |
| CTR | | | | AK102830 |
| | | | | (Os10g33960) |
| Orysa_Rev | Oryza sativa | 198 | 199 | AK102830 |
| | | | | (Os10g33960) |
| Orysa_HOX10 | Oryza sativa | 200 | 201 | AY425991.1 |
| Arath_REV | Arabidopsis | 202 | 203 | AF188994 |
| | thaliana | | | |
| Zeama_HDIII | Zea mays | 204 | 205 | AY501430.1 |
| RLD1 | | | | |
| (rolled leaf1) | | | | |
| Poptr_HDIII | Populus | 206 | 207 | AY919617 |
| | trichocarpa | | | |
| Medtr_HDIII | Medicago | 208 | 209 | Spliced from |
| | trunculata | | | AC138171.17 |
| Sacof_HDIII | Saccharum | 210 | 211 | contig of |
| Partial | officinarum | | | CA125167.1 |
| | | | | CA217027.1 |
| | | | | CA241276.1 |
| | | | | CA124509.1 |
| Triae_HDIII | Triticum | 212 | 213 | contig of |
| Partial | aestivum | | | CD905903 |
| | | | | BM135681.1 |
| | | | | BQ578798.1 |
| | | | | CJ565259.1 |
| Horvu_HDIII | Hordeum | 214 | 215 | compiled from |
| Partial | vulgare | | | BU996988.1 |
| | | | | BJ452342.1 |
| | | | | BJ459891.1 |
| Phypr_HDIII | Phyllostachys | 216 | 217 | DQ013803 |
| Partial | praecox | | | |
| Orysa_REV | Oryza sativa | 223 | | Variant of SEQ |
| | | | | ID NO: 196 |
| Brara | Brassica rapa | 224 | 225 | AC189324.1 |
| Revoluta | | | | |
| Ginbi | Ginkgo biloba | 226 | 227 | DQ385525 |
| Revoluta | | | | |
| Gosba | Gossypium | 228 | 229 | AY966446.1 |
| Revoluta | barbadense | | | |
| Lyces | Lycopersicon | 230 | 231 | BT013577 |
| Revoluta | esculentum | | | |

### Example 28

### Determination of Global Similarity and Identity Between the CTR of REV Polypeptides

Global percentages of similarity and identity between the CTR of REV polypeptides were determined using one of the methods available in the art, the MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT software generates similarity/identity matrices for DNA or protein sequences without needing pre-alignment of the data. The program performs a series of pair-wise alignments using the Myers and Miller global alignment algorithm (with a gap opening penalty of 12, and a gap extension penalty of 2), calculates similarity and identity using for example Blosum 62 (for polypeptides), and then places the results in a distance matrix. Sequence similarity is shown in the bottom half of the dividing line and sequence identity is shown in the top half of the diagonal dividing line.

Parameters used in the comparison were:
Scoring matrix: Blosum62
First Gap: 12
Extending gap: 2

Results of the software analysis are shown in Table AA for the global similarity and identity between the CTR of REV polypeptides. Percentage identity is given above the diagonal and percentage similarity is given below the diagonal. Percentage identity between the CTR of REV polypeptide paralogues and orthologues ranges between 30 and 70%, reflecting the lower sequence identity conservation between them outside of the HDZip and START domains.

TABLE AA
MatGAT results for global similarity and identity between the CTR of REV
polypeptide orthologues and paralogues.

| Global similarity and identity over | | | | | | | | | | |
|---|---|---|---|---|---|---|---|---|---|---|
| the CTR of REV polypeptide | | | | | | | | | | |
| orthologues and paralogues | 1 | 2 | 3 | 4 | 5 | 6 | 7 | 8 | 9 | 10 |
| 1. CTR_Horvu_HDIII | | 61.1 | 57.2 | 81.1 | 88.2 | 89.7 | 60 | 79.6 | 96.5 | 79.8 |
| 2. CTR_Arath_REV | 76.9 | | 70 | 61.8 | 63 | 63.2 | 73.3 | 62.5 | 62.9 | 63.4 |
| 3. CTR_Medtr_REV | 73.2 | 86.5 | | 59.2 | 60.3 | 60.8 | 70.2 | 59.4 | 58.6 | 60.3 |
| 4. CTR_Orysa_HOX10 | 89 | 80.7 | 76.6 | | 83.9 | 84.8 | 59.8 | 88.2 | 83 | 89.7 |
| 5. CTR_Orysa_REV | 93.5 | 79.6 | 76.2 | 92.5 | | 92.7 | 61.9 | 83.2 | 90.7 | 83.9 |
| 6. CTR_Phypr_HDIII | 93.3 | 79.8 | 76.2 | 92.7 | 96.3 | | 61.9 | 84.5 | 91.8 | 84.2 |
| 7. CTR_Poptr_HDIII | 75.1 | 86.4 | 82.8 | 76.1 | 76.1 | 76.5 | | 60.4 | 61.6 | 61.3 |
| 8. CTR_Sacof_HDIII | 88.6 | 80.3 | 75.9 | 94.2 | 91.8 | 91.8 | 77.4 | | 81.9 | 94.8 |
| 9. CTR_Triae_HDIII | 97.4 | 79 | 74.9 | 90.8 | 95.7 | 95.1 | 76.3 | 90.5 | | 81.8 |
| 10. CTR_Zeama_HDIII_LRD1 | 88.8 | 81.3 | 77.8 | 95.7 | 92.5 | 92.3 | 77.8 | 97 | 90.6 | |

All REV polypeptides comprise a CTR having, in increasing order of preference, at least 50%, 55%, 60%, 65%, 70%, 75%, 80%, 85%, 90%, 95% or 98% sequence identity to the CTR of a REV polypeptide as represented by SEQ ID NO: 197.

### Example 29

### Gene Cloning

DNA manipulation: unless otherwise stated, recombinant DNA techniques are performed according to standard protocols described in (Sambrook (2001) Molecular Cloning: a laboratory manual, 3rd Edition, Cold Spring Harbor Laboratory Press, CSH, New York) or in Volumes 1 and 2 of Ausubel et al. (1994), Current Protocols in Molecular Biology, Current Protocols. Standard materials and methods for plant molecular work are described in Plant Molecular Biology Labfax (1993) by R. D. D. Croy, published by BIOS Scientific Publications Ltd (UK) and Blackwell Scientific Publications (UK).

The Oryza sativa Orysa_REV full-length gene SEQ ID NO: 198 was amplified by PCR using as template a custom-made Oryza sativa cDNA library (Invitrogen, Paisley, UK). After reverse transcription of RNA extracted from seedlings, the cDNAs were cloned into pCMV Sport 6.0. After plasmid extraction, 200 ng of template was used in a 50 μl PCR mix. Primers prmO1983 (SEQ ID NO: 221; sense, start codon in bold, AttB1 site in italic:

5′-ggggacaagtttgtacaaaaaagcaggcttaaacaatggcggcggcg
gtgg-3′)

and prm01984 (SEQ ID NO: 222; reverse, complementary, AttB2 site in italic:

5′-ggggaccactttgtacaagaaagctgggtggattttgggtcacacga
aggacca -3′),

which include the AttB sites for Gateway recombination, were used for PCR amplification. PCR was performed using Hifi Taq DNA polymerase in standard conditions. The amplified PCR fragment was purified also using standard methods. The first step of the Gateway procedure, the BP reaction, was then performed, during which the PCR fragment recombines with the pDONR201 plasmid to produce, according to the Gateway terminology, an “entry clone”, p04562. Plasmid pDONR201 was purchased from Invitrogen, as part of the Gateway® technology.

The Oryza sativa partial CTR (REV ΔHDZip/START) of the Orysa_REV gene SEQ ID NO: 194 was amplified by PCR as above, with primers prmO3263 (SEQ ID NO: 219:

5′ ggggacaagtttgtacaaaaaagcaggcttgtgctaaggcatccatg
ctac3′)

and prmO3264 (SEQ ID NO: 220:

5′ ggggaccactttgtacaagaaagctgggtgcaccttccatgctacag
cttg3′).

After cloning, the resulting entry clone number was p04436.

### Example 30

### Vector Construction

The entry clones p04562 and p04436 were subsequently used in an LR reaction with p01519, a destination vector used for Oryza sativa transformation for the hairpin construct. This vector contain as functional elements within the T-DNA borders: a plant selectable marker; a screenable marker expression cassette; and two Gateway cassettes cloned as inverted repeats and separated by a MAR (fragment of around 300 bp of a Nicotiana tabacum matrix attachment region), intended for LR in vivo recombination such that the sequence of interest from the entry clone is integrated in both sense and antisense orientations to form the hairpin secondary structure. A rice GOS2 promoter (SEQ ID NO: 58) for constitutive expression of the genetic construct (PRO0129) was located upstream of these Gateway cassettes.

After the LR recombination step, the resulting expression vectors, p0443 (with the partial CTR of Orysa_REV; SEQ ID NO: 194) and p0448 (with the full length Orysa_REV; comprised in SEQ ID NO: 198) (see FIG. 20) were separately transformed into Agrobacterium strain LBA4044, and subsequently separately to Oryza sativa plants. Transformed rice plants were allowed to grow and were then examined for the parameters described in Example 31.

### Example 31

### Evaluation Procedure

### 31.1 Evaluation Setup

Approximately 15 to 20 independent T0 rice transformants were generated. The primary transformants were transferred from a tissue culture chamber to a greenhouse for growing and harvest of T1 seed. Five events for the hairpin construct comprising the full length Orysa_REV and six events for the hairpin construct comprising the partial CTR of the Orysa_REV, of which the T1 progeny segregated 3:1 for presence/absence of the transgene, were retained. For each of these events, approximately 10 T1 seedlings containing the transgene (hetero- and homozygotes) and approximately 10 T1 seedlings lacking the transgene (nullizygotes) were selected by monitoring visual marker expression. The selected T1 plants were transferred to a greenhouse. Each plant received a unique barcode label to link unambiguously the phenotyping data to the corresponding plant. The selected T1 plants were grown on soil in 10 cm diameter pots under the following environmental settings: photoperiod=11.5 h, daylight intensity=30,000 lux or more, daytime temperature=28° C. or higher, night time temperature=22° C., relative humidity=60-70%. Transgenic plants and the corresponding nullizygotes (control plants) were grown side-by-side at random positions.

Five T1 events were further evaluated (if positive results were obtained in the first evaluation) in the T2 generation following the same evaluation procedure as for the T1 generation but with more individuals per event. From the stage of sowing until the stage of maturity the plants were passed several times through a digital imaging cabinet. At each time point digital images (2048×1536 pixels, 16 million colours) were taken of each plant from at least 6 different angles.

### 31.2 Statistical Analysis: F-Test

A two factor ANOVA (analysis of variants) was used as a statistical model for the overall evaluation of plant phenotypic characteristics. An F-test was carried out on all the parameters measured of all the plants of all the events transformed with the gene of the present invention. The F-test was carried out to check for an effect of the gene over all the transformation events and to verify for an overall effect of the gene, also known as a global gene effect. The threshold for significance for a true global gene effect was set at a 5% probability level for the F-test. A significant F-test value points to a gene effect, meaning that it is not only the mere presence or position of the gene that is causing the differences in phenotype.

### Example 32

### Evaluation Results

The plant aboveground area (or leafy biomass) was determined by counting the total number of pixels on the digital images from aboveground plant parts discriminated from the background. This value was averaged for the pictures taken on the same time point from the different angles and was converted to a physical surface value expressed in square mm by calibration. Experiments show that the aboveground plant area measured this way correlates with the biomass of plant parts above ground. The above ground area is the time point at which the plant had reached its maximal leafy biomass.

The plant parts below ground (in this case essentially the roots) were determined by growing the plants in specially designed pots with transparent bottoms to allow visualization of the roots. A digital camera recorded images through the bottom of the pot during plant growth. Root features such as total projected area (which can be correlated to total root volume), average diameter and length of roots above a certain thickness threshold (length of thick roots, or thick root length) were deduced from the picture using of appropriate software.

The mature primary panicles were harvested, counted, bagged, barcode-labeled and then dried for three days in an oven at 37° C. The panicles were then threshed and all the seeds were collected and counted. The filled husks were separated from the empty ones using an air-blowing device. The empty husks were discarded and the remaining fraction was counted again. The filled husks were weighed on an analytical balance. The number of filled seeds was determined by counting the number of filled husks that remained after the separation step. The total seed yield was measured by weighing all filled husks harvested from a plant. Total seed number per plant was measured by counting the number of husks harvested from a plant. Thousand kernel weight (TKW) is extrapolated from the number of filled seeds counted and their total weight. Individual seed parameters (including width, length, area, weight) were measured using a custom-made device consisting of two main components, a weighing and imaging device, coupled to software for image analysis. The harvest index (HI) in the present invention is defined as the ratio between the total seed yield and the above ground area (mm²), multiplied by a factor 10⁶. The total number of flowers per panicle as defined in the present invention is the ratio between the total number of seeds and the number of mature primary panicles. The seed fill rate as defined in the present invention is the proportion (expressed as a %) of the number of filled seeds over the total number of seeds (or florets).

32.1 Measurement of Yield-Related Parameters for Transformants with Reduced Expression of an Endogenous REV Gene Using a REV ΔHDZip/START Nucleic Acid Sequence as Represented by SEQ ID NO: 194

As presented in Tables BB to EE, the seed yield, the number of filled seeds, the seed fill rate and the harvest index are increased in the transgenic plants with reduced expression of an endogenous REV gene using a REV ΔHDZip/START nucleic acid sequence compared to control plants. Results from the T1 and the T2 generations are shown.

Table BB shows the number of transgenic events with an increase in total seed yield (total seed weight), the percentage of this increase, as well as the statistical relevance of this increase according to the F-test.

TABLE BB
Number of transgenic events with an increase in seed yield, the
percentage of the increase, and P value of the F-test in T1 and T2
generation of transgenic rice with reduced expression of an endogenous
REV gene using a REV ΔHDZip/START nucleic acid sequence.
Seed weight

| | Number of events | % | P value of |
|---|---|---|---|
| | showing an increase | Difference | F test |
| T1 generation | 4 out of 6 | 16 | 0.058 |
| T2 generation | 3 out of 4 | 4 | 0.0133 |

Table CC shows the number of transgenic events with an increase in the number of filled seeds, the percentage of this increase, as well as the statistical relevance of this increase according to the F-test.

TABLE CC
Number of transgenic events with an increase in number of filled
seeds, the percentage of the increase, and P value of the F-test in
T1 and T2 generation of transgenic rice with reduced expression
of an endogenous REV gene using a REV ΔHDZip/START
nucleic acid sequence.
Number of filled seeds

| | Number of events | % | P value of |
|---|---|---|---|
| | showing an increase | Difference | F test |
| T1 generation | 4 out of 6 | 14 | 0.0884 |
| T2 generation | 3 out of 4 | 4 | 0.0156 |

Table DD shows the number of transgenic events with an increase in the seed fill rate, the percentage of this increase, as well as the statistical relevance of this increase according to the F-test.

TABLE DD
Number of transgenic events with an increase in seed fill rate, the
percentage of the increase, and P value of the F-test in T1 and T2
generation of transgenic rice with reduced expression of an endogenous
REV gene using a REV ΔHDZip/START nucleic acid sequence.
Seed fill rate

| | Number of events | % | P value of |
|---|---|---|---|
| | showing an increase | Difference | F test |
| T1 generation | 5 out of 6 | 19 | 0.0002 |
| T2 generation | 4 out of 4 | 22 | <0.0001 |

Table EE shows the number of transgenic events with an increase in the harvest index, the percentage of this increase, as well as the statistical relevance of this increase according to the F-test.

TABLE EE
Number of transgenic events with an increase in harvest index,
the percentage of the increase, and P value of the F-test in T1 and T2
generation of transgenic rice with reduced expression of an endogenous
REV gene using a REV ΔHDZip/START nucleic acid sequence.
Harvest index

| | Number of events | % | P value of |
|---|---|---|---|
| | showing an increase | Difference | F test |
| T1 generation | 4 out of 6 | 14 | 0.0284 |
| T2 generation | 3 out of 4 | 9 | 0.0187 |

Two additional parameters were measured for only one evaluation:
1) the average individual seed length
2) the average root thickness

As shown on Table FF, both the average individual seed length and the average root thickness were significantly increased in transgenic rice with reduced expression of an endogenous REV gene using a REV ΔHDZip/START nucleic acid sequence compared to control plants.

TABLE FF
Number of transgenic events with an increase in harvest index and
P value of the F-test in a single generation of transgenic rice with
reduced expression of an endogenous REV gene using a REV
ΔHDZip/START nucleic acid sequence.

| | Number of events showing | |
|---|---|---|
| | an increase | P value of F test |
| Average individual seed | 5 out of 6 | 0.009 |
| length (T2 seeds) | | |
| Average root thickness | 4 out of 4 | <0.0001 |
| (T2 plants) | | |

32.2 Measurement of Yield-Related Parameters for Transformants with Reduced Expression of an Endogenous REV Polypeptide Using a Nucleic Acid Encoding the Full Length Orysa_REV Polypeptide, as Represented by SEQ ID NO: 198:

The same evaluation procedure as described hereinabove was performed for transgenic rice having reduced expression of an endogenous REV polypeptide using a nucleic acid sequence encoding the full length Orysa_REV polypeptide. All of the parameters measured were strongly and significantly negative, as shown in Table GG.

TABLE GG
Number of transgenic events with a DECREASE in aboveground
biomass, total seed yield, number of filled seeds, number of flowers
per panicle, harvest index, number of primary panicles and plant
height, the percentage of the increase, and P value of the F-test in
T1 generation of transgenic rice with reduced expression of an
endogenous REV gene using a nucleic acid sequence encoding
the full length Orysa_REV polypeptide.

| | Number of events | | P value |
|---|---|---|---|
| Parameter | showing a decrease | % Difference | of F test |
| Aboveground biomass | 5 out of 5 | −40 | <0.0001 |
| Total seed yield | 5 out of 5 | −65 | <0.0001 |
| Number of filled seeds | 5 out of 5 | −64 | <0.0001 |
| Number of flowers per | 5 out of 5 | −39 | <0.0001 |
| panicle | | | |
| Harvest index | 5 out of 5 | −53 | <0.0001 |
| Number of primary | 5 out of 5 | −39 | <0.0001 |
| panicles | | | |
| Plant height | 5 out of 5 | −13 | 0.0006 |

By including conserved regions in the hairpin construct (such as the HDZip and START domains) for reducing endogenous REV gene expression, the expression of other class III HDZip genes may also be reduced.

### CLE

### Example 33

### Identification of Sequences Related to the Nucleic Acid Sequence Used in the Methods of the Invention

Sequences (full length cDNA, ESTs or genomic) related to the nucleic acid sequence used in the methods of the present invention were identified amongst those maintained in the Entrez Nucleotides database at the National Center for Biotechnology Information using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). The program is used to find regions of local similarity between sequences by comparing nucleic acid or polypeptide sequences to sequence databases and by calculating the statistical significance of matches. The polypeptide encoded by the nucleic acid sequence of the present invention was used for the TBLASTN algorithm, with default settings and the filter to ignore low complexity sequences set off. The output of the analysis was viewed by pairwise comparison, and ranked according to the probability score (E-value), where the score reflects the probability that a particular alignment occurs by chance (the lower the E-value, the more significant the hit). In addition to E-values, comparisons were also scored by percentage identity. Percentage identity refers to the number of identical nucleotides (or amino acids) between the two compared nucleic acid (or polypeptide) sequences over a particular length. In some instances, the default parameters may be adjusted to modify the stringency of the search

Table HH provides a list of nucleic acid sequences related to the nucleic acid sequence used in the methods of the present invention.

TABLE HH
Nucleic acid sequences related to the nucleic acid sequence
(SEQ ID NO: 232) used in the methods of the present invention,
and the corresponding deduced polypeptides.

| | Nucleic acid | Protein |
|---|---|---|
| Plant Source | SEQ ID NO: | SEQ ID NO: |
| Saccharum officinarum CLE- | 232 | 233 |
| like | | |
| Populus trichocarpa X | 239 | 240 |
| Populus deltoides CLE-like | | |
| Oryza sativa CLE-like | 241 | 242 |
| Saccharum officinarum CLE- | 243 | 244 |
| like | | |
| Arabidopsis thaliana CLE2- | 245 | 246 |
| like | | |
| Brassica napus CLE-like | 247 | 248 |
| Arabidopsis thaliana | 249 | 250 |
| CLAVATA3 | | |

### Example 34

### Gene Cloning

The sugarcane CLE-like gene was amplified by PCR with primers prmO5843 (SEQ ID NO: 234; sense, start codon in bold, AttB1 site in italic:

5′-ggggacaagtttgtacaaaaaagcaggcttaaacaatgaggatgttc
ttccgg-3′)

and prm05844 (SEQ ID NO: 235; reverse, complementary, AttB2 site in italic:

5′-ggggaccactttgtacaagaaagctgggttcctctcatctgttgtgg
ag-3′),

which include the AttB sites for Gateway recombination, were used for PCR amplification. PCR was performed using Hifi Taq DNA polymerase in standard conditions. The PCR fragment was purified also using standard methods. The first step of the Gateway procedure, the BP reaction, was then performed, during which the PCR fragment recombines in vivo with the pDONR201 plasmid to produce, according to the Gateway terminology, an “entry clone”, p066. Plasmid pDONR201 was purchased from Invitrogen, as part of the Gateway® technology.

### Example 35

### Vector Construction

The entry clone p066 was subsequently used in an LR reaction with p01519, a destination vector used for Oryza sativa transformation for the antisense construct. This vectors contains as functional elements within the T-DNA borders: a plant selectable marker; a screenable marker expression cassette; and a Gateway cassette intended for LR in vivo recombination such that the sequence of interest from the entry clone is integrated in sense or anti sense orientation. A rice prolamine promoter (SEQ ID NO: 236) for seed specific expression (PRO090) was located upstream of this Gateway cassette.

After the LR recombination step, the resulting expression vector, p068 (FIG. 23) was transformed into Agrobacterium strain LBA4044 and subsequently to Oryza sativa plants. Transformed rice plants were allowed to grow and were then examined for the parameters described in Example 36.

### Example 36

### Evaluation Methods of Plants Transformed with CLE-Like in Anti Sense Orientation

Approximately 15 to 20 independent T0 rice transformants were generated. The primary transformants were transferred from a tissue culture chamber to a greenhouse for growing and harvest of T1 seed. Six events for which the T1 progeny segregated 3:1 for presence/absence of the transgene, were retained. For each of these events, approximately 10 T1 seedlings containing the transgene (hetero- and homozygotes) and approximately 10 T1 seedlings lacking the transgene (nullizygotes) were selected by monitoring visual marker expression. The selected T1 plants were transferred to a greenhouse. Each plant received a unique barcode label to link unambiguously the phenotyping data to the corresponding plant. The selected T1 plants were grown on soil in 10 cm diameter pots under the following environmental settings: photoperiod=11.5 h, daylight intensity=30,000 lux or more, daytime temperature=28° C., night time temperature=22° C., relative humidity=60-70%. Transgenic plants and the corresponding nullizygotes were grown side-by-side at random positions. From the stage of sowing until the stage of maturity the plants were passed several times through a digital imaging cabinet. At each time point digital images (2048×1536 pixels, 16 million colours) were taken of each plant from at least 6 different angles.

The plant aboveground area (or leafy biomass) was determined by counting the total number of pixels on the digital images from aboveground plant parts discriminated from the background. This value was averaged for the pictures taken on the same time point from the different angles and was converted to a physical surface value expressed in square mm by calibration. Experiments show that the aboveground plant area measured this way correlates with the biomass of plant parts above ground. The Areamax is the above ground area at the time point at which the plant had reached its maximal leafy biomass.

The mature primary panicles were harvested, bagged, barcode-labelled and then dried for three days in the oven at 37° C. The panicles were then threshed and all the seeds collected. The filled husks were separated from the empty ones using an air-blowing device. After separation, both seed lots were then counted using a commercially available counting machine. The empty husks were discarded. The filled husks were weighed on an analytical balance and the cross-sectional area of the seeds was measured using digital imaging. This procedure resulted in the set of the following seed-related parameters:

The flowers-per-panicle is a parameter estimating the average number of florets per panicle on a plant, derived from the number of total seeds divided by the number of first panicles. The tallest panicle and all the panicles that overlapped with the tallest panicle when aligned vertically, were considered as first panicles and were counted manually. The number of filled seeds was determined by counting the number of filled husks that remained after the separation step. The total seed yield (total seed weight) was measured by weighing all filled husks harvested from a plant. Total seed number per plant was measured by counting the number of husks harvested from a plant and corresponds to the number of florets per plant. These parameters were derived in an automated way from the digital images using image analysis software and were analysed statistically. Individual seed parameters (including width, length, area, weight) were measured using a custom-made device consisting of two main components, a weighing and imaging device, coupled to software for image analysis. The harvest index in the present invention is defined as the ratio between the total seed yield (g) and the above ground area (mm²), multiplied by a factor 10⁶.

A two factor ANOVA (analyses of variance) corrected for the unbalanced design was used as statistical model for the overall evaluation of plant phenotypic characteristics. An F-test was carried out on all the parameters measured of all the plants of all the events transformed with that gene. The F-test was carried out to check for an effect of the gene over all the transformation events and to verify for an overall effect of the gene, also named herein “global gene effect”. If the value of the F test shows that the data are significant, than it is concluded that there is a “gene” effect, meaning that not only presence or the position of the gene is causing the effect. The threshold for significance for a true global gene effect is set at 5% probability level for the F test.

To check for an effect of the genes within an event, i.e., for a line-specific effect, a t-test was performed within each event using data sets from the transgenic plants and the corresponding null plants. “Null plants” or “null segregants” or “nullizygotes” are the plants treated in the same way as the transgenic plant, but from which the transgene has segregated. Null plants can also be described as the homozygous negative transformed plants. The threshold for significance for the t-test is set at 10% probability level. The results for some events can be above or below this threshold. This is based on the hypothesis that a gene might only have an effect in certain positions in the genome, and that the occurrence of this position-dependent effect is not uncommon. This kind of gene effect is also named herein a “line effect of the gene”. The p-value is obtained by comparing the t-value to the t-distribution or alternatively, by comparing the F-value to the F-distribution. The p-value then gives the probability that the null hypothesis (i.e., that there is no effect of the transgene) is correct.

### Example 37

### Measurement of Yield-Related Parameters for Anti Sense Construct Transformants

Upon analysis of the seeds as described above, the inventors found that plants transformed with the anti sense CLE-like gene construct had a higher seed yield, expressed as number of filled seeds, total weight of seeds, total number of seeds and Harvest Index, compared to plants lacking the CLE-like transgene. In particular the total seed weight and total seed number was significantly increased in both the T1 and T2 generation plants.

### SYR NUE

### Example 38

### Identification of Sequences Related to SEQ ID NO: 251 and SEQ ID NO: 252

Sequences (full length cDNA, ESTs or genomic) related to SEQ ID NO: 251 and/or protein sequences related to SEQ ID NO: 252 were identified amongst those maintained in the Entrez Nucleotides database at the National Center for Biotechnology Information (NCBI) using database sequence search tools, such as the Basic Local Alignment Tool (BLAST) (Altschul et al. (1990) J. Mol. Biol. 215:403-410; and Altschul et al. (1997) Nucleic Acids Res. 25:3389-3402). The program is used to find regions of local similarity between sequences by comparing nucleic acid or polypeptide sequences to sequence databases and by calculating the statistical significance of matches. The polypeptide encoded by SEQ ID NO: 251 was used for the TBLASTN algorithm, with default settings and the filter to ignore low complexity sequences set off. The output of the analysis was viewed by pairwise comparison, and ranked according to the probability score (E-value), where the score reflects the probability that a particular alignment occurs by chance (the lower the E-value, the more significant the hit). In addition to E-values, comparisons were also scored by percentage identity. Percentage identity refers to the number of identical nucleotides (or amino acids) between the two compared nucleic acid (or polypeptide) sequences over a particular length. In some instances, the default parameters may be adjusted to modify the stringency of the search.

In addition to the publicly available nucleic acid sequences available at NCBI, proprietary sequence databases are also searched following the same procedure as described herein above.

Table II provides a list of nucleic acid and protein sequences related to the nucleic acid sequence as represented by SEQ ID NO: 251 and the protein sequence represented by SEQ ID NO: 252.

TABLE II
Nucleic acid sequences related to the nucleic acid sequence (SEQ ID NO: 251)
useful in the methods of the present invention, and the corresponding deduced polypeptides.

| | | Poly- | | Database | |
|---|---|---|---|---|---|
| | Source | peptide SEQ | Nucleic acid | accession | |
| Name | organism | ID NO: | SEQ ID NO: | number | Status |
| OsSYR | Oryza sativa | 252 | 251 | / | Full length |
| | | | | | or partial |
| rice SYR | Oryza sativa | 262 | 277 | XP_472637 | Full length |
| homologue 1 | | | | | |
| rice SYR | Oryza sativa | 263 | | AP008218 | Full length |
| homologue 2 | | | | | |
| corn SYR | Zea mays | 264 | 278 | AY110705 | partial |
| homologue | | | | | |
| wheat SYR | Triticum | 265 | | / | Full length |
| homologue | aestivum | | | | |
| barley SYR | Hordeum | 266 | 286 | CB871444 | Full length |
| homologue | vulgare | | | | |
| sugar cane SYR | Saccharum | 267 | 287 | CA165713 | partial |
| homologue 1 | officinarum | | | | |
| sugar cane SYR | Saccharum | 268 | 288 | CA242805 | Full length |
| homologue 2 | officinarum | | | | |
| sorghum SYR | Sorghum bicolor | 269 | 289 | CX611532 | Full length |
| homologue | | | | | |
| AtSYR | Arabidopsis | 270 | 290 | NM_115853 | Full length |
| homologue 1 | thaliana | | | | |
| AtSYR | Arabidopsis | 271 | 291 | NM_180078 | Full length |
| homologue 2 | thaliana | | | | |
| grape SYR | Vitis vinifera | 272 | 279 | CF404276 | Full length |
| homologue | | | | | |
| Citrus SYR | Citrus reticulata | 273 | 280 | CF830612 | partial |
| homologue | | | | | |
| tomato SYR | Lycopersicon | 274 | 282 | AI774560 | Full length |
| homologue 1 | esculentum | | | | |
| tomato SYR | Lycopersicon | 275 | 281 | BG125370 | Full length |
| homologue 2 | esculentum | | | | |

### Example 39

### Alignment of Relevant Polypeptide Sequences

AlignX from the Vector NTI (Invitrogen) is based on the popular Clustal algorithm of progressive alignment (Thompson et al. (1997) Nucleic Acids Res 25:4876-4882; Chema et al. (2003). Nucleic Acids Res 31:3497-3500). A phylogenetic tree can be constructed using a neighbour-joining clustering algorithm. Default values are for the gap open penalty of 10, for the gap extension penalty of 0.1 and the selected weight matrix is Blosum 62 (if polypeptides are aligned).

The result of the multiple sequence alignment using polypeptides relevant in identifying the ones useful in performing the methods of the invention is shown in FIG. 25. The leucine rich repeat and the conserved motifs can be easily discriminated in the various sequences.

### Example 40

### Calculation of Global Percentage Identity Between Polypeptide Sequences Useful in Performing the Methods of the Invention

Global percentages of similarity and identity between full length polypeptide sequences useful in performing the methods of the invention were determined using one of the methods available in the art, the MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT software generates similarity/identity matrices for DNA or protein sequences without needing pre-alignment of the data. The program performs a series of pair-wise alignments using the Myers and Miller global alignment algorithm (with a gap opening penalty of 12, and a gap extension penalty of 2), calculates similarity and identity using for example Blosum 62 (for polypeptides), and then places the results in a distance matrix. Sequence similarity is shown in the bottom half of the dividing line and sequence identity is shown in the top half of the diagonal dividing line.

Parameters used in the comparison were:
Scoring matrix: Blosum62
First Gap: 12
Extending gap: 2

Results of the software analysis are shown in Table JJ for the global similarity and identity over the full length of the polypeptide sequences (excluding the partial polypeptide sequences). Percentage identity is given above the diagonal and percentage similarity is given below the diagonal.

The percentage identity between the polypeptide sequences useful in performing the methods of the invention can be as low as 27% amino acid identity compared to SEQ ID NO: 252.

TABLE JJ
MatGAT results for global similarity and identity over the full length of the polypeptide sequences.

| | 1 | 2 | 3 | 4 | 5 | 6 | 7 | 8 | 9 | 10 | 11 | 12 | 13 | 14 | 15 | 16 | 17 |
|---|---|---|---|---|---|---|---|---|---|---|---|---|---|---|---|---|---|
| 1. SEQID2 | | 29.8 | 46.8 | 55.2 | 67.0 | 66.1 | 66.7 | 71.4 | 63.6 | 36.8 | 34.6 | 35.5 | 39.7 | 39.0 | 41.0 | 27.6 | 32.1 |
| 2. SEQID12 | 40.4 | | 29.8 | 23.0 | 26.8 | 28.1 | 23.6 | 25.3 | 28.7 | 30.3 | 28.1 | 30.9 | 32.0 | 28.1 | 24.7 | 16.3 | 17.4 |
| 3. SEQID13 | 57.9 | 39.3 | | 42.9 | 46.0 | 47.6 | 44.4 | 47.6 | 45.2 | 31.9 | 33.3 | 33.1 | 34.1 | 37.3 | 34.1 | 24.8 | 28.3 |
| 4. SEQID14 | 59.0 | 32.0 | 50.8 | | 57.1 | 55.4 | 77.4 | 77.4 | 83.2 | 25.4 | 26.7 | 26.6 | 30.2 | 32.2 | 33.3 | 21.6 | 23.9 |
| 5. SEQID15 | 80.9 | 41.0 | 57.9 | 69.1 | | 89.1 | 63.4 | 67.9 | 66.1 | 36.9 | 31.9 | 33.1 | 40.5 | 37.3 | 40.9 | 24.8 | 27.9 |
| 6. SEQID16 | 79.1 | 38.2 | 59.5 | 65.5 | 95.5 | | 61.6 | 66.1 | 62.5 | 36.4 | 32.6 | 36.0 | 40.5 | 38.8 | 38.2 | 24.0 | 28.8 |
| 7. SEQID17 | 69.5 | 34.8 | 57.1 | 78.1 | 72.7 | 69.1 | | 94.9 | 81.3 | 30.8 | 29.6 | 31.7 | 34.1 | 34.7 | 39.4 | 25.5 | 29.0 |
| 8. SEQID18 | 74.3 | 37.1 | 60.3 | 80.0 | 77.3 | 73.6 | 94.9 | | 85.0 | 33.1 | 31.9 | 33.8 | 36.5 | 37.3 | 42.4 | 28.2 | 32.0 |
| 9. SEQID19 | 69.2 | 39.3 | 56.3 | 86.0 | 78.2 | 74.5 | 84.1 | 88.8 | | 36.9 | 32.6 | 36.7 | 38.1 | 39.8 | 40.2 | 28.8 | 29.6 |
| 10. SEQID20 | 54.6 | 41.6 | 56.9 | 46.2 | 57.7 | 60.8 | 50.0 | 53.1 | 54.6 | | 66.2 | 46.9 | 51.9 | 44.3 | 42.7 | 26.3 | 26.9 |
| 11. SEQID21 | 51.9 | 44.4 | 56.3 | 47.4 | 54.8 | 54.8 | 50.4 | 53.3 | 52.6 | 77.8 | | 49.0 | 46.8 | 41.1 | 39.3 | 28.7 | 27.2 |
| 12. SEQID22 | 54.0 | 43.8 | 54.7 | 45.3 | 53.2 | 54.0 | 49.6 | 51.8 | 54.7 | 65.5 | 65.5 | | 61.9 | 45.1 | 40.3 | 24.0 | 22.9 |
| 13. SEQID23 | 58.7 | 45.5 | 55.6 | 50.0 | 60.3 | 59.5 | 54.8 | 57.1 | 63.5 | 66.9 | 66.7 | 77.7 | | 53.8 | 44.4 | 27.0 | 27.6 |
| 14. SEQID24 | 61.9 | 42.7 | 57.9 | 55.1 | 58.5 | 63.6 | 61.0 | 63.6 | 62.7 | 66.9 | 64.4 | 68.3 | 77.0 | | 73.7 | 27.9 | 29.4 |
| 15. SEQID25 | 62.9 | 35.4 | 50.0 | 53.3 | 60.0 | 58.2 | 66.7 | 69.7 | 61.7 | 56.2 | 54.8 | 54.7 | 60.3 | 73.7 | | 36.7 | 38.6 |
| 16. SEQID34 | 45.7 | 25.3 | 38.1 | 38.1 | 39.1 | 40.0 | 45.5 | 48.5 | 44.9 | 40.0 | 40.7 | 36.0 | 41.3 | 41.5 | 56.3 | | 42.0 |
| 17. SEQID35 | 50.5 | 30.3 | 45.2 | 40.0 | 46.4 | 44.5 | 47.5 | 50.5 | 45.8 | 34.6 | 42.2 | 36.7 | 40.5 | 42.4 | 55.2 | 57.7 | |

### Example 41

### Topology Prediction of the Polypeptide Sequences Useful in Performing the Methods of the Invention (Subcellular Localization, Transmembrane . . . )

TargetP 1.1 predicts the subcellular location of eukaryotic proteins. The location assignment is based on the predicted presence of any of the N-terminal pre-sequences: chloroplast transit peptide (cTP), mitochondrial targeting peptide (mTP) or secretory pathway signal peptide (SP). Scores on which the final prediction is based are not really probabilities, and they do not necessarily add to one. However, the location with the highest score is the most likely according to TargetP, and the relationship between the scores (the reliability class) may be an indication of how certain the prediction is. The reliability class (RC) ranges from 1 to 5, where 1 indicates the strongest prediction. TargetP is maintained at the server of the Technical University of Denmark.

For the sequences predicted to contain an N-terminal presequence a potential cleavage site can also be predicted.

A number of parameters were selected, such as organism group (non-plant or plant), cutoff sets (none, predefined set of cutoffs, or user-specified set of cutoffs), and the calculation of prediction of cleavage sites (yes or no).

The results of TargetP 1.1 analysis of the polypeptide sequence as represented by SEQ ID NO: 252 are presented Table KK. The “plant” organism group has been selected, no cutoffs defined, and the predicted length of the transit peptide requested. The subcellular localization of the polypeptide sequence as represented by SEQ ID NO: 252 may be the mitochondrion; however it should be noted that the reliability class is 5 (i.e. the lowest reliability class).

TABLE KK
TargetP 1.1 analysis of the polypeptide sequence as represented
by SEQ ID NO: 252

| | |
|---|---|
| Length (AA) | 105 |
| Chloroplastic transit peptide | 0.025 |
| Mitochondrial transit peptide | 0.552 |
| Secretory pathway signal peptide | 0.009 |
| Other subcellular targeting | 0.416 |
| Predicted Location | mitochondrion |
| Reliability class | 5 |

Two transmembrane domains are identified by the TMHMM program, hosted on the server of the Center for Biological Sequence Analysis, Technical University of Denmark. The probability that the N-terminus is located inside is 0.997. Further details on the orientation are given in Table LL:

TABLE LL
results of TMHMM 2.0

| Orientation | begin - end residue |
|---|---|
| inside | 1 42 |
| TMhelix | 43 65 |
| outside | 66 74 |
| TMhelix | 75 92 |
| inside | 93 105 |

Many other algorithms can be used to perform such analyses, including:
ChloroP 1.1 hosted on the server of the Technical University of Denmark;
Protein Prowler Subcellular Localisation Predictor version 1.2 hosted on the server of the Institute for Molecular Bioscience, University of Queensland, Brisbane, Australia;
PENCE Proteome Analyst PA-GOSUB 2.5 hosted on the server of the University of Alberta, Edmonton, Alberta, Canada;

### Example 42

### Gene Cloning

DNA manipulation: unless otherwise stated, recombinant DNA techniques are performed according to standard protocols described in (Sambrook (2001) Molecular Cloning: a laboratory manual, 3rd Edition, Cold Spring Harbor Laboratory Press, CSH, New York) or in Volumes 1 and 2 of Ausubel et al. (1994), Current Protocols in Molecular Biology, Current Protocols. Standard materials and methods for plant molecular work are described in Plant Molecular Biology Labfax (1993) by R. D. D. Croy, published by BIOS Scientific Publications Ltd (UK) and Blackwell Scientific Publications (UK).

The Oryza sativa SYR gene was amplified by PCR using as template an Oryza sativa seedling cDNA library (Invitrogen, Paisley, UK). After reverse transcription of RNA extracted from seedlings, the cDNAs were cloned into pCMV Sport 6.0. Average insert size of the bank was 1.5 kb and the original number of clones was of the order of 1.59×10⁷ cfu. Original titer was determined to be 9.6×10⁵ cfu/ml after first amplification of 6×10¹¹ cfu/ml. After plasmid extraction, 200 ng of template was used in a 50 μl PCR mix. Primers prm08170 (SEQ ID NO: 253; sense, start codon in bold, AttB1 site in italic:

5′-ggggacaagtttgtacaaaaaagcaggcttaaacaatggaaggtgta
ggtgctagg-3′)

and prm08171 (SEQ ID NO: 254; reverse, complementary, AttB2 site in italic:

5′-ggggaccactttgtacaagaaagctgggtcaaaaacaaaaataaatt
cccc-3′),

which include the AttB sites for Gateway recombination, were used for PCR amplification. PCR was performed using Hifi Taq DNA polymerase in standard conditions. A PCR fragment of the correct size was amplified and purified also using standard methods. The first step of the Gateway procedure, the BP reaction, was then performed, during which the PCR fragment recombines in vivo with the pDONR201 plasmid to produce, according to the Gateway terminology, an “entry clone”, pSYR. Plasmid pDONR201 was purchased from Invitrogen, as part of the Gateway® technology.

### Example 43

### Vector Construction

The entry clone pSYR was subsequently used in an LR reaction with a destination vector used for Oryza sativa transformation. This vector contains as functional elements within the T-DNA borders: a plant selectable marker; a screenable marker expression cassette; and a Gateway cassette intended for LR in vivo recombination with the sequence of interest already cloned in the entry clone. A rice GOS2 promoter (SEQ ID NO: 58) for constitutive expression was located upstream of this Gateway cassette. A similar vector construct was prepared, but with the high mobility group protein promoter (HMGP, SEQ ID NO: 283 or SEQ ID NO: 293) instead of the GOS promoter

After the LR recombination step, the resulting expression vectors, pGOS2::SYR (with the GOS2 promoter) and pHMGP::SYR (with the HMGP promoter), both for constitutive SYR expression (FIG. 25) were transformed into Agrobacterium strain LBA4044 and subsequently to Oryza sativa plants.

### Example 44

### Evaluation Methods of Plants Transformed with SYR Under the Control of the Rice GOS2 Promoter or the HMGP Promoter

### 44.1 Evaluation Set-Up

Approximately 15 to 20 independent T0 rice transformants were generated. The primary transformants were transferred from a tissue culture chamber to a greenhouse for growing and harvest of T1 seed. Eight events, of which the T1 progeny segregated 3:1 for presence/absence of the transgene, were retained. For each of these events, approximately 10 T1 seedlings containing the transgene (hetero- and homo-zygotes) and approximately 10 T1 seedlings lacking the transgene (nullizygotes) were selected by monitoring visual marker expression. The selected T1 plants were transferred to a greenhouse. Each plant received a unique barcode label to link unambiguously the phenotyping data to the corresponding plant. The selected T1 plants were grown on soil in 10 cm diameter pots under the following environmental settings: photoperiod=11.5 h, daylight intensity=30,000 lux or more, daytime temperature=28° C. or higher, night time temperature=22° C., relative humidity=60-70%. Transgenic plants and the corresponding nullizygotes were grown side-by-side at random positions. From the stage of sowing until the stage of maturity the plants were passed several times through a digital imaging cabinet. At each time point digital images (2048×1536 pixels, 16 million colours) were taken of each plant from at least 6 different angles.

### Nitrogen Use Efficiency Screen

Rice plants from T2 seeds were grown in potting soil under normal conditions except for the nutrient solution. The pots were watered from transplantation to maturation with a specific 15 nutrient solution containing reduced N nitrogen (N) content, usually between 7 to 8 times less. The rest of the cultivation (plant maturation, seed harvest) was the same as for plants not grown under abiotic stress. Growth and yield parameters are recorded as detailed for growth under normal conditions.

### 44.2 Statistical Analysis: F Test

A two factor ANOVA (analysis of variants) was used as a statistical model for the overall evaluation of plant phenotypic characteristics. An F test was carried out on all the parameters measured of all the plants of all the events transformed with the gene of the present invention. The F test was carried out to check for an effect of the gene over all the transformation events and to verify for an overall effect of the gene, also known as a global gene effect. The threshold for significance for a true global gene effect was set at a 5% probability level for the F test. A significant F test value points to a gene effect, meaning that it is not only the mere presence or position of the gene that is causing the differences in phenotype.

Because two experiments with overlapping events were carried out, a combined analysis was performed. This is useful to check consistency of the effects over the two experiments, and if this is the case, to accumulate evidence from both experiments in order to increase confidence in the conclusion. The method used was a mixed-model approach that takes into account the multilevel structure of the data (i.e. experiment-event-segregants). P values were obtained by comparing likelihood ratio test to chi square distributions.

### 44.3 Parameters Measured

### Biomass-Related Parameter Measurement

The plant aboveground area (or leafy biomass) was determined by counting the total number of pixels on the digital images from aboveground plant parts discriminated from the background. This value was averaged for the pictures taken on the same time point from the different angles and was converted to a physical surface value expressed in square mm by calibration. Experiments show that the aboveground plant area measured this way correlates with the biomass of plant parts above ground. The above ground area is the area measured at the time point at which the plant had reached its maximal leafy biomass. Increase in root biomass is expressed as an increase in total root biomass (measured as maximum biomass of roots observed during the lifespan of a plant).

### Seed-Related Parameter Measurements

The mature primary panicles were harvested, counted, bagged, barcode-labelled and then dried for three days in an oven at 37° C. The panicles were then threshed and all the seeds were collected and counted. The filled husks were separated from the empty ones using an air-blowing device. The empty husks were discarded and the remaining fraction was counted again. The filled husks were weighed on an analytical balance. The number of filled seeds was determined by counting the number of filled husks that remained after the separation step. The total seed yield was measured by weighing all filled husks harvested from a plant. Thousand Kernel Weight (TKW) is extrapolated from the number of filled seeds counted and their total weight.

### Example 45

### Measurement of Yield-Related Parameters for pGOS2::SYR Transformants Grown Under Conditions of Nutrient Deficiency

Upon analysis of the seeds as described above, the inventors found that plants transformed with the pGOS2::SYR gene construct and grown under nutrient deficiency stress, had a higher seed yield, expressed as number of filled seeds (increase of more than 5%), total weight of seeds (increase of more than 5%) and TKW (increase of more than 2.5%), compared to plants lacking the SYR transgene. There was also observed an increase in shoot biomass (more than 5%) and root biomass (several lines more than 5%).

1.-156. (canceled)

157. A method for increasing abiotic stress resistance in plants relative to control plants, comprising modulating expression in a plant of a nucleic acid encoding a Seed Yield Regulator (SYR) polypeptide, which SYR polypeptide comprises a leucine rich domain, preceded by a conserved tripeptide motif 1 comprising one of SEQ ID NO: 256, 257, 258 or 259, and followed by a conserved motif 2 (SEQ ID NO: 260), wherein said increased abiotic stress resistance is increased nutrient uptake efficiency relative to control plants.

158. The method of claim 157, wherein the SYR polypeptide has, in increasing order of preference, at least 27%, 30%, 35%, 40%, 45%, 50%, 55%, 60%, 65%, 70%, 75%, 80%, 85%, 90%, 95% or more sequence identity to the polypeptide represented by SEQ ID NO: 252.

159. The method of claim 157, wherein the nucleic acid encoding a SYR polypeptide comprises the nucleic acid sequence of SEQ ID NO: 251, 277, 278, 279, 280, 281, 282, 286, 287, 288, 289, 290, or 291 or a portion thereof, or a sequence capable of hybridising with any one of said nucleic acid sequences.

160. The method of claim 158, wherein the nucleic acid encoding a SYR polypeptide comprises the nucleic acid sequence of SEQ ID NO: 251, 277, 278, 279, 280, 281, 282, 286, 287, 288, 289, 290, or 291 or a portion thereof, or a sequence capable of hybridising with any one of said nucleic acid sequences.

161. The method of claim 157, wherein said nucleic acid encodes an orthologue or paralogue of SEQ ID NO: 252, 262, 263, 264, 265, 266, 267, 268, 269, 270, 271, 272, 273, 274, or 275.

162. The method of claim 157, wherein the SYR polypeptide further comprises a conserved motif 3 (SEQ ID NO: 261).

163. The method of claim 157, wherein the nutrient uptake efficiency results in increased seed yield and/or increased biomass.

164. The method of claim 163, wherein the increased seed yield comprises at least increased total weight of seeds, Thousand Kernel Weight and/or increased number of filled seeds.

165. The method of claim 163, wherein the increased biomass is increased shoot biomass and/or increased root biomass.

166. The method of claim 157, wherein the increased nutrient uptake efficiency occurs under mild drought conditions.

167. The method of claim 157, wherein the modulated expression is effected by introducing and expressing in a plant a nucleic acid encoding a SYR polypeptide.

168. The method of claim 167, wherein the nucleic acid is operably linked to a constitutive promoter.

169. The method of claim 157, wherein the nucleic acid encoding a SYR polypeptide is of plant origin.

170. A method for making plants having increased abiotic stress resistance, comprising introducing into a plant or plant cell a construct comprising
(a) a nucleic acid encoding a SYR polypeptide,
a. wherein the SYR polypeptide comprises
i. a leucine rich domain, preceded by a conserved tripeptide motif I comprising one of SEQ ID NO: 256, 257, 258 or 259, and followed by a conserved motif 2 (SEQ ID NO: 260);
ii. a leucine rich domain, preceded by a conserved tripeptide motif 1 comprising one of SEQ ID NO: 256, 257, 258 or 259, followed by a conserved motif2 (SEQ ID NO: 260) and a conserved motif 3 (SEQ ID NO: 261); or
iii. a polypeptide having at least 27%, 30%, 35%, 40%, 45%, 50%, 55%, 60%, 65%, 70%, 75%, 80%, 85%, 90%, 95% or more sequence identity to the polypeptide represented by SEQ ID NO: 252; or
iv. the polypeptide sequence of SEQ ID NO. 252, 262, 263, 264, 265, 266, 267, 268, 269, 270, 271, 272, 273, 274, or 275; or
b. wherein the nucleic acid comprises the nucleic acid sequence of SEQ ID NO: 251, 277, 278, 279, 280, 281, 282, 286, 287, 288, 289, 290, or 291;
(b) one or more control sequences capable of driving expression of the nucleic acid sequence of (a); and optionally
(c) a transcription termination sequence,
and wherein one of the control sequences is a constitutive promoter and wherein the increased abiotic stress resistance is increased nutrient uptake efficiency relative to control plants.

171. The method of claim 168, wherein the constitutive promoter is a GOS2 promoter.

172. The method of claim 157, wherein the nucleic acid encoding a SYR polypeptide is from a monocotyledonous plant.

173. The method of claim 172, wherein the monocotyledonous plant is from the family Poaceae.

174. The method of claim 173, wherein the plant is Oryza sativa.
